SEQUENCE LISTING (165 sequences)

SEQ ID NO: 1 - DNA, 2862 bp, synthetic construct
  misc_feature 1..2862: note = Synthetic: CasM DNA sequence from Eubacterium siraeum, modified for expression in Escherichia coli
  source 1..2862: mol_type = other DNA
atggggaagaagattcatgcgcgcgatttacgcgaacaacgcaaaacggatcgcactgagaaatttgcggatcaaaacaaaaagcgcgaggccgagcgcgctgttcctaaaaaggacgccgcagtctcggttaagagtgtatcgtccgtgtcttcaaaaaaggacaacgtcactaaaagcatggcgaaggccgctggtgtaaagtctgtatttgccgtaggtaacacggtatacatgacatcgttcggccgcggcaacgacgctgtactggagcaaaagatcgtggatacatcccatgaaccacttaacatcgacgatccagcatatcaattgaacgttgttacaatgaacggttattccgtcaccggccaccgcggagagaccgtttctgcagtaacggacaaccctttacgccgtttcaatggccgcaaaaaggacgaacctgagcaatcggttccaactgacatgctttgtcttaaacctacgttagagaagaagttcttcggcaaggagtttgacgacaacatccacatccagttgatttataacattttagatattgagaagatcttagcagtttattcaaccaatgcaatttacgctttgaacaacatgagcgccgacgaaaacatcgaaaattcggattttttcatgaaacgtaccacagacgaaacctttgacgactttgaaaagaaaaaagaatctactaactcacgcgaaaaggcagacttcgacgcgtttgaaaaatttattggaaactaccgtcttgcgtacttcgcggatgctttctatgtcaataaaaaaaaccctaagggaaaggctaagaatgttctgcgtgaagataaggagctttactcggtcttaactcttatcggtaaactgcgccattggtgcgtacatagcgaggagggacgtgcagagttctggctgtataagttagacgagttaaaagacgattttaaaaatgtattggacgtcgtgtacaaccgtcccgtggaagaaatcaacaaccgctttattgagaataacaaagttaatatccaaattctggggagcgtgtacaaaaacacagacatcgctgaacttgtgcgctcgtattacgaattcttgattaccaaaaaatacaaaaatatgggcttttctattaagaaacttcgtgaatcaatgttggaaggtaaaggttacgcagacaaggaatatgactccgtccgtaataagttgtaccaaatgacagacttcattctgtatacgggatacatcaacgaagactcagatcgtgcagacgatctggtcaataccctgcgctcttctctgaaggaggatgataagacgactgtatactgtaaagaggccgactatttgtggaagaagtatcgcgaatcgatccgtgaggttgcggatgcactggatggtgataacatcaagaagttgagtaagtcgaacatcgagatccaagaggataaacttcgtaagtgcttcattagttatgcagactccgtttcagagttcacaaaactgatctacctgctgacccgcttcctgagcggaaaggaaattaatgacctggtaactactcttatcaataaatttgataacatccgctcttttcttgagattatggacgagctgggattagatcgtacgtttaccgccgaatattcgttctttgaaggctcaacgaaatacttggcggagcttgtagagttaaattcttttgtaaaatcttgctcttttgatattaacgccaagcgcacaatgtatcgcgacgccttagacattttggggattgaatcggacaagactgaagaggatattgaaaagatgattgataatatccttcagattgatgcgaatggcgacaagaaacttaagaaaaataatggcctgcgtaacttcattgcaagtaacgttattgacagtaaccgtttcaaatacttagtacgctacgggaaccctaaaaaaatccgcgaaacagctaagtgcaaaccggctgttcgcttcgtgttgaacgagatccccgacgcacagatcgagcgctattacgaggcatgctgtccaaagaacacagccctttgctcagcgaacaagcgtcgcgagaagttagctgacatgattgccgagattaagttcgagaacttctctgacgctggaaattatcaaaaagctaacgttacctcgcgcacatcagaggcggaaatcaaacgtaaaaaccaggcgattattcgcttgtatttgacggtcatgtacattatgctgaagaacttagtcaacgtgaacgctcgttacgtgatcgcatttcactgtgtggagcgtgatactaagttgtatgccgaatctggattggaggttgggaacattgaaaagaataaaactaatcttaccatggccgtaatgggagttaagcttgagaatggtatcatcaagactgagtttgataaatcttttgcggaaaacgcagcaaatcgttaccttcgtaacgcacgctggtataaacttatcttagacaatttaaaaaagtcagaacgcgcggtagtaaacgaatttcgtaacacagtatgtcatttaaacgccatccgcaacattaacattaacatcaaggagattaaggaggtagaaaattattttgccttgtaccactatttgatccaaaaacatttggagaaccgtttcgccgacaaaaaagttgaacgcgatacgggtgactttatttccaaattggaagagcataagacgtactgtaaggactttgtaaaagcatactgtacgccgtttggatataatttagtacgttataagaacttgactattgacggacttttcgataaaaactaccctgggaaggatgattctgatgaacagaaa

SEQ ID NO: 2 - DNA, 2757 bp, synthetic construct
  misc_feature 1..2757: note = Synthetic: CasM DNA sequence from Ruminococcus sp., isolate 2789STDY5834971, modified for expression in Escherichia coli
  source 1..2757: mol_type = other DNA
atggcaaagaaaaataaaatgaagccgcgcgagttacgcgaggcccagaagaaagctcgtcaattaaaagcggccgagatcaacaataacgcagccccagcaattgcagcaatgccagcggccgaagtgattgcgccggctgcagagaagaagaagagctcagtcaaggcagcagggatgaagagcatccttgttagcgagaacaagatgtacattacatcttttgggaaaggaaactcagcggtattggaatacgaggttgataacaacgattacaatcagacgcagttatcatccaaggacaacagcaacatccaactgggtggcgtcaatgaggtcaacattactttttcaagcaagcacggctttgaaagtggcgtggaaattaacacttctaatccgacacaccgttcaggagaaagttcccctgttcgtggcgatatgttagggcttaagtcagaactggaaaagcgcttcttcggtaagaccttcgatgataacattcacattcaacttatctacaacatccttgatattgaaaagatccttgcagtgtacgttacgaacatcgtctacgctctgaataatatgttaggtgtcaaggggtctgaatcccatgatgacttcattggttacttgtcgacaaataatatctacgatgtcttcattgatccagataatagttccttgagcgacgacaagaaagcaaacgtacgtaaaagtcttagtaaatttaatgcgttgttaaaaactaaacgtctgggctatttcggattagaggaaccaaagaccaaagacaaccgtgtaagccaggcgtataagaagcgtgtgtatcacatgcttgccattgtcgggcaaattcgtcaatgcgtatttcatgacaaaagcggtgccaaacgttttgatctttattctttcattaacaatattgatccagagtaccgtgacacgcttgattatttggtagaagagcgcctgaagtcaattaacaaagactttattgaagacaacaaagtaaacatcagccttttaattgatatgatgaagggttacgaggcggacgatatcattcgcctgtactacgacttcattgtattaaaatctcagaaaaacctggggttctctattaagaagttacgtgagaagatgctggacgagtatggtttccgtttcaaagataaacaatacgattctgttcgttccaagatgtataaattgatggattttttgcttttttgtaactattaccgcaatgatattgctgcgggggaatctctggtacgtaaactgcgtttttcgatgacagacgatgaaaaggagggcatttatgcggacgaagccgctaaattgtgggggaaatttcgtaatgactttgagaatatcgcggaccacatgaatggcgatgttattaaggagttgggaaaagctgacatggatttcgacgaaaagatcttggattctgagaagaaaaacgcttccgacctgctgtatttttcaaaaatgatttatatgctgacatatttcttagatgggaaagagattaacgacttgctgacgactctgatttcaaaatttgacaatatcaaagagtttttgaaaattatgaagtcttctgcagtcgatgtagagtgtgaacttacagctgggtacaagctgttcaatgacagtcaacgtatcaccaacgaattatttatcgttaaaaatattgcctccatgcgtaagccagccgcaagtgccaagctgacaatgttccgcgatgcactgacgattctgggaattgacgataagattacggatgaccgtatttcaggaatcttgaagcttaaagagaagggcaagggcattcatggacttcgtaacttcatcaccaacaacgtgatcgagagtagccgttttgtttaccttatcaaatatgcgaatgcacaaaagatccgcgaagtggcgaaaaacgagaaggtcgtaatgttcgtattaggtggaattccagatacgcaaattgagcgctattataagtcatgtgtagagttcccggatatgaacagctcattaggagtgaaacgttcagagctggcgcgcatgattaagaatatcagttttgacgatttcaagaacgtgaaacaacaagcgaaaggacgcgaaaacgtcgcaaaagagcgcgccaaggccgtcattgggttgtacttaacggtaatgtacttacttgtcaaaaacctggttaatgttaacgcgcgctatgtcatcgccatccattgtctggaacgtgatttcggtctttataaggagattattcctgaactggcgtcaaagaacctgaaaaacgattaccgcattttatctcagactctgtgtgaactgtgtgataagtctcccaatttgttcttgaagaagaatgagcgcctgcgtaaatgtgttgaagtcgacatcaataatgcagacagctcgatgactcgtaaatatcgcaactgtatcgctcacttgactgtcgtccgtgaattaaaagagtacattggtgatatttgtaccgttgactcttatttcagtatttaccattatgtaatgcaacgctgtatcacaaagcgtgaaaacgataccaagcaggaggaaaaaatcaaatacgaagacgatttgcttaagaatcacggctatacaaaagacttcgtaaaagcattgaactcacctttcggatacaacatcccgcgttttaaaaatctttcaattgagcaactttttgatcgtaacgagtatcttacggaaaaa

SEQ ID NO: 3 - DNA, 2754 bp, synthetic construct
  misc_feature 1..2754: note = Synthetic: CasM DNA sequence from Ruminococcus bicirculans, modified for expression in Escherichia coli
  source 1..2754: mol_type = other DNA
atggcgaaaaagaataaaatgaaacctcgcgaattgcgcgaggcacaaaagaaagcgcgtcaattgaaagcagcggagatcaacaataacgcagttcccgccatcgctgctatgccggccgctgaggccgctgcccccgcagcggagaaaaagaagtcatcggtcaaagcggcagggatgaagtcaatcttagtctccgagaacaagatgtacatcaccagttttggaaaaggtaactcggcggtcttggagtacgaggtagacaataatgactataacaaaactcagttatcctcgaaggataatagcaatattgagttgtgtgatgtggggaaggttaatatcacgttcagctctcgtcgtggctttgaatcgggagtcgagattaatacgagtaacccaacccaccgctccggagagtcgtcgtcagtccgtggggatatgctgggcttgaaaagcgagttggaaaaacgtttttttggcaagaatttcgacgataatatccatattcaacttatttacaacatcttggacatcgagaagatccttgctgtgtatgttacgaacattgtttacgccctgaataatatgcttggcgaaggggatgaatctaactacgactttatggggtatttgagcacattcaacacatataaagtctttacgaatccgaatggttcaacgctgtctgatgacaagaaagagaacattcgcaaatcattatcgaaatttaatgctttgttgaaaacgaagcgcttaggttatttcgggttagaggagcctaaaacaaaggacacgcgcgcatcggaggcttacaagaaacgcgtatatcacatgctggctatcgttgggcaaatccgtcagtgcgtatttcatgataagagcggggccaagcgtttcgacctttattcatttattaataacattgatccagaatatcgtgaaactctggattacttggtcgacgaacgctttgacagtattaataaaggatttatccaaggtaataaagtaaacatcagcttactgatcgatatgatgaagggttacgaggcggatgacatcatccgtctttactacgatttcattgtccttaaatcgcagaaaaacctgggcttcagtatcaaaaagttacgcgaaaagatgttggatgagtatggctttcgtttcaaagataagcaatacgatagcgttcgcagcaagatgtataaattaatggatttcttattattctgcaattactaccgcaacgacattgcagcgggcgaatctcttgtccgcaagctgcgctttagtatgaccgatgatgagaaggaggggatctacgcagatgaggctgcaaaactgtggggcaaatttcgtaacgactttgagaacatcgccgaccacatgaacggtgacgtcattaaagagttggggaaagcagatatggactttgatgaaaagatccttgattccgaaaagaaaaatgcgtcggatctgttgtattttagtaaaatgatttacatgcttacgtattttctggacggaaaagaaatcaacgacttacttactacattaatttcgaagtttgataacattaaggagtttttaaaaatcatgaaaagcagtgcagttgacgttgaatgtgaacttacagcaggttataaattatttaatgacagccaacgcatcacaaatgaattgttcatcgtgaagaatatcgcgtctatgcgcaaacccgctgcttcggcgaagctgacaatgtttcgcgacgctttaacaatcctggggatcgacgataagatcactgatgatcgtatttccgaaatcttaaaattaaaggagaaaggaaaaggtatccatggcttacgcaattttatcactaataatgtaattgaaagtagccgctttgtgtaccttatcaagtacgcaaacgcacaaaaaatccgtgaggtcgccaaaaacgagaaagtcgttatgtttgtcctgggtgggattcccgacacacaaatcgaacgctactacaaaagttgtgtggaattcccggacatgaactcgagtctgggtgttaagcgtagtgaattggcccgtatgatcaagaatatcagttttgacgatttcaagaatgtgaaacagcaggccaaagggcgtgagaacgtcgcaaaggaacgcgctaaagctgtgatcggtttatatctgaccgtgatgtacttgttggtgaagaatttggtgaacgttaacgcgcgttacgttattgccattcattgcttagaacgcgactttggactgtataaggagattattcctgaattagccagcaaaaacctgaaaaacgattatcgtatcctgagccaaaccctttgcgaactttgtgataaaagcccaaacttgtttttaaaaaaaaatgagcgtttacgcaaatgcgtggaggttgatattaataatgctgattcctcgatgacccgcaaataccgtaactgtattgcccatttgacagtagtccgcgagttgaaggagtacattggagatatttgcactgtggacagttacttcagtatttaccattatgtaatgcaacgctgcattacaaagcgcgagaacgacactaagcaggaggaaaaaatcaagtacgaggatgatctgctgaaaaatcatggctacaccaaggactttgttaaggccttgaactctccgttcgggtataacattccccgcttcaaaaatctgagtattgagcagttgtttgatcgtaatgagtatcttacagagaag

SEQ ID NO: 4 - DNA, 2766 bp, synthetic construct
  misc_feature 1..2766: note = Synthetic: CasM DNA sequence from Ruminococcus sp., isolate 2789STDY5608892, modified for expression in Escherichia coli
  source 1..2766: mol_type = other DNA
atggccaaaaagaacaaaatgaagccccgcgaacttcgtgaggcccaaaagaaagctcgccaattaaaagcagccgagatcaacaacaacgcagctccggccattgcagcaatgcctgctgcagaagtgattgcgccagtcgccgaaaagaagaaatccagtgttaaagctgcaggtatgaagtctattttggtttcggagaacaagatgtatatcacaagcttcgggaaaggtaatagtgctgttcttgagtatgaagtagataacaacgactataataaaacccaacttagttctaaggataactctaatattgaattgggggacgttaatgaggtaaatatcacgttctcatcgaagcatggctttggttccggggtggaaatcaatacctctaatcccactcatcgttcgggtgaatcctccccagtccgtggtgatatgttggggcttaaatcggagttagagaaacgcttctttggtaaaacctttgatgataatattcatattcaattgatttataacattttggatatcgagaagattttggctgtatacgttacaaatatcgtgtatgcacttaataatatgttgggtattaaagattctgaatcgtatgatgatttcatgggctatttgagcgcacgcaatacctatgaagtcttcactcatcctgataaaagcaacttaagtgataaggttaaagggaacattaagaagagtttatcaaagttcaatgacttgttaaagaccaagcgccttgggtacttcggtcttgaggaaccgaagaccaaagatacccgcgcttctgaggcgtataagaagcgcgtctaccacatgcttgcaatcgtaggtcaaatccgtcagtgtgtgtttcacgacaaatcaggagcgaaacgtttcgatttgtactccttcattaataacatcgacccagagtatcgcgacactcttgactacttagttgaggaacgtttgaagtcaattaataaggatttcattgagggaaataaagtaaacattagccttcttatcgacatgatgaagggatacgaggccgacgatattattcgcctgtattatgattttattgtgttgaaatcacaaaagaatttggggtttagcattaaaaaattgcgcgagaagatgttggaggagtatgggtttcgctttaaggataaacagtatgactcagtccgctcaaaaatgtataagttaatggacttcctgcttttttgtaattattaccgtaatgacgtcgccgccggtgaagccctggttcgtaaattgcgcttctcaatgactgacgatgagaaggagggaatttatgctgatgaggctgcgaagttatgggggaagtttcgtaacgacttcgaaaatatcgccgaccacatgaatggagatgttatcaaggagcttggcaaggcggatatggattttgatgaaaagatccttgacagcgaaaagaagaatgcctccgatttgctgtacttttcgaaaatgatctacatgcttacctatttcctggacggcaaagagatcaacgatcttttgaccacccttatttctaagttcgataatatcaaagagtttttgaaaatcatgaagagttcggcggtcgatgttgaatgtgaattaacggccgggtataaattatttaacgactcccaacgtattacgaatgaattatttatcgttaaaaacatcgcttctatgcgcaaaccagcagcgtccgccaaacttacgatgtttcgtgacgcccttaccattttgggaatcgacgataacatcacagatgatcgcatttctgagatcttgaagcttaaggaaaagggcaagggcatccatggtttacgtaattttatcacaaacaacgtgatcgagtcgagtcgttttgtctatctgatcaagtatgcaaacgcgcagaaaattcgtgaagtggcaaaaaatgagaaagtagtaatgtttgttttgggtggtatccctgacacccagattgagcgctactacaagtcgtgtgtagaattccctgacatgaatagcagcttagaagctaaacgctctgaacttgcgcgcatgattaaaaatatctcgttcgatgacttcaagaacgttaaacaacaggccaaaggccgtgagaatgttgctaaagaacgcgcgaaggctgtaattggattataccttactgtaatgtatctgttagtgaaaaaccttgtgaacgtcaacgcccgctacgtcattgcgatccattgtttggagcgtgactttgggttatacaaggagatcatcccagaactggcctcaaaaaacttaaaaaatgactaccgtattttgagtcagaccttgtgcgaactgtgcgatgaccgtaacgaatcctcgaacttgttcttgaagaagaataaacgtttgcgcaaatgtgtcgaggtagatatcaacaatgcagacagctctatgacgcgtaagtaccgtaactgtattgctcacttaaccgtagttcgtgaacttaaagaatacattggagacattcgtacagttgatagctacttcagtatttatcactatgtaatgcagcgctgtatcactaagcgtggggatgatacgaagcaagaagagaaaattaagtacgaagatgacctgttgaaaaaccacgggtacactaaggactttgtcaaagctctgaattccccgttcgggtacaatatccctcgttttaagaatctgagtattgaacagttatttgaccgcaacgaataccttacggagaag

SEQ ID NO: 5 - DNA, 2766 bp, synthetic construct
  misc_feature 1..2766: note = Synthetic: CasM DNA sequence from Ruminococcus sp. CAG:57, modified for expression in Escherichia coli
  source 1..2766: mol_type = other DNA
atggctaaaaagaataaaatgaaacctcgcgagttgcgcgaagcccagaaaaaagctcgccagttaaaggcagcggaaattaataataatgcagcacccgccatcgcagcgatgcccgcagctgaagtaatcgcccctgttgctgaaaagaagaaatccagcgtgaaagcggcaggtatgaagtccattttggtcagcgagaataaaatgtacattacgtcgttcgggaaaggcaactccgctgtccttgagtatgaagtagacaacaatgactacaacaaaactcaactgtcaagcaaagacaacagtaacatcgaactgggagacgtgaatgaggtgaatatcacgttttcatcaaaacatgggttcggaagcggtgtggaaatcaatacaagcaatccgacccatcgctcaggggagtcgtcgcctgttcgtggagacatgttgggtcttaagtccgagcttgagaagcgttttttcggcaagacattcgatgacaacatccatattcagttgatttataatattttagatatcgaaaagattttagccgtatatgtgaccaacattgtttatgcgttaaataacatgttagggattaaggactcggaatcgtatgatgatttcatgggttacttaagcgctcgtaatacttatgaagtcttcactcatcccgataagagcaatttgagtgataaagtcaagggcaacatcaaaaagtctttgtcgaaattcaatgacctgttgaaaactaagcgcttgggttacttcgggttggaagaaccgaagaccaaagatacgcgtgccagtgaagcttacaaaaaacgcgtctatcacatgctggcaatcgtgggccaaatccgtcagtgtgtttttcatgacaaaagtggagctaaacgctttgatttgtacagcttcattaataacattgatcctgaatatcgcgacactttggattatttagtagaagaacgccttaaatctattaataaagactttattgaagggaataaggtgaacatcagcttactgatcgacatgatgaagggttacgaggctgacgacattatccgcttgtattatgatttcattgtattaaaatctcagaaaaacctgggattcagtattaagaaattacgcgagaaaatgcttgaggagtacggattccgtttcaaggataaacaatatgattctgtgcgtagtaaaatgtacaaacttatggactttttattgttctgtaactattaccgtaatgacgttgccgcaggcgaagccttggtacgtaagttacgcttcagcatgacagatgacgaaaaggagggcatttacgcggatgaagcagcgaagctgtggggtaaattccgcaacgattttgaaaatattgctgaccacatgaatggtgatgttatcaaagaactgggaaaagccgatatggatttcgacgagaagatcttggacagtgaaaaaaagaatgccagcgatcttttatatttctccaaaatgatctacatgcttacttatttccttgacgggaaagagattaatgatctgctgaccacgctgattagtaagttcgacaacattaaggagtttttaaagatcatgaaatcgtccgctgtggacgtagaatgcgagttgacggcaggttacaaactgttcaacgatagtcaacgcatcaccaatgaacttttcatcgtcaaaaacattgcctccatgcgcaagcccgcggctagcgctaaattaacgatgttccgtgacgccttgacgattttaggcatcgacgacaacatcacggacgatcgcatttcggaaatccttaaacttaaggaaaaggggaaaggtatccatggtctgcgcaattttatcactaacaatgtaattgaatcatcacgcttcgtttacttaatcaaatacgcgaatgctcaaaagattcgtgaagtagccaaggatgaaaaggttgtcatgtttgtcctgggcgggattccagacacccaaattgaacgttattacaagtcttgtgtggaattccccgatatgaatagctccttggaggccaaacgctctgagttagcccgcatgattaagaacatttccttcgacgattttaaaaatgtcaaacaacaggcaaaaggccgcgagaatgtagccaaggagcgtgccaaggcagtaatcggattgtatcttactgtcatgtatttgcttgttaagaatcttgttaacgttaacgcgcgctatgtaatcgctattcattgcttagaacgcgactttggcctttataaggagattattcccgagcttgcatccaaaaatcttaagaacgactaccgtattttgtcacaaaccttatgcgagttatgcgatgaccgcaacgagtcttccaatctgtttcttaaaaaaaacaaacgtcttcgcaaatgcgtggaagtggacatcaacaacgccgacagtagtatgactcgtaagtatcgtaactgtattgcgcacttgactgtagtgcgcgagttgaaggagtatattggggatatccgcaccgtggattcatacttcagtatctaccactacgtcatgcaacgttgcatcacgaaacgtggagacgacaccaaacaagaggaaaagattaagtatgaagacgaccttttgaagaaccacggctacaccaaagattttgttaaggctttgaatagtcccttcgggtataacattccccgtttcaaaaacttgagcattgaacagctgttcgaccgcaatgaatacttgacagaaaag

SEQ ID NO: 6 - DNA, 2799 bp, synthetic construct
  misc_feature 1..2799: note = Synthetic: CasM DNA sequence from Ruminococcus flavefaciens FD-1, modified for expression in Escherichia coli
  source 1..2799: mol_type = other DNA
atgaaaaaaaaaatgtctctgcgtgaaaagcgtgaggcggagaagcaagcaaagaaagccgcgtattccgctgctagtaagaatactgacagcaaacccgcagagaagaaggcggaaacacccaagcccgcagaaattatctcggataactcgcgcaataaaactgctgttaaagccgccggcttgaaatcaactatcatcagtggggataaattatacatgacgtcatttggtaagggaaatgccgccgtgatcgaacagaagattgatattaatgactactctttttctgccatgaaggatacccctagcttagaggttgataaggccgagagcaaggagatctctttttcctctcaccatcccttcgtaaagaatgacaaattgaccacttacaaccccctgtacggcggcaaggacaatccggaaaagccagtgggacgtgacatgctggggttgaaagacaaattggaggaacgttattttggatgcactttcaatgataatctgcacatccagatcatctacaatatcttagacatcgagaaaatcctggctgttcatagcgcaaatatcaccaccgcactggatcacatggtagacgaggatgacgaaaaatacttgaactctgactacattggttacatgaacaccattaatacgtacgacgtatttatggacccgtcaaagaactcttctttgtcgccgaaagatcgcaagaacatcgacaactcccgcgccaagtttgagaagttattgtcaacgaagcgtttaggatactttggttttgactatgatgcgaatggcaaggataagaagaagaacgaggagattaagaagcgtctgtaccatcttaccgcgtttgcgggtcagcttcgtcagtggtcctttcacagcgctggcaattatccacgtacatggctgtacaaacttgatagtttggacaaagaataccttgatacacttgatcactatttcgataaacgcttcaatgacattaatgacgatttcgttacaaagaacgcgacgaatttatatattcttaaggaagtttttccggaggcgaactttaaagatatcgcagatctttattacgacttcatcgtaatcaaatcccacaaaaatatgggtttctctattaaaaaattgcgtgaaaaaatgttagagtgtgatggtgcggatcgcatcaaagaacaagatatggacagcgtacgttcaaagctgtataaacttattgacttttgcattttcaaatattaccatgagttcccggaactgtctgagaagaatgttgatatcttacgtgctgccgtctccgacacgaagaaagataatctttatagcgacgaggccgcgcgtctgtggagtatcttcaaggagaagttcctgggtttctgtgacaaaattgtcgtatgggtgactggtgaacatgaaaaagatatcacttcggtaatcgataaagacgcgtatcgcaaccgtagcaatgtcagttatttttcgaaactgatgtatgcgatgtgctttttccttgatggtaaggaaattaacgatttattgacaaccctgattaataaattcgataatatcgcaaatcagatcaaaacggcaaaggaacttggtattaacacagccttcgtaaagaattatgacttttttaaccactcggagaagtatgtcgacgaactgaatattgtgaaaaacatcgctcgcatgaaaaagcctagtagcaacgctaaaaaagctatgtaccacgatgcattgacgatcttggggattcctgaagatatggatgagaaagccttagatgaggagctggacttgattctggaaaaaaagaccgatccagtaaccgggaagcctttgaaagggaaaaacccgcttcgcaactttatcgctaacaatgtaatcgaaaactctcgcttcatctatttgattaagttttgcaatccggaaaacgtacgtaagattgttaataacaccaaagttacagagtttgtcttgaagcgcatcccagatgcgcagatcgaacgctattacaagtcttgtactgactcggaaatgaaccccccaacggaaaagaaaattacggagttagccgggaaacttaaggacatgaattttggaaacttccgcaacgtgcgtcaaagtgcaaaggagaacatggaaaaggagcgttttaaagcagtgattggtttgtaccttaccgtagtctatcgcgttgtaaaaaatctggttgatgttaattcccgctacatcatggcgtttcattcgctggagcgcgacagtcagttatataatgtctcggtcgacaacgactacctggccttaaccgatacgttagtaaaagagggagataattcccgttcccgttacttagcggggaataaacgcttgcgtgactgtgtgaaacaggatattgataatgctaagaaatggttcgtcagtgataagtacaactctatcacaaaataccgtaataacgtagcacatttaactgcagtacgtaattgcgccgaatttatcggtgacattactaagatcgactcgtattttgcattatatcactaccttattcagcgtcaactggctaagggtttggatcacgagcgttcgggatttgaccgcaactatccgcagtatgctccactttttaagtggcatacttacgtgaaagacgtggttaaagccttaaatgctcccttcggatacaacatcccacgctttaagaatttgtctattgatgctttatttgatcgcaatgagatcaaaaagaatgacggagagaagaagtctgatgat

SEQ ID NO: 7 - DNA, 2832 bp, synthetic construct
  misc_feature 1..2832: note = Synthetic: CasM DNA sequence from Ruminococcus albus strain KH2T6, modified for expression in Escherichia coli
  source 1..2832: mol_type = other DNA
atggcaaagaaatccaaggggatgtcgttacgtgagaaacgcgaattggaaaaacagaagcgcattcaaaaggctgctgttaactccgtcaacgacactcctgaaaagacagaagaggctaacgtggtatcagtgaatgtgcgcacttctgccgaaaacaagcactccaaaaagtcagcggccaaggctttggggctgaaatctggcttggtaattggagatgagctgtatctgacatcgttcggtcgcggcaacgaagccaagttggaaaagaaaatctcaggtgatacggttgagaaattaggtatcggcgcttttgaggtagctgagcgtgacgagtcgacgctgacgcttgaaagtggacgcattaaggacaagacggcgcgtccaaaggacccacgtcacattacggttgatacacaaggtaaattcaaagaggatatgctgggtattcgcagcgtgttagaaaaaaagatttttgggaagacctttgacgataacatccatgtacaactggcatacaacattcttgatgtcgagaaaattatggcacagtatgtcagtgatattgtttatatgctgcacaacacggacaagacggagcgtaatgataacctgatgggttacatgtcaatccgcaacacatacaagacgttctgtgatacttcaaacttgcctgatgatactaaacaaaaagttgaaaaccaaaaacgtgaatttgataaaatcattaagagtggccgtctgggctatttcggggaagcttttatggtaaatagcggcaactctacaaaactgcgcccggaaaaagagatctatcatatttttgcgctgatggcgtcgttacgccaaagttactttcatggttatgtcaaagataccgattaccaagggaccacttgggcgtatacactggaggacaaactgaaggggccctctcacgagttccgcgagacgattgacaaaatctttgacgagggattttccaaaatctcgaaagatttcggcaaaatgaacaaggtgaacctgcaaattttggagcaaatgatcggggagttgtacgggtccattgagcgccaaaacttaacttgtgactactacgatttcatccagttaaagaaacataagtatcttggctttagcattaaacgtttacgcgagacgatgcttgagactactcccgcagagtgctataaggcagagtgctacaactctgagcgccagaaactgtacaagttgatcgactttttaatctacgacctttattacaatcgtaagcccgcacgtatcgaagagatcgtcgataagctgcgtgaatctgtgaatgatgaagaaaaagagtctatttactcagtagaggctaagtatgtctatgaaagcctttcaaaagtccttgacaagagcttgaagaatagtgtttctggggaaaccattaaagaccttcagaaacgttatgatgatgaaacagctaaccgtatttgggacatctcgcaacattcaatcagtggcaacgtcaattgcttctgtaaattaatttacatcatgactcttatgctggacggaaaagaaatcaatgatctgttgacaacgctggttaacaaattcgataacattgccagtttcattgatgtcatggatgagttaggattagagcactcattcactgataactataagatgttcgctgattctaaagctatttgtctggatttgcaatttatcaattcatttgcccgtatgtcgaagatcgatgacgaaaagtcgaaacgtcaactttttcgtgacgcgctggttattttagatattggtaataaggacgagacatggattaataactacttagattccgatatctttaagctggacaaggaaggtaataagttaaagggagcccgccatgattttcgcaactttatcgcaaataacgtgattaagtcttcacgcttcaaatatttagtgaagtattcgagtgcggatggcatgattaaattaaagacaaatgagaagcttattgggttcgttctggataagttaccagagacgcaaatcgaccgttactacgagtcttgcgggttagacaatgccgtcgtggacaaaaaagtccgtattgagaagctgagtgggttaattcgtgatatgaagttcgacgatttttctggcgtaaaaactagtaacaaagctggcgacaatgacaagcaggacaaggccaaatatcaggccattatttcgttataccttatggtgctttaccagatcgtaaagaacatgatttacgtcaactcacgctacgtcattgctttccactgtttagaacgcgattttgggatgtatggcaaggattttggaaaatattaccaggggtgccgcaagctgactgatcacttcatcgaagagaaatacatgaaggaaggaaaattgggatgcaacaaaaaagtaggacgctatcttaaaaataatatttcctgctgcacggatggactgattaacacataccgtaaccaggtggatcatttcgcagtggttcgcaaaattggtaactatgcggcctatatcaaatctatcggaagctggttcgaactttaccattatgtgattcaacgtattgtgtttgatgagtatcgtttcgcacttaacaacacagagtccaactataaaaactccattatcaaacaccatacgtactgtaaagatatggtaaaggcattgaatacgccctttggctacgacctgcctcgctacaagaacttgtcgatcggggacttgttcgaccgtaacaattatttaaacaagacgaaggaatcgattgatgctaattcaagcattgattcacag

SEQ ID NO: 8 - DNA, 2901 bp, synthetic construct
  misc_feature 1..2901: note = Synthetic: CasM DNA sequence from Ruminococcus flavefaciens strain XPD3002, modified for expression in Escherichia coli
  source 1..2901: mol_type = other DNA
atgatcgagaaaaaaaaatcttttgctaagggcatgggcgttaagtccaccttggtttcaggttctaaggtatatatgaccactttcgcagagggatccgacgcacgtctggagaaaattgtcgaaggagattcgatccgttcggtgaatgagggggaggcgttctccgcggagatggcggacaaaaatgcgggttataagattggaaacgctaaattttcccacccgaaaggatacgcagtggtagccaataaccccctttacacagggcctgtgcaacaggacatgttgggattgaaggagactttggaaaagcgctattttggtgagtccgcagatggaaacgataatatctgtatccaggtaattcacaatatcttggatattgaaaagatccttgctgagtacattaccaacgctgcctacgccgtgaataatatctccggcttagacaaggacattattggctttgggaagttcagtaccgtctatacgtatgacgaatttaaggacccagaacaccatcgtgccgccttcaataataatgataagttgatcaatgcaattaaagcccagtacgacgaatttgataacttcttggataatccccgcttaggctacttcgggcaagctttcttcagtaaggaggggcgtaactacattattaattacggcaatgagtgttacgatatccttgcattactttcggggcttcgccactgggttgtacacaataatgaggaagagtcacgcattagccgcacgtggttgtataaccttgataagaaccttgacaatgaatacatctctaccctgaactacttatatgatcgcattacgaatgagttaaccaattcattctcaaagaatagtgcagccaacgtcaactatatcgcagagacgctgggtatcaacccggcggaattcgccgagcagtatttccgcttttcaatcatgaaggaacaaaagaatctgggtttcaatattaccaagttacgtgaagtaatgttggatcgtaaggatatgtctgagattcgcaaaaaccataaagtgtttgacagcatccgtacgaaggtctacactatgatggacttcgttatctaccgctattacatcgaagaggatgccaaagtggcagcggcgaacaaatcccttccagacaacgagaaaagtctttctgagaaagacatctttgtaatcaacttgcgcggttcctttaatgatgaccagaaagatgcgttgtactatgatgaagctaatcgtatttggcgtaagttggaaaacatcatgcataacattaaggagtttcgtgggaacaagacacgtgagtataaaaaaaaggatgctccacgtcttccgcgcattttgcctgcaggacgcgatgtcagtgctttcagcaaattaatgtatgcactgacaatgtttctggacgggaaggaaatcaatgatcttctgactacacttattaacaagtttgataatattcagtccttcttaaaggttatgcctttgattggtgtaaacgcgaaatttgtcgaagagtatgcctttttcaaggatagcgcgaaaattgccgacgaactgcgtcttattaagagtttcgctcgtatgggggagccaatcgctgacgcccgccgcgctatgtacatcgatgctattcgcatcttaggtacaaacttgtcatacgatgaacttaaagctttagcagacaccttttcgctggatgaaaacggaaacaagttgaaaaaggggaagcatggaatgcgcaattttattatcaataacgtgatctcaaataagcgtttccactatcttatccgttatggagatccggcacacctgcatgaaattgccaagaatgaggccgtggtgaaattcgttttagggcgcattgctgatattcagaagaaacaggggcagaatggaaagaatcaaatcgaccgttactatgagacgtgtattggcaaagacaaggggaaatcggtttcggaaaaagttgacgccttgacgaagatcatcacgggcatgaactacgaccagtttgacaaaaaacgctcggtaattgaagataccggacgtgagaatgcggaacgtgagaaatttaaaaagatcatctcgttgtatctgaccgtaatttatcatattttaaaaaatatcgtaaacatcaacgcacgctatgtgatcgggttccactgtgtagaacgcgacgctcaactttataaagaaaaggggtatgatattaacttgaaaaagttagaggagaagggattctcatcagtcaccaagttgtgcgcgggtattgacgaaacggcaccggacaagcgcaaagacgttgaaaaggagatggccgaacgcgccaaggaaagtatcgactcattagaaagcgcaaatcccaagctgtatgccaattatatcaagtatagcgatgagaagaaggcggaggagtttacgcgccagatcaaccgtgaaaaggccaaaactgcattgaatgcctacttgcgcaatacgaaatggaatgtgatcatccgtgaggacctgctgcgtatcgataacaaaacatgtactttatttcgcaataaagcggtacatcttgaagtggcgcgttacgttcacgcgtatatcaatgacattgcagaggttaattcctatttccagctgtatcactacattatgcaacgcattattatgaacgagcgttacgagaaaagcagcggcaaagtatccgaatactttgacgcagttaacgatgagaagaaatataacgaccgcttactgaaattgctgtgtgtaccttttgggtattgcatcccccgttttaaaaacctgagtatcgaagctctgtttgaccgcaacgaggccgccaaatttgataaggaaaagaaaaaggtttcgggaaatagt

SEQ ID NO: 9 - DNA, 2388 bp, synthetic construct
  misc_feature 1..2388: note = Synthetic: CasM DNA sequence from Ruminococcus sp., isolate 2789STDY5834894, modified for expression in Escherichia coli
  source 1..2388: mol_type = other DNA
atggaaatcaacacttcgaaccccacccatcgcagcggtgaaagtagcagtgttcgtggggacatgcttggactgaagtcagagctggagaaacgcttttttggaaagaccttcgacgataacattcatattcaattgatctacaatatcttggacattgaaaaaatcctggccgtgtacgtcactaatattgtatatgcactgaacaatatgctgggagtgaagggcagtgagagctacgatgacttcatgggctatctgtcagcgcagaatacatattacatctttactcatccagataagtcaaacctgagtgacaaagtgaaaggcaacattaaaaagagtctgtccaaatttaatgatctgctgaaaacaaaacgtttgggttattttggactggaggagcccaaaactaaggacaagcgcgtgagcgaagcctacaagaaacgtgtttatcatatgctggcaattgtgggtcagatccgtcaaagcgtcttccatgacaagtctaatgaattggatgagtatctgtactcgtttatcgacattatcgacagcgaatatcgtgacacgctggattatttggttgatgaacgtttcgatagcatcaataagggcttcgtccaggggaataaggtaaacatctcgttactgattgacatgatgaaggggtatgaggccgatgacattatccgcttatactatgacttcatcgtgttgaaatcccaaaagaaccttggcttctccattaaaaaacttcgtgagaagatgcttgatgagtacggtttccgcttcaaggataaacaatacgattcagtgcgtagcaaaatgtacaagttgatggattttttattattctgcaactattatcgtaacgacgtggtagcgggcgaggctcttgtccgtaaactgcgcttctcgatgacagatgacgaaaaagaaggcatctatgccgacgaagccgagaaattgtggggcaagttccgtaatgactttgagaatatcgctgatcatatgaatggagacgttatcaaggaacttggcaaagccgacatggatttcgacgagaagatcctggattctgaaaagaagaacgcgtcggacttgctgtatttttcgaagatgatctatatgcttacttatttcttggatggcaaagaaattaacgacctgttgaccacactgattagcaaatttgataacattaaggagttccttaaaattatgaagtctagcgcagttgacgtggagtgcgagctgactgcgggatacaaattgtttaacgacagtcaacgtatcacgaatgaacttttcattgtgaagaacattgcgtcgatgcgcaagccggctgccagtgcaaagttgaccatgtttcgtgatgctctgaccatcttaggcattgatgacaagattaccgatgaccgcatttccgaaattcttaagttaaaagaaaaagggaaaggaatccatggtcttcgtaactttatcaccaacaatgtgatcgagtcctcgcgttttgtctacttgattaaatatgctaacgcacaaaagattcgcgaagtagctaaaaacgaaaaagttgtgatgtttgttttaggtggcattcccgatacccagattgaacgctactataaaagctgtgtcgaattcccggacatgaactcatctttagaggcaaaatgttcagagttagctcgtatgatcaagaatattagtttcgatgacttcaagaatgtgaaacagcaagcaaagggccgcgaaaatgtagccaaagagcgcgctaaggctgtcatcggattgtatctgacagtcatgtaccttcttgtcaagaatttggtcaacgtaaatgctcgctatgttattgctatccattgtttagaacgcgacttcggcttatataaagaaattattccggagttggcctcaaaaaacttgaagaacgattaccgtattttgagtcagaccctgtgcgaactgtgcgacgaccgcgacgagtcacctaacctgttcttgaagaaaaacaagcgcttacgtaagtgtgtggaggtggacatcaacaatgcggatagctccatgacccgtaaataccgtaattgcattgcccatcttaccgtggttcgcgaattaaaagagtatattggcgatatccgtactgtcgattcttatttcagcatctaccactacgttatgcagcgttgtatcacgaaacgtgaggacgataccaaacaagaggaaaagattaagtacgaagacgatctgctgaaaaaccatgggtatacgaaggacttcgtaaaagcgttgaactcccccttcggctataacattcctcgcttcaagaacttatctatcgagcaactttttgaccgtaacgagtatttaacggagaaa

SEQ ID NO: 10 - DNA, 2862 bp, synthetic construct
  misc_feature 1..2862: note = Synthetic: CasM DNA sequence from Eubacterium siraeum, modified for expression in human cells
  source 1..2862: mol_type = other DNA
atgggcaaaaaaatccacgcccgggacttgagggagcagagaaaaactgatcgcacagaaaaattcgccgatcaaaacaaaaaaagggaagctgagagagccgtccctaagaaagatgcagcggtctcagtgaaaagcgtgagtagcgtttccagtaaaaaagacaatgtaaccaagagtatggccaaggcagccggcgtaaagtcagttttcgcggtgggtaacactgtttacatgacaagttttggtcgaggaaacgacgctgtattggagcagaagattgtggatacaagccatgaacccctgaacattgacgatccagcctatcaactgaatgtggtaaccatgaacggatactcagttacaggccataggggtgagactgtttctgccgttaccgacaacccgttgaggcgctttaatggacgaaaaaaagacgagcctgagcagtccgtaccaaccgatatgctttgcctgaagcccaccctcgagaaaaaattttttgggaaggagttcgatgataatattcacatccagcttatatacaacattctcgacatagaaaagattcttgctgtctactcaacaaatgcgatttacgcactcaataacatgagcgccgacgagaatatcgaaaatagcgattttttcatgaaaaggactacggacgagacattcgatgactttgaaaagaaaaaagagtccacaaacagtagggagaaggcggattttgacgccttcgagaaatttatcggtaactacaggcttgcctattttgcggacgcgttctatgtgaataaaaaaaatcccaaaggaaaagcaaagaatgtgctcagagaggataaagaactgtactcagttttgacgctcatcggtaagctccgccactggtgtgtacattctgaagaggggagagcggagttctggctctataaattggacgagcttaaggacgacttcaagaacgttctcgacgtagtgtacaaccgacctgtggaagagataaataacagatttatcgaaaacaataaggtaaacatccaaatattgggctccgtctacaaaaacacagatattgccgaacttgtcagaagctactacgagtttttgattaccaagaagtataaaaacatgggattttcaattaagaagttgagagaaagcatgctcgagggaaaaggttacgcggataaagagtatgacagcgtgaggaacaaactttaccaaatgacggacttcattctctacacaggttacataaatgaggacagcgacagagcagacgatcttgtaaatacgcttcgctcttccctgaaggaagacgacaagaccactgtgtactgcaaggaggctgattacctctggaagaagtaccgagaatccattcgggaagtagccgacgcacttgacggcgacaatattaaaaagttgagtaaaagcaacattgagattcaggaagataagcttcgcaagtgcttcatctcttatgcggattctgtcagtgaattcacaaagctgatctacttgcttactagattcttgagtggtaaggaaattaatgaccttgttacaactttgatcaataagttcgacaatattagatcctttctcgaaattatggatgagcttggtctggaccgaactttcactgctgagtactcattctttgaaggttcaacaaaatatctggctgaattggttgagctcaactcctttgtcaagagttgtagctttgacatcaatgcaaagcgcacgatgtatcgagatgctttggatatcctgggaatcgagtctgacaaaacggaagaggacatcgaaaaaatgatagacaatatcttgcagattgacgcaaatggggataaaaaactcaaaaagaataacggcttgcgaaattttattgcatctaacgtcatagacagcaaccggttcaaatacctcgtgcgctatggcaatccaaaaaagattagagagaccgcaaagtgcaaaccagcggtccggtttgtgctgaacgaaattcccgacgcacagattgaacggtattatgaagcatgctgccctaaaaacacggctctgtgcagcgcgaataaaagaagggaaaagttggcggatatgatcgcggagattaaattcgagaatttttcagatgcaggcaactatcaaaaagcgaacgttacctcacggacctcagaggctgagataaagaggaaaaaccaggccatcataagactgtatcttactgttatgtacatcatgctgaaaaatctcgtaaatgtgaacgcacggtacgtaatagcgttccattgcgtcgagcgggatacgaagctgtatgcagagtcagggctggaggtaggaaatatcgaaaagaacaagacgaaccttactatggcagtcatgggggtaaaactcgaaaacggtattatcaagactgaattcgacaagtcattcgctgagaacgccgcaaacaggtatctgaggaacgcgagatggtacaagctgatattggataatctgaaaaaaagcgagcgggcggttgtaaacgaattcagaaacacagtatgccatttgaatgctatacgaaacattaacattaacattaaggaaataaaggaagtcgagaattattttgcattgtaccactatcttatacaaaaacacctcgaaaatcgatttgcagacaagaaggttgaaagagataccggggattttatctctaaacttgaagagcacaaaacctattgcaaagactttgtgaaagcctactgcacgccgttcggctataacttggtccgctataaaaacttgaccatcgatggattgttcgacaaaaactacccggggaaagacgatagtgatgagcagaag

SEQ ID NO: 11 - DNA, 2757 bp, synthetic construct
  misc_feature 1..2757: note = Synthetic: CasM DNA sequence from Ruminococcus sp., isolate 2789STDY5834971, modified for expression in human cells
  source 1..2757: mol_type = other DNA
atggcaaaaaagaataaaatgaagccgcgggaacttagggaagctcagaaaaaggcccgacaacttaaagctgccgagataaacaacaacgctgcaccggcgatagccgccatgcctgcagctgaggtgattgcacctgctgccgaaaaaaagaaatcaagcgtgaaagcagccggcatgaaatctatcctcgtgtccgaaaataagatgtatattacgtcttttggaaaagggaatagtgcggttctcgagtacgaagtagataataatgattataatcaaactcaactgtcatccaaggacaatagcaatatacaacttggcggggttaacgaggttaacattaccttttcaagcaagcacggctttgagtcaggtgtagaaataaatacaagtaaccccactcatcgctcaggggaatcatcacctgtacgcggggacatgctcgggcttaagtcagaactggagaaacgcttctttggtaaaacatttgacgacaatattcatatacagctgatctataatattcttgatatagagaaaatcttggctgtatacgtcacaaacatcgtatacgcacttaataatatgctcggggttaaaggcagcgaaagccatgacgacttcattggataccttagcaccaataacatctacgacgtattcatcgacccagacaatagcagtctgagcgatgacaagaaggctaacgtgagaaagtcactctccaaatttaatgccttgcttaaaacaaagagattggggtactttgggcttgaagagcctaagacgaaggataatcgcgtatcacaagcctataagaagcgggtctatcacatgctggcgatcgtgggtcaaattcgccaatgtgttttccacgacaagtctggcgctaagagattcgatctttacagcttcatcaacaacatcgaccccgagtaccgggacaccctggactacctcgtggaggaaagactcaagtcaatcaataaggattttattgaagataacaaggtaaatatatccctcctcatagatatgatgaaaggttacgaggccgatgatatcattcgactgtattacgatttcattgtactgaagagtcaaaaaaatctgggcttctcaatcaaaaaactgcgggagaaaatgctggacgagtatggttttaggttcaaggataagcaatacgacagtgtccgcagcaagatgtacaagctcatggattttttgctcttttgtaattactaccgaaatgacatagctgcaggcgagtctttggtgcgaaaattgcgcttttccatgacagacgatgaaaaggagggcatatatgccgatgaagctgctaaattgtggggaaaatttcggaacgatttcgaaaacatcgccgaccacatgaatggagatgtcatcaaggagcttggtaaagctgatatggactttgacgaaaagatattggacagtgaaaaaaaaaacgctagcgatcttctttatttttccaagatgatatatatgctgacgtattttcttgacggtaaagaaataaacgacctgctgactacattgatttcaaaatttgacaacatcaaggaatttctgaaaataatgaagagttccgcggtagatgtagaatgtgagttgacagccggatacaaattgttcaatgatagtcagaggatcaccaatgagttgttcattgttaagaatattgcgtctatgaggaaaccagcggcaagtgctaagttgacgatgtttcgagacgcgcttacaattcttgggatcgatgacaaaatcactgacgaccggatttcagggatactgaagctcaaggaaaagggaaaaggcattcatgggcttaggaactttatcactaacaatgtaattgaatctagccggttcgtctacttgatcaagtacgccaatgcgcaaaagattagagaagttgccaagaatgaaaaggtcgtgatgttcgtattggggggtattccagatacacagatcgaacgctactacaagtcttgtgttgagttcccggacatgaactcctctctgggggtgaagcgctccgaactggctcggatgattaagaacattagcttcgacgatttcaaaaacgtcaagcaacaagcgaaggggcgcgaaaacgttgccaaggagagggctaaagcagtgatcggtctttatctcacagtgatgtatcttcttgttaagaatcttgtcaatgtcaatgcacggtatgttatagctatacactgtctcgaacgagacttcggtctctacaaagaaattattccagagcttgcaagtaaaaacctgaaaaatgattatcgcatcttgtcacagacgttgtgtgagctgtgcgataagtctccaaacctcttccttaagaaaaacgaacgattgcgaaagtgtgtcgaggtggatatcaataatgcggactcttccatgacccgaaaatatagaaactgtattgcgcacttgaccgtagtcagagaactcaaagagtacataggggacatctgtacggttgactcatattttagtatctaccactatgttatgcaacgctgcataaccaagagggagaatgatacgaagcaagaagaaaagataaagtatgaagatgacctcttgaaaaaccacggttatacgaaggacttcgtaaaagctcttaactcaccatttggttacaatatcccaagattcaagaacctctcaatcgagcaattgttcgatcgaaatgagtatctgacggagaaa

SEQ ID NO: 12 - DNA, 2754 bp, synthetic construct
  misc_feature 1..2754: note = Synthetic: CasM DNA sequence from Ruminococcus bicirculans, modified for expression in human cells
  source 1..2754: mol_type = other DNA
atggcaaagaagaacaaaatgaagccgcgcgagttgcgggaggcccaaaagaaagctcgccagctgaaggccgccgaaatcaataacaacgcagtccctgccatagctgccatgccagcagccgaagccgccgcaccggctgcggaaaagaagaagtcctcagtaaaagctgcgggcatgaaaagtatacttgtgtcagagaacaagatgtatatcaccagttttggaaaaggcaactccgcagtgcttgagtatgaggtagataacaatgattacaacaagacgcagttgtccagcaaagataactcaaacattgaactgtgcgacgttggcaaggttaatataactttcagtagtcgccgcggatttgaatcaggggtggaaatcaatacttctaacccaactcatcggtctggggagagctcttcagtacgcggggatatgttgggacttaaatctgagctcgaaaagagattttttggtaagaacttcgatgataacatccacatccaattgatttataatatcttggatatagagaagatactcgcagtatatgtgactaacatcgtctacgcgcttaacaatatgctcggtgagggagatgagtctaactacgactttatgggctatctgagcacatttaacacctataaagtgttcactaatcccaatggaagtactttgagcgatgacaagaaagaaaacattcgcaagtcactctctaagttcaacgccctcctcaagaccaaacgcttggggtattttggtctggaagaacccaaaacgaaagacactagagcttcagaggcatacaagaaacgagtataccatatgctcgccattgtcgggcagatccgccagtgtgtgtttcatgataagtctggagcaaaacgattcgacctgtatagttttatcaacaatatagaccccgagtatagggaaactttggactaccttgtagatgagcggtttgactccataaacaagggctttatacaaggaaataaagtcaatatcagtctgctcatagatatgatgaaagggtatgaagctgacgacattattcgcctgtactatgactttatcgttcttaagtctcagaaaaatcttggcttcagtataaaaaagctccgcgagaagatgctggatgagtatggatttagattcaaggataagcagtacgacagtgtaagatctaaaatgtataaacttatggattttctgttgttctgcaactactaccggaacgacatcgccgcgggtgagagtttggtgagaaagcttcggttctccatgaccgacgacgaaaaggaagggatatatgcagatgaagcggctaaactctggggcaagtttcgaaatgacttcgaaaacattgcggatcatatgaacggtgatgtgataaaagaacttggaaaagccgatatggactttgatgaaaagatactggactcagaaaagaaaaacgccagtgacctcctttacttcagcaagatgatctacatgctcacctactttctggatgggaaagaaatcaatgatttgcttacaaccttgatctctaagttcgataatataaaggaatttttgaagatcatgaaatctagtgctgtggacgtagagtgtgaactcacagcaggatataagctctttaatgatagccaacgaataacaaacgagcttttcatagtgaaaaacattgccagcatgcggaagccggcggcgtcagcaaaattgaccatgttccgcgatgcactgactattcttgggatcgatgataaaataacggatgatcgcataagcgagattctgaaattgaaggaaaagggtaagggtatacacggtttgcggaacttcattacgaacaacgtcattgaatccagtcgatttgtgtatctgataaagtacgcgaatgcgcagaaaataagggaggttgctaaaaatgagaaggtcgtcatgttcgtacttggcggcattcccgacacacaaatcgaaaggtattacaaaagttgtgtagagttcccagatatgaacagttccttgggagtaaaacggtctgaactggcgagaatgataaagaatatatcattcgacgacttcaaaaatgtaaagcaacaggcgaaaggaagagagaacgtggctaaggaacgggccaaagccgttattggactttaccttacggttatgtacttgttggttaaaaaccttgttaatgtaaacgcacgctatgttatagcaatacattgcctggagagagacttcgggctctacaaggaaataattcccgaactcgcttcaaagaaccttaaaaacgattaccgcattcttagtcaaacgctctgcgagctctgcgacaaatcccctaacctgttcctcaaaaaaaatgagagactcaggaagtgcgtcgaggttgacatcaataatgcagattctagtatgactcgaaagtatcggaactgtatcgcgcacttgacagttgtgcgcgaactgaaagaatacataggcgatatctgtaccgtagactcatatttctcaatttaccactatgtgatgcaaagatgcataaccaagagggagaacgacacgaaacaggaggaaaagattaagtacgaggatgacttgttgaaaaaccacggttatacaaaagattttgtcaaggcactgaatagtccttttgggtataatatcccgaggttcaaaaacctttcaattgaacaactcttcgataggaacgagtacctgacggagaag

SEQ ID NO: 13 - DNA, 2766 bp, synthetic construct
  misc_feature 1..2766: note = Synthetic: CasM DNA sequence from Ruminococcus sp., isolate 2789STDY5608892, modified for expression in human cells
  source 1..2766: mol_type = other DNA
atggcaaaaaagaacaagatgaagccccgagagttgcgggaagcgcagaaaaaagcgaggcagcttaaggccgctgaaatcaacaacaatgccgctcccgcaatagctgcgatgcctgccgcggaggtgattgcaccagtagcggagaagaagaaaagttctgtaaaagctgcaggtatgaaaagcatattggtaagtgaaaacaagatgtatataactagtttcggcaaaggtaattctgccgtgttggaatatgaggttgataataacgattacaataaaacccaactctcctctaaagacaattcaaatatagagctcggcgacgtaaatgaagtgaacattacgttctccagcaaacacggtttcggctcaggggtggaaattaatacttctaacccgacacaccggagtggtgagtcatctccagtgagaggagatatgctcggattgaaatccgaactcgagaaacggttcttcggcaagacattcgacgacaacatccatatccagttgatttataacatactcgacatcgagaaaattttggccgtgtatgtgacaaacattgtttatgcattgaacaacatgctgggtataaaagattcagagagctatgacgactttatggggtacttgagtgcacgcaatacctacgaggtgtttacgcacccagacaagagtaatttgtctgacaaggtgaagggtaatattaagaagtccctttcaaaatttaacgacttgctgaaaactaaacgcttggggtactttggactcgaagaaccaaaaaccaaggatacaagggcatcagaagcctacaagaagagggtgtaccatatgctggctatagtaggtcagattcggcagtgcgtattccacgacaagtcaggtgcaaagagatttgatctttactcattcataaacaacattgatccggaataccgggatacgctggactatctggtagaagagcgattgaagtcaatcaataaagattttattgaaggaaacaaagtgaatattagcctgctgatcgacatgatgaaagggtatgaagctgatgacatcatacggctctactacgacttcatagtactcaagagtcagaagaacctgggtttttccatcaaaaaactgcgagaaaagatgttggaagaatacggctttcgcttcaaagacaaacagtatgattccgtccgaagcaaaatgtataagcttatggatttcctgctcttctgcaattattacagaaatgacgtagccgcgggagaagccctggtacgaaagttgagattctctatgacggatgacgagaaggaaggcatctatgctgacgaggcagcgaagctgtggggaaaattccgcaacgacttcgaaaacatagcggatcatatgaatggggacgttataaaagaactcggaaaagcggatatggactttgatgagaagatcctggattctgagaaaaaaaacgctagtgatcttctctatttctctaagatgatttacatgctcacgtattttttggatggcaaagaaattaatgatctcctcactaccctcatttctaagttcgacaatattaaggaattccttaagatcatgaagagttcagcggtcgacgtagaatgtgagcttactgccggatacaaattgtttaacgatagccagcgaatcacgaatgagctgttcattgtcaagaatatcgccagtatgaggaagcccgctgcgtctgcaaaattgactatgttccgcgatgctcttaccattctgggcattgacgacaatataactgacgaccgcatcagtgagatcctgaagctcaaggagaaggggaaggggatccacggattgcggaatttcatcacaaataacgtaattgagagttcccggttcgtgtatcttattaaatatgccaatgctcaaaagataagagaagtagcaaaaaacgagaaggtggtcatgtttgtactgggcggaatacccgacacccaaatcgaacggtattataaatcttgtgtagaattcccagacatgaacagttcactcgaagcgaagagatcagaactcgcgcggatgattaaaaacatttccttcgacgacttcaaaaacgtcaaacagcaggcgaaaggtagggagaatgttgcgaaagaaagagctaaagcggtaattggtctgtatctgaccgtcatgtacctgttggtgaaaaatcttgtcaacgtaaatgcgcgatacgtcatcgcgatccattgtcttgagcgagacttcgggctctataaggagattatccctgagttggccagtaaaaatcttaaaaacgactacagaatccttagccagacgctttgtgagctttgtgacgacaggaacgagtcttccaatctgtttctcaagaaaaataagaggctcagaaaatgtgtagaggttgatatcaataacgctgatagctctatgactcgaaagtatcggaattgtattgcacaccttacggtagttagggagctgaaagaatatatcggcgatatacgaacagtagacagctatttcagtatataccattatgtcatgcaacgctgcattaccaagaggggggacgataccaagcaggaggagaaaatcaaatacgaagatgacttgctcaagaatcacggttatactaaggattttgttaaagcgctcaatagtccttttggctacaacatcccccgattcaagaacctgagtattgaacaacttttcgatagaaacgagtaccttactgagaaa

SEQ ID NO: 14 - DNA, 2766 bp, synthetic construct
  misc_feature 1..2766: note = Synthetic: CasM DNA sequence from Ruminococcus sp. CAG:57, modified for expression in human cells
  source 1..2766: mol_type = other DNA
atggccaaaaaaaataagatgaaaccacgcgaattgcgggaagctcagaaaaaggctagacagttgaaggccgcggagataaacaacaatgcagcacctgctatcgccgccatgccagctgccgaggtgattgcccccgtagcggaaaagaagaaatcctccgtaaaagcggcggggatgaagagcatccttgtgagcgagaacaaaatgtacattacaagctttggtaaagggaactcagctgtgttggagtacgaagtcgacaataacgactacaacaagacccagctgtcctctaaagacaatagcaacatagaactgggcgacgtaaacgaggtaaatataacgttctcttctaagcatggctttggcagtggtgtggagataaatacttccaaccccactcatcgaagcggggaaagtagcccggttaggggagacatgctcggcttgaaatcagagctggagaagagattttttgggaaaacattcgacgataatatacacatccagctgatatataacattctggatatagaaaaaatacttgcagtgtacgttacgaacattgtctatgctttgaacaatatgctcggaattaaggattccgagtcctacgatgatttcatgggttacctgagcgcccgaaacacgtacgaggtgttcactcatccggacaaatccaatctcagtgataaagtgaagggcaacataaagaaatccctttctaaatttaacgatctcctcaagacgaaaagactcgggtactttgggctggaggaacctaaaacgaaagacactagagccagcgaggcttataaaaaaagagtctaccacatgctcgctatagttggacaaattaggcaatgtgtgtttcatgacaaaagtggtgcaaaacggttcgatctgtactcatttatcaacaacattgatccagagtaccgagacactctcgactatttggttgaggaacgattgaaatctataaacaaggatttcattgaggggaacaaggtaaatataagccttctcattgatatgatgaaggggtacgaagccgacgatataatccgcctctactatgattttattgtgctgaaaagtcagaagaatctggggtttagtattaaaaagcttagggagaagatgctggaagaatatggttttcggtttaaagataaacaatatgactccgtgaggagtaaaatgtacaaacttatggatttcctcctgttctgtaactattatcggaatgatgttgcagcaggcgaagcactcgtccgcaaacttagattcagtatgacagatgatgagaaggaaggaatatacgctgacgaagcggcgaaactgtgggggaaatttcgcaacgactttgagaacatagctgaccatatgaatggcgacgttatcaaagagctcggtaaggcggacatggacttcgacgagaaaattctcgacagtgagaaaaagaacgccagtgatctgctgtattttagcaaaatgatatacatgctcacatactttctcgatggtaaagagatcaacgacttgttgaccacgcttattagcaaatttgataacatcaaagagttcttgaaaataatgaagtccagtgccgtggatgtggagtgcgagctcacggcaggttataaactttttaacgatagtcaacggatcactaatgagctgttcattgtcaagaatattgcaagcatgcgcaagcccgcggcaagtgcaaagcttacgatgtttcgggacgccctcacgatattgggtatagatgacaatataactgatgatagaatcagtgagatacttaagctcaaggaaaaggggaaagggatacacggtctgcgcaacttcataacgaataacgtgattgagagctcccgatttgtctatctgataaagtacgccaatgcccaaaagataagggaagtagctaaagatgaaaaagtggtcatgttcgtccttggcgggattcccgacacgcagattgagaggtactacaagtcttgtgtggagtttccggatatgaacagctccctcgaggctaagcgcagtgagctggctagaatgattaagaatatttcctttgatgattttaaaaatgtaaagcaacaagctaagggacgggagaacgtcgccaaagaacgggcgaaagcagtgattgggctttatctcacggtcatgtatctgcttgttaagaacttggtcaacgtcaatgcaagatatgttatagcgatccactgccttgaacgagatttcgggttgtacaaagaaatcatcccggagttggcatctaaaaaccttaagaatgactatcgaatactgtcacaaaccttgtgcgaactctgcgatgaccgaaacgaatcatctaacctcttccttaaaaaaaacaagagactcagaaagtgtgtggaggtggatatcaataatgccgattccagtatgactagaaaataccgcaactgcatcgcacacctgactgtggtcagagaacttaaggagtacattggagatattagaacggtcgactcatattttagcatctatcattatgtcatgcagaggtgtatcaccaagagaggagatgatacaaagcaggaagagaagataaagtacgaggacgatcttcttaagaaccatggctacactaaggacttcgtaaaagcgttgaactccccgttcgggtataacatacctaggtttaagaatctttcaattgagcaattgtttgaccgcaatgagtaccttacagagaag

SEQ ID NO: 15 - DNA, 2799 bp, synthetic construct
  misc_feature 1..2799: note = Synthetic: CasM DNA sequence from Ruminococcus flavefaciens FD-1, modified for expression in human cells
  source 1..2799: mol_type = other DNA
atgaaaaagaaaatgtccttgcgagaaaaaagggaagctgaaaaacaagcaaagaaggccgcgtactcagcagcttccaagaataccgactccaaaccagcggaaaagaaggcagaaaccccgaagccggcagagataataagtgacaacagtcggaataaaacggctgtgaaagctgcgggccttaaatctaccattatatctggagataagctgtacatgacatcatttggtaaggggaacgctgcggttattgaacagaagatcgacatcaatgactatagcttctctgctatgaaagatacaccatccctggaagtggacaaggctgaaagcaaggaaatttcatttagcagccaccacccgttcgtgaaaaatgataaactgaccacctacaacccattgtatggtgggaaagataatccggaaaaaccagtaggaagagacatgctgggactgaaggacaagcttgaagaacggtatttcggatgcaccttcaatgataacttgcatattcagattatatataacatactcgatatcgaaaagatacttgcagtgcactccgcaaacatcacgaccgcgctggatcacatggtggacgaagatgatgagaaatatcttaacagtgattacatcgggtacatgaacacaattaacacatacgacgtatttatggacccttctaaaaattccagcctctcacctaaggaccgcaagaatatcgacaacagtcgagccaagtttgaaaaactgttgagcacgaaaaggcttggatatttcggattcgattatgacgccaatggtaaggacaaaaaaaagaatgaagagataaaaaaacggctgtatcatttgactgcattcgctggccaactgagacagtggtccttccattctgctgggaactaccctcgcacgtggctctacaaattggacagcttggacaaggaataccttgacacgctggaccattactttgataaacggttcaatgatattaacgatgattttgttaccaaaaacgccactaacttgtatatactcaaggaagtatttccggaggcaaatttcaaagacatagccgacctttactacgactttattgttatcaagagccacaagaacatggggttttccattaaaaaactccgcgagaagatgctcgaatgcgatggtgctgaccgcatcaaggagcaggatatggactcagtaaggagtaagctttacaaactgatcgacttttgtatttttaagtattaccacgaatttcctgagttgtcagagaagaacgtcgacatacttcgagcagcggtttctgatacgaaaaaggataacctttattcagacgaggctgctcggctgtggagcatattcaaagaaaagttcctcggcttttgtgacaaaattgtggtttgggtcaccggagagcacgaaaaggacatcacgtcagtgattgataaagacgcatatcgaaatcgcagtaacgtttcttacttctccaagcttatgtacgcaatgtgtttctttcttgatggtaaggagataaacgacctcctcacgacccttatcaataagttcgacaatatagcaaatcagattaagacggccaaagaactcggaataaacactgcatttgtaaagaactacgacttcttcaatcatagcgagaaatacgtagacgagctgaatatcgtgaaaaatatcgctcggatgaaaaaacccagttcaaacgcaaaaaaggcaatgtatcatgacgcattgacgatattgggaatcccagaggacatggatgagaaggctctcgacgaagaattggacctcattttggagaaaaagactgatccggtgactggcaaaccactgaaaggcaaaaaccctctgcgaaatttcatagccaacaacgtaatcgaaaacagtagattcatataccttattaagttctgcaaccccgagaatgtccgcaagatagtcaacaacacaaaggtcacggaattcgttctgaagcgcattcctgatgcccaaatcgagcggtactacaagagttgtactgatagtgagatgaacccccccacggaaaaaaagattacggagctcgctggtaagctgaaagatatgaattttgggaacttcaggaacgtaaggcaatctgcaaaggaaaacatggaaaaggagcgcttcaaagcagtgattggcctgtatctcaccgttgtgtaccgagtcgtcaagaatcttgtagatgtgaacagtcgatacatcatggcttttcacagtctggaacgggatagtcagctgtacaacgtctccgtggataacgattacctcgcacttacggacactcttgtcaaggaaggcgacaattcccggtcacgatatctggccggaaataaacgccttcgagattgtgtaaagcaggatattgataacgcaaagaagtggtttgtgagcgacaagtacaatagcataactaaataccgaaacaatgtagctcaccttaccgctgtaaggaattgcgcggaatttatcggtgatattactaagattgattcctatttcgcactgtatcattatctgatacagaggcaacttgccaagggcctggaccatgaacggagtggctttgatcgaaactatccccaatacgcaccattgtttaaatggcatacttacgttaaggacgttgtgaaggctcttaatgctcctttcggttacaatatacctagattcaaaaatctgagcatcgatgcactgttcgaccgcaatgagattaaaaagaacgacggagagaaaaagtccgacgat

SEQ ID NO: 16 - DNA, 2832 bp, synthetic construct
  misc_feature 1..2832: note = Synthetic: CasM DNA sequence from Ruminococcus albus strain KH2T6, modified for expression in human cells
  source 1..2832: mol_type = other DNA
atggctaaaaaatcaaaaggaatgagcctccgcgaaaagcgggaactcgagaagcaaaagaggattcaaaaggccgcggttaattcagtgaacgatacacccgaaaaaacggaggaagctaatgtcgtcagcgtcaacgttcgaacctcagctgagaataagcactccaaaaaatctgcggccaaagctctgggccttaagagtggtctggttataggagacgaactttacttgacgagcttcggtcgcggtaatgaagcgaagttggagaaaaagattagcggcgacacggtcgagaagctggggatcggcgccttcgaggttgctgaaagggacgaatctactctcacgctcgagagcggtcgcatcaaagacaagacagccagaccaaaagatccacggcatattactgttgatacacaaggaaaattcaaagaagatatgttgggtatccggagcgtactcgagaaaaaaatatttggcaaaacttttgatgataacatccacgtacaactggcgtataacattcttgacgttgaaaaaatcatggctcagtacgtctcagacatagtatacatgttgcacaacacggataagaccgagcgcaatgataacctgatgggatatatgtccattcgaaacacatataagacattttgcgacactagcaatctgcctgacgacacaaagcaaaaagttgaaaaccaaaagagagagttcgataagataatcaagtccggccgactcggatattttggagaagcatttatggtaaattcaggcaatagtacgaagctccgacctgagaaagaaatctaccatattttcgcgcttatggcatccctgcgccaaagctactttcatggttacgtcaaggatacagattaccagggtaccacgtgggcgtatacgcttgaagacaaactcaagggtccatctcatgagtttcgagaaacgatcgataagatttttgacgaggggttttcaaaaatcagtaaagatttcggaaagatgaacaaggttaatctccagattttggaacaaatgataggcgagctgtatggctccatcgagcgccaaaaccttacgtgtgactattatgattttatacagcttaaaaaacacaaatatctgggtttctccataaaacgcctcagggaaacgatgcttgagacaacacctgcggaatgttataaggcagaatgttataactctgagaggcaaaaactgtacaagctgatcgacttcctgatctacgatctctactacaatcgcaagccagcacgaattgaagagatagtcgataagctgcgggagagcgtgaacgacgaggagaaggagtccatatactcagttgaggcaaagtatgtctatgagtccttgtcaaaagtgctcgacaagagtctcaaaaactctgtgagcggtgagacgatcaaagaccttcagaaacggtatgacgatgagacggccaaccggatctgggacatctcccagcattccatatccggtaacgtgaactgtttctgtaagcttatctacatcatgacactgatgctcgacggcaaggaaatcaatgatctcctgactacacttgttaacaagttcgataacattgcttctttcatagacgttatggatgagcttgggctggagcacagttttaccgataactataagatgtttgcagattccaaggccatatgcttggatctgcaatttataaattccttcgctagaatgtctaagattgatgacgaaaaatctaaacgacagcttttcagggatgcgctcgtaattcttgacatcggaaataaagatgagacctggataaacaactacttggattccgacatattcaagttggataaggaaggaaacaaactcaagggtgcccggcatgactttaggaactttattgcgaacaacgtcatcaagtcctcccggtttaagtatctcgttaagtactctagcgctgacgggatgataaagctgaaaacgaacgagaaactcatcggattcgtcctggacaagctgcctgagacgcagatagatcgatattatgaatcatgcggccttgacaatgcggtcgtcgacaagaaagtgcgaatagagaagttgagcggacttatcagggacatgaagtttgatgacttctccggcgtgaagacttctaacaaggccggagacaatgataaacaagataaggcgaagtaccaggctattattagtttgtatctgatggtactgtaccagatagtaaaaaacatgatttacgtcaattcccgctatgtcattgctttccactgccttgaacgcgactttgggatgtatggcaaagattttggaaagtactaccagggctgtcggaagttgaccgaccacttcatagaagaaaagtacatgaaggaaggaaagttggggtgcaacaaaaaggtcgggcggtacctgaaaaacaatatttcctgctgtacggacggattgataaatacttaccgaaatcaggtggaccattttgcggtagtccgaaagataggaaactacgcagcctacattaagtcaataggctcttggtttgaactgtaccactacgtaattcagaggattgtcttcgacgaatacagattcgctcttaacaacaccgagtcaaattataagaattccatcatcaaacatcacacgtattgtaaggatatggtgaaggcgctgaacacgccgtttggttatgatttgccacggtacaaaaatctctccattggggatcttttcgaccgcaataactatctcaacaaaactaaggaaagcatcgacgctaatagttcaatagattctcaa

SEQ ID NO: 17 - DNA, 2901 bp, synthetic construct
  misc_feature 1..2901: note = Synthetic: CasM DNA sequence from Ruminococcus flavefaciens strain XPD3002, modified for expression in human cells
  source 1..2901: mol_type = other DNA
atgatagagaaaaaaaaaagctttgcgaaagggatgggcgtaaaaagtacactggtatcaggctctaaggtctacatgacaacgttcgcagaaggaagcgatgcacgcctcgaaaagattgttgagggagatagcattaggtccgtcaatgaaggagaagcctttagtgcagaaatggcagacaagaacgctggatacaagattggaaacgcaaaattttcccatccaaagggatacgcagttgtagctaacaatcccctctataccgggcccgtccagcaagacatgcttggcctcaaagagacgcttgagaagaggtattttggagagagtgctgatggtaatgacaatatctgtatccaagttattcataacatcctcgacatagagaaaatccttgcagaatatatcaccaacgccgcatatgcagtgaataatatatccggtctggataaagacataatcggattcggcaagtttagtacagtatatacctatgacgagttcaaagacccggagcatcatcgagccgctttcaataacaacgacaaacttatcaatgccattaaggctcaatatgacgagttcgataattttttggacaatcccagacttgggtatttcggccaggccttcttttctaaggaaggcaggaattacatcattaattacggaaacgaatgttacgatatcctcgctttgctctctggcctgcgccactgggttgtacacaacaacgaggaggaatctcgaatttcacgaacttggctgtacaatttggataaaaacttggataatgaatacatcagtactctgaactatctctacgataggatcaccaacgaacttacgaattcattttcaaaaaattccgccgcaaacgttaattacatcgctgagacgttgggcataaatccggccgagttcgccgagcaatattttaggttcagtatcatgaaggagcaaaagaatttggggttcaacatcacgaaactccgagaagtcatgctcgaccgaaaagatatgtccgaaattcggaagaaccataaggtattcgacagcatccgcacaaaagtgtacacaatgatggatttcgttatatacaggtattatatagaggaagatgcaaaagttgccgccgcaaacaaaagtcttccagataatgaaaagagcttgagtgaaaaagatatttttgttataaaccttcgcggttccttcaatgatgaccaaaaggatgctctgtactacgacgaggcaaaccgaatctggcgaaaactggaaaacatcatgcataatataaaggaatttcgcgggaacaaaacgagggagtataagaagaaggatgctcctcgcctccccaggatactccctgcgggcagagacgtctccgcatttagcaaactgatgtatgctctcactatgtttttggatgggaaggagataaacgatcttctgactacgttgattaacaaatttgacaacattcagagttttctcaaggtcatgccacttatcggcgtaaatgcaaagtttgttgaggaatacgccttctttaaagactccgctaaaatagcggacgagctccgcctgattaaatccttcgcccgaatgggtgaaccgatagcggatgcccggcgagctatgtacatcgatgctatcaggatccttggaactaacttgagctacgacgaacttaaggctctggcggacactttcagtttggacgagaatgggaacaagctgaaaaagggaaagcacgggatgagaaacttcataataaataatgtcatttccaacaagaggttccattatttgattcggtatggtgatcctgcgcaccttcatgaaattgcgaagaatgaagctgtggttaaatttgttcttggcagaattgccgacatccaaaaaaaacaggggcaaaatggtaagaaccaaattgatagatactacgaaacttgcataggtaaagacaaaggtaaaagtgtctctgaaaaggtggatgccctgacgaaaatcatcacaggtatgaactatgaccaattcgacaaaaagagaagtgtaattgaggatactggtcgggaaaacgctgaaagagagaagtttaagaagattattagtctctatcttaccgttatttatcacattctcaaaaacatagtcaacatcaatgccagatatgtcatcggattccactgcgttgaacgagatgctcagttgtacaaggagaaaggctacgacatcaacctcaaaaaactggaggaaaaggggtttagttccgttacaaagttgtgcgccggaattgacgagacggccccagataaacgaaaggacgttgagaaagaaatggcggaacgagcgaaagagtccatcgactctcttgagtcagctaatcctaaattgtatgcaaactatattaaatactctgatgagaagaaagcggaggaattcacacgacagatcaatcgggagaaagcaaaaacggcactgaatgcatacttgaggaacacgaagtggaacgtgattatcagagaggacctgttgaggatcgacaataaaacgtgtaccctgtttagaaataaagccgttcatctcgaggtggcccggtacgtgcacgcctatattaatgacattgcggaagttaattcttattttcaactgtaccattacatcatgcagagaattatcatgaatgaacgatacgaaaagagcagcggcaaagtgtctgagtattttgatgccgtcaatgatgagaaaaaatacaatgacaggctgttgaagctgctgtgcgtaccatttggttattgtattcctcggtttaaaaatcttagtattgaggctctttttgatcggaatgaagccgcaaagtttgataaggagaagaaaaaggtatccggtaacagc

SEQ ID NO: 18 - DNA, 2388 bp, synthetic construct
  misc_feature 1..2388: note = Synthetic: CasM DNA sequence from Ruminococcus sp., isolate 2789STDY5834894, modified for expression in human cells
  source 1..2388: mol_type = other DNA
atggaaattaatactagtaatcccactcataggtccggtgaatcttctagcgtacgaggagacatgcttggtctcaaatcagagctcgagaagagatttttcgggaaaacatttgatgataatatccacattcaacttatatataatatccttgatatcgagaagatccttgcagtctatgtgactaatattgtctacgcacttaacaatatgctcggtgtaaaaggctcagagtcctatgacgactttatgggctatctttcagcacagaatacgtactacatatttacacatcccgacaagagcaacttgagcgataaagtgaagggcaatattaagaaatctcttagtaaattcaatgaccttctgaagacgaagcgacttggctattttgggctggaggagcccaaaaccaaagataagcgagtgtctgaagcttataaaaaacgagtgtatcacatgctggctatagtgggtcaaattcgccagtcagtctttcacgacaagtccaacgaattggatgagtacttgtattcctttatagacatcatcgatagcgagtatcgagacacattggactacctggttgatgaacgatttgattccattaacaaaggattcgttcaggggaataaggtaaacatctccttgcttatcgacatgatgaagggctacgaggctgatgatataataagattgtactatgactttattgtcctcaagtctcaaaagaatctgggtttcagtataaaaaaattgcgggagaagatgctcgacgagtatggatttaggtttaaggacaagcagtatgatagcgttcgctctaagatgtataaacttatggactttcttctgttctgtaactactatcggaacgacgtagtcgcaggggaggcactggttaggaaactgaggtttagcatgaccgacgacgagaaagaaggtatttatgcggacgaagcggagaagctttggggaaagtttaggaatgactttgagaacatcgccgatcacatgaacggtgatgtgataaaggagctcgggaaggcggatatggactttgacgagaaaatactggattctgaaaagaagaatgcaagtgacctcctttacttcagcaaaatgatctacatgttgacgtattttttggatggtaaagagatcaacgatctgcttacaacgcttatttctaaatttgataacataaaggagtttttgaagatcatgaaatcctccgccgtggatgtagagtgtgagctgaccgcgggctataaactgtttaacgattctcaacggataacgaacgagctcttcatagtgaagaacatcgcttccatgcgcaagccggcggcttcagccaaattgactatgttccgcgatgcgctgacaatactcgggattgacgataaaattacggacgaccgaatatcagaaattcttaaattgaaggaaaagggcaagggcatccatggcctgcggaacttcatcacgaacaacgttatcgagtctagtcggtttgtttatcttataaaatacgcgaatgcgcagaaaattcgggaggtcgcaaaaaatgaaaaggtggtaatgtttgtgctcggggggattcctgacacacagattgagcggtactataaaagttgcgttgagttccctgacatgaattcttcactcgaagccaagtgcagtgagctggcacggatgatcaagaatatctccttcgatgattttaagaacgtaaaacaacaagctaaaggacgcgaaaatgtggcgaaagagagggccaaggcagtcatcggtctctaccttacagttatgtacctccttgtgaaaaaccttgtaaacgtcaatgctcggtatgtaatagcaatccactgtttggagagagatttcggcctctataaggagatcatcccggagctcgcttcaaaaaacttgaaaaatgattatcgcattctttctcaaactctttgtgaactttgtgatgacagggacgagagtcctaacctgttcttgaagaagaacaaaagactgcggaaatgtgtggaggtcgatataaacaatgcggattctagcatgacccggaaataccggaattgcattgcacaccttacagtggtacgcgagctcaaggaatacatcggtgatatacgcaccgtcgactcctacttttctatctaccactatgttatgcaacggtgtatcaccaaaagggaggatgatactaagcaagaagaaaaaatcaagtatgaagatgacctgcttaagaaccatggatacacgaaagattttgtgaaagcccttaatagtccattcgggtacaatattccgcgattcaaaaacctttccatcgaacaactcttcgatcgaaatgagtaccttaccgagaaa

SEQ ID NO: 19 - DNA, 2862 bp, synthetic construct
  misc_feature 1..2862: note = Synthetic: CasM DNA sequence from Eubacterium siraeum, modified for expression in Zea mays cells
  source 1..2862: mol_type = other DNA
atgggtaaaaagatacatgcacgggacttgcgcgagcagaggaagacggaccggaccgaaaaattcgcagaccaaaacaaaaaaagagaggctgaacgggcagtccccaagaaagatgcagccgtgtcagtcaaatcggtctcaagcgtctcatccaaaaaggacaatgttaccaaatctatggcgaaagccgccggagtcaagtctgttttcgctgttggcaatacggtctacatgacatccttcgggcgcgggaatgatgcggttcttgaacagaaaattgttgatacttcacacgaaccactcaacattgatgacccagcttatcaactcaatgtggttacgatgaatgggtattcagtgaccgggcataggggagaaacggtctcggcagtcacagacaatcccttgagaagattcaacggcagaaaaaaggacgagccggaacaatcagtgccgactgacatgttgtgtctcaaaccaaccctggaaaagaaattttttggcaaagagttcgacgacaatatccacattcagttgatatataacatcctggatattgagaaaattttggccgtctactcgaccaacgccatatacgctctcaacaacatgtcagcagatgagaacattgagaactcagacttttttatgaaacgcaccacggatgagaccttcgatgacttcgagaaaaagaaagagtccacgaacagcagagagaaagctgatttcgatgcgttcgaaaagttcatcggcaactacaggctggcgtatttcgcagatgcattttatgtcaacaagaaaaatcccaagggtaaggccaaaaatgtcctccgcgaagacaaggaactctactcagtgctcacattgatcggaaagttgcggcattggtgcgttcattccgaggagggtcgggcagagttctggctttataaactggacgaattgaaggacgattttaagaacgtgcttgatgtcgtctacaatagaccagtcgaagaaattaataaccgctttattgaaaacaataaggtcaacatacaaatcttgggatcggtctataaaaacaccgacatcgcagagctggtcagaagctactacgagtttctgataactaaaaagtacaagaacatgggcttctcaataaaaaaactgcgcgaatcaatgcttgaaggtaagggatatgcggataaagaatacgattctgttagaaacaagctctaccagatgactgacttcattctctataccggttatataaacgaagatagcgacagggctgatgacctggtcaacacactgcggagctccctgaaagaggacgataagaccacagtgtactgtaaggaggccgattacctgtggaagaaataccgcgagtctattagggaggtcgcggacgccctggacggtgacaatattaaaaaactctctaaaagcaatatcgagatacaagaagacaaactgcgcaagtgttttatatcttatgcggattcagtctcggagttcacgaaactgatatatctcctgacacgctttctgagcgggaaggagattaatgacttggtgacaactttgattaacaagttcgacaacataaggagctttcttgaaatcatggatgagctgggcctcgatagaacgttcaccgcggagtactcgttcttcgagggttcaacaaaatatcttgcggaactcgttgaattgaattcgttcgtgaaaagctgttcttttgatataaatgccaaaagaacaatgtaccgggacgcgcttgatatcctgggcatagaatcggataaaaccgaggaagatatcgaaaagatgatagacaatatcctgcaaatcgacgcaaatggtgacaagaagcttaaaaagaataacggcttgcgcaattttatcgcttcgaatgtcatcgattcgaacaggttcaaatatctggttcggtacggtaacccgaagaagattagagaaacagctaagtgtaagccagcggtcagatttgtcttgaacgaaataccggatgcgcagatcgaaagatattacgaagcctgctgccctaagaacaccgcattgtgtagcgcgaataagcggcgggagaaactcgctgatatgatagcggagattaaattcgaaaatttctcggacgcgggcaactaccaaaaagctaacgttacttcccgcacttcggaggcggagattaaacggaagaatcaagcgataattagactttatctgaccgtcatgtacattatgcttaagaatctcgtcaacgttaatgctagatatgtcatcgcctttcactgcgtggaacgcgatactaaactgtatgccgaatcgggtcttgaagtcgggaacatagaaaaaaataagaccaaccttactatggccgtgatgggtgtcaaactggagaacggcattatcaaaactgaatttgataaaagcttcgccgaaaacgcagcgaatcgctatctgcggaacgcaagatggtataagcttatactcgataatcttaagaagtcggaaagggccgtggtcaacgagttccggaataccgtttgccacttgaacgcgatccggaatattaacatcaatatcaaagaaattaaagaagtcgaaaactactttgcgctctatcattacttgatacagaagcatctcgagaatcgcttcgccgataaaaaggtggagagggacacaggtgactttatttccaagctcgaagagcataaaacctattgcaaggattttgttaaagcatattgtacgccattcggttataatcttgttaggtacaagaatctgacaatcgacggcttgttcgataaaaattatccgggcaaggacgatagcgatgagcagaag

SEQ ID NO: 20 - DNA, 2757 bp, synthetic construct
  misc_feature 1..2757: note = Synthetic: CasM DNA sequence from Ruminococcus sp., isolate 2789STDY5834971, modified for expression in Zea mays cells
  source 1..2757: mol_type = other DNA
atggccaagaagaataaaatgaagccacgcgagctgagggaggctcaaaaaaaagcccggcagcttaaggctgcggagatcaataataatgctgcccccgctatcgcagcaatgcccgccgcagaggtcattgcgccggccgccgaaaagaaaaaaagctcagtgaaggctgcaggaatgaagtcaattttggttagcgagaataagatgtatattacctcgtttggcaagggaaacagcgccgtgctggaatacgaagttgataacaatgactataaccagacacagctttcatcgaaggataattccaacatccaattggggggcgtgaacgaagttaatataacgttttcttcaaaacatggtttcgaatctggagtcgaaataaatacgtctaatccgactcataggtccggtgagtccagccctgtccggggggacatgctcggtctcaagtccgaactcgaaaaacggtttttcggtaagactttcgatgataatattcatattcagcttatatacaatatcttggatatagagaaaattctggcggtgtatgtcacaaatatagtgtatgctctgaataatatgctcggtgtgaaaggttcggagagccatgatgatttcatcggatatctttctacaaataacatctacgatgtgtttatagacccggataactcttctctgagcgatgacaaaaaagccaatgtgagaaagagcctttcgaagtttaacgccctgctcaaaacaaaacgcttgggctattttggattggaagaaccgaagacaaaagacaatcgggtttcgcaggcctacaaaaagcgcgtgtatcacatgcttgcaatcgtcgggcaaatcaggcaatgtgtctttcacgacaaaagcggggcaaaacgcttcgacctgtactcttttattaataacatagatccggaatatagggatacacttgattacctggtcgaagaacgccttaaatccataaacaaagactttatagaagacaataaagtgaatatttctttgctgatcgacatgatgaagggctacgaagcggacgacataataaggttgtattatgactttatcgttcttaagtcccagaaaaatctggggttttcaattaaaaagcttagggaaaaaatgttggatgagtatggtttccggttcaaagataagcaatacgattcagtcagatccaaaatgtacaagctcatggactttcttctgttctgtaattactaccgcaatgacatagcagctggtgaaagcctcgtgaggaagttgagattttccatgaccgacgatgagaaagagggtatttatgcagatgaggcagccaagctctggggaaagtttagaaatgacttcgagaatatcgccgaccatatgaacggggatgtcatcaaagagctgggaaaggcggatatggacttcgacgagaaaatactggattctgaaaaaaaaaatgcgagcgacctcctttacttctccaagatgatctatatgcttacttatttcctcgatggaaaggagataaacgacctgctgactacacttatatcgaaattcgacaatatcaaagaattcctcaaaataatgaagtcttcagcggttgatgtggagtgcgaattgaccgctggttacaagctgtttaacgattcgcagcggatcaccaatgaattgtttattgtcaaaaatatcgcctctatgagaaaacctgctgcatctgcgaagctcaccatgttcagggatgcactcaccatattgggcattgacgataagatcaccgatgacaggatttctggtatattgaagcttaaggaaaagggtaagggaatacatggtctcagaaactttatcactaacaacgtcatcgaatcctcgcgctttgtctacctgataaaatatgctaacgctcagaagatccgggaggttgcgaagaatgaaaaagtcgtcatgttcgttttgggggggattcccgatacgcaaattgagaggtattataagtcgtgtgtcgaatttcctgacatgaactcatcacttggcgtcaaacgctccgaattggcacggatgatcaaaaacatttcattcgacgacttcaaaaacgtcaaacagcaagctaagggccgcgagaacgttgcaaaggaaagggcaaaggcagtcataggactttaccttactgttatgtacctgctcgttaagaacctggtcaatgtcaacgcgcggtatgtcattgccattcattgcttggaacgggacttcggactttacaaagagattatccctgaactggcgtcgaagaacttgaaaaacgactaccggattctgagccagacgctctgtgaactttgcgacaagagccctaacctttttcttaaaaaaaacgagcggcttaggaaatgtgtggaggtggatattaacaacgctgatagctcgatgactcggaagtaccggaattgtattgcgcacctgacagtcgttcgggaactgaaggaatacataggtgatatatgcacggttgactcatacttttccatatatcattacgttatgcaaagatgcataacgaaaagagagaacgatactaaacaggaggaaaagataaagtatgaagatgacttgcttaaaaatcacggctacactaaagactttgttaaagcactcaatagcccttttggctacaacatacctagattcaaaaatctgtcaattgagcagctttttgacagaaacgaatatctgacagaaaag

SEQ ID NO: 21 - DNA, 2754 bp, synthetic construct
  misc_feature 1..2754: note = Synthetic: CasM DNA sequence from Ruminococcus bicirculans, modified for expression in Zea mays cells
  source 1..2754: mol_type = other DNA
atggcaaaaaagaataagatgaagccgcgggagcttcgcgaggcccagaaaaaggcgcggcagcttaaagcggctgaaattaataataatgctgtcccagcgatagccgcaatgcctgcggctgaagcggcggctcccgcggccgagaagaaaaaatcatctgttaaagccgccgggatgaaaagcatcctcgtgtcggagaataagatgtacattacgtcgttcggtaaggggaattcggcggtccttgaatacgaagttgataacaatgattataacaaaactcagctttccagcaaagacaattcgaatattgagctctgtgacgtcgggaaagtgaatataacgttttcttcccggaggggtttcgagagcggtgtggaaatcaatacaagcaatccaactcatcggtcgggcgagtcctcctctgtgcggggcgacatgttggggcttaagtcggaacttgaaaagcggttttttggaaaaaatttcgacgacaatatacacatccaacttatctacaacatactggacatagagaagattttggcagtgtatgtgaccaatatagtctacgccctcaacaacatgctgggtgagggcgacgaatcaaattacgactttatgggttatctgtcaacttttaacacatataaggtctttacaaacccgaatgggtctacattgtccgacgataagaaagaaaatataaggaagtccctttctaaattcaacgcgctccttaaaacaaagagattgggctacttcggccttgaagagcccaagacaaaggacactcgggcctcagaagcttataagaagagagtctaccacatgctcgccatagtgggccaaattaggcagtgcgtcttccacgacaagtctggtgcaaagagatttgatctgtactcattcattaataatatcgatccagagtaccgcgagacattggattatcttgtcgacgaaaggttcgattctatcaataagggttttatccaaggtaataaagtcaacatctccctcctgattgacatgatgaaaggctatgaagccgatgacatcattaggctgtactacgactttatagttctcaaatcacagaaaaacctggggttctctattaagaagcttagagagaaaatgttggacgaatacggtttccgcttcaaagataagcaatacgactcagtgaggtctaaaatgtacaaactcatggattttcttctgttctgtaactactatcggaatgatatcgcagccggtgaatctctcgtcagaaaactcaggttttcgatgacggacgacgagaaagaagggatatacgcggacgaagccgctaagttgtggggaaaatttcgcaacgattttgaaaatatagctgatcacatgaatggggacgttataaaagagcttggaaaagccgacatggattttgacgagaagatattggactctgagaagaagaatgcgtcagacttgctttatttttcaaaaatgatatatatgctcacgtacttcttggacgggaaggagataaacgatctgttgacgacgctgattagcaaattcgacaatatcaaagagttcctgaaaataatgaagagctcagctgtcgatgtcgagtgtgaactgacggctggctacaaattgtttaacgattcgcaacgcattacgaatgagctgtttatagtgaaaaacattgcatctatgcgcaaaccagctgccagcgctaagcttacaatgtttcgggacgctctgacgattttgggcatcgacgataaaattactgacgataggatcagcgagatactgaaattgaaagagaaagggaaagggattcacggcctcagaaactttattactaataatgtcatcgaatcgtcaaggtttgtgtacttgattaaatatgcaaatgcacaaaagattcgggaagtcgctaaaaatgaaaaggttgttatgtttgtcctcggggggatacccgatacccaaattgagcggtattacaagagctgcgtggagtttccagacatgaactcgtctctgggggtgaaacggtccgaactcgctcgcatgattaaaaacatatccttcgacgactttaagaacgtgaagcaacaagctaaggggcgcgagaacgtcgcgaaagaaagggccaaagcggttatcggtctgtaccttacggtcatgtacttgttggtgaaaaaccttgtgaatgtgaacgctcggtacgtgatcgcgatccactgtctggagcgcgattttgggctgtataaagagatcatcccggagctggcttccaaaaacctgaaaaatgactaccgcatactgtcccagacactttgcgagttgtgcgacaagagcccgaatctgtttctgaaaaaaaacgagcgcctgcggaagtgcgttgaggttgatataaacaacgccgactcctcaatgacgagaaagtacagaaattgcatagctcatttgaccgtcgtcagggagctcaaagaatacataggggacatttgcactgtggactcgtatttttccatctaccactacgtgatgcaaaggtgtatcactaagcgggaaaacgataccaaacaagaggagaagatcaagtacgaggatgaccttttgaaaaatcacggttatacgaaggacttcgtgaaggcattgaactctccgttcggttataatatccctaggttcaagaatttgtccatagaacagctcttcgatcgcaatgagtatcttacagaaaaa

SEQ ID NO: 22 - DNA, 2766 bp, synthetic construct
  misc_feature 1..2766: note = Synthetic: CasM DNA sequence from Ruminococcus sp., isolate 2789STDY5608892, modified for expression in Zea mays cells
  source 1..2766: mol_type = other DNA
atggcaaagaagaacaaaatgaagccacgcgaactgagagaagctcaaaagaaggcgagacagcttaaagctgcggagatcaataataacgcagctccggccattgccgcaatgcccgccgctgaagtgatagctccagttgcggagaagaagaaatcttcagttaaagcagctggaatgaaatccattctcgtctcggagaataaaatgtatattacgtccttcggaaaaggaaattccgcggttctcgagtatgaggtggacaacaacgactacaacaagactcaactgtcgagcaaagacaactcaaatattgaactcggggacgttaacgaagtcaatataacattttcctcaaagcatggattcggcagcggtgtcgaaattaatacttcaaatccgacacataggtctggagaatcgtcgcctgtcaggggcgatatgcttggtttgaagtccgaactggagaagcggttctttgggaagacttttgacgataacattcatatacaactgatctacaacatactggatatcgagaaaatcctcgcagtgtatgtcactaatattgtttacgccttgaacaacatgctgggcattaaagactctgaatcatatgatgacttcatggggtatctcagcgccaggaacacatatgaagtgtttacgcacccggacaagtctaatctgtctgataaggtcaagggtaatattaagaagtcactcagcaagttcaacgacttgcttaagacgaagcgcctcggctactttgggcttgaggaaccaaaaacgaaggacaccagagcctctgaggcttataagaaaagagtgtatcatatgctcgcgatagtcggtcaaattagacagtgtgttttccacgataaatctggagcaaagaggttcgacctttactcatttataaacaatatcgaccctgaatatagagacacgctggattaccttgtggaggagcggctgaagtcgattaataaggactttatagaaggcaataaagtcaatatctctctcctcatagacatgatgaaaggttatgaagccgacgacataataaggctttattacgattttatcgttcttaagtcacagaaaaatttgggtttttcgatcaaaaaacttcgggaaaagatgttggaagaatacgggttcagattcaaagacaagcagtacgatagcgtgaggtcaaaaatgtacaagctgatggacttcctgctgttttgcaattactacagaaatgatgtcgccgccggggaggcgttggttcgcaagcttcgcttttcaatgacagatgatgaaaaagaggggatttatgcggatgaggccgccaagctctggggcaaatttaggaatgattttgaaaacattgctgatcatatgaatggcgatgtgattaaggaactgggcaaagcagacatggattttgatgaaaagatcctcgactcagaaaagaagaatgccagcgatttgttgtatttctcaaagatgatctacatgctgacgtattttttggacggtaaagagataaacgatctgctcacgacgttgatttctaaattcgacaatattaaggagtttcttaagattatgaagtcttcggcagttgacgttgaatgcgaactgactgctggctacaaactcttcaacgactcacaacgcatcaccaatgaactttttatcgttaaaaatatagccagcatgcggaagccggcagcttctgccaagctcaccatgtttcgcgatgctttgaccatcttgggcattgatgacaatattacagatgatcggatatctgagatactcaaacttaaggagaaaggcaagggcatacatggccttcggaatttcattactaataacgtgatagaaagcagccgctttgtttacctcattaaatacgcaaatgcccaaaaaataagggaagttgctaaaaacgaaaaagtggtgatgttcgtgcttggaggaatacctgacacacaaatcgagcgctattacaagtcgtgtgtcgaattccccgatatgaattcttccttggaggctaaacggtcagagctcgccagaatgatcaagaacatttcctttgatgacttcaaaaatgtgaaacagcaagctaagggtcgcgaaaacgtcgctaaagagagggccaaggctgttatcggcctctatcttacggtgatgtatttgttggtgaagaacctcgttaatgtcaacgccaggtatgttatagcaatacattgcctcgaacgggattttggtctttacaaagagattatcccagaattggcgtccaagaacctcaagaacgactatcgcatattgtctcagacgctttgtgaattgtgcgatgaccgcaatgagtcttccaacttgttcttgaaaaagaataagcggttgcgcaagtgcgttgaagtggacataaataacgccgactcttcaatgactcgcaagtacagaaattgtatagcgcacctcactgtcgtgcgggaattgaaagaatacatcggagacataaggaccgtcgatagctattttagcatttaccactatgtcatgcaaaggtgtataactaaacgcggtgatgataccaaacaggaagaaaagatcaaatacgaagacgatctgctcaagaatcatggctacaccaaagatttcgttaaagcattgaatagccctttcgggtataatattcccagatttaaaaacctcagcattgaacaactgttcgaccgcaacgaatacctcacggaaaag

SEQ ID NO: 23 - DNA, 2766 bp, synthetic construct
  misc_feature 1..2766: note = Synthetic: CasM DNA sequence from Ruminococcus sp. CAG:57, modified for expression in Zea mays cells
  source 1..2766: mol_type = other DNA
atggcgaagaagaacaaaatgaaaccacgcgaactcagagaggcacaaaagaaagcccggcagttgaaggccgccgagataaacaacaacgcggcaccggcaattgcggcaatgccagctgcggaggtcatcgctcccgtcgccgagaagaagaagagctcggtcaaggcagccgggatgaaatctattctggtgtcagagaataagatgtacattacgtctttcggcaagggaaatagcgcagtcttggagtatgaagttgacaacaacgactataacaaaacacaactttctagcaaagacaactcgaatatagaattgggagatgtcaatgaggtcaacataacctttagctccaagcatggctttggctcgggtgtggaaattaacacgtccaatcctacccatcggtcgggcgagtcgtcgccagttaggggggacatgctgggtctcaagagcgagttggagaaaagatttttcggtaagaccttcgatgataacattcatatccaacttatctataacatcttggacatagaaaaaatacttgcagtgtacgtcactaatatcgtttatgccttgaataatatgttgggaattaaggactctgaatcctatgacgattttatgggctatctgagcgctcggaatacctacgaagtgtttactcatccagataaaagcaaccttagcgataaggtcaagggcaacataaaaaagtccctgtcaaagtttaacgatcttctcaaaaccaaacggctgggctactttggactcgaggagcctaagacgaaagacacgcgggcatctgaggcatacaagaaaagggtttatcatatgctggcaatagtcggtcaaatcaggcagtgcgtctttcacgacaagagcggagcgaagcggtttgacctttattctttcatcaataacatcgatccggaataccgcgacacattggattacctggtcgaggaaaggttgaagtccataaacaaggacttcatcgagggaaacaaggttaacatttcacttctgattgacatgatgaaaggctacgaggctgacgatatcataagactttattatgactttatcgtgctgaaatcgcagaaaaatttgggattttctatcaaaaagctcagagagaagatgcttgaggagtatggatttagatttaaggacaagcagtacgattctgtgcgctctaaaatgtacaagctcatggattttctcctcttttgcaattactacaggaacgatgttgccgcaggcgaggctcttgtccggaagctccgcttctccatgacggacgacgaaaaggaaggcatatacgcggatgaggcagcgaaattgtggggtaagttcaggaatgattttgaaaatatagctgatcacatgaacggtgacgtcatcaaggagctggggaaagccgatatggattttgatgagaaaatcctggattcggaaaagaaaaatgcgagcgacttgctctactttagcaaaatgatttatatgttgacctatttcctcgatggcaaagagatcaacgatttgcttacgactctgataagcaaattcgataatataaaagagtttttgaaaataatgaagtcctcagcggttgatgttgaatgcgaactgacagccggctataagcttttcaatgattcacagaggattaccaacgaactttttatagtgaaaaacatcgcctcaatgaggaaacccgccgcgagcgcgaagttgacaatgtttagggacgctctgacgattttgggaatcgacgataatatcactgacgacaggatttcggagatcctcaaattgaaagagaagggcaaagggatccacgggttgagaaattttataaccaataacgttatagaatcatcgaggtttgtgtatctgatcaaatacgcgaatgctcaaaagatcagggaagtggcaaaggacgagaaggttgtcatgttcgtcctgggtgggatccctgacacccagatagaaagatactataagtcctgcgtggaattccctgatatgaattcttccctcgaggctaaaagatctgagttggcacggatgatcaagaatatttcgtttgacgatttcaaaaacgtgaagcaacaagctaaagggcgggaaaacgttgccaaggaacgggctaaagctgtcattggcctttacctcactgtgatgtatttgctcgttaagaatctcgtgaacgttaacgcaagatacgtgatcgctatccactgcttggagcgcgatttcggactgtacaaggagattataccagagcttgcttccaagaatcttaagaatgactatcgcatattgtcccaaactctttgcgagttgtgcgacgatcggaacgagtcttccaatctgttccttaagaaaaataaaaggctgcggaaatgcgtcgaagtcgacattaacaatgcggattcttctatgacgagaaagtaccgcaactgcatcgcccatctcacggttgtcagggagctcaaggaatacataggagacattagaacggtggactcatatttttcaatataccattatgttatgcaaaggtgtattacaaaacggggggatgacacaaaacaagaggaaaagattaaatatgaagacgatttgcttaagaaccatggttacacgaaagatttcgttaaagcgcttaattcgccatttggttataatattccgagattcaaaaatttgagcatagagcagcttttcgatagaaatgaatacttgaccgagaag

SEQ ID NO: 24 - DNA, 2799 bp, synthetic construct
  misc_feature 1..2799: note = Synthetic: CasM DNA sequence from Ruminococcus flavefaciens FD-1, modified for expression in Zea mays cells
  source 1..2799: mol_type = other DNA
atgaaaaaaaagatgagcttgcgggaaaaaagagaggcagaaaagcaggccaagaaagctgcatacagcgctgcgtctaagaacactgattccaaaccagcggagaaaaaagcggagactccaaaacctgccgaaattatatctgataactcacgcaataagacggcggtcaaggcagcgggactcaagtcgacgatcatatcaggcgataaattgtatatgaccagctttggcaagggcaatgcagctgtgatagaacaaaagatagacatcaatgactattcttttagcgcaatgaaggacaccccaagccttgaagtcgacaaggcagaatctaaggaaatatccttttcgtcccatcatccctttgtgaagaacgacaagttgacgacatataatcctctttacggtgggaaggataacccagagaagccggttgggcgcgatatgttggggttgaaagataaacttgaggaacggtactttggttgtacattcaatgacaacctccacattcagatcatttacaatattttggatattgagaagatcctcgctgttcattccgcaaatattacgacagctcttgatcatatggtggatgaggacgatgagaaataccttaactctgactatatcggctacatgaacacgatcaacacctacgacgtcttcatggatccctctaagaattcctctttgtcgccaaaagacaggaaaaacatcgacaattcgagggcgaagtttgagaagctcctctctacaaaaaggttggggtactttgggttcgactatgacgcgaacgggaaagacaaaaagaagaatgaggaaattaaaaagcggctttaccacttgacggcatttgcaggccagctgaggcagtggtccttccactcagcaggaaactatcccagaacctggttgtataaattggactccctggataaagagtatctggacacgctcgaccactatttcgataagaggtttaatgatataaatgacgattttgtcactaaaaacgcaacgaacctgtatatactgaaggaggttttccctgaggctaactttaaagatattgcggacttgtattatgactttattgtcatcaagtcacacaagaacatgggattctcgatcaagaaacttcgggaaaaaatgctcgagtgcgatggagctgaccgcatcaaagaacaggatatggattctgtccgctccaagctctacaagctcattgatttttgcatattcaagtattaccatgagttcccagagctcagcgagaagaacgtcgacatcctgagggctgccgtgagcgatactaagaaggacaatctctactcagatgaagctgctcggttgtggtcaattttcaaggaaaaatttctcggattttgtgacaaaattgttgtctgggtgaccggagagcatgagaaagatatcacgtctgtcattgataaagacgcctacaggaacagaagcaatgtctcgtatttttcaaagctcatgtacgcaatgtgtttttttcttgatgggaaggagataaacgaccttctgactaccttgattaacaagtttgacaatatcgccaaccagattaagacagcaaaggaattggggatcaacacggcgttcgttaaaaactatgacttcttcaaccattctgagaaatatgtcgacgaattgaacatagtgaaaaatatcgctcggatgaaaaaaccctcttcaaacgcgaaaaaagctatgtaccatgacgcccttactattcttggcattcctgaagatatggacgaaaaggctttggatgaagaactcgaccttatactcgaaaaaaagaccgatcccgtcacaggtaaaccgctgaagggtaagaatcctttgcgcaattttatagctaacaacgttatagagaactctcggttcatctaccttataaaattctgtaatccggaaaacgtgagaaaaattgtgaataacactaaggtgacagagttcgtgctgaaacgcataccagatgcccaaattgagaggtattacaaatcttgtacggatagcgagatgaaccctccgactgaaaaaaaaattaccgagttggctggtaaacttaaagacatgaacttcggcaacttccggaatgtccggcagtctgcaaaagagaatatggagaaagagaggtttaaagccgtcattggactgtaccttaccgttgtgtacagggtggttaagaatctcgtcgacgtgaactcaagatacattatggcattccattcactcgagagagactcccaattgtataacgtctcagtcgacaacgattatctggcactgaccgatacactggtcaaagagggtgacaactcacgctcacggtacttggccgggaataaaagattgcgggattgtgtcaaacaggatattgataacgcaaaaaagtggtttgttagcgataaatataattccataaccaagtataggaacaatgtggcgcacctgaccgccgttcggaactgtgccgaatttataggcgacataacgaagattgactcctacttcgccctctaccactaccttatccagcggcaactcgccaaaggtctcgatcatgagaggtcaggttttgaccgcaattatccacagtacgcaccactgttcaagtggcatacttatgtgaaagatgttgtgaaagcgctgaatgcacctttcggttataatattccaaggttcaagaatctttccattgacgcactcttcgaccggaatgagatcaagaagaatgatggagaaaagaaatctgacgac

SEQ ID NO: 25 - DNA, 2832 bp, synthetic construct
  misc_feature 1..2832: note = Synthetic: CasM DNA sequence from Ruminococcus albus strain KH2T6, modified for expression in Zea mays cells
  source 1..2832: mol_type = other DNA
atggccaaaaaatctaaaggcatgtccctgagggaaaaacgcgagctggagaagcaaaagcggatccagaaagctgcagtgaactctgtcaacgacactcccgaaaagaccgaggaagcaaacgttgtttctgtcaatgtgagaacgtctgcggaaaacaagcacagcaagaagagcgctgctaaagctcttggacttaaatcggggttggttattggggacgaattgtacctcacatcatttggcagaggaaatgaggcgaaactcgaaaagaaaataagcggggataccgtggaaaaattgggcattggtgctttcgaagtggcggaaagggatgagtctacactcacacttgaatctgggcgcattaaagataaaactgccagaccgaaagatcccagacatattacagtggacacacaagggaagtttaaggaagatatgctcggaatacgctctgtgcttgagaaaaagatatttggtaagaccttcgatgacaacatccatgtccaacttgcgtacaatatcctcgatgtcgagaagatcatggcacagtacgtctctgacattgtttacatgctccacaacaccgataagacggaacgcaatgacaacctgatggggtatatgtccatcaggaatacttacaaaaccttttgtgatacttccaaccttccggacgatacaaaacaaaaggtcgagaatcaaaaacgggaattcgacaagataattaagtctgggcgcttgggatactttggcgaggcatttatggtcaactccggcaactctacaaaattgcggcctgagaaagaaatctatcatattttcgctctcatggcctcacttaggcagtcctacttccacgggtatgtgaaggacacggactaccaaggaacaacgtgggcgtacacattggaggacaagttgaagggcccgtcacacgagttcagagaaacaattgataagatatttgatgaaggattctctaagatatcaaaagacttcgggaaaatgaacaaagttaatctgcaaattctggagcagatgataggcgagctgtacggttctattgagcgccagaatctcacatgtgattactacgacttcatccaattgaagaaacataagtacttggggttctctataaagcggttgagagaaacgatgttggaaacgacaccggcggaatgttacaaggcagaatgctacaatagcgagcggcagaagctttacaaacttatagattttctgatctatgatttgtactataaccgcaagccggcgcggatcgaggaaattgtcgataagcttagggagtctgtgaacgatgaggagaaagaatcgatttatagcgtcgaagctaagtatgtctatgagtccctctccaaagtgctggataagtccctcaagaactccgtttccggggagaccatcaaagatctccagaaaaggtatgatgacgaaactgctaatagaatatgggacatctcgcaacactcgatttctgggaacgtcaactgtttctgcaaattgatctacataatgaccctcatgctggacgggaaagaaattaacgacctccttacaacgctcgtgaacaaattcgataatattgcttcattcattgatgttatggacgaattgggtttggaacactcatttactgataattataaaatgtttgcagattcaaaggctatctgccttgatcttcaatttattaattcgtttgcacggatgagcaaaatcgacgatgaaaaatctaagcgccaattgtttagggacgctctggttatcctcgacataggcaataaggacgagacctggataaataactacttggactccgatattttcaaattggataaagagggaaataagttgaagggcgcaaggcatgactttcggaactttattgctaacaacgtgattaagtcgtcacggtttaaataccttgttaaatactcgtcagcagatggtatgataaaactgaaaactaacgaaaagcttataggctttgtcctggacaagctccctgagacacagatagatagatactacgaatcgtgtggacttgataatgctgttgtcgacaaaaaagtcaggatcgagaagctgtcagggcttatacgcgacatgaaatttgatgatttctccggtgtcaaaacatcaaataaggcgggcgataacgataagcaagacaaagcaaagtatcaggcaattatcagcttgtaccttatggttctgtaccaaattgtgaaaaacatgatctatgtcaattcacggtacgtgatcgcgttccattgccttgagagggatttcggcatgtacggaaaagacttcgggaaatattaccagggatgtagaaaattgactgaccatttcatagaagagaaatatatgaaggaagggaaacttggttgcaataagaaggtgggaaggtatctcaaaaataatatttcatgctgtacggatggtctgatcaatacctataggaaccaagtggaccatttcgctgttgttcggaagatagggaattatgcagcatatatcaaatctatcggctcatggtttgaactgtatcactacgtcattcagaggatcgtgtttgatgagtacagatttgcactgaataatacggagagcaactacaagaattcaatcattaagcaccatacttattgcaaagacatggtgaaggctctcaatacgccttttgggtatgacctccccagatataagaatctctccatcggggatcttttcgatagaaacaattatcttaataagacgaaggaatcgatagatgctaattccagcattgactcacag

SEQ ID NO: 26 - DNA, 2901 bp, synthetic construct
  misc_feature 1..2901: note = Synthetic: CasM DNA sequence from Ruminococcus flavefaciens strain XPD3002, modified for expression in Zea mays cells
  source 1..2901: mol_type = other DNA
atgatcgagaagaaaaagtctttcgcaaaaggaatgggagtcaagtctacattggtttctggttcgaaggtttatatgacgacgttcgccgagggctctgacgcgcgcttggagaagatagtggagggggattcaatacggtctgtgaacgaaggcgaagctttttcggccgagatggcggacaagaatgcagggtataaaattgggaatgcaaagttttcgcaccccaaaggttacgcagtcgttgcgaataacccgctctatactggtccagtccagcaagatatgctcgggctgaaagagaccctcgagaaacgctattttggggagagcgcggatgggaatgacaatatatgtatccaagttatacataatattctggatatcgaaaagatccttgctgaatacattaccaacgctgcttatgcggtcaacaatatttcgggacttgataaagatataatcggcttcggtaaattcagcactgtctatacatacgatgagttcaaggatccagagcatcatagagcggcgttcaataataacgacaaactgattaacgcaattaaagcgcaatatgacgagttcgacaattttctcgacaacccacggcttggctactttggccaggcatttttctcgaaggagggtaggaactacataatcaattatggcaatgaatgctatgacatacttgctctgctttcaggtctcagacattgggtcgttcacaataacgaagaagaatctcggatctctcggacttggctctataaccttgacaagaaccttgataacgagtacatctctacgctgaactacctttacgacagaatcactaacgagctcaccaattcattctccaaaaattctgccgcaaacgtcaactacatcgcggaaacccttgggatcaacccagcagagtttgctgaacagtattttcgcttctcaatcatgaaagaacagaaaaatctgggcttcaatataacgaaactgcgcgaggtcatgttggatagaaaagatatgtccgaaatcaggaaaaaccataaagtcttcgactcaataaggaccaaagtgtataccatgatggattttgtcatctaccgctattacatagaggaggatgcaaaagtcgctgccgctaacaagagccttccagataatgaaaagtctctgtcggaaaaggatatatttgtgattaatctccggggaagctttaacgacgatcaaaaggatgccctgtactacgatgaggcaaacagaatttggaggaagctggaaaacattatgcataacattaaggagttccgcgggaataaaacgagggaatataagaagaaagatgctccgaggttgcctcggattcttcctgctggtagggatgtttcggcattctcgaagctgatgtacgcactcaccatgttccttgacggtaaagagatcaacgatctcttgacaacgcttattaataagtttgataatatacagtctttccttaaggttatgccccttattggagttaatgctaaattcgtggaagagtatgctttcttcaaggacagcgcgaaaattgctgacgaactgcgccttatcaagtccttcgcgcggatgggagagcctatagctgacgctcgcagggcaatgtatatcgacgccatccgcatccttggcaccaatctgagctatgatgagcttaaagccctcgccgacaccttcagcctggacgaaaacggcaacaaactcaagaagggcaagcacggcatgcgcaatttcattatcaataacgtgatctcgaataagagatttcactatctgatacggtatggcgacccggcccacctccatgagattgcgaaaaacgaagctgttgtgaaatttgtgcttggtagaattgcggacatacaaaaaaaacaaggccaaaatggcaaaaatcaaattgacagatattacgaaacatgcattggaaaggataagggaaagtctgtgagcgagaaggttgatgcgttgaccaaaataatcacaggaatgaattacgatcagttcgataaaaagaggtcagtgatagaagacacggggcgggaaaacgctgaacgcgaaaaatttaagaaaataatttcgctctatcttacggtcatttatcacatcttgaagaatatagtcaatatcaacgctagatacgtgattggtttccattgtgtggaaagagacgctcaactgtacaaggaaaagggttatgatataaacctcaagaagctggaggaaaagggttttagctcggtgactaaattgtgcgctggaatcgatgaaaccgcgccagataaaaggaaggatgttgagaaggagatggccgagagagcgaaggaatctatcgacagcctggaaagcgcgaatcccaaactttatgccaactacatcaagtactctgacgagaaaaaagcggaagagtttactagacaaatcaatcgggagaaagctaagaccgccctcaatgcttacttgcgcaataccaaatggaacgttatcattcgcgaagacctcttgcgcatagataataaaacatgtacattgtttagaaataaagcagtgcacctcgaggtcgccagatacgttcacgcatatataaatgacatcgctgaggtgaactcgtactttcagctgtaccattacattatgcaaaggatcataatgaacgaaaggtacgagaaatcgtcaggtaaagtttccgaatattttgacgcagtcaatgatgaaaagaagtacaacgaccggcttttgaagttgctttgtgtgcctttcgggtactgtatccctcggttcaaaaacctgtccatagaggcattgtttgacaggaacgaggcagcaaagttcgacaaggaaaagaaaaaggtgtcgggtaactcg

SEQ ID NO: 27 - DNA, 2388 bp, synthetic construct
  misc_feature 1..2388: note = Synthetic: CasM DNA sequence from Ruminococcus sp., isolate 2789STDY5834894, modified for expression in Zea mays cells
  source 1..2388: mol_type = other DNA
atggaaattaatacgtccaatccgacacacagatcaggcgaatcttcctcagttagaggtgatatgttgggacttaaatccgaattggaaaagaggttttttggcaagacattcgatgataacattcacatacaacttatatataacatccttgatatagaaaagatacttgctgtgtatgtgacaaacatagtttatgcactgaacaacatgcttggcgtgaagggatcagaaagctacgatgatttcatggggtacctctccgctcagaacacctattacatattcacgcacccagataaatctaacctgtcggataaagttaaggggaatattaagaagtcgctttctaaatttaacgaccttcttaagacaaaaagactgggctactttgggcttgaggagccaaagacgaaagacaaacgggttagcgaggcatataaaaagagggtttatcatatgcttgccatagtgggccagatacgccagtccgtctttcatgataaatctaacgagttggacgagtatctttactctttcatcgacatcatcgactccgaatatagagacacgctcgactatcttgtcgacgaacggtttgattcgataaataagggttttgtccaaggcaacaaagtcaatatatcactcctcatagatatgatgaaaggatacgaagcagacgatataatcagactttattacgactttattgttcttaagagccagaaaaatcttggattctcaataaagaaactgagggagaaaatgttggacgagtatgggtttcggtttaaagataaacaatatgactcggtcaggtccaagatgtacaagcttatggactttcttttgttctgtaattactataggaatgacgttgttgccggggaggccttggttagaaaattgagattcagcatgaccgatgacgaaaaagaaggcatctatgcggatgaggcagagaagttgtgggggaaatttaggaatgactttgaaaacatagccgatcatatgaatggcgatgtcataaaggagttggggaaagctgacatggattttgacgaaaaaatcctggatagcgaaaaaaagaatgcttccgatctgttgtatttctctaagatgatctatatgctcacttactttctggacggtaaagagatcaacgaccttcttactacccttatttcaaagttcgataacattaaggaatttctgaaaataatgaaatcctcggctgtcgacgttgaatgcgaacttactgcagggtacaagctgtttaacgactcgcaaaggattactaatgaactgttcattgtcaagaacatagcgtccatgagaaagcctgcagcaagcgcaaagctgacgatgttccgcgatgctctcaccattctgggaattgatgacaagattaccgatgaccgcatttcggagatccttaagcttaaggaaaaggggaaggggattcacggactgagaaattttatcaccaataacgtgatcgaatcgtctaggtttgtctatttgataaagtatgccaatgcgcaaaaaattcgcgaagtcgccaagaatgagaaggtcgttatgttcgtgctcggaggaattcccgatacacagattgaacggtactataaatcctgtgtggaattcccggatatgaactcatccctcgaggccaaatgctctgagcttgcgaggatgatcaagaatatctcctttgatgattttaaaaacgtgaagcagcaggcgaagggccgggagaatgtggcgaaggagcgggctaaagctgtgatagggctttatcttactgttatgtaccttctcgtgaaaaacctggtgaatgtgaacgccaggtacgttatagcgatccattgtcttgagcgcgacttcggtttgtataaggagataattccagagctggcatcgaagaacctgaaaaacgattacagaattctgtcacaaactctctgtgaactctgcgatgaccgcgatgagtcaccgaatctcttcctcaaaaaaaacaagaggctgaggaaatgtgtggaagttgacatcaataacgcggattcgagcatgacacgcaagtaccggaattgtattgctcatctcacagtcgtccgcgagctcaaagagtatataggtgatatccggaccgttgattcttatttttctatctatcattacgttatgcagcggtgcattacaaaaagggaagatgataccaaacaagaagaaaaaataaagtatgaggatgacttgttgaaaaatcatggatatactaaagactttgtcaaggctctcaactcaccgttcggttacaacatacccagatttaaaaacttgtcaattgaacagttgtttgaccggaacgaatacctgacagaaaaa

SEQ ID NO: 28 - DNA, 2865 bp, Eubacterium siraeum
  misc_feature 1..2865: note = native CasM DNA sequence from Eubacterium siraeum
  source 1..2865: mol_type = genomic DNA
atgggtaagaaaatacacgcacgagatctcagagaacaaagaaagaccgatagaacggaaaaatttgcagatcagaacaaaaaacgtgaagcagagagggcagttccgaaaaaagacgcagccgtttctgtaaaatcagtttcttctgtttcatcaaaaaaagacaatgtaacaaaatctatggctaaagccgcaggcgtgaagtcggtttttgctgtaggaaatactgtttatatgacttcattcggcagaggaaacgatgctgtacttgagcagaaaatagtcgatacatcgcacgaaccgctgaatattgacgatcctgcatatcagttgaacgttgtcacaatgaacggttattcggttaccggtcacagaggtgaaacggtatctgccgtaacggataatccgctgcgccgttttaacggaagaaagaaagatgaaccggaacagtctgtgcctacggatatgctgtgcctgaaaccgactcttgaaaagaaattcttcggcaaagaattcgatgataatatacatatccagcttatttacaatattcttgacattgaaaaaatactggcggtttattcgaccaacgctatttacgcattgaataatatgagtgctgacgaaaatatcgaaaacagcgatttcttcatgaaacgtaccaccgatgaaacctttgacgattttgaaaagaaaaaggagagtacaaacagtcgagagaaagccgattttgacgcatttgaaaaattcatcggcaattacaggctggcttattttgccgatgcattttatgtaaataaaaagaatcccaaaggtaaagcaaaaaatgttctgcgtgaggataaagaactttactccgtgctcactctgatcggtaaactgcgtcattggtgtgttcacagtgaggagggcagagcagaattctggctgtataagctcgatgaacttaaagatgatttcaaaaatgtactcgacgttgtttataaccgtcctgttgaagaaataaacaaccgctttatagaaaacaataaggtaaacatacagatactgggctcggtatacaagaacaccgatattgccgaacttgtaaggtcatattacgaatttcttatcacaaagaagtataaaaatatgggcttttcaataaagaagctccgtgagagtatgctcgaaggtaaaggttacgccgataaagaatatgattctgtaaggaataagctgtatcagatgacggatttcatcttatacacaggatatatcaacgaagacagcgatagagccgacgatcttgtgaacactttgagaagttcgctcaaagaggatgataagacaaccgtatattgcaaggaagcggattatctgtggaaaaaataccgtgaatccataagagaggttgccgatgcgcttgatggcgataacattaaaaagctgagcaaatcgaatattgaaattcaggaagacaagctgagaaaatgttttatcagctatgccgacagcgtatcggaatttaccaagcttatttatctgctgacaagatttttaagcggtaaggagatcaacgatcttgtcacaacgctgataaacaagtttgacaatatcagaagcttccttgaaataatggacgagcttgggcttgacaggaccttcaccgccgagtacagcttctttgaaggcagtacaaagtatcttgccgagcttgtcgagcttaacagctttgtgaaatcgtgttcgtttgatataaacgcaaaaagaacaatgtatcgcgatgcgctggatattctcggcattgaatcggataagaccgaagaagatattgagaagatgatcgataatatccttcagatcgacgcaaacggtgataaaaagctcaagaaaaacaacggtctgagaaatttcattgcaagtaacgttatagattcaaaccgattcaagtaccttgtgcggtacggaaatccaaagaagattcgtgaaacggcaaaatgcaagcccgctgtaaggtttgtgctgaatgagatcccggacgcacagatcgaaagatattatgaggcttgttgcccaaaaaatacagctttatgctctgcaaataagagacgtgagaaactggctgatatgatagctgaaataaagtttgagaatttttcggatgccggcaattatcagaaagcaaatgtcacatcaagaacgtctgaagctgaaatcaagcggaagaatcaggctataatccgtctttatcttaccgttatgtacattatgctgaagaaccttgtaaatgtgaacgccagatacgttatcgctttccattgcgttgaaagggatacgaagctgtatgcggaaagcggtctggaagtcggtaatatagaaaaaaacaagacaaatcttactatggctgtaatgggagtcaagctcgaaaacggaatcataaaaacggaatttgacaagagctttgcagaaaatgccgcaaacagatatctcaggaatgcacgctggtacaagctgatactggataatttaaagaagtcggaaagagcggttgtcaatgagttcagaaatactgtctgccatctgaatgcgataaggaatatcaatatcaatatcaaggaaataaaagaggtcgagaactactttgctctgtaccactacctcattcagaaacatctcgaaaatcgttttgccgataaaaaagtagaaagagacaccggcgattttataagcaagctcgaagaacacaagacttactgcaaggactttgtaaaagcatattgtacgcctttcggatataaccttgtgagatataaaaaccttacgatagacgggctgtttgataagaattaccccggaaaagacgattctgatgaacagaaataa

SEQ ID NO: 29 - DNA, 2760 bp, Ruminococcus sp.
  misc_feature 1..2760: note = native CasM DNA sequence from Ruminococcus sp., isolate 2789STDY5834971
  source 1..2760: mol_type = genomic DNA
atggcaaaaaagaataaaatgaagcctagagagctgcgtgaggctcagaaaaaagccagacagctcaaagcggctgagataaataataacgctgctcctgcaatcgctgccatgcctgctgcagaggtcattgcacctgcggcagagaagaaaaaatcctccgtaaaggcggcaggaatgaagtctattcttgtcagcgaaaataaaatgtacataacctctttcggcaagggcaattctgctgtgcttgaatatgaggtggataataatgactacaaccaaactcagctttcttcaaaggacaacagcaatatccagcttggtggtgtaaacgaagtaaacatcactttttcaagcaagcatggctttgagagcggagtggaaataaacacttcaaaccctactcacagaagcggtgaaagctcgcctgtaagaggggatatgctggggcttaaatcggagcttgaaaagcgctttttcggcaaaacttttgatgataatatacatatccagcttatttacaacattctggatatcgaaaagatacttgcggtgtatgtaacgaatatcgtttatgcgctgaacaatatgctcggtgtaaagggttcagaaagtcatgacgattttattgggtatctttccacaaataatatttatgatgtttttattgaccctgataacagcagtttatctgatgataagaaagcgaatgtcagaaaaagccttagcaagttcaatgccctgctgaaaactaagcgccttggctatttcggtcttgaagagccaaagacgaaagataatagagtttcgcaagcttacaaaaagcgtgtttatcatatgcttgcaattgtgggtcagataagacagtgtgtttttcatgataaatcgggtgcaaaaagatttgacctttacagttttattaacaatattgatcccgaatacagagacactcttgactatcttgttgaggaacgcttaaagtccataaacaaggactttatcgaggacaacaaggtcaatatcagcttgcttattgatatgatgaaaggctatgaggctgatgatatcatacgcctttattacgatttcattgtgcttaaatctcagaaaaatctcggtttttctatcaaaaagcttcgtgagaaaatgctggacgaatacggcttcagatttaaggacaagcaatatgactctgtgcgctcaaagatgtacaagcttatggattttctgcttttctgcaactactacagaaatgacattgccgcaggcgaatctcttgtgcgcaaactgcgtttttcaatgaccgatgatgaaaaagaggggatatatgctgatgaagcggcaaagctttggggcaaattcaggaatgattttgaaaatatcgccgaccacatgaacggtgacgttatcaaggagcttggcaaggctgacatggattttgatgagaaaattcttgacagcgaaaagaagaatgcgtctgaccttttgtatttctccaaaatgatatatatgctcacatattttcttgacggcaaggagataaacgaccttcttacaacgcttatcagcaagtttgataacatcaaggagtttttgaagataatgaaaagctctgctgttgatgttgagtgtgaacttacggcgggctacaagctgttcaatgacagccagaggataaccaacgagctttttatcgtaaagaacattgcttccatgagaaagcctgcggcttcggcgaagcttacgatgttccgtgacgcactgactatactcggtatagacgacaagatcacggacgataggataagcgggattctaaaacttaaagaaaaaggcaagggcatacatggcctgagaaatttcataacaaacaatgttatcgagtcctctcggtttgtataccttatcaagtatgcgaacgctcagaagataagagaagtggctaagaatgagaaagttgtcatgtttgttcttgggggtatccctgacacgcagatagagcgttattacaagagttgtgtggaatttcctgacatgaacagttctttgggagtaaagcgcagtgagcttgcgagaatgataaagaacatcagctttgatgatttcaaaaatgtgaaacagcaggcaaagggcagagaaaacgtggctaaggagagggcaaaggctgttatcgggctttatcttacggtcatgtatctgctggtgaaaaatcttgtgaatgtcaatgcaaggtatgttattgcgatacactgccttgaacgtgattttgggctgtataaggagataattcctgagttggcttcaaagaacttgaaaaatgactacaggatactttcacagacgctttgtgaactttgtgataagtcgccgaatttgttcttgaaaaagaacgagcggctgcgcaagtgcgttgaagttgatatcaataatgcagacagcagcatgacaagaaaataccgcaactgtattgctcatcttactgtagttcgtgaactgaaagaatacataggagatatttgtacagtggattcttacttctccatttatcattatgttatgcagcgctgtatcacgaaaagggaaaatgacacaaagcaagaagagaaaataaagtatgaggacgatcttttaaaaaatcacggctatacgaaagactttgtaaaggctctcaactcgccgtttggatacaacattccgaggtttaaaaatctttcaattgagcagttgtttgacagaaatgaatatcttactgaaaagtag

SEQ ID NO: 30 - DNA, 2757 bp, Ruminococcus bicirculans
  misc_feature 1..2757: note = native CasM DNA sequence from Ruminococcus bicirculans
  source 1..2757: mol_type = genomic DNA
atggcaaaaaagaataaaatgaagcctagagagctgcgtgaggctcagaaaaaagccagacagctcaaagcggctgagataaataataacgctgttcctgcaatcgctgccatgcctgctgcagaggctgctgcacctgcggcagagaagaaaaaatcctccgtaaaggcggcaggaatgaagtctattcttgtcagtgaaaataaaatgtacataacctctttcggcaagggcaattctgcggtgcttgaatatgaggtggataataatgactacaacaaaactcagctttcctcaaaggacaacagtaatatcgagctctgtgatgtaggcaaagtaaacatcactttttcgagcagacgtggctttgagagcggtgtggagataaacacttcaaaccctactcacagaagcggtgaaagctcgtctgtaagaggggatatgctggggcttaaatcggagcttgaaaagcgctttttcggcaagaattttgatgataatatacatatccagcttatttacaacattctggatatcgaaaagatacttgcagtgtatgtgacgaatatcgtttatgcactgaacaatatgcttggggaaggcgatgagagcaattacgatttcatggggtatctttccacatttaacacttataaagtttttactaatcctaatggcagcactttatccgacgataagaaagagaatatcagaaaaagtcttagcaaattcaatgccctgctgaaaactaagcgtcttggctatttcggccttgaagagccaaagacaaaggatacaagagcttcggaagcatacaaaaagcgtgtttatcatatgcttgcaattgtggggcagataagacagtgtgtttttcatgataaatcgggtgcaaaaagatttgacctttacagttttattaacaatattgatcccgaatacagagaaacccttgactatcttgtagatgagagatttgattctataaataagggctttatccagggcaacaaggtcaatatcagcttgcttattgatatgatgaaaggctatgaggctgatgatatcatacgcctttattacgatttcattgtgcttaaatctcagaaaaatctcggtttttctatcaaaaagcttcgtgagaaaatgctggacgaatacggcttcagatttaaggacaagcaatatgactctgtgcgctcaaagatgtacaagcttatggattttctgcttttctgcaactactacagaaatgacattgccgcaggcgaatctcttgtgcgcaaactgcgtttttcaatgaccgatgatgaaaaagaggggatatatgctgatgaagcggcaaagctttggggcaaattcaggaatgattttgaaaatatcgccgaccacatgaacggtgacgttatcaaggagcttggcaaggctgacatggattttgatgagaaaattcttgacagcgaaaagaagaatgcgtctgaccttttgtatttctccaaaatgatatatatgctcacatattttcttgacggcaaggagataaacgaccttcttacaacgcttatcagcaagtttgataacatcaaggagtttttgaagataatgaaaagctctgctgttgatgttgagtgtgaacttacggcgggctacaagctgttcaatgacagccagaggataaccaacgagctttttatcgtaaagaacattgcttccatgagaaagcctgcggcttcggcgaagcttacgatgttccgtgacgcactgactatactcggtatagacgacaagatcacggacgataggataagcgagattctaaaacttaaagaaaaaggcaagggcatacatggcctgagaaatttcataacaaacaatgttatcgagtcctctcggtttgtataccttatcaagtatgcgaacgctcagaagataagagaagtggctaagaatgagaaagttgtcatgtttgttcttgggggtatccctgacacgcagatagagcgttattacaagagttgtgtggaatttcctgacatgaacagttctttgggagtaaagcgcagtgagcttgcgagaatgataaagaacatcagctttgatgatttcaaaaatgtgaaacagcaggcaaagggcagagaaaacgtggctaaggagagggcaaaggctgttatcgggctttatcttacggtcatgtatctgctggtgaaaaatcttgtgaatgtcaatgcaaggtatgttattgcgatacactgccttgaacgtgattttgggctgtataaggagataattcctgagttggcttcaaagaacttgaaaaatgactacaggatactttcacagacgctttgtgaactttgtgataagtcgccgaatttgttcttgaaaaagaacgagcggctgcgcaagtgcgttgaagttgatatcaataatgcagacagcagcatgacaagaaaataccgcaactgtattgctcatcttactgtagttcgtgaactgaaagaatacataggagatatttgtacagtggattcttacttctccatttatcattatgttatgcagcgctgtatcacgaaaagggaaaatgacacaaagcaagaagagaaaataaagtatgaggacgatcttttaaaaaatcacggctatacgaaagactttgtaaaggctctcaactcgccgtttggatacaacattccgaggtttaaaaatctttcaattgagcagttgtttgacagaaatgaatatcttactgaaaagtag

SEQ ID NO: 31 - DNA, 2769 bp, Ruminococcus sp.
  misc_feature 1..2769: note = native CasM DNA sequence from Ruminococcus sp., isolate 2789STDY5608892
  source 1..2769: mol_type = genomic DNA
atggcaaaaaagaataaaatgaagcctagagagctgcgtgaggctcagaaaaaagccagacagctcaaagcggctgagataaataataacgctgctcctgcgatcgctgccatgcctgctgcagaggtcattgcacctgtggcagagaagaaaaaatcctccgtaaaggcggcaggaatgaagtctattcttgtcagcgaaaataaaatgtacataacctctttcggcaagggcaattctgctgtgcttgaatatgaggtggacaataatgactacaacaaaactcagctttcttcaaaggacaacagcaatatcgagcttggtgatgtaaacgaggtaaacatcactttttcaagcaagcatggctttgggagcggagtggagataaatacttcaaaccctactcacagaagcggtgaaagctcgcctgtaagaggggatatgctggggcttaaatcggagcttgaaaagcgctttttcggcaaaacttttgatgataatatacatatccagcttatttacaacattctggatatcgaaaagatacttgcggtgtatgtaacgaatatcgtttatgcgctgaacaatatgcttggtataaaggattctgaaagttatgatgattttatggggtatctttctgcaagaaatacttatgaagtttttactcaccctgacaaaagcaatctttccgataaggtaaagggtaatatcaagaaaagccttagcaagtttaatgacttgctgaaaactaagcgccttggctatttcggccttgaagagccaaagacaaaagacacaagagcttcggaagcatacaaaaagcgtgtttatcatatgcttgcaattgtggggcagataagacagtgtgtttttcatgataaatcgggtgcaaaaagatttgacctttacagttttattaacaatattgatcccgaatacagagatactcttgactatcttgttgaggagcgtttaaagtccataaacaaggactttatcgagggtaacaaggtcaatatcagcctgcttattgatatgatgaaaggctatgaggctgatgatatcatacgcctttattacgatttcattgtgcttaaatctcagaaaaatctcggcttttctatcaaaaagcttcgtgagaaaatgctggaggaatacggtttcagatttaaggacaagcaatatgactctgtgcgctcaaagatgtacaagcttatggatttcctgcttttctgcaactactacagaaatgacgttgccgcaggcgaagctcttgtgcgtaaactgcgtttttcaatgaccgatgatgaaaaagaggggatatatgctgatgaagcggcaaagctttggggcaaattcaggaatgattttgaaaatatcgccgaccacatgaacggtgacgttatcaaggagcttggcaaggctgacatggattttgatgagaaaattcttgacagtgaaaagaagaatgcgtctgaccttttgtatttctccaaaatgatatatatgctcacatattttcttgacggcaaggagataaacgatcttcttacaacgcttatcagcaagtttgataacatcaaggagtttttgaagataatgaaaagctctgctgttgatgttgagtgtgagcttacggcgggctacaagctgttcaatgacagccagaggataaccaacgagctttttatcgtaaagaacattgcttccatgagaaagcctgcggcttcagcgaagcttacgatgttccgtgacgcactgactatactcggtatagacgacaatatcacggacgataggataagcgagattctaaaacttaaagaaaaaggcaagggcatacatggtctgagaaattttataacaaacaatgttatcgagtcctctcggtttgtataccttatcaagtatgcgaacgctcagaagataagagaagtggctaagaatgagaaagttgtcatgtttgttcttgggggtatccctgacacgcagatagagcgttattacaagagttgtgtggagtttcctgacatgaatagttctttggaagcaaagcgcagtgagcttgcgagaatgataaagaacatcagctttgatgatttcaaaaatgtgaaacagcaggcaaagggcagagaaaacgtggctaaggagagggcaaaggctgttatcgggctttatcttacggtcatgtatctgctggtgaaaaatcttgtgaatgtcaatgcaaggtatgttattgcgatacactgccttgaacgtgattttgggctgtataaggagataattcctgagttggcttcaaagaacttgaaaaatgactacaggatactttcacagacgctttgtgaactttgtgatgatcgtaatgagtcgtcgaatttgttcttgaaaaagaacaagcggctgcgcaagtgcgttgaagttgatatcaataatgcagacagcagcatgacaagaaaataccgcaactgtattgctcatcttactgtagttcgtgaactgaaagaatacataggagatattcgtacagtggattcttacttctccatttatcattatgttatgcagcgttgtatcacgaaaaggggagatgacacaaagcaagaagagaaaataaagtatgaggacgatcttttaaaaaatcacggctatacgaaagactttgtaaaggctctcaactcgccgtttggatacaacattccgaggtttaaaaatctttcaattgagcagttgtttgacagaaatgaatatcttactgaaaagtag

SEQ ID NO: 32 - DNA, 2769 bp, Ruminococcus sp.
  misc_feature 1..2769: note = native CasM DNA sequence from Ruminococcus sp. CAG:57
  source 1..2769: mol_type = genomic DNA
atggcaaaaaagaataaaatgaagcctagagagctgcgtgaggctcagaaaaaagccagacagctcaaagcggctgagataaataataacgctgctcctgcgatcgctgccatgcctgctgcagaggtcattgcacctgtggcagagaagaaaaaatcctccgtaaaggcggcaggaatgaagtctattcttgtcagcgaaaataaaatgtacataacctctttcggcaagggcaattctgctgtgcttgaatatgaggtggacaataatgactacaacaaaactcagctttcttcaaaggacaacagcaatatcgagcttggtgatgtaaacgaggtaaacatcactttttcaagcaagcatggctttgggagcggagtggagataaatacttcaaaccctactcacagaagcggtgaaagctcgcctgtaagaggggatatgctggggcttaaatcggagcttgaaaagcgctttttcggcaaaacttttgatgataatatacatatccagcttatttacaacattctggatatcgaaaagatacttgcggtgtatgtaacgaatatcgtttatgcgctgaacaatatgcttggtataaaggattctgaaagttatgatgattttatggggtatctttctgcaagaaatacttatgaagtttttactcaccctgacaaaagcaatctttccgataaggtaaagggtaatatcaagaaaagccttagcaagtttaatgacttgctgaaaactaagcgccttggctatttcggccttgaagagccaaagacaaaagacacaagagcttcggaagcatacaaaaagcgtgtttatcatatgcttgcaattgtggggcagataagacagtgtgtttttcatgataaatcgggtgcaaaaagatttgacctttacagttttattaacaatattgatcccgaatacagagatactcttgactatcttgttgaggagcgtttaaagtccataaacaaggactttatcgagggtaacaaggtcaatatcagcctgcttattgatatgatgaaaggctatgaggctgatgatatcatacgcctttattacgatttcattgtgcttaaatctcagaaaaatctcggcttttctatcaaaaagcttcgtgagaaaatgctggaggaatacggtttcagatttaaggacaagcaatatgactctgtgcgctcaaagatgtacaagcttatggatttcctgcttttctgcaactactacagaaatgacgttgccgcaggcgaagctcttgtgcgtaaactgcgtttttcaatgaccgatgatgaaaaagaggggatatatgctgatgaagcggcaaagctttggggcaaattcaggaatgattttgaaaatatcgccgaccacatgaacggtgacgttatcaaggagcttggcaaggctgacatggattttgatgagaaaattcttgacagtgaaaagaagaatgcgtctgaccttttgtatttctccaaaatgatatatatgctcacatattttcttgacggcaaggagataaacgatcttcttacaacgcttatcagcaagtttgataacatcaaggagtttttgaagataatgaaaagctctgctgttgatgttgagtgtgagcttacggcgggctacaagctgttcaatgacagccagaggataaccaacgagctttttatcgtaaagaacattgcttccatgagaaagcctgcggcttcagcgaagcttacgatgttccgtgacgcactgactatactcggtatagacgacaatatcacggacgataggataagcgagattctaaaacttaaagaaaaaggcaagggcatacatggtctgagaaattttataacaaacaatgttatcgagtcctctcggtttgtataccttatcaagtatgcgaacgctcagaagataagagaagtggctaaggatgagaaagttgtcatgtttgttcttgggggtatccctgacacgcagatagagcgttattacaagagttgtgtggagtttcctgacatgaatagttctttggaagcaaagcgcagtgagcttgcgagaatgataaagaacatcagctttgatgatttcaaaaatgtgaaacagcaggcaaagggcagagaaaacgtggctaaggagagggcaaaggctgttatcgggctttatcttacggtcatgtatctgctggtgaaaaatcttgtgaatgtcaatgcaaggtatgttattgcgatacactgccttgaacgtgattttgggctgtataaggagataattcctgagttggcttcaaagaacttgaaaaatgactacaggatactttcacagacgctttgtgaactttgtgatgatcgtaatgagtcgtcgaatttgttcttgaaaaagaacaagcggctgcgcaagtgcgttgaagttgatatcaataatgcagacagcagcatgacaagaaaataccgcaactgtattgctcatcttactgtagttcgtgaactgaaagaatacataggagatattcgtacagtggattcttacttctccatttatcattatgttatgcagcgttgtatcacgaaaaggggagatgacacaaagcaagaagagaaaataaagtatgaggacgatcttttaaaaaatcacggctatacgaaagactttgtaaaggctctcaactcgccgtttggatacaacattccgaggtttaaaaatctttcaattgagcagttgtttgacagaaatgaatatcttactgaaaagtag

SEQ ID NO: 33 - DNA, 2802 bp, Ruminococcus flavefaciens
  misc_feature 1..2802: note = native CasM DNA sequence from Ruminococcus flavefaciens FD-1
  source 1..2802: mol_type = genomic DNA
atgaaaaagaaaatgtctctccgtgaaaagcgtgaagccgagaaacaggctaaaaaagctgcatattcagcagcttcaaaaaatacagattctaagcctgcggaaaagaaagcagaaactccaaagcctgcggagattatttccgataattccagaaataagaccgctgtaaaggcggctggtctgaaatcaacaattatcagcggcgataagctgtatatgacatctttcggcaagggtaacgctgctgttattgagcagaaaatagatatcaatgattattctttttcagctatgaaagatactccgtcgcttgaagttgataaagcagaatcaaaagagatctctttttcaagtcaccatccttttgtaaagaatgataagctgacaacatataaccctttatacggcggcaaggataaccccgaaaagcctgtcggcagggatatgctcggcttaaaagataagcttgaagaacgctatttcggatgtacattcaatgataatcttcacatccagattatctataacatacttgacatcgagaagattttagctgttcattctgcaaatatcacaactgcgcttgaccacatggttgatgaagacgatgaaaaatatcttaacagcgattatatcggctacatgaataccataaatacatatgacgtgtttatggatccttcaaagaattcttcattaagccctaaagatagaaagaatattgacaacagccgtgcaaaatttgagaaactgctttcaactaagcgccttggctattttggatttgactatgatgcaaacggtaaggacaagaaaaagaacgaggaaataaaaaagcgtttatatcatctcacagcttttgcaggtcagctccgtcagtggagttttcatagtgctggcaattatccgagaacatggctttacaagctcgattcactggataaggaatatcttgatactcttgaccattacttcgataaacgttttaacgatataaacgatgatttcgtaactaagaatgctaccaatctctatattctgaaagaagtatttcccgaagcaaacttcaaggatattgccgatctttattacgatttcatagttataaagtcgcacaaaaatatgggattctccataaaaaagctgagggagaagatgcttgaatgtgatggtgcagacaggataaaagaacaggatatggactctgttcgctcaaagctgtataagctcatagacttttgcattttcaagtattatcacgaatttcctgaacttagtgaaaagaatgtggatatactcagagcggctgtatccgatacaaaaaaagataacctttattctgatgaggctgcacgtttatggagcatatttaaagaaaaattcctcggcttctgtgataagatagttgtatgggtaacaggagagcatgagaaagatatcacatccgttattgataaggatgcttacaggaacaggagcaatgtttcatatttctcaaagctgatgtatgcaatgtgctttttccttgacggaaaagagataaatgaccttctcactactcttatcaacaaattcgataatatcgctaaccagataaaaacagccaaagaacttggcattaatactgcttttgtaaagaattacgatttcttcaatcacagcgagaaatatgtcgatgaactgaacatcgtcaagaatattgcaagaatgaagaagccttcaagtaatgccaaaaaagctatgtatcatgatgcgcttactattctcggaatacctgaggatatggatgaaaaagctcttgatgaggaactggatttaattcttgaaaaaaagacagacccagtaactggcaagccactgaaaggtaagaatcctttacgtaattttatcgcaaacaatgtgatagagaattcaagattcatatatcttatcaagttctgcaatcctgagaatgtacgtaaaatcgtgaataatacaaaggtcactgagtttgtgttaaagcgtattcccgatgctcagatcgaacgctattataagtcgtgtacagattctgaaatgaatccgcctactgaaaagaagatcaccgaacttgctggtaagttaaaggatatgaactttggcaacttccgaaatgtgagacagtctgctaaagagaatatggagaaggagcgcttcaaagctgttatagggctttatctcacggtagtatatcgtgttgtcaagaatcttgttgatgtaaactcacgatatatcatggcttttcattcgcttgaacgtgattcacaactgtataacgtatctgttgataatgattatcttgcacttaccgatactcttgttaaggagggagataattccagaagcagatatcttgcaggcaacaagcgtctgagagattgtgtgaagcaggatatcgataatgcaaaaaagtggtttgttagtgataagtacaatagcataaccaagtacaggaataacgttgcccatcttaccgctgtacgtaactgcgctgaattcatcggagatataacgaagatagactcctattttgcattgtatcattatctcattcagagacagcttgcgaaaggtcttgaccatgagcgaagtggctttgacagaaactatccacagtatgcaccgctgtttaagtggcatacgtatgtaaaggatgttgtcaaggctctgaatgctccatttggctacaatatccctcgtttcaagaatctcagcatagatgcactttttgaccgcaacgaaataaagaagaatgacggcgagaaaaaatccgatgattga

SEQ ID NO: 34 - DNA, 2835 bp, Ruminococcus albus
  misc_feature 1..2835: note = native CasM DNA sequence from Ruminococcus albus strain KH2T6
  source 1..2835: mol_type = genomic DNA
atggcaaaaaaatcgaaaggtatgagccttagagaaaaacgtgaacttgaaaagcagaaaaggatacaaaaggcagctgtgaattcagttaatgatacacctgaaaaaacagaagaagcaaatgtcgtatctgtaaatgtcaggacatcggctgagaataagcatagtaaaaaatctgctgccaaagctttgggactgaaatccgggctggttatcggtgatgagctgtaccttacttcattcggcagaggtaacgaagcaaagcttgaaaagaagatatccggtgacactgtcgaaaaacttggcattggtgcttttgaagtcgccgaacgtgacgaatcaacgcttaccctcgaaagtggcaggataaaggacaagaccgccagacccaaagaccccagacatataaccgtcgatacacaaggtaaattcaaggaagatatgcttgggatacgcagtgtactggagaaaaagatatttggcaaaacatttgatgataatatccatgttcagcttgcgtacaatatcctggatgtcgaaaagataatggcacagtatgtcagcgatatcgtatatatgctgcataatactgataaaacagaaagaaacgataatcttatggggtatatgagcatcaggaatacctataagacattttgtgatacgtcaaatcttcccgatgatacaaaacaaaaagttgaaaatcagaagagagagtttgacaagatcataaaaagcggcagacttgggtatttcggcgaagcttttatggtaaacagcggcaatagtaccaagcttagacccgagaaagagatatatcatatctttgcgcttatggcgagcctgaggcagagttactttcacggatatgtaaaagataccgattatcagggaaccacatgggcatatactcttgaggacaagctgaaaggtccgagccatgagttcagggaaaccattgataagatatttgatgagggattcagcaagatcagcaaggactttggcaagatgaacaaggtcaaccttcagatacttgaacagatgattggtgaactgtatggcagtatagaacgacaaaacctcacttgcgattactatgacttcattcaactgaaaaagcataagtatcttggattttctataaagcgtcttagagagaccatgcttgaaacaacaccggctgaatgttataaagctgaatgctataacagcgagcgtcaaaagctgtataagctgatagatttcctgatatatgatctttactataaccgtaagcctgcacgcatcgaagaaatcgtggacaagctgagggaatctgtgaacgacgaagagaaagaatccatatattcagttgaggcgaagtatgtctatgaatcacttagcaaagttctggataaatcgctgaaaaacagtgtgtctggtgaaacgataaaggatctccaaaagagatatgatgacgaaacagcaaacaggatctgggatatctcacagcacagtataagtggaaatgtcaactgtttctgcaagctaatttatattatgaccctgatgcttgacggcaaggagataaatgatctgctgacaacgctggtaaacaagttcgataacatagcatcatttatagatgttatggacgaacttggcttggagcatagttttacagataactataaaatgtttgccgacagcaaggctatatgccttgatctgcagttcataaacagttttgcacgtatgtcaaagatcgatgatgagaagtcaaaaagacagcttttccgtgatgcgcttgtcatactggatatcggtaataaagatgagacttggataaataattatctggattctgatattttcaaactggacaaagaaggtaacaagttaaagggcgcaaggcatgatttcaggaactttatagccaataatgttataaagtcatcacgtttcaaatacctagtaaaatacagcagtgccgatggtatgataaagctgaaaacgaatgaaaagctgataggctttgttctggataagcttccagaaacgcagatagaccgctactatgaatcatgcggacttgacaatgcggtagtagataagaaagtcaggatagaaaagctatcggggcttatcagagatatgaagttcgatgatttcagcggtgtcaaaacctcaaacaaagcaggagataatgacaaacaggataaggcgaaatatcaggcgataataagcctgtacctcatggtgctgtatcagatagtcaagaacatgatatatgtcaactcacgttatgttatcgctttccattgtcttgaacgtgactttggtatgtatggaaaagattttggaaagtattatcaaggctgccgaaaacttacagatcattttattgaagaaaagtacatgaaagagggtaaacttggctgcaataaaaaagtcggcagatatctgaaaaataatatttcctgctgcactgatggactgataaatacctaccgtaatcaggttgatcactttgcagtggtaaggaagataggcaactatgcggcatatatcaagagtatcggttcgtggtttgaactttatcactatgtaatacagaggatagtttttgacgaatacagatttgcacttaacaacactgaaagcaactataagaacagcatcatcaagcaccatacctactgtaaggatatggtcaaggcactgaacacacctttcggttatgacctgccgagatacaagaatctttctatcggtgatctgtttgatcgcaataattatctgaataaaacaaaagagtcaatagatgcaaatagctctattgacagtcagtga

SEQ ID NO: 35 - DNA, 2904 bp, Ruminococcus flavefaciens
  misc_feature 1..2904: note = native CasM DNA sequence from Ruminococcus flavefaciens strain XPD3002
  source 1..2904: mol_type = genomic DNA
atgatcgaaaagaagaagtcatttgcaaagggcatgggagtaaaatcaacacttgtatccggttcaaaggtatacatgacgacgttcgcagaaggaagcgatgccagacttgaaaagatcgttgaaggcgattctatcagatctgtcaacgaaggagaagcgttctcagctgaaatggctgataagaatgcaggctacaagatcggtaacgcaaagttcagccacccaaagggctatgctgtagttgcaaacaaccccttatacaccggaccggtacagcaggatatgctcggtctgaaggaaacgcttgaaaagagatattttggagagtctgccgacggaaatgataatatctgtattcaggtcatccataatatcctcgatatcgaaaagatcctcgctgaatatataaccaatgctgcttatgcggtaaacaatatttccggtcttgataaggatatcatcggttttggtaagttcagtacggtctatacttatgatgagttcaaggatcctgaacatcacagagcagctttcaacaataacgataagttaattaatgccatcaaggcacagtatgatgaatttgacaatttccttgataatcctcgtctcggctactttggacaggcttttttcagtaaggaaggcagaaattacattatcaattacggcaacgagtgttatgatattcttgctttactcagcggattgcgtcactgggtagtacataataatgaggaagaatcaaggatttcccgtacatggctttataatctcgacaagaatcttgacaacgaatatatctctactctcaattatctgtatgatagaattacaaacgaattaacaaattccttctcaaagaatagtgcagccaacgtaaactatatcgctgaaacccttggtattaatcctgctgaatttgcagagcagtatttcagattcagtatcatgaaggaacagaagaatctcggtttcaatattactaagctgagagaagtaatgcttgacagaaaggatatgtctgagatccgtaaaaatcataaggtctttgattcaatccgtactaaggtctatactatgatggatttcgttatctacagatattacattgaagaggatgcaaaggttgctgctgccaacaagtctctgccggataacgaaaaaagcctcagtgaaaaggatatctttgttataaatctcagaggaagctttaacgatgatcagaaggatgccctttattatgatgaggccaatcgtatttggagaaagctcgaaaacattatgcacaatatcaaggaattcagaggcaataagacacgtgaatacaagaagaaggatgctccaagactccccagaattcttcctgccggaagggatgtttccgcgttctcaaagttgatgtacgctcttaccatgttccttgatggtaaggagatcaatgatcttctcaccacgctcatcaataagttcgataacatccagagtttcctcaaggtaatgcctcttatcggagtgaatgcaaagtttgttgaggaatatgccttcttcaaggacagcgcaaagattgctgacgaactcaggctgattaagagctttgccagaatgggagaacctatcgcagatgcaagacgtgctatgtatatcgatgctatcaggattctcggaacaaacctcagctatgatgagcttaaggcccttgccgatactttttcgcttgatgaaaacggcaacaagcttaagaagggcaagcacggcatgagaaacttcatcattaataatgtaatcagtaacaagcgcttccattatctcattcgttacggtgatcctgcacatctccatgagatcgccaagaatgaagctgttgtaaagttcgtcctcggcaggatagctgatatccagaagaagcagggacagaacggaaagaatcagatcgacaggtactatgagacctgtatcggcaaggacaagggcaagtctgtctccgaaaaggttgatgccctcacaaagattatcaccggtatgaactacgatcagttcgataagaagagaagcgttattgaggatactggaagagaaaacgctgagagagaaaagttcaagaagatcatcagcctctatcttactgtcatttatcacatccttaagaatattgttaatatcaatgcgcgttacgttatcggcttccattgcgttgagcgtgatgcacagctctataaggaaaagggctatgatatcaacctcaagaagctcgaagaaaaggggttttcatcagtcacaaagctgtgtgcaggtattgatgagactgctcctgacaagcgtaaggatgttgaaaaggaaatggctgagcgtgcaaaggaatctatcgatagccttgaatctgcaaatcctaagctttacgcaaactatatcaagtattctgacgagaagaaggctgaggaatttactagacagatcaaccgtgagaaggcaaagaccgctctgaatgcatatctcagaaatactaagtggaatgtgataatcagggaagatcttcttagaatcgataataagacatgtacgctctttagaaataaggccgttcatcttgaagttgcaagatatgttcatgcatatatcaacgatattgccgaagtaaacagctatttccagctttatcattacatcatgcagagaatcatcatgaacgaaagatatgaaaagtcttctggaaaggtaagcgaatacttcgatgctgtgaacgatgaaaagaagtacaacgacaggcttctgaagctgttgtgcgttccatttggttactgcatcccgagattcaagaatctctccattgaagctttgttcgacaggaacgaagcagctaagtttgacaaggaaaagaagaaagtatcaggtaattcatag

SEQ ID NO: 36 - DNA, 2391 bp, Ruminococcus sp.
  misc_feature 1..2391: note = native CasM DNA sequence from Ruminococcus sp., isolate 2789STDY5834894
  source 1..2391: mol_type = genomic DNA
gtggagataaacacttcaaaccctactcacagaagcggtgaaagctcgtctgtaagaggggatatgctggggcttaaatcggagcttgaaaagcgctttttcggcaagacttttgatgataatatacatatccagcttatttacaacattctggatatcgaaaagatacttgcagtgtatgtgacgaatatcgtttatgcactgaacaatatgcttggtgtaaagggttctgaaagttatgatgattttatggggtatctttctgcccaaaatacttattatatttttactcaccctgacaaaagtaatctttccgataaggtaaagggtaatatcaagaaaagccttagcaagtttaatgacctgctgaaaactaagcgtcttggctattttggtcttgaagagcctaagacgaaagataaaagagtttcggaggcatacaaaaagcgtgtttatcatatgcttgcaattgtggggcagataaggcagagtgttttccatgataagtcaaatgagcttgatgagtacctttacagctttattgacattattgattccgaatacagagacactcttgactatcttgtagatgagagatttgattctataaataagggctttgtccagggcaacaaggtcaatatcagcttgcttattgatatgatgaaaggctatgaggctgatgatatcatacgcctttattatgatttcattgtgcttaaatctcagaaaaatctcggtttttctatcaaaaagcttcgtgagaaaatgctggacgaatacggcttcagatttaaggacaagcaatatgactctgtgcgctcaaagatgtacaagcttatggattttctgcttttctgcaactattacagaaatgacgttgtcgcaggcgaagctcttgtgcgcaaactgcgtttttcaatgaccgatgatgaaaaagaggggatatatgctgatgaagcggaaaagctttggggcaaattcaggaatgattttgaaaatatcgccgaccacatgaacggtgacgttatcaaggagcttggcaaggctgacatggattttgatgagaaaattcttgacagcgaaaagaagaatgcgtctgaccttttgtatttctccaaaatgatatatatgctcacatattttcttgacggcaaggagataaacgatcttcttacaacgcttatcagcaagtttgataacatcaaggagtttttgaagataatgaaaagctctgctgttgatgttgagtgtgagcttacggcgggctacaagctgttcaatgacagccagaggataaccaacgagctttttatcgtaaagaacattgcttccatgagaaagcctgcggcttcggcgaagcttacgatgttccgtgacgcactgactatactcggtatagacgacaagatcacggacgataggataagcgagattttaaaacttaaagaaaaaggcaagggcatacatggtctgagaaattttataacaaacaatgttatcgagtcctctcggtttgtataccttatcaagtatgcgaacgctcagaagataagagaagtggctaagaatgagaaagttgtcatgtttgttcttgggggtatccctgacacgcagatagagcgttattacaagagttgtgtggaatttcctgacatgaacagttctttggaagcaaagtgcagtgagcttgcgagaatgataaagaacatcagctttgatgatttcaaaaatgtgaaacagcaggcaaagggcagagaaaacgtggctaaggagagggcaaaggctgttatcgggctttatcttacggtcatgtatctgctggtgaaaaatcttgtgaatgtcaatgcaaggtatgttattgcgatacactgccttgaacgtgattttgggctgtataaggagataattcctgagttggcttcaaagaacttgaaaaatgactacaggatactttcacagacgctttgtgaactttgtgatgatcgtgatgagtcgccgaatttgttcttgaaaaagaacaagcggctgcgcaagtgcgttgaagttgatatcaataatgcagacagcagcatgacaagaaaataccgcaactgtattgctcatcttactgtagttcgtgaactgaaagaatacataggagatattcgtacagtggattcttacttctccatttatcattatgttatgcagcgctgtatcacgaaaagggaagatgacacaaagcaagaagagaaaataaagtatgaggacgatcttttaaaaaatcacggctatacgaaagactttgtaaaggctctcaactcgccgtttggatacaacattccgaggtttaaaaatctttcaattgagcagttgtttgacagaaatgaatatcttactgaaaagtag

SEQ ID NO: 37 - protein, 954 aa, Eubacterium siraeum
  REGION 1..954: note = misc_feature - native CasM protein sequence from Eubacterium siraeum
MGKKIHARDLREQRKTDRTEKFADQNKKREAERAVPKKDAAVSVKSVSSVSSKKDNVTKSMAKAAGVKSVFAVGNTVYMTSFGRGNDAVLEQKIVDTSHEPLNIDDPAYQLNVVTMNGYSVTGHRGETVSAVTDNPLRRFNGRKKDEPEQSVPTDMLCLKPTLEKKFFGKEFDDNIHIQLIYNILDIEKILAVYSTNAIYALNNMSADENIENSDFFMKRTTDETFDDFEKKKESTNSREKADFDAFEKFIGNYRLAYFADAFYVNKKNPKGKAKNVLREDKELYSVLTLIGKLRHWCVHSEEGRAEFWLYKLDELKDDFKNVLDVVYNRPVEEINNRFIENNKVNIQILGSVYKNTDIAELVRSYYEFLITKKYKNMGFSIKKLRESMLEGKGYADKEYDSVRNKLYQMTDFILYTGYINEDSDRADDLVNTLRSSLKEDDKTTVYCKEADYLWKKYRESIREVADALDGDNIKKLSKSNIEIQEDKLRKCFISYADSVSEFTKLIYLLTRFLSGKEINDLVTTLINKFDNIRSFLEIMDELGLDRTFTAEYSFFEGSTKYLAELVELNSFVKSCSFDINAKRTMYRDALDILGIESDKTEEDIEKMIDNILQIDANGDKKLKKNNGLRNFIASNVIDSNRFKYLVRYGNPKKIRETAKCKPAVRFVLNEIPDAQIERYYEACCPKNTALCSANKRREKLADMIAEIKFENFSDAGNYQKANVTSRTSEAEIKRKNQAIIRLYLTVMYIMLKNLVNVNARYVIAFHCVERDTKLYAESGLEVGNIEKNKTNLTMAVMGVKLENGIIKTEFDKSFAENAANRYLRNARWYKLILDNLKKSERAVVNEFRNTVCHLNAIRNININIKEIKEVENYFALYHYLIQKHLENRFADKKVERDTGDFISKLEEHKTYCKDFVKAYCTPFGYNLVRYKNLTIDGLFDKNYPGKDDSDEQK

SEQ ID NO: 38 - protein, 919 aa, Ruminococcus sp.
  REGION 1..919: note = misc_feature - native CasM protein sequence from Ruminococcus sp., isolate 2789STDY5834971
MAKKNKMKPRELREAQKKARQLKAAEINNNAAPAIAAMPAAEVIAPAAEKKKSSVKAAGMKSILVSENKMYITSFGKGNSAVLEYEVDNNDYNQTQLSSKDNSNIQLGGVNEVNITFSSKHGFESGVEINTSNPTHRSGESSPVRGDMLGLKSELEKRFFGKTFDDNIHIQLIYNILDIEKILAVYVTNIVYALNNMLGVKGSESHDDFIGYLSTNNIYDVFIDPDNSSLSDDKKANVRKSLSKFNALLKTKRLGYFGLEEPKTKDNRVSQAYKKRVYHMLAIVGQIRQCVFHDKSGAKRFDLYSFINNIDPEYRDTLDYLVEERLKSINKDFIEDNKVNISLLIDMMKGYEADDIIRLYYDFIVLKSQKNLGFSIKKLREKMLDEYGFRFKDKQYDSVRSKMYKLMDFLLFCNYYRNDIAAGESLVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISGILKLKEKGKGIHGLRNFITNNVIESSRFVYLIKYANAQKIREVAKNEKVVMFVLGGIPDTQIERYYKSCVEFPDMNSSLGVKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDKSPNLFLKKNERLRKCVEVDINNADSSMTRKYRNCIAHLTVVRELKEYIGDICTVDSYFSIYHYVMQRCITKRENDTKQEEKIKYEDDLLKNHGYTKDFVKALNSPFGYNIPRFKNLSIEQLFDRNEYLTEK

SEQ ID NO: 39 - protein, 918 aa, Ruminococcus bicirculans
  REGION 1..918: note = misc_feature - native CasM protein sequence from Ruminococcus bicirculans
MAKKNKMKPRELREAQKKARQLKAAEINNNAVPAIAAMPAAEAAAPAAEKKKSSVKAAGMKSILVSENKMYITSFGKGNSAVLEYEVDNNDYNKTQLSSKDNSNIELCDVGKVNITFSSRRGFESGVEINTSNPTHRSGESSSVRGDMLGLKSELEKRFFGKNFDDNIHIQLIYNILDIEKILAVYVTNIVYALNNMLGEGDESNYDFMGYLSTFNTYKVFTNPNGSTLSDDKKENIRKSLSKFNALLKTKRLGYFGLEEPKTKDTRASEAYKKRVYHMLAIVGQIRQCVFHDKSGAKRFDLYSFINNIDPEYRETLDYLVDERFDSINKGFIQGNKVNISLLIDMMKGYEADDIIRLYYDFIVLKSQKNLGFSIKKLREKMLDEYGFRFKDKQYDSVRSKMYKLMDFLLFCNYYRNDIAAGESLVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISEILKLKEKGKGIHGLRNFITNNVIESSRFVYLIKYANAQKIREVAKNEKVVMFVLGGIPDTQIERYYKSCVEFPDMNSSLGVKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDKSPNLFLKKNERLRKCVEVDINNADSSMTRKYRNCIAHLTVVRELKEYIGDICTVDSYFSIYHYVMQRCITKRENDTKQEEKIKYEDDLLKNHGYTKDFVKALNSPFGYNIPRFKNLSIEQLFDRNEYLTEK

SEQ ID NO: 40 - protein, 922 aa, Ruminococcus sp.
  REGION 1..922: note = misc_feature - native CasM protein sequence from Ruminococcus sp., isolate 2789STDY5608892
MAKKNKMKPRELREAQKKARQLKAAEINNNAAPAIAAMPAAEVIAPVAEKKKSSVKAAGMKSILVSENKMYITSFGKGNSAVLEYEVDNNDYNKTQLSSKDNSNIELGDVNEVNITFSSKHGFGSGVEINTSNPTHRSGESSPVRGDMLGLKSELEKRFFGKTFDDNIHIQLIYNILDIEKILAVYVTNIVYALNNMLGIKDSESYDDFMGYLSARNTYEVFTHPDKSNLSDKVKGNIKKSLSKFNDLLKTKRLGYFGLEEPKTKDTRASEAYKKRVYHMLAIVGQIRQCVFHDKSGAKRFDLYSFINNIDPEYRDTLDYLVEERLKSINKDFIEGNKVNISLLIDMMKGYEADDIIRLYYDFIVLKSQKNLGFSIKKLREKMLEEYGFRFKDKQYDSVRSKMYKLMDFLLFCNYYRNDVAAGEALVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDNITDDRISEILKLKEKGKGIHGLRNFITNNVIESSRFVYLIKYANAQKIREVAKNEKVVMFVLGGIPDTQIERYYKSCVEFPDMNSSLEAKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDDRNESSNLFLKKNKRLRKCVEVDINNADSSMTRKYRNCIAHLTVVRELKEYIGDIRTVDSYFSIYHYVMQRCITKRGDDTKQEEKIKYEDDLLKNHGYTKDFVKALNSPFGYNIPRFKNLSIEQLFDRNEYLTEK

SEQ ID NO: 41 - protein, 922 aa, Ruminococcus sp.
  REGION 1..922: note = misc_feature - native CasM protein sequence from Ruminococcus sp. CAG:57
MAKKNKMKPRELREAQKKARQLKAAEINNNAAPAIAAMPAAEVIAPVAEKKKSSVKAAGMKSILVSENKMYITSFGKGNSAVLEYEVDNNDYNKTQLSSKDNSNIELGDVNEVNITFSSKHGFGSGVEINTSNPTHRSGESSPVRGDMLGLKSELEKRFFGKTFDDNIHIQLIYNILDIEKILAVYVTNIVYALNNMLGIKDSESYDDFMGYLSARNTYEVFTHPDKSNLSDKVKGNIKKSLSKFNDLLKTKRLGYFGLEEPKTKDTRASEAYKKRVYHMLAIVGQIRQCVFHDKSGAKRFDLYSFINNIDPEYRDTLDYLVEERLKSINKDFIEGNKVNISLLIDMMKGYEADDIIRLYYDFIVLKSQKNLGFSIKKLREKMLEEYGFRFKDKQYDSVRSKMYKLMDFLLFCNYYRNDVAAGEALVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDNITDDRISEILKLKEKGKGIHGLRNFITNNVIESSRFVYLIKYANAQKIREVAKDEKVVMFVLGGIPDTQIERYYKSCVEFPDMNSSLEAKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDDRNESSNLFLKKNKRLRKCVEVDINNADSSMTRKYRNCIAHLTVVRELKEYIGDIRTVDSYFSIYHYVMQRCITKRGDDTKQEEKIKYEDDLLKNHGYTKDFVKALNSPFGYNIPRFKNLSIEQLFDRNEYLTEK

SEQ ID NO: 42 - protein, 933 aa, Ruminococcus flavefaciens
  REGION 1..933: note = misc_feature - native CasM protein sequence from Ruminococcus flavefaciens FD-1
MKKKMSLREKREAEKQAKKAAYSAASKNTDSKPAEKKAETPKPAEIISDNSRNKTAVKAAGLKSTIISGDKLYMTSFGKGNAAVIEQKIDINDYSFSAMKDTPSLEVDKAESKEISFSSHHPFVKNDKLTTYNPLYGGKDNPEKPVGRDMLGLKDKLEERYFGCTFNDNLHIQIIYNILDIEKILAVHSANITTALDHMVDEDDEKYLNSDYIGYMNTINTYDVFMDPSKNSSLSPKDRKNIDNSRAKFEKLLSTKRLGYFGFDYDANGKDKKKNEEIKKRLYHLTAFAGQLRQWSFHSAGNYPRTWLYKLDSLDKEYLDTLDHYFDKRFNDINDDFVTKNATNLYILKEVFPEANFKDIADLYYDFIVIKSHKNMGFSIKKLREKMLECDGADRIKEQDMDSVRSKLYKLIDFCIFKYYHEFPELSEKNVDILRAAVSDTKKDNLYSDEAARLWSIFKEKFLGFCDKIVVWVTGEHEKDITSVIDKDAYRNRSNVSYFSKLMYAMCFFLDGKEINDLLTTLINKFDNIANQIKTAKELGINTAFVKNYDFFNHSEKYVDELNIVKNIARMKKPSSNAKKAMYHDALTILGIPEDMDEKALDEELDLILEKKTDPVTGKPLKGKNPLRNFIANNVIENSRFIYLIKFCNPENVRKIVNNTKVTEFVLKRIPDAQIERYYKSCTDSEMNPPTEKKITELAGKLKDMNFGNFRNVRQSAKENMEKERFKAVIGLYLTVVYRVVKNLVDVNSRYIMAFHSLERDSQLYNVSVDNDYLALTDTLVKEGDNSRSRYLAGNKRLRDCVKQDIDNAKKWFVSDKYNSITKYRNNVAHLTAVRNCAEFIGDITKIDSYFALYHYLIQRQLAKGLDHERSGFDRNYPQYAPLFKWHTYVKDVVKALNAPFGYNIPRFKNLSIDALFDRNEIKKNDGEKKSDD

SEQ ID NO: 43 - protein, 944 aa, Ruminococcus albus
  REGION 1..944: note = misc_feature - native CasM protein sequence from Ruminococcus albus strain KH2T6
MAKKSKGMSLREKRELEKQKRIQKAAVNSVNDTPEKTEEANVVSVNVRTSAENKHSKKSAAKALGLKSGLVIGDELYLTSFGRGNEAKLEKKISGDTVEKLGIGAFEVAERDESTLTLESGRIKDKTARPKDPRHITVDTQGKFKEDMLGIRSVLEKKIFGKTFDDNIHVQLAYNILDVEKIMAQYVSDIVYMLHNTDKTERNDNLMGYMSIRNTYKTFCDTSNLPDDTKQKVENQKREFDKIIKSGRLGYFGEAFMVNSGNSTKLRPEKEIYHIFALMASLRQSYFHGYVKDTDYQGTTWAYTLEDKLKGPSHEFRETIDKIFDEGFSKISKDFGKMNKVNLQILEQMIGELYGSIERQNLTCDYYDFIQLKKHKYLGFSIKRLRETMLETTPAECYKAECYNSERQKLYKLIDFLIYDLYYNRKPARIEEIVDKLRESVNDEEKESIYSVEAKYVYESLSKVLDKSLKNSVSGETIKDLQKRYDDETANRIWDISQHSISGNVNCFCKLIYIMTLMLDGKEINDLLTTLVNKFDNIASFIDVMDELGLEHSFTDNYKMFADSKAICLDLQFINSFARMSKIDDEKSKRQLFRDALVILDIGNKDETWINNYLDSDIFKLDKEGNKLKGARHDFRNFIANNVIKSSRFKYLVKYSSADGMIKLKTNEKLIGFVLDKLPETQIDRYYESCGLDNAVVDKKVRIEKLSGLIRDMKFDDFSGVKTSNKAGDNDKQDKAKYQAIISLYLMVLYQIVKNMIYVNSRYVIAFHCLERDFGMYGKDFGKYYQGCRKLTDHFIEEKYMKEGKLGCNKKVGRYLKNNISCCTDGLINTYRNQVDHFAVVRKIGNYAAYIKSIGSWFELYHYVIQRIVFDEYRFALNNTESNYKNSIIKHHTYCKDMVKALNTPFGYDLPRYKNLSIGDLFDRNNYLNKTKESIDANSSIDSQ

SEQ ID NO: 44 - protein, 967 aa, Ruminococcus flavefaciens
  REGION 1..967: note = misc_feature - native CasM protein sequence from Ruminococcus flavefaciens strain XPD3002
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 45 - protein, 796 aa, Ruminococcus sp.
  REGION 1..796: note = misc_feature - native CasM protein sequence from Ruminococcus sp., isolate 2789STDY5834894
MEINTSNPTHRSGESSSVRGDMLGLKSELEKRFFGKTFDDNIHIQLIYNILDIEKILAVYVTNIVYALNNMLGVKGSESYDDFMGYLSAQNTYYIFTHPDKSNLSDKVKGNIKKSLSKFNDLLKTKRLGYFGLEEPKTKDKRVSEAYKKRVYHMLAIVGQIRQSVFHDKSNELDEYLYSFIDIIDSEYRDTLDYLVDERFDSINKGFVQGNKVNISLLIDMMKGYEADDIIRLYYDFIVLKSQKNLGFSIKKLREKMLDEYGFRFKDKQYDSVRSKMYKLMDFLLFCNYYRNDVVAGEALVRKLRFSMTDDEKEGIYADEAEKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISEILKLKEKGKGIHGLRNFITNNVIESSRFVYLIKYANAQKIREVAKNEKVVMFVLGGIPDTQIERYYKSCVEFPDMNSSLEAKCSELARMIKNISFDDFKNVKQQAKGRENVAKERAKAVIGLYLTVMYLLVKNLVNVNARYVIAIHCLERDFGLYKEIIPELASKNLKNDYRILSQTLCELCDDRDESPNLFLKKNKRLRKCVEVDINNADSSMTRKYRNCIAHLTVVRELKEYIGDIRTVDSYFSIYHYVMQRCITKREDDTKQEEKIKYEDDLLKNHGYTKDFVKALNSPFGYNIPRFKNLSIEQLFDRNEYLTEK

SEQ ID NO: 46 - RNA, 96 nt, synthetic construct
  misc_feature 1..96: note = Synthetic: CRISPR Arrays Component
  source 1..96: mol_type = other RNA
tgatactgctttgatgtcagcattgcatatctactatactggtgcgaatttgcactagtctaaaatctataaccataagttcttctgcgttcatat

SEQ ID NO: 47 - RNA, 66 nt, synthetic construct
  misc_feature 1..66: note = Synthetic: CRISPR Arrays Component
  source 1..66: mol_type = other RNA
tgatactgctttgatgtcagcattgcatatctactatactggtgcgaatttgcactagtctaaaat

SEQ ID NO: 48 - RNA, 66 nt, synthetic construct
  misc_feature 1..66: note = Synthetic: CRISPR Arrays Component
  source 1..66: mol_type = other RNA
ctactatactggtgcgaatttgcactagtctaaaattgatactgctttgatgtcagcattgcatat

SEQ ID NO: 49 - DNA, 45 bp, synthetic construct
  misc_feature 1..45: note = Synthetic: Forward primer
  source 1..45: mol_type = other DNA
cgaaattaatacgactcactataggtttcgattatgcggccgtgt

SEQ ID NO: 50 - DNA, 22 bp, synthetic construct
  misc_feature 1..22: note = Synthetic: Reverse primer
  source 1..22: mol_type = other DNA
aggagatataccatgggcagca

SEQ ID NO: 51 - RNA, 36 nt, synthetic construct
  misc_feature 1..36: note = Synthetic: CasM repeat sequence
  source 1..36: mol_type = other RNA
ctactatactggtgcgaatttgcactagtctaaaat

SEQ ID NO: 52 - protein, 824 aa, Eubacterium siraeum
  REGION 1..824: note = misc_feature - WYL Eubacterium siraeum
MKKTEKFDDVQSGYEYKYFLESIDKYRAAVQNIYTYGCFNQKQLSEQCNCSDQTIKKAFNFYNLCLANYIKKKKGTLSKKAKGRPTEAKYLEYDRFTLNENYLYNIYLWARITKKQMWAFSYFRRHTSLLINASRTEIKNQLSDFFLYFSEYMDRSKKAENSQDLGYIIDMTAPTEKNMLISSMCDALAVFGRKAPYSVPAYSISHKLKKLCGNDSKSLWSFMYDNYDRILYDEAVYTIRQAIRDRKLIGYQTVGTEKQKSVNYVVPLKIMYEYNLGRCYLLYSPLNSDSIIKSIRLDKLYKVAAYEPDSIINYEKLYDVLAVAENEIWLSGDYTKKDCLSRIVLKNVKPQAFSLIEKYGVCYTEDREAKTVTFNIRKADDIKPFIRTLGGDAVISEEDNPGLFREFAYDARIGRQMYYDDSFADCPAEKDSQPAKDSKTASGNDNIKKYASYPTLRLFNKYGSFMNILAEELAEHIFSEIIRMPVEKRAGQIEYSSNRLERVLNSYFKIYGFDELRTEASNITEWFTKATEELSDSDYSSWFSVNGGKFEAVADLNEYEHKQLLTNIEYEYLRLMLGDPDARAIIGNEYCEKLSEYVGSADTTLDEFFTVRYANRNEKTIENKHSVLRTIMRAMNNEKKADIEYKGKHYICSAYRFTYSLRERKHRLMVFDGNYIMQINLCDIKDAQMTKEPSLSDEEMNKLLTERKKYIEIAIPQNADAQQRNVFERALRLFGGFERYSWNDAKNGEYVIAVAYYEPDISVSSSADRRIYRRDTVAADIMSLGRYARVMKQPGFELDGVRYDSSLYDYISKNYSGTAARYEK

SEQ ID NO: 53 - protein, 389 aa, Ruminococcus sp.
  REGION 1..389: note = misc_feature - WYL Ruminococcus sp.isolate 2789STDY5834971
MLILPSTFLPKRDKNVPYIAEVQSIPLSPSAYSVIIKDKSIFETSLSPNGSVSMSSFLTSIFDSAYIASLKYKSEKYNGIPLLNAFVKWQIEEINDGLDDKSKEIIKSYLISKLSAKYEKTKTENAVRVRLSICRDLYDTLSSDDLYYENKVYSSTLRRFLKAVYEDYALLSDCERERLIFADNIIKINEVIKQNGSRYYSFIYAYSNMYSREKRRIRLIPYRIVSDEYKMYNYLVCLSDEKSAGKEFKADSYRISRLSGLSIAEKLSQKEYSSVTEYERLKEGHVKSVKHLLSDPRFGSDESDISKVYLTEKGVEMFGKILYQRPILKGNEKPKPNAVNEFISPPIQVKYYFNKFGKDGVILSPSDSFEEMRTLYVEGAEAYNREVEM

SEQ ID NO: 54 - protein, 392 aa, Ruminococcus bicirculans
  REGION 1..392: note = misc_feature - WYL Ruminococcus bicirculans
MSMTPSTFLPKREDGVPYIAEVQSIPLSPSAYSVIIKDKSIFETSLSPNGSVSMSSFLTSIFDSAYIASLKYKSDDNYKYIGIPLLNAFVKWQIEEIDDSLDDKSKEIIKSYLISKLSAKYEKTKTENAVRVRLSICRDLYDTLSSDDLYYENKVYSSTLRRFLKAVYEDYALLSDCERERLIFADNIIKINEVIKQNGSRYYSFIYAYSNMYSREKRRIRLIPYRIVSDEYKMYNYLVCLSDEKSAGKEFKADSYRISRLSGLSIAEKLSQKEYSSVTEYERLKEGHVKSVKHLLSDPRFGSDESDISKVYLTEKGVEMFGKILYQRPILKGNEKPKPNAVNEFISPPIQVKYYFNKFGKDGVILSPSDSFEEMRTLYVEGAEAYNREVEM

SEQ ID NO: 55 - protein, 392 aa, Ruminococcus sp.
  REGION 1..392: note = misc_feature - WYL Ruminococcus sp. isolate 2789STDY5608892
MLIPPSTFLPKRDKNVPYIAEVQSIPLSPSAYSVIIKDKSIFETSLSPNGSVSMSSFLTSIFDSAYIASLKYKSDDNYKYIGIPLLNAFVEWQIEEIDDSLDDKSKEIIKSYLISKLSAKYEKTKTENAVRVRLSICRDLYDTLSSDDLYYENKVYSLTLRRFLKAVYEDYALLSDCERERLIFADNIIKINEVIKQNGSRYYSFIYAYSNMYSREKRRIRLIPYRIVSDEYKMYNYLVCLSDEKSAGKEFKADSYRISRLSGLSIAEKLSQKEYSSVTEYERLKEGHVKSVKHLLSDPRFGSDESDISKVYLTEKGVEMFGKILYQRPILKGNEKPKPNTVNEFISPPIQVKYYFNKFGKDGVILSPSDSFEEMRTLYVEGAEAYNREVEM

SEQ ID NO: 56 - protein, 392 aa, Ruminococcus sp.; identical to SEQ ID NO: 55
  REGION 1..392: note = misc_feature - WYL Ruminococcus sp. CAG:57

SEQ ID NO: 57 - protein, 280 aa, Ruminococcus flavefaciens
  REGION 1..280: note = misc_feature - WYL Ruminococcus flavefaciens FD-1
MIIAINQWKRRFSLVIYGKSEGETIVKIKLLLISLAYLISIYLLCSPGCIGIFTHGMLTTVIGVVTMLAATGTYGMYLYSSAIGERSLPEIPMNKETEYSRYKELENWFRAFRYLDRNNNFAMLSSDLATSYHDGLIRDNPFRNTELGDRLQTTSSDISIKYDQTLKILSESFEKNDITYQNYLSVLDNVLKLSSSHLKAIKKRVCVFDYRTWADNKNDEMCRKYIEEVKSSVIRLEEIEGKFDNLLHELICLSEISEDPLLEMQDLIETTSDYKSIEDQ

SEQ ID NO: 58 - protein, 226 aa, Ruminococcus albus
  REGION 1..226: note = misc_feature - WYL Ruminococcus albus strain KH2T6
MCTWYYAEAKSLSFFIDKASQLPLSDIIMNTMSKSKAMSGNIRPTDMAAVLAPNKQGNVAVFPMIWGFTHESTSKPVINCRIESADTKPLWKDSWYRRRCVIPASWYYEWGVPPSEGELYHKNEYNKIQKEKYAIQPEGAEITYLAGLYRFEEHRGVQVPMFAVITRESVEPVSSIHDRMPLILGKDSLSEWIHPNGDPNKIAKTALTKMVMEKAIDYPEPEPSFM

SEQ ID NO: 59 - protein, 314 aa, Ruminococcus flavefaciens
  REGION 1..314: note = misc_feature - WYL Ruminococcus flavefaciens strain XPD3002
MELFNEYRNKSLRAFLKLAERISYGEELSIDEFEAEYYRLSGDNKKITSVFYKNTLYNDKLPIFDTREGKVRLFGEPDKCSNKHISDTLLKSEITWLHNALNDKLSKLFLSDEERISIDAKLSDYTEYYKNIDDMWRSNEDISEEVEKNFKIILKAINEKQALSYTFKNKNCEGFPVRIEYDERTCRIYMIIYDGNRFVKSDISKLSDIYITENSIDTIPEIKDDMLNKKAYLPVVFTVTDDKNRKAIDRALLAFSVYDHVVEPIDEKTARFTIQYYTMDLDLLIKDILAFGSDIKVESPRYVVKRITDILRKV

SEQ ID NO: 60 - protein, 412 aa, Eubacterium siraeum
  REGION 1..412: note = misc_feature - RtcB Eubacterium siraeum
MIVLEIIGERNTAVVYGEIIDECAVSQIEEICNHPAFENSRIRIMPDCHAGKGCVIGFTCVTSNRMIVPNIVGVDIGCGILTTVFTADREIDYRALDTFIRSNIPSGMEIHDSVSDTVAENTALIAKVNGICDAIGESADVDYHLRSIGTLGGGNHFIEIDRLNNGNYALTVHTGSRNLGKRICGYFQSNASVIDTELRRSILLRHRSATTSEEHEEIDRRAAQIAPVSKELAFITGERYDSYIGCMLDAKALAAFNRTVISDRIMSFLADEYGVEIKDRFDTVHNYIDWYDDTHTSVVIRKGAISARKGERIVIPLNMRDGIIIAHGRGNEEWNCSAPHGSGRAYSRSDARRTFTLEEYVEEMDGVNTWSVSESTIDECPMAYKPSEMIIGSIGDTVEIESIAHTVYNFKA

SEQ ID NO: 61 - protein, 831 aa, synthetic construct
  REGION 1..831: note = Synthetic: WYL Eubacterium siraeum + C-term NLS
MKKTEKFDDVQSGYEYKYFLESIDKYRAAVQNIYTYGCFNQKQLSEQCNCSDQTIKKAFNFYNLCLANYIKKKKGTLSKKAKGRPTEAKYLEYDRFTLNENYLYNIYLWARITKKQMWAFSYFRRHTSLLINASRTEIKNQLSDFFLYFSEYMDRSKKAENSQDLGYIIDMTAPTEKNMLISSMCDALAVFGRKAPYSVPAYSISHKLKKLCGNDSKSLWSFMYDNYDRILYDEAVYTIRQAIRDRKLIGYQTVGTEKQKSVNYVVPLKIMYEYNLGRCYLLYSPLNSDSIIKSIRLDKLYKVAAYEPDSIINYEKLYDVLAVAENEIWLSGDYTKKDCLSRIVLKNVKPQAFSLIEKYGVCYTEDREAKTVTFNIRKADDIKPFIRTLGGDAVISEEDNPGLFREFAYDARIGRQMYYDDSFADCPAEKDSQPAKDSKTASGNDNIKKYASYPTLRLFNKYGSFMNILAEELAEHIFSEIIRMPVEKRAGQIEYSSNRLERVLNSYFKIYGFDELRTEASNITEWFTKATEELSDSDYSSWFSVNGGKFEAVADLNEYEHKQLLTNIEYEYLRLMLGDPDARAIIGNEYCEKLSEYVGSADTTLDEFFTVRYANRNEKTIENKHSVLRTIMRAMNNEKKADIEYKGKHYICSAYRFTYSLRERKHRLMVFDGNYIMQINLCDIKDAQMTKEPSLSDEEMNKLLTERKKYIEIAIPQNADAQQRNVFERALRLFGGFERYSWNDAKNGEYVIAVAYYEPDISVSSSADRRIYRRDTVAADIMSLGRYARVMKQPGFELDGVRYDSSLYDYISKNYSGTAARYEKPKKKRKV

SEQ ID NO: 62 - protein, 396 aa, synthetic construct
  REGION 1..396: note = Synthetic: WYL Ruminococcus sp.isolate 2789STDY5834971 + C-term NLS
MLILPSTFLPKRDKNVPYIAEVQSIPLSPSAYSVIIKDKSIFETSLSPNGSVSMSSFLTSIFDSAYIASLKYKSEKYNGIPLLNAFVKWQIEEINDGLDDKSKEIIKSYLISKLSAKYEKTKTENAVRVRLSICRDLYDTLSSDDLYYENKVYSSTLRRFLKAVYEDYALLSDCERERLIFADNIIKINEVIKQNGSRYYSFIYAYSNMYSREKRRIRLIPYRIVSDEYKMYNYLVCLSDEKSAGKEFKADSYRISRLSGLSIAEKLSQKEYSSVTEYERLKEGHVKSVKHLLSDPRFGSDESDISKVYLTEKGVEMFGKILYQRPILKGNEKPKPNAVNEFISPPIQVKYYFNKFGKDGVILSPSDSFEEMRTLYVEGAEAYNREVEMPKKKRKV

SEQ ID NO: 63 - protein, 399 aa, synthetic construct
  REGION 1..399: note = Synthetic: WYL Ruminococcus bicirculans + C-term NLS
MSMTPSTFLPKREDGVPYIAEVQSIPLSPSAYSVIIKDKSIFETSLSPNGSVSMSSFLTSIFDSAYIASLKYKSDDNYKYIGIPLLNAFVKWQIEEIDDSLDDKSKEIIKSYLISKLSAKYEKTKTENAVRVRLSICRDLYDTLSSDDLYYENKVYSSTLRRFLKAVYEDYALLSDCERERLIFADNIIKINEVIKQNGSRYYSFIYAYSNMYSREKRRIRLIPYRIVSDEYKMYNYLVCLSDEKSAGKEFKADSYRISRLSGLSIAEKLSQKEYSSVTEYERLKEGHVKSVKHLLSDPRFGSDESDISKVYLTEKGVEMFGKILYQRPILKGNEKPKPNAVNEFISPPIQVKYYFNKFGKDGVILSPSDSFEEMRTLYVEGAEAYNREVEMPKKKRKV

SEQ ID NO: 64 - protein, 399 aa, synthetic construct
  REGION 1..399: note = Synthetic: WYL Ruminococcus sp. isolate 2789STDY5608892 + C-term NLS
MLIPPSTFLPKRDKNVPYIAEVQSIPLSPSAYSVIIKDKSIFETSLSPNGSVSMSSFLTSIFDSAYIASLKYKSDDNYKYIGIPLLNAFVEWQIEEIDDSLDDKSKEIIKSYLISKLSAKYEKTKTENAVRVRLSICRDLYDTLSSDDLYYENKVYSLTLRRFLKAVYEDYALLSDCERERLIFADNIIKINEVIKQNGSRYYSFIYAYSNMYSREKRRIRLIPYRIVSDEYKMYNYLVCLSDEKSAGKEFKADSYRISRLSGLSIAEKLSQKEYSSVTEYERLKEGHVKSVKHLLSDPRFGSDESDISKVYLTEKGVEMFGKILYQRPILKGNEKPKPNTVNEFISPPIQVKYYFNKFGKDGVILSPSDSFEEMRTLYVEGAEAYNREVEMPKKKRKV

SEQ ID NO: 65 - protein, 399 aa, synthetic construct; identical to SEQ ID NO: 64
  REGION 1..399: note = Synthetic: WYL Ruminococcus sp. CAG:57 + C-term NLS

SEQ ID NO: 66 - protein, 287 aa, synthetic construct
  REGION 1..287: note = Synthetic: WYL Ruminococcus flavefaciens FD-1 + C-term NLS
MIIAINQWKRRFSLVIYGKSEGETIVKIKLLLISLAYLISIYLLCSPGCIGIFTHGMLTTVIGVVTMLAATGTYGMYLYSSAIGERSLPEIPMNKETEYSRYKELENWFRAFRYLDRNNNFAMLSSDLATSYHDGLIRDNPFRNTELGDRLQTTSSDISIKYDQTLKILSESFEKNDITYQNYLSVLDNVLKLSSSHLKAIKKRVCVFDYRTWADNKNDEMCRKYIEEVKSSVIRLEEIEGKFDNLLHELICLSEISEDPLLEMQDLIETTSDYKSIEDQPKKKRKV

SEQ ID NO: 67 - protein, 233 aa, synthetic construct
  REGION 1..233: note = Synthetic: WYL Ruminococcus albus strain KH2T6 + C-term NLS
MCTWYYAEAKSLSFFIDKASQLPLSDIIMNTMSKSKAMSGNIRPTDMAAVLAPNKQGNVAVFPMIWGFTHESTSKPVINCRIESADTKPLWKDSWYRRRCVIPASWYYEWGVPPSEGELYHKNEYNKIQKEKYAIQPEGAEITYLAGLYRFEEHRGVQVPMFAVITRESVEPVSSIHDRMPLILGKDSLSEWIHPNGDPNKIAKTALTKMVMEKAIDYPEPEPSFMPKKKRKV

SEQ ID NO: 68 - protein, 321 aa, synthetic construct
  REGION 1..321: note = Synthetic: WYL Ruminococcus flavefaciens strain XPD3002 + C-term NLS
MELFNEYRNKSLRAFLKLAERISYGEELSIDEFEAEYYRLSGDNKKITSVFYKNTLYNDKLPIFDTREGKVRLFGEPDKCSNKHISDTLLKSEITWLHNALNDKLSKLFLSDEERISIDAKLSDYTEYYKNIDDMWRSNEDISEEVEKNFKIILKAINEKQALSYTFKNKNCEGFPVRIEYDERTCRIYMIIYDGNRFVKSDISKLSDIYITENSIDTIPEIKDDMLNKKAYLPVVFTVTDDKNRKAIDRALLAFSVYDHVVEPIDEKTARFTIQYYTMDLDLLIKDILAFGSDIKVESPRYVVKRITDILRKVPKKKRKV

SEQ ID NO: 69 - protein, 419 aa, synthetic construct
  REGION 1..419: note = Synthetic: RtcB Eubacterium siraeum + C-term NLS
MIVLEIIGERNTAVVYGEIIDECAVSQIEEICNHPAFENSRIRIMPDCHAGKGCVIGFTCVTSNRMIVPNIVGVDIGCGILTTVFTADREIDYRALDTFIRSNIPSGMEIHDSVSDTVAENTALIAKVNGICDAIGESADVDYHLRSIGTLGGGNHFIEIDRLNNGNYALTVHTGSRNLGKRICGYFQSNASVIDTELRRSILLRHRSATTSEEHEEIDRRAAQIAPVSKELAFITGERYDSYIGCMLDAKALAAFNRTVISDRIMSFLADEYGVEIKDRFDTVHNYIDWYDDTHTSVVIRKGAISARKGERIVIPLNMRDGIIIAHGRGNEEWNCSAPHGSGRAYSRSDARRTFTLEEYVEEMDGVNTWSVSESTIDECPMAYKPSEMIIGSIGDTVEIESIAHTVYNFKAPKKKRKV

SEQ ID NO: 70 - RNA, 66 nt, synthetic construct
  misc_feature 1..66: note = Synthetic: CasM EGFR mRNA knockdown experiment target 1
  source 1..66: mol_type = other RNA
ctactatactggtgcgaatttgcactagtctaaaatcttacatctttcctcctcatccagcaaaat

SEQ ID NO: 71 - RNA, 66 nt, synthetic construct
  misc_feature 1..66: note = Synthetic: CasM EGFR mRNA knockdown experiment target 2
  source 1..66: mol_type = other RNA
ctactatactggtgcgaatttgcactagtctaaaatcacaatcctgaagtaagtgaagctacagac

SEQ ID NO: 72 - RNA, 66 nt, synthetic construct
  misc_feature 1..66: note = Synthetic: CasM EGFR mRNA knockdown experiment target 3
  source 1..66: mol_type = other RNA
ctactatactggtgcgaatttgcactagtctaaaatctgtcaaaaatcacaatcctgaagtaagtg

SEQ ID NO: 73 - RNA, 66 nt, synthetic construct
  misc_feature 1..66: note = Synthetic: CasM EGFR mRNA knockdown experiment target 4
  source 1..66: mol_type = other RNA
ctactatactggtgcgaatttgcactagtctaaaatctctgtcaaaaatcacaatcctgaagtaag

SEQ ID NO: 74 - RNA, 66 nt, synthetic construct
  misc_feature 1..66: note = Synthetic: CasM EGFR mRNA knockdown experiment target 5
  source 1..66: mol_type = other RNA
ctactatactggtgcgaatttgcactagtctaaaattctcttcacgagattcactaggaccttcag

SEQ ID NO: 75 - RNA, 66 nt, synthetic construct
  misc_feature 1..66: note = Synthetic: CasM EGFR mRNA knockdown experiment target 6
  source 1..66: mol_type = other RNA
ctactatactggtgcgaatttgcactagtctaaaatctcctctcttcacgagattcactaggacct

SEQ ID NO: 76 - RNA, 66 nt, synthetic construct
  misc_feature 1..66: note = Synthetic: CasM EGFR mRNA knockdown experiment target 7
  source 1..66: mol_type = other RNA
ctactatactggtgcgaatttgcactagtctaaaatgtcctctaggtccatgttacagccagaccc

SEQ ID NO: 77 - RNA, 66 nt, synthetic construct
  misc_feature 1..66: note = Synthetic: CasM EGFR mRNA knockdown experiment target 8
  source 1..66: mol_type = other RNA
ctactatactggtgcgaatttgcactagtctaaaatatgtcctctaggtccatgttacagccagac

SEQ ID NO: 78 - RNA, 66 nt, synthetic construct
  misc_feature 1..66: note = Synthetic: CasM EGFR mRNA knockdown experiment target 9
  source 1..66: mol_type = other RNA
ctactatactggtgcgaatttgcactagtctaaaatcgccaggagcgctgccccggccgtcccgga

SEQ ID NO: 79 - RNA, 66 nt, synthetic construct
  misc_feature 1..66: note = Synthetic: CasM EGFR mRNA knockdown experiment target 10
  source 1..66: mol_type = other RNA
ctactatactggtgcgaatttgcactagtctaaaatgcgccaggagcgctgccccggccgtcccgg

SEQ ID NO: 80 - RNA, 66 nt, synthetic construct
  misc_feature 1..66: note = Synthetic: CasM EGFR mRNA knockdown experiment target 11
  source 1..66: mol_type = other RNA
ctactatactggtgcgaatttgcactagtctaaaatgcagcgccaggagcgctgccccggccgtcc

SEQ ID NO: 81 - RNA, 66 nt, synthetic construct
  misc_feature 1..66: note = Synthetic: CasM EGFR mRNA knockdown experiment target 12
  source 1..66: mol_type = other RNA
ctactatactggtgcgaatttgcactagtctaaaatagcagcgccaggagcgctgccccggccgtc

SEQ ID NO: 82 - RNA, 66 nt, synthetic construct
  misc_feature 1..66: note = Synthetic: CasM EGFR mRNA knockdown experiment target 13
  source 1..66: mol_type = other RNA
ctactatactggtgcgaatttgcactagtctaaaatcagcagcgccaggagcgctgccccggccgt

SEQ ID NO: 83 - RNA, 66 nt, synthetic construct
  misc_feature 1..66: note = Synthetic: CasM EGFR mRNA knockdown experiment target 14
  source 1..66: mol_type = other RNA
ctactatactggtgcgaatttgcactagtctaaaatccagcagcgccaggagcgctgccccggccg

SEQ ID NO: 84 - RNA, 66 nt, synthetic construct
  misc_feature 1..66: note = Synthetic: CasM EGFR mRNA knockdown experiment target 15
  source 1..66: mol_type = other RNA
ctactatactggtgcgaatttgcactagtctaaaatgccagcagcgccaggagcgctgccccggcc

SEQ ID NO: 85 - RNA, 66 nt, synthetic construct
  misc_feature 1..66: note = Synthetic: CasM EGFR mRNA knockdown experiment target 16
  source 1..66: mol_type = other RNA
ctactatactggtgcgaatttgcactagtctaaaatcagccagcagcgccaggagcgctgccccgg

SEQ ID NO: 86 - RNA, 66 nt, synthetic construct
  misc_feature 1..66: note = Synthetic: CasM EGFR mRNA knockdown experiment target 17
  source 1..66: mol_type = other RNA
ctactatactggtgcgaatttgcactagtctaaaatgcagccagcagcgccaggagcgctgccccg

SEQ ID NO: 87 - RNA, 66 nt, synthetic construct
  misc_feature 1..66: note = Synthetic: CasM EGFR mRNA knockdown experiment target 18
  source 1..66: mol_type = other RNA
ctactatactggtgcgaatttgcactagtctaaaatcgcagccagcagcgccaggagcgctgcccc

SEQ ID NO: 88 - RNA, 66 nt, synthetic construct
  misc_feature 1..66: note = Synthetic: CasM EGFR mRNA knockdown experiment target 19
  source 1..66: mol_type = other RNA
ctactatactggtgcgaatttgcactagtctaaaatgcgcagccagcagcgccaggagcgctgccc

SEQ ID NO: 89 - RNA, 66 nt, synthetic construct
  misc_feature 1..66: note = Synthetic: CasM EGFR mRNA knockdown experiment target 20
  source 1..66: mol_type = other RNA
ctactatactggtgcgaatttgcactagtctaaaatagcgcagccagcagcgccaggagcgctgcc

SEQ ID NO: 90 - RNA, 66 nt, synthetic construct
  misc_feature 1..66: note = Synthetic: CasM EGFR mRNA knockdown experiment target 21
  source 1..66: mol_type = other RNA
ctactatactggtgcgaatttgcactagtctaaaatgagcgcagccagcagcgccaggagcgctgc

SEQ ID NO: 91 - RNA, 66 nt, synthetic construct
  misc_feature 1..66: note = Synthetic: CasM EGFR mRNA knockdown experiment target 22
  source 1..66: mol_type = other RNA
ctactatactggtgcgaatttgcactagtctaaaatagagcgcagccagcagcgccaggagcgctg

SEQ ID NO: 92 - RNA, 66 nt, synthetic construct
  misc_feature 1..66: note = Synthetic: CasM EGFR mRNA knockdown experiment target 23
  source 1..66: mol_type = other RNA
ctactatactggtgcgaatttgcactagtctaaaatcagagcgcagccagcagcgccaggagcgct

SEQ ID NO: 93 - RNA, 66 nt, synthetic construct
  misc_feature 1..66: note = Synthetic: CasM EGFR mRNA knockdown experiment target 24
  source 1..66: mol_type = other RNA
ctactatactggtgcgaatttgcactagtctaaaatgcagagcgcagccagcagcgccaggagcgc

SEQ ID NO: 94 - RNA, 66 nt, synthetic construct
  misc_feature 1..66: note = Synthetic: CasM EGFR mRNA knockdown experiment target 25
  source 1..66: mol_type = other RNA
ctactatactggtgcgaatttgcactagtctaaaatgggcagagcgcagccagcagcgccaggagc

SEQ ID NO: 95 - RNA, 66 nt, synthetic construct
  misc_feature 1..66: note = Synthetic: CasM EGFR mRNA knockdown experiment target 26
  source 1..66: mol_type = other RNA
ctactatactggtgcgaatttgcactagtctaaaatcgggcagagcgcagccagcagcgccaggag

SEQ ID NO: 96 - RNA, 66 nt, synthetic construct
  misc_feature 1..66: note = Synthetic: CasM EGFR mRNA knockdown experiment target 27
  source 1..66: mol_type = other RNA
ctactatactggtgcgaatttgcactagtctaaaatgccgggcagagcgcagccagcagcgccagg

SEQ ID NO: 97 - RNA, 66 nt, synthetic construct
  misc_feature 1..66: note = Synthetic: CasM EGFR mRNA knockdown experiment target 28
  source 1..66: mol_type = other RNA
ctactatactggtgcgaatttgcactagtctaaaatcgccgggcagagcgcagccagcagcgccag

SEQ ID NO: 98 - RNA, 66 nt, synthetic construct
  misc_feature 1..66: note = Synthetic: CasM EGFR mRNA knockdown experiment target 29
  source 1..66: mol_type = other RNA
ctactatactggtgcgaatttgcactagtctaaaattcgccgggcagagcgcagccagcagcgcca

SEQ ID NO: 99 - RNA, 66 nt, synthetic construct
  misc_feature 1..66: note = Synthetic: CasM EGFR mRNA knockdown experiment target 30
  source 1..66: mol_type = other RNA
ctactatactggtgcgaatttgcactagtctaaaatctcgccgggcagagcgcagccagcagcgcc

SEQ ID NO: 100 - RNA, 66 nt, synthetic construct
  misc_feature 1..66: note = Synthetic: CasM EGFR mRNA knockdown experiment target 31
  source 1..66: mol_type = other RNA
ctactatactggtgcgaatttgcactagtctaaaatactcgccgggcagagcgcagccagcagcgc

SEQ ID NO: 101 - RNA, 66 nt, synthetic construct
  misc_feature 1..66: note = Synthetic: CasM EGFR mRNA knockdown experiment target 32
  source 1..66: mol_type = other RNA
ctactatactggtgcgaatttgcactagtctaaaatgactcgccgggcagagcgcagccagcagcg

SEQ ID NO: 102 - RNA, 66 nt, synthetic construct
  misc_feature 1..66: note = Synthetic: CasM EGFR mRNA knockdown experiment target 33
  source 1..66: mol_type = other RNA
ctactatactggtgcgaatttgcactagtctaaaataaaagtgcccaactgcgtgagcttgttact

SEQ ID NO: 103 - RNA, 66 nt, synthetic construct
  misc_feature 1..66: note = Synthetic: CasM EGFR mRNA knockdown experiment target 34
  source 1..66: mol_type = other RNA
ctactatactggtgcgaatttgcactagtctaaaatatcttcaaaagtgcccaactgcgtgagctt

SEQ ID NO: 104 - RNA, 66 nt, synthetic construct
  misc_feature 1..66: note = Synthetic: CasM EGFR mRNA knockdown experiment target 35
  source 1..66: mol_type = other RNA
ctactatactggtgcgaatttgcactagtctaaaatatgatcttcaaaagtgcccaactgcgtgag

SEQ ID NO: 105 - RNA, 66 nt, synthetic construct
  misc_feature 1..66: note = Synthetic: CasM EGFR mRNA knockdown experiment target 36
  source 1..66: mol_type = other RNA
ctactatactggtgcgaatttgcactagtctaaaatcctctggaggctgagaaaatgatcttcaaa

SEQ ID NO: 106 - RNA, 66 nt, synthetic construct
  misc_feature 1..66: note = Synthetic: CasM EGFR mRNA knockdown experiment target 37
  source 1..66: mol_type = other RNA
ctactatactggtgcgaatttgcactagtctaaaatacatcctctggaggctgagaaaatgatctt

SEQ ID NO: 107 - RNA, 66 nt, synthetic construct
  misc_feature 1..66: note = Synthetic: CasM EGFR mRNA knockdown experiment target 38
  source 1..66: mol_type = other RNA
ctactatactggtgcgaatttgcactagtctaaaatagttattgaacatcctctggaggctgagaa

SEQ ID NO: 108 - RNA, 66 nt, synthetic construct
  misc_feature 1..66: note = Synthetic: CasM EGFR mRNA knockdown experiment target 39
  source 1..66: mol_type = other RNA
ctactatactggtgcgaatttgcactagtctaaaatacagttattgaacatcctctggaggctgag

SEQ ID NO: 109 - RNA, 66 nt, synthetic construct
  misc_feature 1..66: note = Synthetic: CasM EGFR mRNA knockdown experiment target 40
  source 1..66: mol_type = other RNA
ctactatactggtgcgaatttgcactagtctaaaatcacagttattgaacatcctctggaggctga

SEQ ID NO: 110 - RNA, 66 nt, synthetic construct
  misc_feature 1..66: note = Synthetic: CasM EGFR mRNA knockdown experiment target 41
  source 1..66: mol_type = other RNA
ctactatactggtgcgaatttgcactagtctaaaatacctcacagttattgaacatcctctggagg

SEQ ID NO: 111 - RNA, 66 nt, synthetic construct
  misc_feature 1..66: note = Synthetic: CasM EGFR mRNA knockdown experiment target 42
  source 1..66: mol_type = other RNA
ctactatactggtgcgaatttgcactagtctaaaataggaccacctcacagttattgaacatcctc

SEQ ID NO: 112 - RNA, 66 nt, synthetic construct
  misc_feature 1..66: note = Synthetic: CasM EGFR mRNA knockdown experiment target 43
  source 1..66: mol_type = other RNA
ctactatactggtgcgaatttgcactagtctaaaatcaaggaccacctcacagttattgaacatcc

SEQ ID NO: 113 - RNA, 66 nt, synthetic construct
  misc_feature 1..66: note = Synthetic: CasM EGFR mRNA knockdown experiment target 44
  source 1..66: mol_type = other RNA
ctactatactggtgcgaatttgcactagtctaaaatttcccaaggaccacctcacagttattgaac

SEQ ID NO: 114 - RNA, 66 nt, synthetic construct
  misc_feature 1..66: note = Synthetic: CasM EGFR mRNA knockdown experiment target 45
  source 1..66: mol_type = other RNA
ctactatactggtgcgaatttgcactagtctaaaataaattcccaaggaccacctcacagttattg

SEQ ID NO: 115 - RNA, 66 nt, synthetic construct
  misc_feature 1..66: note = Synthetic: CasM EGFR mRNA knockdown experiment target 46
  source 1..66: mol_type = other RNA
ctactatactggtgcgaatttgcactagtctaaaatcaaattcccaaggaccacctcacagttatt

SEQ ID NO: 116 - RNA, 66 nt, synthetic construct
  misc_feature 1..66: note = Synthetic: CasM EGFR mRNA knockdown experiment target 47
  source 1..66: mol_type = other RNA
ctactatactggtgcgaatttgcactagtctaaaatccaaattcccaaggaccacctcacagttat

SEQ ID NO: 117 - RNA, 66 nt, synthetic construct
  misc_feature 1..66: note = Synthetic: CasM EGFR mRNA knockdown experiment target 48
  source 1..66: mol_type = other RNA
ctactatactggtgcgaatttgcactagtctaaaattttccaaattcccaaggaccacctcacagt

SEQ ID NO: 118 - RNA, 66 nt, synthetic construct
  misc_feature 1..66: note = Synthetic: CasM EGFR mRNA knockdown experiment target 49
  source 1..66: mol_type = other RNA
ctactatactggtgcgaatttgcactagtctaaaattaatttccaaattcccaaggaccacctcac

SEQ ID NO: 119 - RNA, 66 nt, synthetic construct
  misc_feature 1..66: note = Synthetic: CasM EGFR mRNA knockdown experiment target 50
  source 1..66: mol_type = other RNA
ctactatactggtgcgaatttgcactagtctaaaatgtaatttccaaattcccaaggaccacctca

SEQ ID NO: 120 - RNA, 66 nt, synthetic construct
  misc_feature 1..66: note = Synthetic: CasM EGFR mRNA knockdown experiment target 51
  source 1..66: mol_type = other RNA
ctactatactggtgcgaatttgcactagtctaaaataggtaatttccaaattcccaaggaccacct

SEQ ID NO: 121 - RNA, 66 nt, synthetic construct
  misc_feature 1..66: note = Synthetic: CasM EGFR mRNA knockdown experiment target 52
  source 1..66: mol_type = other RNA
ctactatactggtgcgaatttgcactagtctaaaatcataggtaatttccaaattcccaaggacca

SEQ ID NO: 122 - RNA, 66 nt, synthetic construct
  misc_feature 1..66: note = Synthetic: CasM EGFR mRNA knockdown experiment target 53
  source 1..66: mol_type = other RNA
ctactatactggtgcgaatttgcactagtctaaaatctgcacataggtaatttccaaattcccaag

SEQ ID NO: 123 - RNA, 66 nt, synthetic construct
  misc_feature 1..66: note = Synthetic: CasM EGFR mRNA knockdown experiment target 54
  source 1..66: mol_type = other RNA
ctactatactggtgcgaatttgcactagtctaaaatctctgcacataggtaatttccaaattccca

SEQ ID NO: 124 - RNA, 66 nt, synthetic construct
  misc_feature 1..66: note = Synthetic: CasM EGFR mRNA knockdown experiment target 55
  source 1..66: mol_type = other RNA
ctactatactggtgcgaatttgcactagtctaaaataattcctctgcacataggtaatttccaaat

SEQ ID NO: 125 - RNA, 66 nt, synthetic construct
  misc_feature 1..66: note = Synthetic: CasM EGFR mRNA knockdown experiment target 56
  source 1..66: mol_type = other RNA
ctactatactggtgcgaatttgcactagtctaaaatagatcataattcctctgcacataggtaatt

SEQ ID NO: 126 - RNA, 66 nt, synthetic construct
  misc_feature 1..66: note = Synthetic: CasM EGFR mRNA knockdown experiment target 57
  source 1..66: mol_type = other RNA
ctactatactggtgcgaatttgcactagtctaaaatatgaggacataaccagccacctcctggatg

SEQ ID NO: 127 - RNA, 66 nt, synthetic construct
  misc_feature 1..66: note = Synthetic: CasM EGFR mRNA knockdown experiment target 58
  source 1..66: mol_type = other RNA
ctactatactggtgcgaatttgcactagtctaaaatgcaatgaggacataaccagccacctcctgg

SEQ ID NO: 128 - RNA, 66 nt, synthetic construct
  misc_feature 1..66: note = Synthetic: CasM EGFR mRNA knockdown experiment target 59
  source 1..66: mol_type = other RNA
ctactatactggtgcgaatttgcactagtctaaaataattcgctccactgtgttgagggcaatgag

SEQ ID NO: 129 - RNA, 66 nt, synthetic construct
  misc_feature 1..66: note = Synthetic: CasM EGFR mRNA knockdown experiment target 60
  source 1..66: mol_type = other RNA
ctactatactggtgcgaatttgcactagtctaaaatgttttccaaaggaattcgctccactgtgtt

SEQ ID NO: 130 - RNA, 66 nt, synthetic construct
  misc_feature 1..66: note = Synthetic: CasM EGFR mRNA knockdown experiment target 61
  source 1..66: mol_type = other RNA
ctactatactggtgcgaatttgcactagtctaaaattctgcaggttttccaaaggaattcgctcca

SEQ ID NO: 131 - RNA, 66 nt, synthetic construct
  misc_feature 1..66: note = Synthetic: CasM EGFR mRNA knockdown experiment target 62
  source 1..66: mol_type = other RNA
ctactatactggtgcgaatttgcactagtctaaaatgatctgcaggttttccaaaggaattcgctc

SEQ ID NO: 132 - RNA, 66 nt, synthetic construct
  misc_feature 1..66: note = Synthetic: CasM EGFR mRNA knockdown experiment target 63
  source 1..66: mol_type = other RNA
ctactatactggtgcgaatttgcactagtctaaaatgatgatctgcaggttttccaaaggaattcg

SEQ ID NO: 133 - RNA, 66 nt, synthetic construct
  misc_feature 1..66: note = Synthetic: CasM EGFR mRNA knockdown experiment target 64
  source 1..66: mol_type = other RNA
ctactatactggtgcgaatttgcactagtctaaaattgatgatctgcaggttttccaaaggaattc

SEQ ID NO: 134 - RNA, 66 nt, synthetic construct
  misc_feature 1..66: note = Synthetic: CasM EGFR mRNA knockdown experiment target 65
  source 1..66: mol_type = other RNA
ctactatactggtgcgaatttgcactagtctaaaattctgatgatctgcaggttttccaaaggaat

SEQ ID NO: 135 - RNA, 66 nt, synthetic construct
  misc_feature 1..66: note = Synthetic: CasM EGFR mRNA knockdown experiment target 66
  source 1..66: mol_type = other RNA
ctactatactggtgcgaatttgcactagtctaaaatatttcctctgatgatctgcaggttttccaa

SEQ ID NO: 136 - RNA, 66 nt, synthetic construct
  misc_feature 1..66: note = Synthetic: CasM EGFR mRNA knockdown experiment target 67
  source 1..66: mol_type = other RNA
ctactatactggtgcgaatttgcactagtctaaaatatatttcctctgatgatctgcaggttttcc

SEQ ID NO: 137 - RNA, 66 nt, synthetic construct
  misc_feature 1..66: note = Synthetic: CasM EGFR mRNA knockdown experiment target 68
  source 1..66: mol_type = other RNA
ctactatactggtgcgaatttgcactagtctaaaattttcgtagtacatatttcctctgatgatct

SEQ ID NO: 138 - RNA, 66 nt, synthetic construct
  misc_feature 1..66: note = Synthetic: CasM EGFR mRNA knockdown experiment target 69
  source 1..66: mol_type = other RNA
ctactatactggtgcgaatttgcactagtctaaaattaggaattttcgtagtacatatttcctctg

SEQ ID NO: 139 - RNA, 66 nt, synthetic construct
  misc_feature 1..66: note = Synthetic: CasM EGFR mRNA knockdown experiment target 70
  source 1..66: mol_type = other RNA
ctactatactggtgcgaatttgcactagtctaaaatcataggaattttcgtagtacatatttcctc

SEQ ID NO: 140 - RNA, 66 nt, synthetic construct
  misc_feature 1..66: note = Synthetic: CasM EGFR mRNA knockdown experiment target 71
  source 1..66: mol_type = other RNA
ctactatactggtgcgaatttgcactagtctaaaatctgctaaggcataggaattttcgtagtaca

SEQ ID NO: 141 - RNA, 66 nt, synthetic construct
  misc_feature 1..66: note = Synthetic: CasM EGFR mRNA knockdown experiment target 72
  source 1..66: mol_type = other RNA
ctactatactggtgcgaatttgcactagtctaaaatgataagactgctaaggcataggaattttcg

SEQ ID NO: 142 - RNA, 66 nt, synthetic construct
  misc_feature 1..66: note = Synthetic: CasM EGFR mRNA knockdown experiment target 73
  source 1..66: mol_type = other RNA
ctactatactggtgcgaatttgcactagtctaaaatatagttagataagactgctaaggcatagga

SEQ ID NO: 143 - RNA, 66 nt, synthetic construct
  misc_feature 1..66: note = Synthetic: CasM EGFR mRNA knockdown experiment target 74
  source 1..66: mol_type = other RNA
ctactatactggtgcgaatttgcactagtctaaaatatcatagttagataagactgctaaggcata

SEQ ID NO: 144 - RNA, 66 nt, synthetic construct
  misc_feature 1..66: note = Synthetic: CasM EGFR mRNA knockdown experiment target 75
  source 1..66: mol_type = other RNA
ctactatactggtgcgaatttgcactagtctaaaattatttgcatcatagttagataagactgcta

SEQ ID NO: 145 - RNA, 66 nt, synthetic construct
  misc_feature 1..66: note = Synthetic: CasM EGFR mRNA knockdown experiment target 76
  source 1..66: mol_type = other RNA
ctactatactggtgcgaatttgcactagtctaaaatagtccggttttatttgcatcatagttagat

SEQ ID NO: 146 - RNA, 66 nt, synthetic construct
  misc_feature 1..66: note = Synthetic: CasM EGFR mRNA knockdown experiment target 77
  source 1..66: mol_type = other RNA
ctactatactggtgcgaatttgcactagtctaaaatcttcagtccggttttatttgcatcatagtt

SEQ ID NO: 147 - RNA, 66 nt, synthetic construct
  misc_feature 1..66: note = Synthetic: CasM EGFR mRNA knockdown experiment target 78
  source 1..66: mol_type = other RNA
ctactatactggtgcgaatttgcactagtctaaaattgggcagctccttcagtccggttttatttg

SEQ ID NO: 148 - RNA, 66 nt, synthetic construct
  misc_feature 1..66: note = Synthetic: CasM EGFR mRNA knockdown experiment target 79
  source 1..66: mol_type = other RNA
ctactatactggtgcgaatttgcactagtctaaaatcatgggcagctccttcagtccggttttatt

SEQ ID NO: 149 - RNA, 66 nt, synthetic construct
  misc_feature 1..66: note = Synthetic: CasM EGFR mRNA knockdown experiment target 80
  source 1..66: mol_type = other RNA
ctactatactggtgcgaatttgcactagtctaaaattaaatttctcatgggcagctccttcagtcc

SEQ ID NO: 150 - RNA, 66 nt, synthetic construct
  misc_feature 1..66: note = Synthetic: CasM EGFR mRNA knockdown experiment target 81
  source 1..66: mol_type = other RNA
ctactatactggtgcgaatttgcactagtctaaaattagcccccagcgccacgacctccgagctac

SEQ ID NO: 151 - RNA, 66 nt, synthetic construct
  misc_feature 1..66: note = Synthetic: CasM EGFR mRNA knockdown experiment target 82
  source 1..66: mol_type = other RNA
ctactatactggtgcgaatttgcactagtctaaaatgcctcccgacagagcgctggtgctagcccc

SEQ ID NO: 152 - RNA, 66 nt, synthetic construct
  misc_feature 1..66: note = Synthetic: CasM EGFR mRNA knockdown experiment target 83
  source 1..66: mol_type = other RNA
ctactatactggtgcgaatttgcactagtctaaaatttccagcaccgagcgccctggccggtgagt

SEQ ID NO: 153 - RNA, 66 nt, synthetic construct
  misc_feature 1..66: note = Synthetic: CasM EGFR mRNA knockdown experiment target 84
  source 1..66: mol_type = other RNA
ctactatactggtgcgaatttgcactagtctaaaatagaaaaaagaagagggataaaacccggatc

SEQ ID NO: 154 - RNA, 66 nt, synthetic construct
  misc_feature 1..66: note = Synthetic: CasM EGFR mRNA knockdown experiment target 85
  source 1..66: mol_type = other RNA
ctactatactggtgcgaatttgcactagtctaaaatgggaagtagagcaatctccccaagccgtcg

SEQ ID NO: 155 - RNA, 66 nt, synthetic construct
  misc_feature 1..66: note = Synthetic: CasM EGFR mRNA knockdown experiment target 86
  source 1..66: mol_type = other RNA
ctactatactggtgcgaatttgcactagtctaaaatggggaggaggtggtagctggggctgggggc

SEQ ID NO: 156 - RNA, 66 nt, synthetic construct
  misc_feature 1..66: note = Synthetic: CasM EGFR mRNA knockdown experiment target 87
  source 1..66: mol_type = other RNA
ctactatactggtgcgaatttgcactagtctaaaatcaccccgcctccgggcgcgggctccggccc

SEQ ID NO: 157 - RNA, 66 nt, synthetic construct
  misc_feature 1..66: note = Synthetic: CasM EGFR mRNA knockdown experiment target 88
  source 1..66: mol_type = other RNA
ctactatactggtgcgaatttgcactagtctaaaatcacggctcctccgaagcgagaacagcccag

SEQ ID NO: 158 - RNA, 66 nt, synthetic construct
  misc_feature 1..66: note = Synthetic: CasM EGFR mRNA knockdown experiment target 89
  source 1..66: mol_type = other RNA
ctactatactggtgcgaatttgcactagtctaaaattccgggacggccggggcagcgctcctggcg

SEQ ID NO: 159 - RNA, 66 nt, synthetic construct
  misc_feature 1..66: note = Synthetic: CasM EGFR mRNA knockdown experiment target 90
  source 1..66: mol_type = other RNA
ctactatactggtgcgaatttgcactagtctaaaatccgggacggccggggcagcgctcctggcgc

SEQ ID NO: 160 - RNA, 66 nt, synthetic construct
  misc_feature 1..66: note = Synthetic: CasM EGFR mRNA knockdown experiment target 91
  source 1..66: mol_type = other RNA
ctactatactggtgcgaatttgcactagtctaaaatggacggccggggcagcgctcctggcgctgc

SEQ ID NO: 161 - RNA, 66 nt, synthetic construct
  misc_feature 1..66: note = Synthetic: CasM EGFR mRNA knockdown experiment target 92
  source 1..66: mol_type = other RNA
ctactatactggtgcgaatttgcactagtctaaaatgacggccggggcagcgctcctggcgctgct

SEQ ID NO: 162 - RNA, 66 nt, synthetic construct
  misc_feature 1..66: note = Synthetic: CasM EGFR mRNA knockdown experiment target 93
  source 1..66: mol_type = other RNA
ctactatactggtgcgaatttgcactagtctaaaatacggccggggcagcgctcctggcgctgctg

SEQ ID NO: 163 - RNA, 66 nt, synthetic construct
  misc_feature 1..66: note = Synthetic: CasM EGFR mRNA knockdown experiment target 94
  source 1..66: mol_type = other RNA
ctactatactggtgcgaatttgcactagtctaaaatcggccggggcagcgctcctggcgctgctgg

SEQ ID NO: 164 - RNA, 66 nt, synthetic construct
  misc_feature 1..66: note = Synthetic: CasM EGFR mRNA knockdown experiment target 95
  source 1..66: mol_type = other RNA
ctactatactggtgcgaatttgcactagtctaaaatggccggggcagcgctcctggcgctgctggc

SEQ ID NO: 165 - RNA, 66 nt, synthetic construct
  misc_feature 1..66: note = Synthetic: CasM EGFR mRNA knockdown experiment target 96
  source 1..66: mol_type = other RNA
ctactatactggtgcgaatttgcactagtctaaaatccggggcagcgctcctggcgctgctggctg

(19) United States
(12) Patent Application Publication
(10) Pub. No.: US 2025/0043257 A1
(43) Pub. Date: Feb. 6, 2025

(54) NOVEL CRISPR-ASSOCIATED (CAS) PROTEIN

(71) Applicant: Locanabio, Inc., San Diego, CA (US)

(72) Inventors: Matthew Merrill CARTER, San Diego, CA (US); Paul Daniel DONOHOUE, San Diego, CA (US)

(21) Appl. No.: 18/435,579

(22) Filed: Feb. 7, 2024

Related U.S. Application Data
(63) Continuation of application No. 17/319,849, filed on May 13, 2021, now Pat. No. 11,932,883.
Application No. 17/319,849 is a continuation of application No. 16/693,182, filed on Nov. 22, 2019, now Pat. No. 11,028,381.
Application No. 16/693,182 is a division of application No. 15/937,840, filed on Mar. 27, 2018, now Pat. No. 10,876,101.
(60) Provisional application No. 62/629,641, filed on Feb. 12, 2018.
(60) Provisional application No. 62/477,494, filed on Mar. 28, 2017.

(51) Int. Cl. C12N 9/22; C12N 15/11; C12N 15/113; C12N 15/85

(52) U.S. Cl. CPC C12N 9/22; C12N 15/11; C12N 15/111; C12N 15/1136; C12N 15/85; C12N 2310/20; C12N 2800/22

(57) ABSTRACT

A new CRISPR-associated (Cas) protein, termed “CasM,” is described, as well as polynucleotides encoding the same and methods of using CasM for site-specific genome engineering. CasM proteins are capable of targeting and cleaving single-stranded RNA.

### CROSS REFERENCE TO RELATED APPLICATIONS

This application is a continuation of U.S. patent application Ser. No. 17/319,849, filed May 13, 2021, which is a continuation of U.S. patent application Ser. No. 16/693,182, filed Nov. 22, 2019, now issued as U.S. Pat. No. 11,028,381, which is a divisional of U.S. patent application Ser. No. 15/937,840, filed Mar. 27, 2018, now issued as U.S. Pat. No. 10,876,101, which claims the benefit under 35 U.S.C. § 1 19 (e) (1) of U.S. Provisional Application Nos. 62/477,494, filed Mar. 28, 2017, and 62/629,641, filed Feb. 12, 2018, which applications are incorporated herein by reference in their entireties.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SEQUENCE LISTING

The instant application contains a Sequence Listing which has been submitted in XML format via Patent Center and is hereby incorporated by reference in its entirety. Said XML copy, created on Feb. 7, 2024 is named “LOCN-006_C04US_SeqList_ST26” and is 335,673 bytes in size.

### TECHNICAL FIELD

The present invention relates to Clustered Regularly Interspaced Short Palindromic Repeats (CRISPR) systems. In particular, the invention relates to a new CRISPR-associated (Cas) protein, termed “CasM,” and the uses of CasM for site-specific nucleic acid engineering.

### BACKGROUND OF THE INVENTION

Clustered Regularly Interspaced Short Palindromic Repeats (CRISPR) and CRISPR-associated (Cas) proteins are found in prokaryotic immune systems. These systems provide resistance against exogenous genetic elements, such as viruses and plasmids, by targeting their nucleic acids for degradation, in a sequence-specific manner.

There are several different CRISPR-Cas systems and the nomenclature and classification of these have changed as the systems have been characterized. In particular, CRISPR-Cas systems have now been reclassified into two classes, containing several types and subtypes (Makarova et al., Nature Reviews Microbiology (2015) 13:1-15; Shmakov et al., Nature Reviews Microbiology (2017) 15:169-182). This classification is based upon identifying all cas genes in a CRISPR-Cas locus and then determining the signature genes in each CRISPR-Cas locus, thereby determining whether the CRISPR-Cas systems should be placed in either Class 1 or Class 2 based upon the genes encoding the effector module, i.e., the proteins involved in the interference stage.

There remains a need to discover and characterize new CRISPR-associated (Cas) proteins, and their potential use for site-specific nucleic acid engineering.

### SUMMARY

The present invention is based on the discovery of a new Cas protein, termed “CasM” herein. This protein shares no homology to any known Cas protein or to any known protein family.

Accordingly, in one aspect, the invention is directed to an isolated CasM protein capable of producing a single-strand break at an RNA target site when guided to the RNA target site by a cognate nucleic acid guide. In certain embodiments, the cognate nucleic acid guide comprises RNA, such as crRNA. In additional embodiments, the CasM protein comprises an amino acid sequence selected from the group consisting of the amino acid sequence of SEQ ID NOS: 37-44 or 45; an ortholog of the amino acid sequence of SEQ ID NOS: 37-44 or 45, i.e., a CasM sequence from a species other than the species producing the reference sequence; and a variant of the amino acid sequence of SEQ ID NOS: 37-44 or 45, e.g., an active homolog of the reference amino acid sequence.

In further embodiments, the invention is directed to a complex comprising a CasM protein, and a cognate nucleic acid guide. In certain embodiments, the cognate nucleic acid guide in the complex comprises a repeat sequence and a spacer sequence, wherein the repeat sequence and the spacer sequence do not naturally occur together. In certain embodiments, the cognate nucleic acid guide comprises a modified base analog.

In additional embodiments, the cognate nucleic acid guide comprises RNA, such as, but not limited to, crRNA. In some embodiments, the cognate nucleic acid guide, such as crRNA, comprises a spacer sequence that is complementary to a DNA or RNA target sequence that occurs in a prokaryotic or eukaryotic cell.

In further embodiments, the crRNA/CasM protein complex is capable of binding to a first RNA target sequence complementary to the crRNA spacer sequence, wherein binding of the crRNA/CasM protein complex results in the cleavage of a first RNA target. In additional embodiments, after cleavage of the first RNA target sequence by the crRNA/CasM protein complex, the complex is capable of non-specific endonuclease activity toward any single-stranded RNA in a sequence independent manner.

In further embodiments, the complex modifies the transcription or translation of a target locus in cell.

In additional embodiments, the invention is directed to an isolated polynucleotide encoding a CasM protein, wherein the CasM protein is capable of producing a single-strand break at an RNA target site when guided to the RNA target site by a cognate nucleic acid guide. In certain embodiments, the cognate nucleic acid guide comprises RNA, such as crRNA. In additional embodiments, the CasM protein encoded by the polynucleotide comprises an amino acid sequence selected from the group consisting of the amino acid sequence of SEQ ID NOS: 37-44 or 45; an ortholog of the amino acid sequence of SEQ ID NOS: 37-44 or 45, i.e., a CasM sequence from a species other than the species producing the reference sequence; and a variant of the amino acid sequence of SEQ ID NOS: 37-44 or 45, e.g., an active homolog of the reference amino acid sequence.

In further embodiments, the invention is directed to a modified polynucleotide encoding a CasM protein, wherein the CasM protein is capable of producing a single-strand break at an RNA target site when guided to the RNA target site by a cognate nucleic acid guide, wherein the polynucleotide is modified relative to its native sequence, such as modified for expression in a selected host cell. In additional embodiments, the CasM protein encoded by the polynucleotide comprises an amino acid sequence selected from the group consisting of the amino acid sequence of SEQ ID NOS: 37-44 or 45; an ortholog of the amino acid sequence of SEQ ID NOS: 37-44 or 45, i.e., a CasM sequence from a species other than the species producing the reference sequence; and a variant of the amino acid sequence of SEQ ID NOS: 37-44 or 45, e.g., an active homolog of the reference amino acid sequence.

In certain embodiments, the polynucleotide is modified for expression in a bacterial cell, such as for expression in an Escherichia coli cell. In certain embodiments, the polynucleotide comprises the sequence of SEQ ID NOS: 2-8 or 9.

In other embodiments, the polynucleotide is modified for expression in a eukaryotic cell, e.g., a mammalian cell, such as a human cell. In certain embodiments, the polynucleotide comprises the sequence of SEQ ID NOS: 10-17 or 18.

In additional embodiments, the polynucleotide is modified for expression in a plant cell, such as for expression in a Zea mays (corn) cell. In certain embodiments the polynucleotide comprises the sequence of SEQ ID NOS: 19-26 or 27.

In further embodiments, the invention is directed to a recombinant vector comprising a polynucleotide or modified polynucleotide as described herein, and at least one control element operably linked to the polynucleotide, whereby a CasM coding sequence in the polynucleotide is capable of being transcribed and translated in a host cell. In certain embodiments, at least one of the control elements is heterologous to the coding system.

In additional embodiments, the CasM protein encoded by the polynucleotide comprises an amino acid sequence selected from the group consisting of the amino acid sequence of SEQ ID NOS: 37-44 or 45; an ortholog of the amino acid sequence of SEQ ID NOS: 37-44 or 45; and a variant of the amino acid sequence of SEQ ID NOS: 37-44 or 45.

In further embodiments, the invention is directed to a host cell transformed with a recombinant vector described herein. In certain embodiments, the host cell is a prokaryotic or eukaryotic cell.

In additional embodiments, the invention is directed to a method of producing a CasM protein comprising providing a population of host cells transformed with a recombinant vector as described herein; and culturing the population of cells under conditions whereby the CasM protein encoded by the polynucleotide present in the recombinant vector is expressed.

In further embodiments, the invention is directed to a eukaryotic host cell comprising a CasM protein of a complex comprising the CasM protein, as described herein.

In additional embodiments, the invention is directed to a method of directing a CasM protein to a selected nucleic acid target sequence, comprising contacting the selected nucleic acid target sequence with a cognate nucleic acid guide/CasM complex that targets said selected nucleic acid target sequence, whereby the CasM protein is delivered to the nucleic acid target sequence. In certain embodiments, the nucleic acid target sequence comprises RNA, such as mRNA. In further embodiments, the method comprises producing one or more single- or double-strand breaks in the target sequence.

In additional embodiments, the method is performed in a cell, such as a prokaryotic or eukaryotic cell. In certain embodiments, the cell constitutively expresses the CasM protein. In other embodiments, e.g., when the cell does not constitutively express the CasM protein, the cognate nucleic acid guide is complexed to the CasM protein prior to delivery to the nucleic acid target sequence. In other embodiments, the cell constitutively expresses the CasM protein and the cognate nucleic acid guide. In additional embodiments, the complex modifies the transcription or translation of a selected nucleic acid sequence in a host cell, such as a RNA sequence.

In further embodiments of the methods, the CasM protein comprises an amino acid sequence selected from the group consisting of the amino acid sequence of SEQ ID NOS: 37-44 or 45; an ortholog of the amino acid sequence of SEQ ID NOS: 37-44 or 45; and a variant of the amino acid sequence of SEQ ID NOS: 37-44 or 45.

In other embodiments of the methods, a donor polynucleotide is delivered to the region of the selected nucleic acid target sequence.

In additional embodiments, the CasM protein is capable of processing the CRISPR repeat-spacer array into individual repeat-spacer elements. The CasM protein cleaves the array within 5′ region of each repeat sequence, giving rise to a processed crRNA comprising, in a 5′ to 3′ direction, a repeat sequence and a spacer element.

In some embodiments the repeat sequence comprises a secondary structure that is recognized by the CasM protein. The secondary structure of the repeat may comprise a stem, a stem-loop duplex, a pseudoknot, or a tripartite duplex. CasM protein homologs may only recognize the repeat sequence or secondary structure of their cognate repeat elements.

Alternatively, CasM protein homologs may recognize the repeat sequence or secondary structure of non-cognate repeat elements.

In some embodiments, the crRNA/CasM complex is capable of sequence-specific single-stranded RNA activity. Recognition and cleavage of an initial ssRNA complementary to the crRNA target sequence activates the CasM protein to carry out endonuclease activity toward any single-stranded RNA in a sequence-independent manner. The sequence-specific recognition of RNA of the crRNA/CasM complex facilitates the target knockdown of gene transcripts perturbing translation of a specific protein. The non-specific endonuclease activity of an activated crRNA/CasM complex in a cellular environment can result in cell death due to depletion of RNA encoding for essential gene transcripts. The specific RNA targeting and collateral endonuclease activity of an activated crRNA/CasM complex enables the sequence-specific selection of cells expressing a RNA transcript.

In a further aspect, the present invention relates to a method of screening and killing cells that have not been modified by a DNA targeting nuclease (e.g., a Type II Cas9 nuclease). This method comprises contacting a crRNA/Cas9 complex to a locus of interest in a population of cells. Contacting the NATNA/Cas9 complex results in DNA cleavage and subsequent repair of the break by the endogenous cellular repair machine and the introduction of insertion and deletions (“indels”) at the break site. The targeting of the NATNA/Cas9 to a targeted locus that encodes an RNA transcript results in indels in an RNA transcript sequence. This modified RNA transcript sequence is different compared to a transcript from an unmodified cell (a wild-type cell). A cognate nucleic acid guide/CasM complex can then be targeted to the unmodified transcript, wherein recognition of the unmodified transcript by the complex results in activation of the sequence independent, single-stranded RNA targeting activity of the CasM protein and subsequent cell death. Alternatively, this method can be adapted to screen for the incorporation of a donor-polynucleotide into NATNA/Cas9 break site.

These aspects and other embodiments of the present disclosure will readily occur to those of ordinary skill in the art in view of the disclosure herein.

### BRIEF DESCRIPTION OF THE FIGURES

FIG. 1 depicts a representative CasM operon from Eubacterium siraeum (NCBI Accession No. NZ_DS499551.1).

FIG. 2 shows a structure of a CasM repeat sequence (SEQ ID NO:51).

FIG. 3 shows the results of the in vitro CRISPR array cleavage assay described in the Examples.

FIG. 4 shows a depiction of a synthetic CasM CRISPR array.

FIG. 5 shows a representation of the results of the in vitro CRISPR array cleavage assay results shown in FIG. 3 and described in the Examples.

FIG. 6 shows the results of the CasM ssRNA cleavage assay described in the Examples.

### DETAILED DESCRIPTION OF THE INVENTION

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to a “guide/Cas complex” includes one or more such complexes, reference to “a polynucleotide” includes one or more polynucleotides, etc.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, 2014, Cold Spring Harbor Laboratory Press, ISBN 978-1-936113-81-1; Culture of Animal Cells: A Manual of Basic Technique and Specialized Applications, 6th Edition, R. I. Freshney, 2010, Wiley-Blackwell, ISBN 978-0-470-52812-9; Transgenic Animal Technology, Third Edition: A Laboratory Handbook, 2014, C. A. Pinkert, Elsevier, ISBN 978-0124104907; The Laboratory Mouse, Second Edition, 2012, H. Hedrich, Academic Press, ISBN 978-0123820082; Manipulating the Mouse Embryo: A Laboratory Manual, 2013, R. Behringer, et al., Cold Spring Harbor Laboratory Press, ISBN 978-1936113019; PCR 2: A Practical Approach, 1995, M. J. McPherson, et al., IRL Press, ISBN 978-0199634248; Methods in Molecular Biology (Series), J. M. Walker, ISSN 1064-3745, Humana Press; RNA: A Laboratory Manual, 2010, D. C. Rio, et al., Cold Spring Harbor Laboratory Press, ISBN 978-0879698911; Methods in Enzymology (Series), Academic Press; Molecular Cloning: A Laboratory Manual (Fourth Edition), 2012, M. R. Green, et al., Cold Spring Harbor Laboratory Press, ISBN 978-1605500560; Bioconjugate Techniques, Third Edition, 2013, G. T. Hermanson, Academic Press, ISBN 978-0123822390; Methods in Plant Biochemistry and Molecular Biology, 1997, W. V. Dashek, CRC Press, ISBN 978-0849394805; Plant Cell Culture Protocols (Methods in Molecular Biology), 2012, V. M. Loyola-Vargas, et al., Humana Press, ISBN 978-1617798177; Plant Transformation Technologies, 2011, C. N. Stewart, et al., Wiley-Blackwell, ISBN 978-0813821955; Recombinant Proteins from Plants (Methods in Biotechnology), 2010, C. Cunningham, et al., Humana Press, ISBN 978-1617370212; Plant Genomics: Methods and Protocols (Methods in Molecular Biology), 2009, D. J. Somers, et al., Humana Press, ISBN 978-1588299970; Plant Biotechnology: Methods in Tissue Culture and Gene Transfer, 2008, R. Keshavachandran, et al., Orient Blackswan, ISBN 978-8173716164.

As used herein, “a CasM protein” refers to a CRISPR protein capable of targeting RNA and causing single-strand RNA breaks when guided to a target site by a crRNA, without the necessity of association with a tracrRNA. CasM proteins typically include two or more higher eukaryotic and prokaryotic nucleotide-binding (HEPN) domains found in protein family PF05168 in the C-terminal region of the CasM sequence. CasM proteins show synteny with one or more WYL domain-containing proteins and sometimes with RtcB (RNA 3′-terminal phosphate cyclase, group B) domain-containing proteins. Based on the foregoing characteristics, CasM may be classified as a Class 2 Type VI CRISPR-Cas system because it is a single effector protein containing two HEPN domains used for targeted ssRNA interference. However, CasM has a very low degree of sequence similarity to other Type VI subtypes. Exemplary CasM proteins are shown in SEQ ID NOS: 37-45, and are encoded by polynucleotides shown in SEQ ID NOS: 28-36, respectively. These proteins display approximately 13.59% to 99.82% sequence identity to each other and show less than 8% sequence identity with other known CRISPR-Cas proteins. As used herein, the term “CasM protein” refers to a CasM protein derived from any species, subspecies, or strain of bacteria that encodes the CasM protein, as well as an ortholog of the CasM protein, i.e., a CasM protein from a species other than the species producing the reference CasM protein. For example, CasM orthologs of Eubacterium siraeum CasM, shown in Table 1, display approximately 13.59% to 99.82% sequence identity to each other. Thus, CasM orthologs are identified based on the CasM characteristics detailed herein. Reference to a CasM protein also encompasses a variant of the reference CasM protein, e.g., an active homolog of the reference amino acid sequence. Thus, CasM proteins include, but are not limited to, those proteins depicted in SEQ ID NOS: 37-45, orthologs thereof, or variants thereof. Non-limiting examples of such proteins include CasM proteins from Eubacterium siraeum; Ruminococcus sp., such as from Ruminococcus bicirculans; Ruminococcus flavefaciens, such as, but not limited to, FD-1 and strain XPD3002; Ruminococcus albus such as, but not limited to, strain KH2T6; Ruminococcus sp. isolates, such as but not limited to, isolates 2789STDY5834971, 2789STDY5608892 and 2789STDY5834894.

By “dCasM protein” is meant a deactivated CasM protein lacking activity, such as catalytic and/or binding activity, also termed “dead CasM.” Such molecules lack all or a portion of biological activity, such as nuclease and/or binding activity, and are therefore unable to bind and/or cleave a target nucleic acid of interest, respectively. In some embodiments, these deactivated CasM proteins can be used to regulate genes in a nucleic acid-guided manner. This is accomplished by introducing mutations that inactivate CasM nuclease function and typically involves mutating catalytic residues of the gene encoding CasM. dCasM can be used alone or in fusions to synthetically repress (CRISPR interference or CRISPRi) or activate (CRISPR activation or CRISPRa) gene expression. CRISPRi can work independently of host cellular machineries. In some embodiments a dCasM protein and a customized nucleic acid-targeting nucleic acid, i.e., a cognate nucleic acid guide designed with a complementary region to any gene of interest, are used to direct dCasM to a chosen genomic location. In other embodiments, dCasM can be fused to a transcription factor, such as a repressor, and the fused dCasM-transcription factor can then work in concert with cellular machineries. CRISPRa is carried out by dCasM-transcription factor (activator) fusions.

A “nucleic acid-targeting nucleic acid” (NATNA), as used herein, refers to one or more polynucleotides that guide a protein, such as a CasM protein, to preferentially target a nucleic acid target sequence present in a polynucleotide (relative to a polynucleotide that does not comprise the nucleic acid target sequence). Such NATNAs are also known herein as “cognate nucleic acid guides,” or “cognate guides.” NATNAs can comprise ribonucleotide bases (e.g., RNA), deoxyribonucleotide bases (e.g., DNA), combinations of ribonucleotide bases and deoxyribonucleotide bases (e.g., RNA/DNA), nucleotides, nucleotide analogs, modified nucleotides, and the like, as well as synthetic, naturally occurring, and non-naturally occurring modified backbone residues or linkages. Thus, a NATNA as used herein site-specifically guides a CasM, or a deactivated CasM, to a target nucleic acid. Many such NATNAs are known, such as but not limited to sgRNA (including miniature and truncated single-guide RNAs), crRNA, dual-guide RNA, including but not limited to, crRNA/tracrRNA molecules, as described herein, and the like, the use of which depends on the particular Cas protein. For a non-limiting description of exemplary NATNAs, see, e.g., PCT Publication No. WO 2014/150624 to May et al., published Sep. 29, 2014; PCT Publication No. WO 2015/200555 to May et al., published Mar. 10, 2016; PCT Publication No. WO 2016/201155 to Donohoue et al., published Dec. 15, 2016; PCT Publication No. WO 2017/027423 to Donohoue et al., published Feb. 16, 2017; and PCT Publication No. WO 2016/123230 to May et al., published Aug. 4, 2016.

With reference to a NATNA or a cognate nucleic acid guide, a “spacer,” “spacer sequence,” or “spacer element,” as used herein, refers to the polynucleotide sequence that can specifically hybridize to a target nucleic acid sequence. The spacer element interacts with the target nucleic acid sequence through hydrogen bonding between complementary base pairs (i.e., paired bases). A spacer element binds to a selected nucleic acid target sequence. Accordingly, the spacer element is the nucleic acid target-binding sequence. The spacer element determines the location of a Cas protein's site-specific binding and nucleolytic cleavage. Spacer elements range from approximately 17 to approximately 84 nucleotides in length and have an average length of 36 nucleotides (see, e.g., Marraffini, et al., “CRISPR interference: RNA-directed adaptive immunity in bacteria and archaea,” Nature reviews Genetics (2010) 11:181-190). Variability of the functional length for a spacer element is known in the art (e.g., U.S. Patent Publication 2014/0315985 to May et al., published Oct. 23, 2014). The terms “nucleic acid target binding sequence” and “spacer sequence” are used interchangeably herein. The term “sgRNA” typically refers to a single-guide RNA (i.e., a single, contiguous polynucleotide sequence) that essentially comprises a crRNA connected at its 3′ end to 5′ end of a tracrRNA through a “loop” sequence (see, e.g., U.S. Published Patent Application No. 2014/0068797 to Doudna et al., published 6 Mar. 2014, incorporated herein by reference in its entirety). sgRNA interacts with a cognate Cas protein essentially as described for tracrRNA/crRNA polynucleotides. Similar to crRNA, sgRNA has a spacer, a region of complementarity to a potential DNA or RNA target sequence, adjacent a second region that forms base-pair hydrogen bonds that form a secondary structure, typically a stem structure. The term includes truncated single-guide RNAs (tru-sgRNAs) of approximately 17-18 nucleotides (nt) (see, e.g., Fu et. al., Nat Biotechnol. (2014) 32:279-284). The term also encompasses functional miniature sgRNAs with expendable features removed, but that retain an essential and conserved module termed the “nexus” located in the portion of sgRNA that corresponds to tracrRNA (not crRNA). See, e.g., U.S. Patent Publication 2014/0315985 to May et al., published Oct. 23, 2014, incorporated herein by reference in its entirety; Briner et al., “Guide RNA Functional Modules Direct Cas9 Activity and Orthogonality,” Molecular Cell (2014) 56:333-339.

As used herein, “dual-guide RNA” refers to a two-component RNA system for a polynucleotide component capable of associating with a cognate Cas protein. A representative CRISPR Class 2 Type II CRISPR-Cas-associated dual-guide RNA includes a Cas-crRNA and Cas-tracrRNA, paired by hydrogen bonds to form secondary structure (see, e.g., U.S. Published Patent Application No. 2014/0068797 to Doudna et al., published 6 Mar. 2014, incorporated herein by reference in its entirety; see also Jinek M., et al., Science 337:816-21 (2012)). A Cas-dual-guide RNA is capable of forming a nucleoprotein complex with a cognate Cas protein, wherein the complex is capable of targeting a nucleic acid target sequence complementary to the spacer sequence.

As used herein, the term “cognate” typically refers to a Cas protein (e.g., CasM protein) and one or more polynucleotides (e.g., a CRISPR-CasM-associated cognate nucleic acid guide) capable of forming a nucleoprotein complex for site-directed binding to a nucleic acid target sequence complementary to the nucleic acid target binding sequence present in one of the one or more polynucleotides.

The terms “wild-type,” “naturally-occurring,” “native,” and “unmodified” are used herein to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in and can be isolated from a source in nature. The wild-type form, appearance, phenotype, or strain serve as the original parent before an intentional modification. Thus, mutant, variant, engineered, recombinant, and modified forms are not wild-type forms.

As used herein, the terms “engineered,” “genetically engineered,” “recombinant,” “modified,” and “non-naturally occurring” are interchangeable and indicate intentional human manipulation.

“Covalent bond,” “covalently attached,” “covalently bound,” “covalently linked,” “covalently connected,” and “molecular bond” are used interchangeably herein, and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to hydrogen bonding, ionic interactions (e.g., Na⁺Cl⁻), van der Waals interactions, and hydrophobic bonds.

As used herein, “hydrogen bonding,” “hydrogen base pairing,” and “hydrogen bonded” are used interchangeably and refer to canonical hydrogen bonding and non-canonical hydrogen bonding including, but not limited to, “Watson-Crick-hydrogen-bonded base pairs” (W—C-hydrogen-bonded base pairs or W—C hydrogen bonding); “Hoogsteen-hydrogen-bonded base pairs” (Hoogsteen hydrogen bonding); and “wobble-hydrogen-bonded base pairs” (wobble hydrogen bonding). W—C hydrogen bonding, including reverse W—C hydrogen bonding, refers to purine-pyrimidine base pairing, that is, adenine: thymine, guanine: cytosine, and uracil: adenine. Hoogsteen hydrogen bonding, including reverse Hoogsteen hydrogen bonding, refers to a variation of base pairing in nucleic acids wherein two nucleobases, one on each strand, are held together by hydrogen bonds in the major groove. This non-W—C hydrogen bonding can allow a third strand to wind around a duplex and form triple-stranded helices. Wobble hydrogen bonding, including reverse wobble hydrogen bonding, refers to a pairing between two nucleotides in RNA molecules that does not follow Watson-Crick base pair rules. There are four major wobble base pairs: guanine: uracil, inosine (hypoxanthine): uracil, inosine-adenine, and inosine-cytosine. Rules for canonical hydrogen bonding and non-canonical hydrogen bonding are known to those of ordinary skill in the art (see, e.g., The RNA World, Third Edition (Cold Spring Harbor Monograph Series), R. F. Gesteland, Cold Spring Harbor Laboratory Press, ISBN 978-0879697396 (2005); The RNA World, Second Edition (Cold Spring Harbor Monograph Series), R. F. Gesteland, et al., Cold Spring Harbor Laboratory Press, ISBN 978-0879695613 (1999); The RNA World (Cold Spring Harbor Monograph Series), R. F. Gesteland, et al., Cold Spring Harbor Laboratory Press, ISBN 978-0879694562 (1993) (see, e.g., Appendix 1: Structures of Base Pairs Involving at Least Two Hydrogen Bonds, I. Tinoco); Principles of Nucleic Acid Structure, W. Saenger, Springer International Publishing AG, ISBN 978-0-387-90761-1 (1988); Principles of Nucleic Acid Structure, First Edition, S. Neidle, Academic Press, ISBN 978-01236950791 (2007)).

“Connect,” “connected,” and “connecting” are used interchangeably herein, and refer to a covalent bond or a non-covalent bond between two macromolecules (e.g., polynucleotides, proteins, and the like). Thus, CasM and a cognate nucleic acid guide are “connected” in a cognate nucleic acid guide/CasM complex.

As used herein, the terms “nucleic acid,” “nucleic acid sequence,” “nucleotide sequence,” “oligonucleotide,” and “polynucleotide” are interchangeable and refer to a polymeric form of nucleotides. The nucleotides may be deoxyribonucleotides (DNA), ribonucleotides (RNA), analogs thereof, or combinations thereof, and may be of any length. Polynucleotides may perform any function and may have any secondary and tertiary structures. The terms encompass known analogs of natural nucleotides and nucleotides that are modified in the base, sugar and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, which are synthetic, naturally occurring, and non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Inc., Woburn, MA) nucleosides, glycol nucleic acid, bridged nucleic acids, and morpholino structures.

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3 to 5 nucleotides at 5′-end or 3′-end sequences of a polynucleotide sequence to inhibit exonuclease degradation. Placement of phosphorothioate bonds throughout an entire oligonucleotide helps reduce degradation by nucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., ucalgary.ca/dnalab/synthesis/-modifications/linkages). A 3′-3′ linkage at a terminus of a polynucleotide stabilizes the polynucleotide to exonuclease degradation by creating an oligonucleotide having two 5′-OH termini but lacking a 3′-OH terminus. Typically, such polynucleotides have phosphoramidite groups on 5′-OH position and a dimethoxytrityl (DMT) protecting group on 3′-OH position. Normally, the DMT protecting group is on 5′-OH and the phosphoramidite is on 3′-OH.

Polynucleotide sequences are displayed herein in the conventional 5′ to 3′ orientation unless otherwise indicated.

As used herein, the term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Sequence-specific binding, as used herein, typically refers to one or more cognate nucleic acid guides (i.e., NATNAs) capable of forming a complex with a protein (e.g., a CasM protein) to cause the protein to bind a nucleic acid sequence (e.g., a RNA or DNA sequence) comprising a nucleic acid target sequence (e.g., a RNA or DNA target sequence) preferentially relative to a second nucleic acid sequence (e.g., a second RNA or DNA sequence) without the nucleic acid target binding sequence (e.g., the RNA or DNA target binding sequence). All components of a binding interaction do not need to be sequence-specific, such as contacts of a protein with phosphate residues in a DNA backbone. Binding interactions can be characterized by a dissociation constant (Kd). “Binding affinity” refers to the strength of the binding interaction. An increased binding affinity is correlated with a lower Kd.

As used herein, a Cas protein (e.g., a CasM protein) is said to “target” a polynucleotide if a cognate nucleic acid/Cas protein nucleoprotein complex associates with, binds and/or cleaves a polynucleotide at the nucleic acid target sequence within the polynucleotide.

As used herein, “single-strand break” (SSB) refers to cleavage of a single strand of RNA or DNA. A “double-strand break” (DSB) refers to both strands of a double-stranded segment of nucleic acid being severed. In some instances, if such a break occurs, one strand can be said to have a “sticky end” wherein nucleotides are exposed and not hydrogen bonded to nucleotides on the other strand. In other instances, a “blunt end” can occur wherein both strands remain fully base paired with each other.

As used herein, the term “recombination” refers to a process of exchange of genetic information between two polynucleotides.

As used herein, “nucleic acid repair,” such as but not limited to DNA repair, encompasses any process whereby cellular machinery repairs damage to a nucleic acid molecule contained in the cell. The damage repaired can include single-strand breaks or double-strand breaks (DSBs). At least three mechanisms exist to repair DSBs: homology-directed repair (HDR), classical non-homologous end joining (c-NHEJ), and microhomology-mediated end joining (MMEJ), all defined below. “Nucleic acid repair” is also used herein to refer to nucleic acid repair resulting from human manipulation, wherein a target locus is modified, e.g., by inserting, deleting, or substituting nucleotides, all of which represent forms of genome editing.

As used herein, the term “homology-directed repair” or “HDR” refers to nucleic acid repair that takes place in cells, for example, during repair of double-strand and single-strand breaks in a nucleic acid molecule, such as DNA. HDR requires nucleotide sequence homology and uses a “donor template” (donor template nucleic acid, such as DNA, polynucleotide donor, or oligonucleotide (used interchangably herein) to repair the sequence where the double-strand break occurred (e.g., DNA target sequence). This results in the transfer of genetic information from, for example, the donor template DNA to the DNA target sequence. HDR may result in alteration of the nucleic acid target sequence (e.g., insertion, deletion, mutation) if the donor template sequence or oligonucleotide sequence differs from the target sequence and part or all of the donor template polynucleotide or oligonucleotide is incorporated into the target sequence. In some embodiments, an entire donor template polynucleotide, a portion of the donor template polynucleotide, or a copy of the donor polynucleotide is copied or integrated at the site of the target sequence.

By “donor polynucleotide” is meant a polynucleotide that can be directed to, and inserted into a target site of interest, such as an integration locus, to modify the target nucleic acid. All or a portion of the donor polynucleotide can be inserted into the target nucleic acid. The donor polynucleotide can be used for repair of the break in the target nucleic acid sequence resulting in the transfer of genetic information (i.e., polynucleotide sequences) from the donor at the site or in close proximity of the break. Accordingly, new genetic information (i.e., polynucleotide sequences) may be inserted or copied at a target site. The donor polynucleotide can be double- or single-stranded RNA, DNA, a vector, plasmid, or the like. Thus, a donor polynucleotide can be an insertion cassette, a recombinase expression vector, and the like. Non-symmetrical polynucleotide donors can also be used that are composed of two oligonucleotides. They are partially complementary, and each can include a flanking region of homology. The donor can be used to insert or replace polynucleotide sequences in a target sequence, for example, to introduce a polynucleotide that encodes a protein or functional RNA (e.g., siRNA), to introduce a protein tag, to modify a regulatory sequence of a gene, or to introduce a regulatory sequence to a gene (e.g. a promoter, an enhancer, an internal ribosome entry sequence, a start codon, a stop codon, a localization signal, or polyadenylation signal), to modify a nucleic acid sequence (e.g., introduce a mutation), and the like.

Targeted nucleic acid modifications using donor polynucleotides for large changes (e.g., more than 100 base pair (bp) insertions or deletions) traditionally use plasmid-based donor templates that contain homology arms flanking the site of alteration. Each arm can vary in length, but is typically longer than about 100 bp, such as 100-1500 bp, e.g., 100 . . . 200 . . . 300 . . . 400 . . . 500 . . . 600 . . . 700 . . . 800 . . . 900 . . . 1000 . . . 1500 bp or any integer between these values. However, these numbers can vary, depending on the size of the donor polynucleotide and the target polynucleotide. This method can be used to generate large modifications, including insertion of reporter genes such as fluorescent proteins or antibiotic resistance markers. For transfection in cells, such as HEK cells, approximately 100-1000 nanograms (ng), e.g., 100 . . . 200 . . . 300 . . . 400 . . . 500 . . . 600 . . . 700 . . . 800 . . . 900 . . . 1000 ng or any integer between these values, of a typical size donor plasmid (e.g., approximately 5 kb) containing a NATNA/Cas vector, can be used for one well in 24-well plate. (See, e.g., Yang et al., “One Step Generation of Mice Carrying Reporter and Conditional Alleles by CRISPR/Cas-Mediated Genome Engineering” Cell (2013) 154:1370-1379).

Single-stranded and partially double-stranded oligonucleotides, such as DNA oligonucleotides, have been used in place of targeting plasmids for short modifications (e.g., less than 50 bp) within a defined locus without cloning. To achieve high HDR efficiencies, single-stranded oligonucleotides containing flanking sequences on each side that are homologous to the target region can be used, and can be oriented in either the sense or antisense direction relative to the target locus. The length of each arm can vary, but the length of at least one arm is typically longer than about 10 bases, such as from 10-150 bases, e.g., . . . 20 . . . 30 . . . 40 . . . 50 . . . 60 . . . 70 . . . 80 . . . 90 . . . 100 . . . 110 . . . 120 . . . 130 . . . 140 . . . 150, or any integer within these ranges. However, these numbers can vary, depending on the size of the donor polynucleotide and the target polynucleotide. In some embodiments, the length of at least one arm is 10 bases or more. In other embodiments, the length of at least one arm is 20 bases or more. In yet other embodiments, the length of at least one arm is 30 bases or more. In some embodiments, the length of at least one arm is less than 100 bases. In further embodiments, the length of at least one arm is greater than 100 bases. In some embodiments, the length of at least one arm is zero bases. For single-stranded oligonucleotide design, typically an oligonucleotide with around 100-150 bp total homology is used. The mutation is introduced in the middle, giving 50-75 bp homology arms for a donor designed to be symmetrical about the target site. In other cases, no homology arms are required, and the donor polynucleotide is inserted using non-homologous repair mechanisms.

A “genomic region” is a segment of a chromosome in the genome of a host cell that is present on either side of the nucleic acid target sequence site or, alternatively, also includes a portion of the nucleic acid target sequence site. The homology arms of the donor polynucleotide have sufficient homology to undergo homologous recombination with the corresponding genomic regions. In some embodiments, the homology arms of the donor polynucleotide share significant sequence homology to the genomic region immediately flanking the nucleic acid target sequence site; it is recognized that the homology arms can be designed to have sufficient homology to genomic regions farther from the nucleic acid target sequence site.

As used herein the terms “classical non-homologous end joining” or “c-NHEJ” refer to the repair of double-strand breaks in DNA by direct ligation of one end of the break to the other end of the break without a requirement for a donor template DNA. NHEJ in the absence of a donor template DNA often results in small insertions or deletions of nucleotides at the site of the double-strand break, also referred to as “indels.” This DNA repair pathway is genetically defined and requires the activity of Ligase IV, DNA-PKcs, Polμ, Polλ, and the Ku70/80 heterodimer, among other proteins (see, e.g., Sfeir and Symington, Trends Biochem Sci (2015) 40:701-714).

“Microhomology-mediated end joining (MMEJ),” a form of alternative nonhomologous end-joining (alt-NHEJ), is another pathway for repairing double-strand breaks in DNA. MMEJ is associated with deletions flanking a DSB and involves alignment of microhomologous sequences internal to the broken ends before joining. The proposed mechanism entails 5′-3′ resection of the DNA ends at a DSB, annealing of the microhomologies (1-16 nucleotides of homology), removal of heterologous flaps, gap filling DNA synthesis, and ligation. MMEJ is genetically defined and requires the activity of CtIP, PARP1, Pole, Lig1 and Lig3, among other proteins (see, e.g., Sfeir and Symington, “Microhomology-Mediated End Joining: A Back-up Survival Mechanism or Dedicated Pathway?” Trends Biochem Sci (2015) 40:701-714).

Alternative mechanisms of nucleic acid insertion that do not require sequence homology between the donor and the target sequence can also be used for nucleic acid insertion. These mechanisms involve various components of the cellular repair machinery and it is to be understood that the scope of the invention is not bound by the use of any particular mechanism for insertion of nucleic acid after target nucleic acid is cut or nicked by a site-specific polynucleotide.

“Gene,” as used herein, refers to a polynucleotide sequence comprising exon(s) and related regulatory sequences. A gene may further comprise intron(s) and/or untranslated region(s) (UTR(s)).

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product(s).” Expression may include splicing the mRNA in a eukaryotic cell, if the polynucleotide is derived from genomic DNA.

As used herein, the term “modulate” refers to a change in the quantity, degree or amount of a function. For example, a cognate nucleic acid guide/CasM protein complex, as disclosed herein, may modulate the activity of a promoter sequence by binding to a nucleic acid target sequence at or near the promoter. Depending on the action occurring after binding, the cognate nucleic acid guide/CasM protein complex can induce, enhance, suppress, or inhibit transcription of a gene operatively linked to the promoter sequence. Thus, “modulation” of gene expression includes both gene activation and gene repression.

Modulation can be assayed by determining any characteristic directly or indirectly affected by the expression of the target gene. Such characteristics include, e.g., changes in RNA or protein levels, protein activity, product levels, expression of the gene, or activity level of reporter genes. Accordingly, the terms “modulating expression,” “inhibiting expression,” and “activating expression” of a gene can refer to the ability of a cognate guide/CasM protein complex to change, activate, or inhibit transcription of a gene.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes. An expression vector typically comprises an expression cassette.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used herein, a “targeting vector” is a recombinant DNA or RNA construct typically comprising tailored DNA or RNA arms, homologous to genomic DNA or RNA derived therefrom, that flank elements of a target gene or nucleic acid target sequence (e.g., a SSB or DSB). A targeting vector comprises a donor polynucleotide. Elements of the target sequence can be modified in a number of ways including deletions and/or insertions. A defective target gene can be replaced by a functional target gene, or in the alternative a functional gene can be knocked out. Optionally, the donor polynucleotide of a targeting vector comprises a selection cassette comprising a selectable marker that is introduced into the target gene. Targeting regions (i.e., nucleic acid target sequences) adjacent or within a target gene or region can be used to affect regulation of gene expression.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like.

Regulatory elements include those that direct constitutive, inducible, and repressible expression of a nucleotide sequence in many types of host cells and those that direct expression of the nucleotide sequence only in certain host cells (e.g., tissue-specific regulatory sequences). In some embodiments, a vector comprises one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart, M., et al., Cell 41:521-530 (1985)), the SV40 promoter, the dihydrofolate reductase promoter, the β-actin promoter, the phosphoglycerol kinase (PGK) promoter, and the EF1α promoter. It will be appreciated by those skilled in the art that the design of an expression vector may depend on such factors as the choice of the host cell to be transformed, the level of expression desired, and the like. A vector can be introduced into host cells to thereby produce transcripts, proteins, or peptides, including fusion proteins or peptides, encoded by nucleic acids as described herein.

As used herein the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Accordingly, some polynucleotide elements may be operably linked but not contiguous.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

As used herein, the term “sequence identity” generally refers to the percent identity of bases or amino acids determined by comparing a first polynucleotide or polypeptide to a second polynucleotide or polypeptide using algorithms having various weighting parameters. Sequence identity between two polypeptides or two polynucleotides can be determined using sequence alignment by various methods and computer programs (e.g., BLAST, CS-BLAST, FASTA, HMMER, L-ALIGN, etc.), available through the worldwide web at sites including GENBANK (ncbi.nlm.nih.gov/genbank/) and EMBL-EBI (ebi.ac.uk.). Sequence identity between two polynucleotides or two polypeptide sequences is generally calculated using the standard default parameters of the various methods or computer programs. Generally, Cas proteins, such as CasM homologs, for use herein will have at least about 75% or more sequence identity to the wild-type or naturally occurring sequence of the Cas protein of interest, such as about 80%, such as about 85%, 90%, 91%, 92%, 93%, 94%, 95%, 96%, 97%, 98%, 99%, or complete identity.

CasM orthologs can vary widely from the reference sequence. For example, CasM orthologs shown in Table 1 display approximately 13.59% to 99.82% sequence identity to each other. Thus, CasM orthologs are identified based on the CasM characteristics detailed herein.

As used herein, “hybridization,” “hybridize,” or “hybridizing” is the process of combining two complementary single-stranded DNA or RNA molecules so as to form a single double-stranded molecule (DNA/DNA, DNA/RNA, RNA/RNA) through hydrogen base pairing. Hybridization stringency is typically determined by the hybridization temperature and the salt concentration of the hybridization buffer; e.g., high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tₘ; moderate stringency, approximately 0.16M to approximately 0.33M salt, hybridization temperature 20° C. to 29° C. below Tₘ; and low stringency, approximately 0.33M to approximately 0.82M salt, hybridization temperature 40° C. to 48° C. below Tₘ. Tₘ of duplex nucleic acids is calculated by standard methods well-known in the art (see, e.g., Maniatis, T., et al., Molecular Cloning: A Laboratory Manual, Cold Spring Harbor Laboratory Press: New York (1982); Casey, J., et al., Nucleic Acids Research 4:1539-1552 (1977); Bodkin, D. K., et al., Journal of Virological Methods 10 (1): 45-52 (1985); Wallace, R. B., et al., Nucleic Acids Research 9 (4): 879-894 (1981)). Algorithm prediction tools to estimate Tₘ are also widely available. High stringency conditions for hybridization typically refer to conditions under which a polynucleotide complementary to a target sequence predominantly hybridizes with the target sequence, and substantially does not hybridize to non-target sequences. Typically, hybridization conditions are of moderate stringency, preferably high stringency.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to an amino acid polymer that has been modified through, for example, acetylation, disulfide bond formation, glycosylation, lipidation, phosphorylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology (see, e.g., standard texts set forth above). Further, essentially any polypeptide or polynucleotide can be custom ordered from commercial sources.

The terms “fusion protein” and “chimeric protein,” as used herein, refer to a single protein created by joining two or more proteins, protein domains, or protein fragments that do not naturally occur together in a single protein. For example, a fusion protein can contain a first domain from a CasM protein and a second domain from a different Cas protein. The modification to include such domains in fusion proteins may confer additional activity on the modified site-directed polypeptides. Such activities can include nuclease activity, methyltransferase activity, demethylase activity, DNA or RNA repair activity, DNA or RNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity) that modifies a polypeptide associated with nucleic acid target sequence (e.g., a histone). A fusion protein can also comprise epitope tags (e.g., histidine tags, FLAG® (Sigma Aldrich, St. Louis, MO) tags, Myc tags), reporter protein sequences (e.g., glutathione-S-transferase, beta-galactosidase, luciferase, green fluorescent protein, cyan fluorescent protein, yellow fluorescent protein), and/or nucleic acid binding domains (e.g., a DNA binding domain, an RNA binding domain). A fusion protein can also comprise activator domains (e.g., heat shock transcription factors, NFKB activators) or repressor domains (e.g., a KRAB domain). As described by Lupo, A., et al., Current Genomics 14 (4): 268-278 (2013), the KRAB domain is a potent transcriptional repression module and is located in the amino-terminal sequence of most C2H2 zinc finger proteins (see, e.g., Margolin, J., et al., Proceedings of the National Academy of Sciences of the United States of America 91:4509-4513 (1994); Witzgall, R., et al., Proceedings of the National Academy of Sciences of the United States of America 91:4514-4518 (1994)). The KRAB domain typically binds to co-repressor proteins and/or transcription factors via protein-protein interactions, causing transcriptional repression of genes to which KRAB zinc finger proteins (KRAB-ZFPs) bind (see, e.g., Friedman J. R., et al., Genes & Development 10:2067-2678 (1996)). In some embodiments, linker nucleic acid sequences are used to join the two or more proteins, protein domains, or protein fragments.

A “moiety,” as used herein, refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description. In some embodiments, a 5′ terminus, a 3′ terminus, or a 5′ terminus and a 3′ terminus (e.g., a non-native 5′ terminus and/or a non-native 3′ terminus in a first stem element) can comprise one or more moieties.

As used herein, the term “isolated” can refer to a nucleic acid or polypeptide that, by the hand of a human, exists apart from its native environment and is therefore not a product of nature. Isolated means substantially pure. An isolated nucleic acid or polypeptide can exist in a purified form and/or can exist in a non-native environment such as, for example, in a recombinant cell.

As used herein, a “host cell” generally refers to a biological cell. A cell is the basic structural, functional and/or biological unit of an organism. A cell can originate from any organism having one or more cells. Examples of host cells include, but are not limited to: a prokaryotic cell, eukaryotic cell, a bacterial cell, an archaeal cell, a cell of a single-cell eukaryotic organism, a protozoal cell, a cell from a plant (e.g., cells from plant crops (such as soy, tomatoes, sugar beets, pumpkin, hay, cannabis, tobacco, plantains, yams, sweet potatoes, cassava, potatoes, wheat, sorghum, soybean, rice, corn, maize, oil-producing Brassica (e.g., oil-producing rapeseed and canola), cotton, sugar cane, sunflower, millet, and alfalfa), fruits, vegetables, grains, seeds, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. agardh, and the like), seaweeds (e.g., kelp), a fungal cell (e.g., a yeast cell or a cell from a mushroom), an animal cell, a cell from an invertebrate animal (e.g., fruit fly, cnidarian, echinoderm, nematode, and the like), a cell from a vertebrate animal (e.g., fish, amphibian, reptile, bird, or mammal), a cell from a mammal (e.g., a pig, a cow, a goat, a sheep, a rodent, a rat, a mouse, a non-human primate, a human, and the like). Furthermore, a cell can be a stem cell or a progenitor cell.

As used herein, “stem cell” refers to a cell that has the capacity for self-renewal, i.e., the ability to go through numerous cycles of cell division while maintaining the undifferentiated state. Stem cells can be totipotent, pluripotent, multipotent, oligopotent, or unipotent. Stem cells can be embryonic, fetal, amniotic, adult, or induced pluripotent stem cells.

As used herein, “induced pluripotent stem cells” refers to a type of pluripotent stem cell that is artificially derived from a non-pluripotent cell, typically an adult somatic cell, by inducing expression of specific genes.

“Plant,” as used herein, refers to whole plants, plant organs, plant tissues, germplasm, seeds, plant cells, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to roots, stems, shoots, leaves, pollens, seeds, tumor tissue, and various forms of cells and culture (e.g., single cells, protoplasts, embryos, and callus tissue). The plant tissue may be in plant or in a plant organ, tissue or cell culture. “Plant organ” refers to plant tissue or a group of tissues that constitute a morphologically and functionally distinct part of a plant.

“Subject,” as used herein, refers to any member of the phylum Chordata, including, without limitation, humans and other primates, including non-human primates such as rhesus macaques, chimpanzees and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as male and female. In some embodiments, a host cell is derived from a subject (e.g., stem cells, progenitor cells, or tissue-specific cells). In some embodiments, the subject is a non-human subject.

As used herein, “transgenic organism” refers to an organism whose genome is genetically modified. The term includes the progeny (any generation) of a transgenic organism, provided that the progeny has the genetic modification.

### CRISPR Systems

The CRISPR (Clustered Regularly Interspaced Short Palindromic Repeats) genomic locus is found in the genomes of many prokaryotes (e.g., bacteria and archaea). CRISPR loci provide resistance to foreign invaders (e.g., virus, phage) in prokaryotes. In this way, the CRISPR system functions as a type of immune system to help defend prokaryotes against foreign invaders. There are three main stages in CRISPR-Cas immune systems: (1) acquisition, (2) expression, and (3) interference. Acquisition involves cleaving the genome of invading viruses and plasmids and integrating segments (termed protospacers) of the genomic DNA into the CRISPR locus of the host organism. The segments that are integrated into the host genome are known as spacers, which mediate protection from subsequent attack by the same (or sufficiently related) virus or plasmid. Expression involves transcription of the CRISPR locus and subsequent enzymatic processing to produce short mature CRISPR RNAs, each containing a single spacer sequence. Interference is induced after the CRISPR RNAs associate with Cas proteins to form effector complexes, which are then targeted to complementary protospacers in foreign genetic elements to induce nucleic acid degradation.

Currently, two classes of CRISPR systems have been described, Class 1 and Class 2, based upon the genes encoding the effector module, i.e., the proteins involved in the interference stage. Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III and Type IV systems. Class 2 systems comprise Type II, Type V and Type VI systems.

To date, there are six types (Types I-VI) and 19 subtypes of CRISPR systems categorized within these classes (Makarova et al., Nature Reviews Microbiology (2015) 13:1-15; Shmakov et al., Nature Reviews Microbiology (2017) 15:169-182).

CRISPR loci are currently characterized as including a number of short repeating sequences referred to as “repeats.” Repeats can form hairpin structures and/or repeats can be unstructured single-stranded sequences. The repeats occur in clusters. Repeats frequently diverge between species. Repeats are regularly interspaced with unique intervening sequences, referred to as “spacers,” resulting in a repeat-spacer-repeat locus architecture. Spacers are identical to or are homologous with known foreign invader sequences. In some instances, a spacer-repeat unit encodes a crisprRNA (crRNA). A crRNA refers to the mature form of the spacer-repeat unit. A crRNA contains a spacer sequence that is involved in targeting a target nucleic acid (e.g., possibly as a surveillance mechanism against foreign nucleic acid). Thus, crRNA has a region of complementarity to a potential DNA or RNA target sequence and in some cases, e.g., in currently characterized Type II systems, a second region that forms base-pair hydrogen bonds with a transactivating CRISPR RNA (tracrRNA) to form a secondary structure, typically to form at least a stem structure. In this context, the tracrRNA and a crRNA interact through a number of base-pair hydrogen bonds to form secondary RNA structures. Complex formation between tracrRNA/crRNA and a Cas protein results in conformational change of the Cas protein that facilitates binding to DNA, nuclease activities of the Cas protein, and crRNA-guided site-specific DNA cleavage by the nuclease. For a Cas protein/tracrRNA/crRNA complex to cleave a DNA target sequence, the DNA target sequence is adjacent to a cognate protospacer adjacent motif (PAM).

A CRISPR locus comprises polynucleotide sequences encoding for CRISPR Associated Genes (cas) genes. Cas genes are involved in the biogenesis and/or the interference stages of crRNA function. Cas genes display extreme sequence (e.g., primary sequence) divergence between species and homologs. Some Cas genes comprise homologous secondary and/or tertiary structures. Cas genes are typically named according to the organism from which they are derived. For example, Cas genes in Staphylococcus epidermidis can be referred to as Csm-type, Cas genes in Streptococcus thermophilus can be referred to as Csn-type, and Cas genes in Pyrococcus furiosus can be referred to as Cmr-type.

The integration stage of a CRISPR system refers to the ability of the CRISPR locus to integrate new spacers into the crRNA array upon being infected by a foreign invader. Acquisition of the foreign invader spacers can help confer immunity to subsequent attacks by the same foreign invader. Integration typically occurs at the leader end of the CRISPR locus. Cas proteins are involved in integration of new spacer sequences. Integration proceeds similarly for some types of CRISPR systems (e.g., Types I-III).

Mature crRNAs are processed from a longer polycistronic CRISPR locus transcript (i.e., pre-crRNA array). A pre-crRNA array comprises a plurality of crRNAs. The repeats in the pre-crRNA array are recognized by cas genes. Cas genes bind to the repeats and cleave the repeats. This action can liberate the plurality of crRNAs. crRNAs can be subjected to further events to produce the mature crRNA form such as trimming (e.g., with an exonuclease). A crRNA may comprise all, some, or none of the CRISPR repeat sequence.

Interference refers to the stage in the CRISPR system that is functionally responsible for combating infection by a foreign invader. CRISPR interference follows a similar mechanism to RNA interference (RNAi: e.g., wherein a target RNA is targeted (e.g., hybridized) by a short interfering RNA (siRNA)), which results in target RNA degradation and/or destabilization. Currently characterized CRISPR systems perform interference of a target nucleic acid by coupling crRNAs and Cas genes, thereby forming CRISPR ribonucleoproteins (RNPs). crRNA of the RNP guides the RNP to foreign invader nucleic acid, (e.g., by recognizing the foreign invader nucleic acid through hybridization). Hybridized target foreign invader nucleic acid-crRNA units are subjected to cleavage by Cas proteins. Target nucleic acid interference typically requires a protospacer adjacent motif (PAM) in a target nucleic acid.

By a “CRISPR-Cas system” as used herein, is meant any of the various CRISPR-Cas classes, types, and subtypes. Class 1 systems comprise Type I, Type III, and Type IV systems. Type I systems are currently characterized as having a Cas3 protein that has helicase activity and cleavage activity. Type I systems are further divided into several subtypes that have a defined combination of signature genes and distinct features of operon organization.

To date, it appears that all Type III systems possess a cas10 gene, which encodes a multidomain protein containing a Palm domain (a variant of the RNA recognition motif (RRM)) that is homologous to the core domain of numerous nucleic acid polymerases and cyclases and that is the largest subunit of Type III crRNA-effector complexes. All Type III loci also encode the small subunit protein, one Cas5 protein and typically several Cas7 proteins. Type III is also further divided into several subtypes.

Type IV systems encode a minimal multisubunit crRNA-effector complex comprising a partially degraded large subunit, Csf1, Cas5, Cas7, and in some cases, a putative small subunit. Type IV systems lack cas1 and cas2 genes. Type IV systems do not have subtypes, but there are two distinct variants. One Type IV variant has a DinG family helicase, whereas a second Type IV variant lacks a DinG family helicase, but has a gene encoding a small α-helical protein. An example of an organism with a Type IV system is Acidithiobacillus ferrooxidans.

Class 2 systems comprise Type II, Type V, and Type VI systems. Type II systems include cas1, cas2 and cas9 genes. There are two strands of RNA in Type II systems, a crRNA and a tracrRNA, that hybridizes to a complementary region of pre-crRNA causing maturation of the pre-crRNA to crRNA. The duplex formed by the tracrRNA and crRNA is recognized by, and associates with a multidomain protein, Cas9, encoded by the cas9 gene, which combines the functions of the crRNA-effector complex with target DNA cleavage. Cas9 is directed to a target nucleic acid by a sequence of the crRNA that is complementary to, and hybridizes with, a sequence in the target nucleic acid.

In Type V systems, nucleic acid target sequence binding involves a Cas12a protein and the crRNA, as does the nucleic acid target sequence cleavage. In Type V systems, the RuvC-like nuclease domain of Cas12a protein cleaves both strands of the nucleic acid target sequence in a sequential fashion (Swarts, et al., Mol. Cell (2017) 66:221-233.e4), producing 5′ overhangs, which contrasts with the blunt ends generated by Cas9 protein cleavage.

The Cas12a protein cleavage activity of Type V systems does not require hybridization of crRNA to tracrRNA to form a duplex; rather Type V systems use a single crRNA that has a stem-loop structure forming an internal duplex. Cas12a protein binds the crRNA in a sequence- and structure-specific manner by recognizing the stem loop and sequences adjacent to the stem loop, most notably the nucleotides 5′ of the spacer sequence, which hybridizes to the nucleic acid target sequence. This stem-loop structure is typically in the range of 15 to 19 nucleotides in length. Substitutions that disrupt this stem-loop duplex abolish cleavage activity, whereas other substitutions that do not disrupt the stem-loop duplex do not abolish cleavage activity.

Type VI systems include the Cas13a protein (also known as Class 2 candidate 2 protein, or C2c2) which does not share sequence similarity with other CRISPR effector proteins (see Abudayyeh, et al., Science (2016) 353: aaf5573). Cas13a proteins have two HEPN domains and possess single-stranded RNA cleavage activity. Cas13a proteins are similar to Cas12a proteins in requiring a crRNA for nucleic acid target sequence binding and cleavage, but not requiring tracrRNA. Also, similar to Cas12a protein, the crRNA for Cas13a proteins forms a stable hairpin, or stem-loop structure, that aids in association with the Cas13a protein. Type VI systems have a single polypeptide RNA endonuclease that utilizes a single crRNA to direct RNA cleavage in a target-dependent fashion. Additionally, after hybridizing to the target RNA complementary to the spacer, Cas13a protein becomes a promiscuous RNA endonuclease exhibiting non-specific endonuclease activity toward any single-stranded RNA in a sequence independent manner (see East-Seletsky, et al., Nature (2016) 538:270-273).

As is readily apparent, the discovery and characterization of CRISPR systems is currently evolving.

### Production of CRISPR Components

In all of the embodiments described herein, the various components can be produced by synthesis, or for example, using expression cassettes encoding CasM, a cognate guide, etc. The various components can be provided to a cell or used in vitro. These components can be present on a single cassette or multiple cassettes, in the same or different constructs. Expression cassettes typically comprise regulatory sequences functional in host cells into which they are introduced. Regulatory sequences are involved in one or more of the following: regulation of transcription, post-transcriptional regulation, and regulation of translation. Expression cassettes can be present in expression vectors and introduced into a wide variety of host cells including bacterial cells, yeast cells, plant cells, and mammalian cells.

In one aspect, all or a portion of the various components for use herein are produced in vectors, including expression vectors, comprising polynucleotides encoding therefor. Vectors useful for producing components for use in the present methods include plasmids, viruses (including phage), and integratable nucleic acid fragments (i.e., fragments integratable into the host genome by homologous recombination). A vector replicates and functions independently of the host genome, or may, in some instances, integrate into the genome itself. Suitable replicating vectors will contain a replicon and control sequences derived from species compatible with the intended expression host cell. In some embodiments, polynucleotides encoding one or more of the various components are operably linked to an inducible promoter, a repressible promoter, or a constitutive promoter. Expression vectors can also include polynucleotides encoding protein tags (e.g., poly-His tags, hemagglutinin tags, fluorescent protein tags, bioluminescent tags, nuclear localization tags). The coding sequences for such protein tags can be fused to the coding sequences or can be included in an expression cassette, for example, in a targeting vector.

General methods for construction of expression vectors are known in the art. Expression vectors for most host cells are commercially available. There are several commercial software products designed to facilitate selection of appropriate vectors and construction thereof, such as insect cell vectors for insect cell transformation and gene expression in insect cells, bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation and gene expression and methods to easily enable cloning of such polynucleotides. SnapGene™ (GSL Biotech LLC, Chicago, Ill.; snapgene.com/resources/plasmid_files/your_time_is_valuable/), for example, provides an extensive list of vectors, individual vector sequences, and vector maps, as well as commercial sources for many of the vectors.

Several expression vectors have been designed for expressing guide polynucleotides. See, e.g., Shen et al. Nat. Methods (2014) 11:399-402. Additionally, vectors and expression systems are commercially available, such as from New England Biolabs (Ipswich, MA) and Clontech Laboratories (Mountain View, CA). Vectors can be designed to simultaneously express a target-specific NATNA using a U2 or U6 promoter, a CasM and/or dCasM, and if desired, a marker protein, for monitoring transfection efficiency and/or for further enriching/isolating transfected cells by flow cytometry.

For example, the various components can be incorporated into mammalian vectors for use in mammalian cells. A large number of mammalian vectors suitable for use with the systems of the present invention are commercially available (e.g., from Life Technologies, Grand Island, NY; NeoBiolab, Cambridge, MA; Promega, Madison, WI; DNA2.0, Menlo Park, CA; Addgene, Cambridge, MA).

Vectors derived from mammalian viruses can also be used for expressing the various components of the present methods in mammalian cells. These include vectors derived from viruses such as adenovirus, papovirus, herpesvirus, polyomavirus, cytomegalovirus, lentivirus, retrovirus, vaccinia and Simian Virus 40 (SV40) (see, e.g., Kaufman, R. J., Molec. Biotech. (2000) 16:151-160; Cooray et al., Methods Enzymol. (2012) 507:29-57). Regulatory sequences operably linked to the components can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Commonly used promoters are constitutive mammalian promoters CMV, EF1a, SV40, PGK1 (mouse or human), Ubc, CAG, CaMKIIa, and beta-Act, and others known in the art (Khan, K. H. Advanced Pharmaceutical Bulletin (2013) 3:257-263). Furthermore, mammalian RNA polymerase III promoters, including H1 and U6, can be used.

Numerous mammalian cell lines have been utilized for expression of gene products including HEK 293 (Human embryonic kidney) and CHO (Chinese hamster ovary). These cell lines can be transfected by standard methods (e.g., using calcium phosphate or polyethyleneimine (PEI), or electroporation). Other typical mammalian cell lines include, but are not limited to: HeLa, U2OS, 549, HT1080, CAD, P19, NIH 3T3, L929, N2a, Human embryonic kidney 293 cells, MCF-7, Y79, SO—Rb50, Hep G2, DUKX-X11, J558L, and Baby hamster kidney (BHK) cells.

Vectors can be introduced into and propagated in a prokaryote. Prokaryotic vectors are well known in the art. Typically a prokaryotic vector comprises an origin of replication suitable for the target host cell (e.g., oriC derived from E. coli, pUC derived from pBR322, pSC101 derived from Salmonella), 15A origin (derived from p15A) and bacterial artificial chromosomes). Vectors can include a selectable marker (e.g., genes encoding resistance for ampicillin, chloramphenicol, gentamicin, and kanamycin). Zeocin™ (Life Technologies, Grand Island, NY) can be used as a selection in bacteria, fungi (including yeast), plants and mammalian cell lines. Accordingly, vectors can be designed that carry only one drug resistance gene for Zeocin for selection work in a number of organisms. Useful promoters are known for expression of proteins in prokaryotes, for example, T5, T7, Rhamnose (inducible), Arabinose (inducible), and PhoA (inducible). Furthermore, T7 promoters are widely used in vectors that also encode the T7 RNA polymerase. Prokaryotic vectors can also include ribosome binding sites of varying strength, and secretion signals (e.g., mal, sec, tat, ompC, and pelB). In addition, vectors can comprise RNA polymerase promoters for the expression of NATNAs. Prokaryotic RNA polymerase transcription termination sequences are also well known (e.g., transcription termination sequences from Streptococcus pyogenes).

Expression of proteins in prokaryotes is typically carried out in E. coli with vectors containing constitutive or inducible promoters directing the expression of either fusion or non-fusion proteins.

In some embodiments, a vector is a yeast expression vector comprising one or more components of the above-described methods. Examples of vectors for expression in Saccharomyces cerivisae include, but are not limited to, the following: pYepSec1, pMFa, pJRY88, pYES2, and picZ. Methods for gene expression in yeast cells are known in the art (see, e.g., Methods in Enzymology, Volume 194, “Guide to Yeast Genetics and Molecular and Cell Biology, Part A,” (2004) Christine Guthrie and Gerald R. Fink (eds.), Elsevier Academic Press, San Diego, CA). Typically, expression of protein-encoding genes in yeast requires a promoter operably linked to a coding region of interest plus a transcriptional terminator. Various yeast promoters can be used to construct expression cassettes for expression of genes in yeast.

### CasM Proteins

CasM, a new CRISPR-Cas protein, is described herein. CasM displays nucleic acid binding activity and produces breaks, such as singe-strand breaks (SSBs) or DSBs, when brought into proximity with a nucleic acid target sequence, e.g., by association with a cognate nucleic acid guide, such as a cognate crRNA. As shown in the Examples herein, CasM targets RNA and is capable of cleaving ssRNA, such as when delivered to a genomic target when complexed with a crRNA, without the necessity of association with a tracrRNA. CasM proteins typically include two or more higher eukaryotic and prokaryotic nucleotide-binding (HEPN) domains found in protein family PF05168, in the C-terminal region of the CasM sequence. CasM proteins show synteny with one or more WYL domain-containing proteins and sometimes with RtcB (RNA 3′-terminal phosphate cyclase, group B) domain-containing proteins. Based on the foregoing characteristics, CasM may be classified as a Class 2 Type VI CRISPR-Cas system. However, CasM has a very low degree of sequence similarity to other Type VI subtypes.

Exemplary CasM proteins are shown in SEQ ID NOS: 37-45, and are encoded by polynucleotides shown in SEQ ID NOS: 28-36, respectively. These proteins display approximately 13.59% to 99.82% sequence identity to each other and show less than 8% sequence identity with other known CRISPR-Cas proteins. CasM has been found in several species and isolates including, without limitation, Eubacterium siraeum; Ruminococcus sp., such as from Ruminococcus bicirculans; Ruminococcus flavefaciens, such as, but not limited to, FD-1 and strain XPD3002; Ruminococcus albus such as, but not limited to, strain KH2T6; Ruminococcus sp. isolates, such as but not limited to, isolates 2789STDY5834971, 2789STDY5608892 and 2789STDY5834894. However, is it to be understood that the term “CasM” refers to a protein derived from any species, subspecies or strain of bacteria that encodes a CasM protein, as well as orthologs thereof, or variants thereof. Representative CasM proteins include, but are not limited to, those proteins depicted as SEQ ID NOS: 37-45 (see Table 1), orthologs thereof, or variants thereof. CasM proteins are approximately 800 to approximately 1000 amino acids in length.

TABLE 1
Representative CasM Proteins

| Species/Isolate | SEQ ID NO |
|---|---|
| Eubacterium siraeum | SEQ ID NO: 37 |
| Ruminococcus sp., isolate 2789STDY5834971 | SEQ ID NO: 38 |
| Ruminococcus bicirculans | SEQ ID NO: 39 |
| Ruminocccus sp., isolate 2789STDY5608892 | SEQ ID NO: 40 |
| Ruminococcus sp. CAG:57 | SEQ ID NO: 41 |
| Ruminococcus flavefaciens FD-1 | SEQ ID NO: 42 |
| Ruminococcus albus strain KH2T6 | SEQ ID NO: 43 |
| Ruminococcus flavefaciens strain XPD3002 | SEQ ID NO: 44 |
| Ruminococcus sp., isolate 2789STDY5834894 | SEQ ID NO: 45 |

Analysis of these CasM protein sequences indicates the presence of two HEPN domains in the C-terminal region of the sequences. The HEPN domain is often involved in nucleic acid binding and can function as a metal-independent RNase in certain instances.

CasM systems display strong synteny with an open reading frame in WYL domain-(protein family PF13280) containing proteins. The sequences for WYL domains in various species that encode CasM proteins are shown as SEQ ID NOS: 52-59 (see Table 2). WYL domains share similarities with CRISPR-associated Rossman fold (CARF) domains and are thought to bind ligands derived from host-virus conflict and regulate CRISPR-Cas systems. A WYL domain protein (sll7009) has been shown to be a negative regulator of the I-D CRISPR-Cas system in Synechocystis sp. (Hein et al., RNA Biol. (2013) 10:852-864. In some instances, the WYL-containing protein contains at least two WYL domains. These duplications are consistent with the hypothesized multimeric assembly of these ligand-binding domains (Schumacher et al., EMBO J. (2002) 21:1210-1218). The N-termini of these WYL domains contain homology to transcriptional repressor CopG and the ParD anti-toxin domain. For use in eukaryotes, the WYL domain-containing proteins can be modified with a N- or C-terminal nuclear localization signal sequence (NLS). SEQ ID NOS: 61-68 present exemplary WYL domain-containing proteins modified with a seven amino acid C-terminal NLS tag derived from the SV40 Large T-antigen.

TABLE 2
WYL domain sequences in various CasM-containing species

| Species/Isolate | SEQ ID NO |
|---|---|
| Eubacterium siraeum | SEQ ID NO: 52 |
| Ruminococcus sp., isolate 2789STDY5834971 | SEQ ID NO: 53 |
| Ruminococcus bicirculans | SEQ ID NO: 54 |
| Ruminococcus sp., isolate 2789STDY5608892 | SEQ ID NO: 55 |
| Ruminococcus sp. CAG:57 | SEQ ID NO: 56 |
| Ruminococcus flavefaciens FD-1 | SEQ ID NO: 57 |
| Ruminococcus albus strain KH2T6 | SEQ ID NO: 58 |
| Ruminococcus flavefaciens strain XPD3002 | SEQ ID NO: 59 |

RtcB (RNA 3′-terminal phosphate cyclase, group B) is a protein domain superfamily and a RtcB homolog (SEQ ID NO. 60) proximal to the CasM loci has been identified. It has previously been reported that the CARF domain has sequence similarity with the N-terminal domain of the RtcR protein, which acts as the regulator of the Rtc RNA repair system. The Rtc system is comprised of 3′-terminal phosphate cyclase RtcA and the RNA ligase RtcB. The RtcB domain-containing proteins can be modified with a N- or C-terminal NLS for use in eukaryotes. A RtcB domain with an associated NLS derived from the SV40 Large T-antigen is shown in SEQ ID NO:69.

A modified CasM protein can have a low degree of sequence identity, a moderate degree of sequence identity, or a high degree of sequence identity over its length to a reference CasM protein, depending on the intended function of the CasM in question. By a “high degree of sequence identity” is meant approximately 90% sequence identity to 100% sequence identity, for example, about 90% . . . 95% . . . 98% sequence identity or higher. A “moderate degree of sequence identity” is typically between about 80% sequence identity to about 85% sequence identity, for example, about 80% identity or higher, such as about 85% sequence identity. A “low degree of sequence identity” is typically between about 50% identity and 75% identity, for example, about 50% identity, preferably about 60% identity to about 75% identity.

In some embodiments, the amino acid sequence of the reference CasM protein may be modified by deletion, insertion, or substitution of one or more amino acid residues (either conservative or non-conservative in nature), such that the activity of the CasM protein is either largely retained, enhanced, or reduced. These modifications may be deliberate, as through site-directed mutagenesis, or may be accidental, such as through mutations of hosts that produce the proteins or errors due to PCR amplification.

Conservative substitutions are generally those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, an aspartate with a glutamate, a threonine with a serine, or a similar conservative replacement of an amino acid with a structurally related amino acid, will not have a major effect on the desired biological activity. For example, the CasM protein may include up to about 5-10 conservative or non-conservative amino acid substitutions, or even up to about 15-100 or more, e.g., 50 or more, conservative or non-conservative amino acid substitutions, or any number between 5-100, so long as the desired function of the molecule remains intact.

In other embodiments, it may be desirable to modify one or more catalytic domains in order to render a nuclease-deactivated CasM protein, also termed “catalytically inactive,” “catalytically dead CasM,” “dead CasM,” or “dCasM,” such that the protein either fails to produce nucleic acid breaks, and/or binds a target sequence but does not cleave it. Such molecules lack all or a portion of nuclease activity and are unable to cleave a nucleic acid of interest and can therefore be used to regulate genes in a nucleic acid-guided manner. These dCasM proteins can be used alone or in fusions to synthetically repress (CRISPRi) or activate (CRISPRa) gene expression.

The CasM proteins can either be directly isolated and purified from bacteria, or synthetically or recombinantly produced using polynucleotides encoding the same.

### CasM Polynucleotides

Nucleic acid sequences encoding representative CasM proteins are shown in SEQ ID NOS: 28-36 (see Table 3) and these polynucleotides can be used to produce CasM proteins as described herein.

TABLE 3
Representative CasM DNA Sequences

| Species/Isolate | SEQ ID NO |
|---|---|
| Eubacterium siraeum | SEQ ID NO: 28 |
| Ruminococcus sp., isolate 2789STDY5834971 | SEQ ID NO: 29 |
| Ruminococcus bicirculans | SEQ ID NO: 30 |
| Ruminococcus sp., isolate 2789STDY5608892 | SEQ ID NO: 31 |
| Ruminococcus sp. CAG:57 | SEQ ID NO: 32 |
| Ruminococcus flavefaciens FD-1 | SEQ ID NO: 33 |
| Ruminococcus albus strain KH2T6 | SEQ ID NO: 34 |
| Ruminococcus flavefaciens strain XPD3002 | SEQ ID NO: 35 |
| Ruminococcus sp., isolate 2789STDY5834894 | SEQ ID NO: 36 |

These polynucleotides can be designed to encode native CasM proteins, such as the proteins depicted in SEQ ID NOS: 37-45 (see Table 1); homologs thereof, including orthologs found in other species; or other variants thereof. Moreover, a casM polynucleotide sequence can be modified to have a low degree of sequence identity, a moderate degree of sequence identity, or a high degree of sequence identity, over its length to a reference casM polynucleotide, depending on the intended function of the encoded CasM in question. By “a high degree of sequence identity” is meant approximately 90% sequence identity to 100% sequence identity, for example, about 90% . . . 95% . . . 98% sequence identity or higher. A “moderate degree of sequence identity” is typically between about 80% sequence identity to about 85% sequence identity, for example, about 80% identity or higher, such as about 85% sequence identity. A “low degree of sequence identity” is typically between about 50% identity and 75% identity, for example, about 50% identity, preferably about 60% identity to about 75% identity.

In some embodiments, the polynucleotide sequences are modified to enhance expression in a selected host cell. Codon usage bias refers to differences in the frequency of occurrence of synonymous codons in coding DNA. For example, for the 20 standard amino acids in the genetic code, there are 64 different codons (61 codons encoding for amino acids, and 3 stop codons). The overabundance in the number of codons allows several amino acids to be encoded by more than one codon. The genetic codes of different organisms are often biased towards the usage of one of the several codons that encode a particular amino acid. Thus, a greater frequency of one codon will be found than expected by chance in particular organisms. Accordingly, in order to enhance expression in a particular host cell, it is often desirable to manipulate polynucleotides to include codons that are biased for expression in the selected host cell. Several software packages are available online for this purpose. For example, a database from Integrated DNA Technologies, Coralville, IA (idtdna.com/CodonOpt), is a tool for producing modified sequences for expression in dozens of organisms. GeneScript, Piscataway, NJ, also provides modification tools through the OptimumGene™ algorithm (genscript.com/codon opt.html?src=google&gclid=CIX3uoqexdICFRSUfgodu3sAIQ). See also, U.S. Pat. No. 8,326,547.

Typically, polynucleotide sequences modified for expression in particular host cells will display from about 50%-99% sequence identity to the native sequences, such as 60%-95%, e.g. 65% . . . 70% . . . 75% . . . 80% . . . 85% . . . 90% . . . 95% or more sequence identity, or any integer between these ranges, to the native sequences.

Using these tools, polynucleotide sequences can be modified for expression in any commonly used host cell, such as but not limited to, bacterial cells and eukaryotic cells, including without limitation, bacterial cells such as E. coli, Lactoccocus lactis, Pseudomonas systems, Streptomyces systems, Bacillus subtilis systems, Brevibacillus systems, coryneform bacteria, and halophilic bacteria; algal cells; yeast and other fungal cells; plant cells; mammalian cells such as human cells; insect cells, and the like.

SEQ ID NOS: 1-9 show representative CasM-encoding polynucleotide sequences modified for expression in E. coli cells (see Table 4). SEQ ID NOS: 10-18 show representative CasM-encoding polynucleotide sequences modified for expression in human cells (see Table 5). SEQ ID NOS: 19-27 show CasM-encoding polynucleotide sequences modified for expression in Zea mays cells (see Table 6).

TABLE 4
Representative casM DNA Sequences
Modified for Expression in E. coli

| Species/Isolate | SEQ ID NO |
|---|---|
| Eubacterium siraeum | SEQ ID NO: 1 |
| Ruminococcus sp., isolate 2789STDY5834971 | SEQ ID NO: 2 |
| Ruminococcus bicirculans | SEQ ID NO: 3 |
| Ruminococcus sp., isolate 2789STDY5608892 | SEQ ID NO: 4 |
| Ruminococcus sp. CAG:57 | SEQ ID NO: 5 |
| Ruminococcus flavefaciens FD-1 | SEQ ID NO: 6 |
| Ruminococcus albus strain KH2T6 | SEQ ID NO: 7 |
| Ruminococcus flavefaciens strain XPD3002 | SEQ ID NO: 8 |
| Ruminococcus sp., isolate 2789STDY5834894 | SEQ ID NO: 9 |

TABLE 5
Representative casM DNA Sequences
Modified for Expression in Human Cells

| Species/Isolate | SEQ ID NO |
|---|---|
| Eubacterium siraeum | SEQ ID NO: 10 |
| Ruminococcus sp., isolate 2789STDY5834971 | SEQ ID NO: 11 |
| Ruminococcus bicirculans | SEQ ID NO: 12 |
| Ruminococcus sp., isolate 2789STDY5608892 | SEQ ID NO: 13 |
| Ruminococcus sp. CAG:57 | SEQ ID NO: 14 |
| Ruminococcus flavefaciens FD-1 | SEQ ID NO: 15 |
| Ruminococcus albus strain KH2T6 | SEQ ID NO: 16 |
| Ruminococcus flavefaciens strain XPD3002 | SEQ ID NO: 17 |
| Ruminococcus sp., isolate 2789STDY5834894 | SEQ ID NO: 18 |

TABLE 6
Representative casM DNA Sequences
Modified for Expression in Zea mays

| Species/Isolate | SEQ ID NO |
|---|---|
| Eubacterium siraeum | SEQ ID NO: 19 |
| Ruminococcus sp., isolate 2789STDY5834971 | SEQ ID NO: 20 |
| Ruminococcus bicirculans | SEQ ID NO: 21 |
| Ruminococcus sp., isolate 2789STDY5608892 | SEQ ID NO: 22 |
| Ruminococcus sp. CAG:57 | SEQ ID NO: 23 |
| Ruminococcus flavefaciens FD-1 | SEQ ID NO: 24 |
| Ruminococcus albus strain KH2T6 | SEQ ID NO: 25 |
| Ruminococcus flavefaciens strain XPD3002 | SEQ ID NO: 26 |
| Ruminococcus sp., isolate 2789STDY5834894 | SEQ ID NO: 27 |

The casM polynucleotides can also be modified to include sequences encoding N- or C-terminal nuclear localization signal sequences (NLS), such as for expression in eukaryotic cells. Such sequences are known, and include, without limitation, an NLS tag derived from the SV40 Large T-antigen. Such as tag is present at the C-terminus of the proteins shown in SEQ ID NOS: 61-69 (i.e., the last seven amino acids in these sequences).

The casM polynucleotides can be used to recombinantly produce CasM proteins using methods well known in the art.

### CasM Complexes

CasM proteins can be complexed to a cognate nucleic acid guide (cognate guide/CasM complex) in order to deliver CasM in proximity with a target nucleic acid sequence. A cognate guide, such as a crRNA, is a polynucleotide that site-specifically guides a CasM nuclease, or a deactivated CasM nuclease, to a target nucleic acid region. The binding specificity is determined jointly by the complementary region on the cognate guide and a short DNA motif (protospacer adjacent motif or PAM) juxtaposed to the complementary region. The spacer present in the guide specifically hybridizes to a target nucleic acid sequence and determines the location of a Cas protein's site-specific binding and nucleolytic cleavage.

Cognate guide/CasM complexes can be produced using methods well known in the art. For example, the guide components of the complexes can be produced in vitro and CasM components can be recombinantly produced and then the guides and CasM proteins can be complexed together using methods known in the art. Additionally, cell lines constitutively expressing CasM proteins can be developed and can be transfected with the guide components, and complexes can be purified from the cells using standard purification techniques, such as but not limited to affinity, ion exchange and size exclusion chromatography. See, e.g., Jinek M., et al., “A programmable dual-RNA-guided DNA endonuclease in adaptive bacterial immunity,” Science (2012) 337:816-821.

Alternatively, the components, i.e., the cognate guides and casM polynucleotides may be provided separately to a cell, e.g., using separate constructs, or together, in a single construct, or in any combination, and complexes can be purified as above.

Methods of designing particular guides, such as for use in the complexes, are known. See, e.g., Briner et al., “Guide RNA Functional Modules Direct Cas9 Activity and Orthogonality,” Molecular Cell (2014) 56:333-339. To do so, the genomic sequence for the gene to be targeted is first identified. The exact region of the selected gene to target will depend on the specific application. For example, in order to activate or repress a target gene using, for example, Cas activators or repressors, cognate guide/CasM complexes can be targeted to the promoter driving expression of the gene of interest. For genetic knockouts, guides are commonly designed to target 5′ constitutively expressed exons which reduces the chances or removal of the targeted region from mRNA due to alternative splicing. Exons near the N-terminus can be targeted because frameshift mutations here will increase the likelihood of the production of a nonfunctional protein product. Alternatively, cognate guides can be designed to target exons that code for known essential protein domains. In this regard, non-frameshift mutations such as insertions or deletions are more likely to alter protein function when they occur in protein domains that are essential for protein function. For gene editing using HDR, the target sequence should be close to the location of the desired edit. In this case, the location where the edit is desired is identified and a target sequence is selected nearby.

The guides can be delivered to a cell. If the cell constitutively expresses a CasM nuclease, the CasM nuclease will then be recruited to the target site to cleave the target nucleic acid. If the cell does not express a CasM nuclease, complexes of cognate guide/CasM can be delivered to the cells to make breaks in the genome, thereby triggering the repair pathways in the cells.

Treated cells are then screened using methods well known in the art, such as using high-throughput screening techniques including, but not limited to, fluorescence-activated cell sorting (FACS)-based screening platforms, microfluidics-based screening platforms, and the like. These techniques are well known in the art. See, e.g., Wojcik et al., Int. J. Molec. Sci. (2015) 16:24918-24945. The cells can then be expanded and re-transfected with additional cognate guide/CasM complexes to introduce further diversity and this process can be repeated iteratively until a population with the desired properties is obtained. Single cell clones are sorted from the population, expanded and sequenced to recover the mutations that resulted in the desired function.

### Applications of CasM

Due to its RNA-targeting abilities, CasM can be used to edit RNA and in some embodiments, to treat diseases caused by toxic RNA or improperly spliced RNA.

In some embodiments cognate guide/CasM complexes, such as, but not limited to crRNA/CasM complexes, are capable of sequence-specific ssRNA activity. Recognition and cleavage of an initial ssRNA complementary to the crRNA target sequence activates the CasM protein to carry out endonuclease activity toward any single-stranded RNA in a sequence-independent manner. The sequence-specific recognition of RNA of the crRNA/CasM complex facilitates the target knockdown of gene transcripts perturbing translation of a specific protein. The non-specific endonuclease activity of an activated crRNA/CasM complex in a cellular environment can result in cell death due to depletion of RNA encoding for essential gene transcripts. Thus, the specific RNA targeting and collateral endonuclease activity of an activated crRNA/CasM complex enables the sequence-specific selection of cells expressing a RNA transcript.

Thus, in further aspects, CasM complexes, such as, but not limited to crRNA/CasM complexes, can be used in methods of screening and killing cells, such as bacterial cells, that have not been modified by a DNA targeting nuclease (i.e., a Type II Cas9 nuclease). This method comprises contacting a NATNA/Cas9 complex to a locus of interest in a population of cells. Contacting the NATNA/Cas9 complex with the locus results in DNA cleavage and subsequent repair of the break by the endogenous cellular repair machine and the introduction of indels at the break site. The targeting of the NATNA/Cas9 complex to a targeted locus that encodes an RNA transcript results in indels in an RNA transcript sequence. This modified RNA transcript sequence is different compared to a transcript from an unmodified cell (a wild-type cell). A crRNA/CasM complex can then be targeted to the unmodified transcript, wherein crRNA/CasM recognition of the unmodified transcript results in activation of the sequence independent, single-stranded RNA targeting activity of the CasM protein and subsequent cell death. Alternatively, this method can be adapted to screen for the incorporation of a donor-polynucleotide into NATNA/Cas9 break site.

In another aspect, CasM complexes can by targeted to a eukaryotic exon coding region to cause exon skipping. This method comprises contacting a crRNA/deactivated CasM complex, such as, but not limited to a crRNA/dCasM complex, with either a donor site (5′ end of an intron), a branch site (proximal to the 3′ end of an intron), or an acceptor site (5′ of an exon) of a pre-mRNA. Contacting the crRNA/dCasM complex to the various regions involved in exon splice events prevents the proper splicing of one of more exons together and causes the target exon to be “skipped”, and thus is not included in the mature mRNA and therefore omitted from the translated polypeptide sequence.

In yet another aspect, CasM complexes are used for the detection of one or more target molecules in vitro. This method comprises contacting a cognate guide/CasM complex, such as a crRNA complex, with a ssRNA target of interest within a pool of nucleic acids. The crRNA/CasM complex can be added to a sample potential containing the ssRNA target of interest, in combination with a quenched fluorescent RNA reporter, for example a RNA hexamer with a 6-Carboxyfluorescein at the 5′ end and a Iowa Black® FQ quencher (Integrated DNA Technologies, Coralville, IA) at 3′. Contacting of the crRNA/CasM complex with the ssRNA target, activates the CasM protein to carry out collateral cleavage of the quenched fluorescent RNA reporter where cleavage of the reporter and resulting in an increase fluorescence that can by read out using a spectrophotometer. The gain in fluorescence is used as a measure of the presents of a ssRNA target of interest.

In another aspect, CasM can be used for the targeted cleavage of an endogenous mRNA transcript while simultaneously delivering an exogenous mRNA transcript in cells. This method comprises contacting a cognate guide/CasM complex, with a disease-associated endogenous mRNA transcript, while simultaneously delivering of a mRNA coding for the non-disease exogenous polypeptide into a cell. Thus, the disease-associated phenotype is repressed while the non-disease phenotype is restored.

The CasM proteins described herein can also be used with associated cognate guides in order to activate or repress a target gene, to knockout a gene, to produce a nonfunctional protein product, or to alter protein function. The present invention includes methods of modulating in vitro or in vivo transcription using the various components and complexes described herein. In one embodiment, a cognate guide/CasM protein complex can repress gene expression by interfering with transcription when the cognate guide directs nucleic acid target binding of the complex to the promoter region of the gene. Use of the complexes to reduce transcription also includes complexes wherein the CasM protein is fused to a known down-regulator of a target gene (e.g., a repressor polypeptide). For example, expression of a gene is under the control of regulatory sequences to which a repressor polypeptide can bind. A cognate guide can direct nucleic acid target-binding of a repressor protein complex to the sequences encoding the regulatory sequences or adjacent the regulatory sequences such that binding of the repressor protein complex brings the repressor protein into operable contact with the regulatory sequences. Similarly, CasM can be fused to an activator polypeptide to activate or increase expression of a gene under the control of regulatory sequences to which an activator polypeptide can bind.

In one embodiment, CasM can be fused with a nuclease, or a mutant or an active portion thereof, as well as a cognate guide, in order to bring the nuclease into proximity with a target nucleic acid sequence, wherein the nuclease can produce a single-strand or double-strand break. In this way, a locus-specific cut in a target nucleic acid can be achieved using a cognate guide in combination with CasM, and the nuclease of interest. For example, it may be desirable to associate CasM with a restriction endonuclease in order to cleave at a particular restriction site in a target nucleic acid sequence. The restriction endonuclease can be selected from any of the various types of restriction endonucleases, such as, but not limited to, type I, II, III or IV. See, e.g., PCT Publication No. WO 2013/098244 to Brouns et al., published 4 Jul. 2013, for methods of producing complexes between a Cas protein and a restriction endonuclease.

Using the methods described herein, any desired nucleic acid sequence, and in particular RNA sequences, for modification can be targeted, including without limitation, protein coding mRNA sequences, in order to reduce or restore the function of the gene product; regions that have a propensity to incorporate nucleotide sequences from a donor template, termed “HDR hotspots” herein; safe harbor regions, i.e., regions where nucleotide sequences can be inserted without disrupting neighboring gene function; non-coding regulatory regions in nucleic acid sequences; and the like.

Protein coding sequences, including RNA such as mRNA, for targeting by the methods described herein include, but are not limited to, mammalian antibodies (ABs) (IgG, IgA, IgM, IgE), antibody fragments such as Fc regions, antibody Fab regions, antibody heavy chains, antibody light chains, antibody CDRs, nanobodies, chimeric antibodies and other IgG domains; T cell receptors (TCR); endonucleases and exonucleases, such as TALENS, CRISPR nucleases such as Cas9, Cas3, Cpf1, ZnFN, meganucleases, nuclease domains such as HNH domain, RuvC domain; recombinases such as Cre, Tre, Brec1, Flp, γ-integrase, IntI4 integrase, XerD recombinase, HP1 integrase; DNA topoisomerases; transposons such as the Tc1/mariner family, Tol2, piggyBac, Sleeping beauty; RAG proteins; retrotransposons such as LTR-retrotransposons and non-LTR retrotransposons (Alu, SINE, LINE); enzymes including but not limited to arginases, glycosydases, proteases, kinases, and glycosylation enzymes such as glycosyltransferase; anticoagulants such as protein C, Protein S and antithrombin; coagulants such as thrombin; nucleases such as DNAses, RNAses, helicases, GTPases; DNA or RNA binding proteins; reporter molecules, such as Green Fluorescent Protein (GFP); cell penetrating peptides and their fusions with cargo proteins; membrane proteins such as GPCRs, pain receptors such as TRP channels and ion channels; cell surface receptors including but not limited to EGFR, FGFR, VEGFR, IGFR and ephrin receptor; cell adhesion molecules like integrins and cadherins; ion channels; rhodopsins; immunoreceptors such as CD28, CD80, PD-1, PD-L1, CTLA-4, CXCR4, CXCR5, B2M, TRACA, TRBC; proteins known to be involved with genetic defects; secreted proteins including but not limited to hormones, cytokines, growth factors; vaccine antigens such as viral proteins from human immunodeficiency virus (HIV), Dengue, cytomegalovirus (CMV), Ebola, Zika and oncolytic viruses; snake toxin proteins and peptides including but not limited to phospholipases and metalloproteases; ribosomal cyclic peptides.

The present invention also encompasses genome engineering methods for preventing or treating diseases, disorders, and conditions using the various methods described herein. In one embodiment, a genome engineering method uses the introduction of nucleic acid sequences into an organism or cells of an organism (e.g., patient) to achieve expression of components of the present invention to provide modification of a target function. For example, cells from an organism may be engineered, ex vivo, by (i) introduction of vectors comprising expression cassettes expressing the various components, (ii) direct introduction of a NATNA and/or donor polynucleotides and CasM proteins, or (iii) introduction of combinations of these components. The engineered cells are provided to an organism (e.g., patient) to be treated.

Examples of genome engineering and techniques for therapy are known in the art (see, e.g., Kay, M. A., Nature Reviews Genetics (2011) 12:316-328; Wang et al., Discov. Med. (2014) 18:67-77; Wang et al., Discov. Med. (2014) 18:151-61; “The Clinibook: Clinical Gene Transfer State of the Art,” Odile Cohen-Haguenauer (Editor), EDP Sciences (Oct. 31, 2012), ISBN-10:2842541715).

In some aspects, components of the present invention are delivered using nanoscale delivery systems, such as nanoparticles. Additionally, liposomes and other particulate delivery systems can be used. For example, vectors including the components of the present methods can be packaged in liposomes prior to delivery to the subject or to cells derived therefrom, such as described in U.S. Pat. Nos. 5,580,859; 5,264,618; 5,703,055. Lipid encapsulation is generally accomplished using liposomes that are able to stably bind or entrap and retain nucleic acid.

The methods described herein can also be used to generate non-human genetically modified organisms, such as mice, plants, and the like.

While preferred embodiments of the present invention have been shown and described herein, it will be obvious to those skilled in the art that such embodiments are provided by way of example only. From the above description and the following Examples, one skilled in the art can ascertain essential characteristics of this invention, and without departing from the spirit and scope thereof, can make changes, substitutions, variations, and modifications of the invention to adapt it to various usages and conditions. Such changes, substitutions, variations, and modifications are also intended to fall within the scope of the present disclosure.

### EXPERIMENTAL

Aspects of the present invention are further illustrated in the following Examples. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric. It should be understood that these Examples, while indicating some embodiments of the invention, are given by way of illustration only.

The following Examples are not intended to limit the scope of what the inventors regard as various aspects of the present invention.

### Example 1

### Discovery of a New CRISPR-Associated (Cas) Protein in Silico

This Example describes the in silico discovery of a new Cas protein, termed “CasM,” from genomic sequencing data. The overall approach used was similar to methods described in Shmakov et al., “Discovery and functional characterization of diverse Class 2 CRISPR-Cas systems” Molecular Cell (2015) 60:385-397. In particular a computational pipeline was used to search sequencing data for CRISPR arrays in whole genomes and metagenic contigs.

Every contig or genome in the data set was inspected to determine if it contained a CRISPR array using Minced (github.com/ctSkennerton/minced) and PILERCR (drive5.com/pilercr/).

Any time a CRISPR array was found in a contig or genome, the surrounding DNA sequence (up to 10 kilobases on either side of the CRISPR array) was further inspected for open reading frames (ORFs) using the tool getorf (emboss.sourceforge.net/apps/cvs/emboss/apps/getorf.html).

The primary amino acid sequence of each predicted ORF was analyzed for potential functional domain annotations using the tool HHPred (homology detection & structure prediction by HMM-HMM comparison; toolkit.tuebingen.mpg.de/hhpred). HHPred allows the user to specify which databases to compare the amino acid sequence against to find similar protein domains. The databases searched included PFAM (which includes a large collection of protein families; http://pfam.xfam.org/), PDB (protein databank; wwpdb.org), CDD (conserved domain database; ncbi.nlm.nih.gov/Structure/cdd/cdd.shtml), and KEGG (Kyoto Encyclopedia of Genes and Genomes; genome.jp/kegg/).

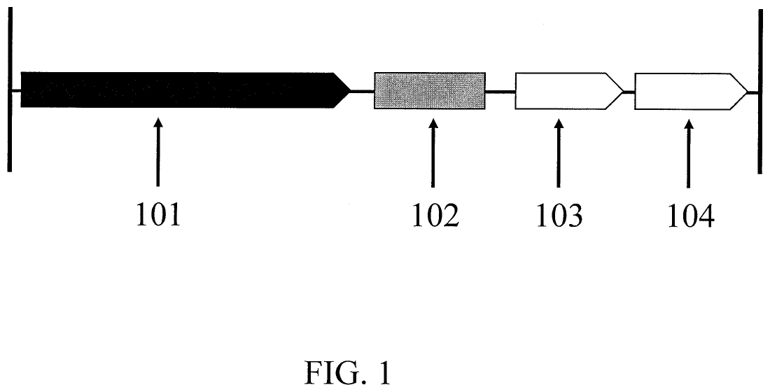

Using these tools, ORFs encoding a new putative CRISPR-associated protein, termed “CasM,” was found in several species in the Clostridia family. The ORFs are proximal to a predicted HTH DNA binding protein with homology to a CRISPR-associated WYL domain. See FIG. 1 for a representative map of a CRISPR operon found in Eubacterium siraeum (Genome Accession No. NZ_DS499551.1, coordinates 211.800-220.497). The various CRISPR locus features are described in Table 7.

TABLE 7
CasM CRISPR locus for Eubacterian siraeum (FIG. 1)

101 corresponds to the CasM open reading frame (ORF) (Protein
accession: WP_005358205.1)
102 corresponds to a CRISPR Array
103 corresponds to an ORF containing a RctB RNA ligase domain
(Protein accession: WP_005358214.1)
104 corresponds to an ORF containing a WYL DNA binding domain
(Protein accession: WP_005358216.1)

The results of HHPred analysis were analyzed to determine if the CasM-encoding ORFs had predicted domains commonly found in CRISPR-associated proteins. No annotations were found for CasM, thus indicating that the protein was novel.

The sequences for the various native CasM proteins are shown in SEQ ID NOS: 37-45 (see Table 1) and the native polynucleotides encoding therefor are shown in SEQ ID NOS: 28-36 (see Table 3). This protein has no significant homology to any known protein families or to any Class 2 Cas effectors.

### Example 2

### Codon Modification of Native casM Sequences

This Example describes the process of codon optimizing CasM coding sequences to improve expression in selected host cells.

Native casM nucleotide sequences were retrieved from the reference genomes or metagenomic contigs of the host microbes as described in Example 1. The amino acid sequences of the coding regions were generated with the ExPASy DNA translation tool (web.expasy.org/translate/). Next, these amino acid sequences were entered into the Integrated DNA Technologies (Coralville, IA) Codon Optimization tool (idtdna.com/CodonOpt). “Amino acid” was chosen for the “Sequence Type” option and “Gene” was chosen for the “Product Type” option. For each native casM sequence, codon modifications were performed to increase expression in E. coli, human, and Zea mays cells.

SEQ ID NOS: 1-9 show the modified sequences for use in E. coli (see Table 4). SEQ ID NOS: 10-19 show the modified sequences for use in human cells (see Table 5). SEQ ID NOS: 20-27 show the modified sequences for use in Z. mays cells (see Table 6). Table 8 shows the percent identity of the modified sequences to the native sequences.

TABLE 8
Percent Sequence Identity to Native casM Sequences

| | modified for | modified for | modified for |
|---|---|---|---|
| casM bacterial strain | E. coli cells | human cells | Z. mays cells |
| Eubacterium siraeum | 75% | 77% | 77% |
| Ruminococcus sp., isolate | 77% | 76% | 76% |
| 2789STDY5834971 | | | |
| Ruminococcus bicirculans | 76% | 76% | 77% |
| Ruminococcus sp., isolate | 76% | 75% | 77% |
| 2789STDY5608892 | | | |
| Ruminococcus sp. CAG:57 | 76% | 77% | 76% |
| Ruminococcus flavefaciens | 76% | 77% | 76% |
| FD-1 | | | |
| Ruminococcus albus strain | 76% | 77% | 77% |
| KH2T6 | | | |
| Ruminococcus flavefaciens | 76% | 77% | 77% |
| strain XPD3002 | | | |
| Ruminococcus sp., isolate | 75% | 76% | 78% |
| 2789STDY5834894 | | | |

### Example 3

### Production of CasM Expression Plasmids for DNA Interference Assays

This Example describes the production of plasmids that express the CasM protein.

The modified casM nucleotide sequences set forth in Example 2 were synthesized in vitro. The DNA sequences were cloned into an appropriate plasmid for expression in E. coli.

For E. coli expression, the E. coli-modified sequences were cloned into a p14A plasmid backbone using appropriate restriction nucleases. The plasmid backbone contained a T7 promoter upstream of the CasM coding sequence to facilitate transcription in cells.

The p14A plasmid backbone also contained a cloning site enabling the insertion of a minimal CRISPR array. The minimal CRISPR array contained one repeat sequence, followed by one spacer sequence, followed by one repeat sequence. The plasmid backbone also contained a T7 promoter upstream of the CRISPR array site, a kanamycin resistance gene, and a ColE1 origin of replication.

Similar techniques are used for preparing plasmids for expression in human and Zea mays cells. Once the plasmids are produced, they are transfected into the selected cell, e.g., E. coli, human, or plant cells (e.g., Zea mays cells).

### Example 4

### Plasmid Interference Assay

This Example describes the use of CasM in an assay to evaluate its ability to cleave double-stranded DNA in the form of a target plasmid. The overall approach is similar to methods used in Burnstein et al., Nature (2016) 542:237-241.

The CasM expression plasmid in Example 3 is transformed into E. coli cells. The cells are grown in a medium containing kanamycin to select only for cells that contain the CasM expression plasmid.

A target plasmid is constructed that contains the spacer sequence contained in the CRISPR array of the CasM expression plasmid. Adjacent to the spacer sequence is a randomized PAM sequence of 7 nucleotides. Plasmid libraries containing randomized PAM sequences are assembled by annealing a DNA oligonucleotide containing a target with a 7 nt randomized PAM region with a primer and extended with Klenow Fragment (New England Biolabs, Ipswitch, MA). The double-stranded DNA is digested with EcoRI and Ncol and ligated into a pUC19 backbone. The ligated library is transformed into E. coli DH5a and cells are harvested, the plasmids extracted and purified. 200 ng of the pooled library is transformed into electro-competent E. coli harboring a CRISPR locus or a control plasmid with no locus. The transformed cells are plated on selective media containing carbenicillin (100 mg L⁻¹) and chloramphenicol (30 mg L⁻¹) for 30 hours at 25° C. Plasmid DNA is extracted and the PAM sequence is amplified with adapters for Illumina sequencing. The 7 nt PAM region is extracted and PAM frequencies calculated for each 7 nt sequence. PAM sequences depleted above the specified threshold are used to generate a sequence logo with WebLogo (weblogo.berkeley.edu). If depleted PAMs are present, this shows that the nuclease is a double-stranded DNA nuclease.

### Example 5

### Targeted Modification of HEK293 Cells Using CasM

This Example illustrates the use of CasM to modify human embryonic kidney (HEK293) cells at specific target locations.

casM polynucleotides are transfected into HEK293 cells constitutively expressing a CasM-GFP fusion (HEK293-CasM-GFP), using the Nucleofector™ 96-well Shuttle System (Lonza, Allendale, NJ) and the following protocol. The casM polynucleotides are designed to target the FUT8 gene. Equal molar amounts of casM polynucleotide components are prepared in an annealing buffer (1.25 mM HEPES, 0.625 mM MgCl₂, 9.375 mM KCl at pH 7.5), incubated for 2 minutes at 95° C., removed from the thermocycler, allowed to equilibrate to room temperature, and dispensed in a 10 μL final volume in a 96-well plate. Culture medium is aspirated from HEK293-CasM-GFP cells, and the cells are washed once with calcium and magnesium-free PBS and then trypsinized by the addition of TrypLE (Life Technologies, Grand Island, NY) followed by incubation at 37° C. for 3-5 minutes. Trypsinized cells are gently pipetted up and down to form a single cell suspension and added to DMEM complete culture medium composed of DMEM culture medium (Life Technologies, Grand Island, NY) containing 10% FBS (Fisher Scientific, Pittsburgh, PA) and supplemented with penicillin and streptomycin (Life Technologies, Grand Island, NY).

The cells are then pelleted by centrifugation for 3 minutes at 200× g, the culture medium aspirated and cells resuspended in PBS. The cells are counted using the Countess™ II Automated Cell Counter (Life Technologies, Grand Island, NY). 2.2×10⁷ cells are transferred to a 50 ml tube and pelleted. The PBS is aspirated and the cells resuspended in Nucleofector™ SF (Lonza, Allendale, NJ) solution to a density of 1×10⁷ cells/mL. 20 μL of the cell suspension are then added to individual wells containing 10 μL of casM polynucleotide components and the entire volume is transferred to the wells of a 96-well Nucleocuvette™ Plate (Lonza, Allendale, NJ). The plate is loaded onto the Nucleofector™ 96-well Shuttle™ (Lonza, Allendale, NJ) and cells are nucleofected using the 96-CM-130 Nucleofector™ program (Lonza, Allendale, NJ). Post-nucleofection, 70 μL DMEM complete culture medium is added to each well and 50 μL of the cell suspension are transferred to a collagen coated 96-well cell culture plate containing 150 μL pre-warmed DMEM complete culture medium. The plate is then transferred to a tissue culture incubator and maintained at 37° C. in 5% CO₂ for 48 hours.

Genomic DNA (gDNA) is prepped using the QuickExtract DNA extraction solution (Illumina, San Diego, CA) pursuant to the manufacturer instructions. Sequencing amplicons of between 150 bp to 200 bp are designed to span the CasM RNP FUT8 target site. Using previously isolated gDNA, a first PCR is performed using Herculase II Fusion DNA Polymerase™ (Agilent, Santa Clara, CA) with primers comprising an adapter sequences and a sequence specific to the region flanking the FUT8 target site. A second PCR is performed using the amplicons of the first round of PCR as template at 1/20th the volume of the PCR reaction volume. The second PCR uses a second set of primers comprising a sequence complementary to the adapter sequence of the first primer pair, a barcode index sequence unique to each sample, and a flow cell adapter sequence. Amplicons are pooled and analyzed on a 2% TBE gel and bands of expected amplicon sizes are gel purified using the QIAEX II Gel extraction kit™ (Qiagen, Venlo, Luxembourg). The concentrations of purified amplicons are evaluated using the double-stranded DNA BR Assay Kit and Qubit system™ (Life Technologies, South San Francisco, CA) and library quality determined using the Agilent DNA100Chip and Agilent Bioanalyzer 2100 system™ (Agilent, Santa Clara, CA). After validation of library quality, the library is sequenced on a MiSeq Benchtop Sequencer™ (Illumina, San Diego, CA) with the MiSeq Reagent Kit v2™ (300 cycles, Illumina, San Diego, CA) per manufacturer instructions for 151 bp paired end reads.

The identity of products in the sequencing data is analyzed based upon the index barcode sequence adapted onto the amplicon in the second round of PCR. A computational script is used to process the MiSeq data by executing the following tasks:
1. Joining paired end reads with the aid of fastq-join (Aronesty 2011: code.google.com/p/ea-utils);
2. Validating the sequence reads for appropriate primer sequences being present at both 5′ and 3′ ends of the read sequence using fastx_barcode_splitter (hannonlab.cshl.edu/fastx_toolkit/index.html); reads lacking correct primer sequences at both ends are discarded.
3. Comparing Read sequences to expected wild type FUT8 sequence; identical read sequences are classified as having the same indel modification.

Other chromosomal loci within HEK293 cells are similarly modified by selection of an appropriate spacer sequence for the CasM RNP. Selection is specific to a specific gene target and the procedure outlined in this Example is readily modifiable by one of ordinary skill in the art for other gene targets.

This procedure can provide data to verify the CasM RNP and to detect nucleic acid-guided nuclease activity at targeted loci in HEK293 cells.

### Example 6

### CasM CRISPR Array Processing Assay

This Example describes the CRISPR array processing activity of a CasM protein. The following method may be practiced with other CasM protein homologs to characterize their CRISPR array processing capabilities.

### A. Identification of the CRISPR Array Repeat Sequence

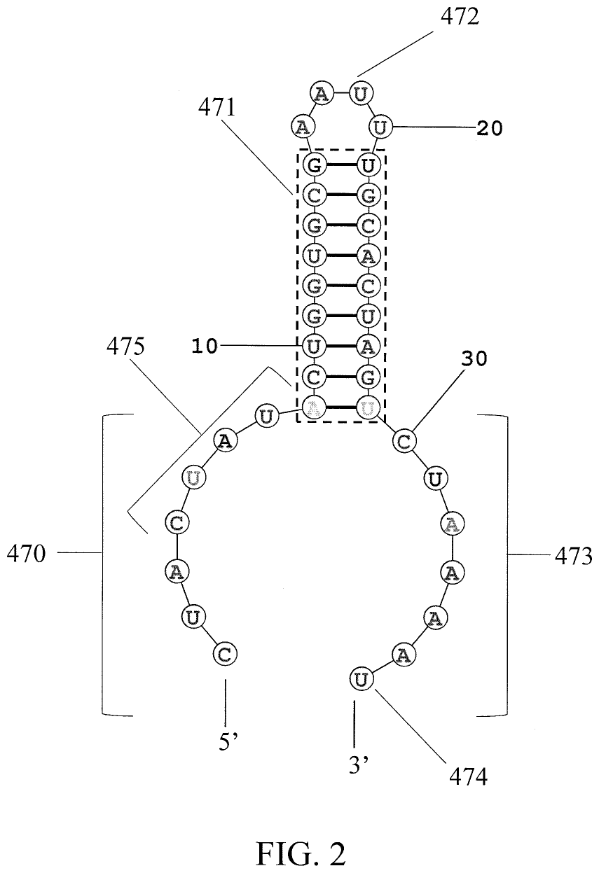

The CRISPR array of the CasM protein homolog (SEQ ID NO:39) was analyzed in silico and the repeat sequence identified. The in silico structure of a CRISPR repeat sequence (SEQ ID NO:51) associated with the CasM protein (SEQ ID NO:39) as predicted using an RNA folding algorithm (rna.urmc.rochester.edu/RNA structureWeb/Servers/Predict1.html) is shown in FIG. 2 (SEQ ID NO:51). The various CRISPR repeat sequence structural components represented in FIG. 2 are described in Table 9.

TABLE 9
Numerical Indicators Used to Illustrate CasM CRISPR Repeat Sequence
Structural Components (FIG. 2)

470 corresponds to a 5′ repeat handle sequence
471 corresponds to a stem-duplex formed by a first stem duplex stand
hybridized to a second stem duplex strand
472 corresponds to a loop sequences
473 corresponds to a 5′ repeat handle sequence
474 corresponds to the 3′ attachment point of a spacer sequence
475 corresponds to the CRISPR repeat processing positions performed
by CasM upon guide binding
10 indicates the tenth nucleotide position
20 indicates the twentieth nucleotide position
30 indicates the thirtieth nucleotide position

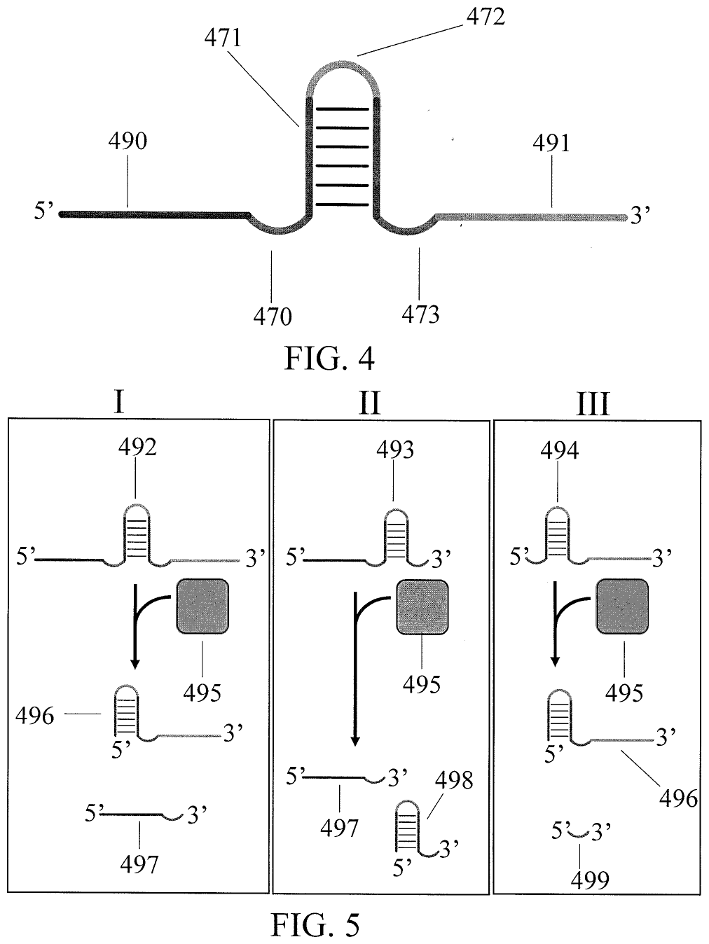

The repeat sequence was used to design a CRISPR array by incorporating spacer sequences 5′,3′, or both 5′ and 3′ of the repeat sequence. The sequences were used for synthesis as RNA. The synthetic CasM CRISPR array is represented in FIG. 4 and structural components 470-473 are detailed in Table 9. Additional components 490 and 491 correspond to a first 5′ and a first 3′ spacer sequence, respectively. The CRISPR array components are shown in Table 10.

TABLE 10
CRISPR Array Components

| | CRISPR | | |
|---|---|---|---|
| SEQ ID | Array Con- | | Size |
| NO: | figuration | Sequence | (nt) |
| SEQ ID | spacer- | UGAUACUGCUUUGAUGUCAGCAUUGC | 96 |
| NO: 46 | repeat- | AUAUCUACUAUACUGGUGCGAAUUUG | |
| | spacer | CACUAGUCUAAAAUCUAUAACCAUAA | |
| | | GUUCUUCUGCGUUCAUAU | |
| SEQ ID | spacer- | UGAUACUGCUUUGAUGUCAGCAUUGC | 66 |
| NO: 47 | repeat | AUAUCUACUAUACUGGUGCGAAUUUG | |
| | | CACUAGUCUAAAAU | |
| SEQ ID | repeat- | CUACUAUACUGGUGCGAAUUUGCACU | 66 |
| NO: 48 | spacer | AGUCUAAAAUUGAUACUGCUUUGAUG | |
| | | UCAGCAUUGCAUAU | |

*CRISPR repeat sequence is underlined

SEQ ID NO:46 comprises, in a 5′ to 3′ orientation, CRISPR array structural components 490, 470-473, and 491. SEQ ID NO:47 comprises, in a 5′ to 3′ orientation, CRISPR array structural components 490 and 470-473. SEQ ID NO:48 comprises, in a 5′ to 3′ orientation, CRISPR array structural components 470-473 and 491.

Alternative to synthesis, CRISPR arrays may be made via PCR using 3′ overlapping primers containing DNA sequences corresponding to CRISPR array components and incorporation of a T7 promoter sequence 5′ of the CRISPR arrays, followed by in vitro transcription.

### B. CasM Purification

The CasM protein coding sequence was codon-optimized for expression in E. coli and

incorporated into a modified pET plasmid backbone downstream of a maltose binding protein (MBP) using appropriate restriction nucleases. The plasmid backbone contained a T7-Lac promoter upstream of the MBP-CasM coding sequence to facilitate transcription in cells. Additionally, the plasmid backbone contained an kanamycin resistance gene and a ColE1 origin of replication.

The CasM expression plasmid was transformed into Rosetta2 (DE3) cells, and cells were grown in two 1 L shake flasks at 37° C. until cells reached an optical density of 0.6, after which protein expression was induced by addition of 0.5 mM IPTG. Cells were then incubated at 16° C. overnight.

Cells were collected via centrifugation and lysed via sonication. Cell debris was pelleted, and the clarified lysate was purified using a combination of HisTrap column chromatography, followed by cleavage of the MBP tag, and finally cation exchange column chromatography. Final purified protein was quantified using a NanoDrop™ 2000 spectrophotometer (ThermoFisher, Waltham, MA), and stored at −80° C.

### C. In Vitro CRISPR Array Processing

Synthetic CRISPR array reagents were resuspended in water to a final concentration of 250 μM and diluted to a working concentration of 250 nM. CRISPR arrays were incubated at 95° C. for two minutes and cooled by 0.5° C./sec in a thermocycler to a final temperature of 25° C.

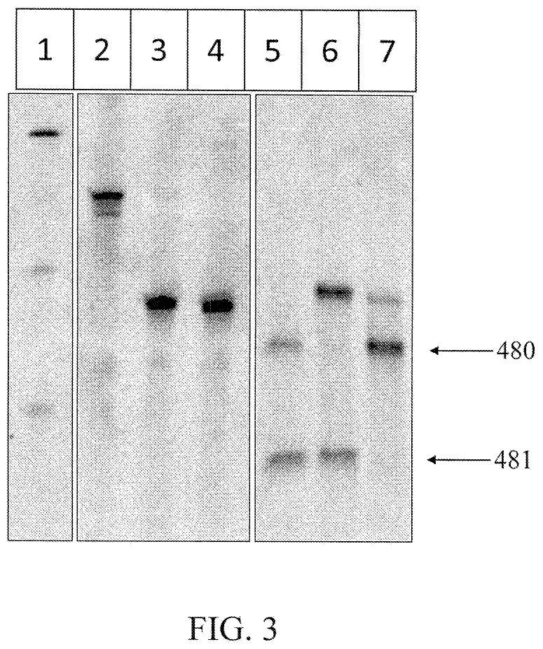

CasM was diluted to a final concentration of 500 nM in 1× cleavage buffer (20 mM HEPES, 100 mM KCl, 5 mM MgCl₂, and 5% glycerol at pH 7.4). The reaction was initiated by addition of CasM protein to denatured CRISPR arrays in a final reaction volume of 12 μL, followed by incubation at 37° C. for 15 minutes. The reaction was terminated by heat inactivation at 95° C. for 2 minutes, and 6 μL of the reaction was mixed with 6 μL of 2× RNA loading buffer (New England Biolabs, Ipswich, MA). Low Range ssRNA Ladder™ (New England Biolabs, Ipswich, MA) was diluted 125-fold in water and 7 μL were mixed with 7 μL of 2× RNA Loading Dye™ (New England Biolabs, Ipswich, MA) and incubated at 90° C. for 4 minutes and then incubated on ice for 5 minutes. CRISPR array processing reactions and ssRNA ladder were analyzed on a Mini-PROTEAN 15% TBE-Urea™ (Bio-RAD, Hercules, CA) run at 200 V for 1 hour in 1× TBE running buffer. The gel was stained using 2× SYBR gold™ (MilliporeSigma, St. Louis, MI) for 15 minutes and visualized using a Gel Doc™ EZ system™ (Bio-RAD, Hercules, CA). The results of the CRISPR array processing reactions are shown in FIG. 3 and lane order is presented in Table 11.

TABLE 11
CRISPR Array Cleavage Gel Lane Order

| | CRISPR Array | | |
|---|---|---|---|
| Lane | Configuration | SEQ ID NO: | CasM |
| 1 | Low Range ssRNA Ladder | | |
| 2 | spacer-repeat-spacer | SEQ ID NO: 46 | − |
| 3 | spacer-repeat | SEQ ID NO: 47 | − |
| 4 | repeat-spacer | SEQ ID NO: 48 | − |
| 5 | spacer-repeat-spacer | SEQ ID NO: 46 | + |
| 6 | spacer-repeat | SEQ ID NO: 47 | + |
| 7 | repeat-spacer | SEQ ID NO: 48 | + |

The results of the CRISPR array cleavage assays (FIG. 3) demonstrated that the CasM protein is capable of processing a cognate CRISPR array. The three bands shown in Lane 1 correspond to 150, 80 and 50 nucleotide standards of the Low Range ssRNA Ladder™ (New England Biolabs, Ipswich, MA), respectively). Indicator 480 in FIG. 3 corresponds to a processed CasM crRNA comprising a portion of the CRISPR repeat sequence and a spacer sequence. Indicator 481 corresponds to RNA species cleaved from the 5′ end of the CRISPR array following addition of CasM.

The CasM cleaved nucleotides in 5′ region of the repeat element (FIG. 3, comparing Lane 2 to 5; comparing Lane 3 to Lane 6), and exhibited no cleavage 3′ of the repeat element (FIG. 3, comparing Lane 4 to Lane 7). crRNA proceeded from the CasM CRISPR array and therefore had a 5′ repeat element and a spacer element 3′ of the repeat. In the absence of CasM, no cleavage of the crRNA was observed (FIG. 3, Lanes 2, 3, and 4).

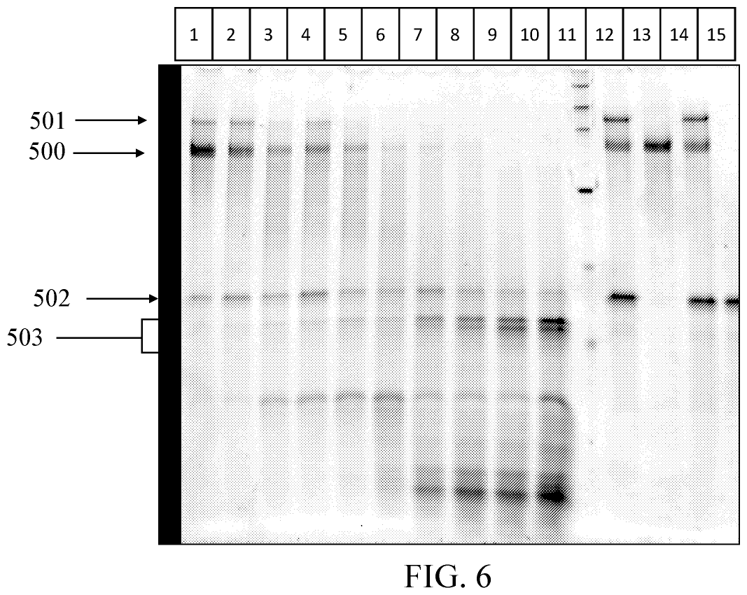

Schematics of the crRNA processing regimes are depicted in FIG. 5. In FIG. 5, panel I corresponds to the reaction in FIG. 3, Lane 5; FIG. 5 panel II corresponds to the reaction in FIG. 3, Lane 6; and FIG. 5 panel III corresponds to the reaction in FIG. 3, Lane 7. The various components represented in FIG. 5 are described in Table 12.

TABLE 12
Numerical Indicators Used to Illustrate the Results of the
in vitro CRISPR Array Cleavage Assay (FIG. 5)

492 corresponds to a spacer-repeat-spacer CRISPR array (SEQ ID.
NO: 46)
493 corresponds to a spacer-repeat CRISPR array (SEQ ID. NO: 47)
494 corresponds to a repeat-spacer CRISPR array (SEQ ID. NO: 48)
495 corresponds to a CasM protein
496 corresponds to a processed crRNA
497 corresponds to a RNA species cleaved from the 5′ end of the
CRISPR array
498 corresponds to a processed CRISPR repeat sequence
499 corresponds to a RNA species cleaved from the 5′ end of the
CRISPR repeat sequence

### Example 7

### CasM ssRNA Cleavage Assay

This Example illustrates the use of a crRNA/CasM protein complex to carry out ssRNA cleavage. The following method may be practiced with other CasM protein and crRNA to cleave ssRNA targets.

A. Generation of ssRNA Target

A ssRNA target was generated via PCR amplification of a 224 nucleotide target sequence from a plasmid. A T7 promoter sequence was incorporated into 5′ end of the reverse PCR primer (SEQ ID NO:50) for transcription. The primers used for ssRNA target DNA template are presented in Table 13.

TABLE 13
ssRNA Target DNA Template Primers

| SEQ ID NO: | Name | Sequence |
|---|---|---|
| SEQ ID NO: 49 | Forward | CGAAATTAATACGACTCACTATA |
| | primer | GGTTTCGATTATGCGGCCGTGT |
| SEQ ID NO: 50 | Reverse | AGGAGATATACCATGGGCAGCA |
| | primer | |

*T7 Promoter sequence underlined.

The primers were present at a concentration of 400 nM each. PCR reactions were performed using Q5 Hot Start High-Fidelity 2× Master Mix™ (New England Biolabs, Ipswich, MA) following the manufacturer's instructions with 10 ng of plasmid template. PCR assembly reactions were carried out using the following thermal cycling conditions: 98° C. for 2 minutes; cycles of 10 seconds at 98° C.; 15 seconds at 60° C.; 30 seconds at 72° C.; and a final extension at 72° C. for 2 minutes. DNA product quality was evaluated after the PCR reaction by agarose gel electrophoresis (1.5%, SYBR® Safe; Life Technologies, Grand Island, NY).

Between 0.1-0.5 μg of the amplified ssRNA target DNA template was used as a template for transcription using T7 High Yield RNA Synthesis Kit™ (New England Biolabs, Ipswich, MA) for approximately 16 hours at 37° C. Transcription reactions were treated with DNase I (New England Biolabs, Ipswich, MA) and purified using GeneJet RNA Cleanup and Concentration Kit™ (Life Technologies, Grand Island, NY). The quality of the transcribed RNA was checked by agarose gel electrophoresis (2%, SYBR® Safe; Life Technologies, Grand Island, NY) and quantified using the Quant-iT™ RNA Assay Kit™ (ThermoFisher, Waltham, MA).

B. Designing CasM crRNA

The 224 nucleotide ssRNA target sequence was probed in silico for a 30 nucleotide target sequence. The target sequence was appended in silico to the 3′ end of the CasM crRNA repeat sequence and the crRNA sequence was provided to a commercial manufacturer for synthesis.

C. ssRNA Cleavage Assay

Synthetic crRNA reagents were resuspended in water to a final concentration of 250 μM and diluted to a suitable working concentration of 250 nM. In vitro transcribed ssRNA target was diluted to 43 ng/μL in water. Both the crRNA and the ssRNA target reagents were separately incubated at 95° C. for two minutes and cooled by 0.5° C./sec in a thermocycler to a final temperature of 25° C. The CasM protein was diluted to various concentrations in water and 1× cleavage buffer. Denatured crRNA was added at various concentrations to the CasM protein and incubated in a thermocycler for 10 minutes at 37° C. The cleavage reactions were initiated by the addition of the ssRNA target to a final concentration of 56.4 nM in a final reaction volume of 12 μL. The concentration of each component in the various reactions is shown in Table 14.

TABLE 14
ssRNA Targeting Reaction Component Concentrations

| | | | | Molar ratio |
|---|---|---|---|---|
| | | nM | nM ssRNA | CasM:crRNA:ssRNA |
| Reaction | nM CasM | crRNA | target | target |
| 1 | 11.3 | 33.8 | 56.4 | 0.2:0.6:1 |
| 2 | 22.5 | 67.6 | 56.4 | 0.4:1.2:1 |
| 3 | 33.8 | 101.5 | 56.4 | 0.6:1.8:1 |
| 4 | 45.1 | 135.3 | 56.4 | 0.8:2.4:1 |
| 5 | 56.4 | 169.1 | 56.4 | 1:3:1 |
| 6 | 112.7 | 338.2 | 56.4 | 2:6:1 |
| 7 | 225.5 | 676.4 | 56.4 | 4:12:1 |
| 8 | 338.2 | 1014.6 | 56.4 | 6:18:1 |
| 9 | 450.9 | 1352.8 | 56.4 | 8:24:1 |
| 10 | 563.7 | 1691.0 | 56.4 | 10:30:1 |
| 11 | 0.0 | 0.0 | 56.4 | 0:3:1 |
| 12 | 56.4 | 0.0 | 56.4 | 1:0:1 |
| 13 | 0.0 | 169.1 | 56.4 | 0:3:1 |
| 14 | 0.0 | 169.1 | 0.0 | 1:3.0 |

Samples were mixed and centrifuged briefly before being incubated for 1 hour at 37° C. Reactions were terminated by incubating the reaction at 95° C. for 2 minutes followed by the addition of 100 U/μL of Proteinase K (New England Biolabs, Ipswich, MA), 4 M urea, 5 μM DTT, 50 μM EDTA and incubation at 37° C. for 15 minutes. 7 μL of each reaction was mixed with 6 μL of 2× RNA Loading Dye (New England Biolabs, Ipswich, MA) and incubated at 90° C. for two minutes. Low Range ssRNA Ladder™ (New England Biolabs, Ipswich, MA) was diluted 125-fold in water and 7 μL were mixed with 7 μL of 2× RNA Loading Dye™ (New England Biolabs, Ipswich, MA) and incubated at 90° C. for 4 minutes and then incubated on ice for 5 minutes. Cleavage reactions and ssRNA ladder were analyzed on a Mini-PROTEAN 15% TBE-Urea™ (BioRAD, Hercules, CA), run at 200 V for 1 hour in 1× TBE running buffer. Gel was stained using 2× SYBR gold™ (Life Technologies, Grand Island, NY) for 15 minutes and visualized with using a Gel Doc EZ system™ (BioRAD, Hercules, CA). The results of the crRNA cleavage assay are shown in FIG. 6 and the components of each lane shown in Table 15. Numerical indicator 500 corresponds to the ssRNA target. Numerical indicator 501 corresponds to a ssRNA target hybridized to the spacer sequences of the CasM crRNA. Numerical indicator 502 corresponds to an unprocessed CasM crRNA. Numerical indicator 503 corresponds to the processed CasM crRNA species.

TABLE 15
ssRNA Cleavage Gel Lane Order

| | Molar ratio |
|---|---|
| | CasM:crRNA:ssRNA |
| Lane | target |
| 1 | 0.2:0.6:1 |
| 2 | 0.4:1.2:1 |
| 3 | 0.6:1.8:1 |
| 4 | 0.8:2.4:1 |
| 5 | 1:3:1 |
| 6 | 2:6:1 |
| 7 | 4:12:1 |
| 8 | 6:18:1 |
| 9 | 8:24:1 |
| 10 | 10:30:1 |
| 11 | ssRNA Ladder |
| 12 | 0:3:1 |
| 13 | 1:0:1 |
| 14 | 0:3:1 |
| 15 | 1:3:0 |

The results of the ssRNA cleavage assay shown in FIG. 6 demonstrated that a CasM: crRNA protein complex was capable of ssRNA target cleavage. The results of this procedure demonstrate that increasing the amount of CasM: crRNA complexes resulted in decreased amounts of ssRNA target (FIG. 6, indicator 500) visualized on the gel (FIG. 6, Lanes 1-10).

### Example 8

### Production of CasM and RtcB Expression Plasmids for MS2 Phage Drop Plaque Assays

This Example describes the production of plasmids for the expression of CasM, RtcB (RNA 3′-terminal phosphate cyclase, group B), and a corresponding CRISPR array in E. coli for use in a MS2 phage drop plaque assay. The following method can be practiced with other CasM, RtcB, and CRISPR array homologs.

The casM and rtcB nucleotide sequence from Eubacterium siraeum (SEQ ID NO: 37 and SEQ ID NO:60, respectively) are selected and codon optimized for expression in E. coli. The E. coli-modified sequences are cloned into a p14A plasmid backbone using appropriate restriction nucleases. The plasmid backbone contains a T7 promoter upstream of each protein coding sequence to facilitate transcription in cells. Two control plasmids, one containing only the casM gene sequence under the control of a T7 promoter and the other plasmid only containing the rtcB gene sequence under the control of a T7 promoter, can also constructed.

A spacer sequence that has homology with the MS2 phage genome is engineered in silico flanked 5′ and 3′ by the Eubacterium siraeum CasM CRISPR repeat sequence. A non-targeting spacer with no homology to the MS2 phage or E. coli genome, is similarly engineered as a control. Both sequences are subcloned into separate plasmids between an upsteam T7 promoter sequence and a downstream transcription terminator sequence.

### Example 9

### MS2 Phage Drop Plaque Assay

This Example describes the use of CasM and RtcB in an assay to evaluate the ability of the RtcB protein to modulate CasM's sequence-specific and collateral nuclease activity in E. coli. The method set forth herein is adapted from Smargon et al., Molec. Cell (2017) 65:618-630. Not all of the following steps are required for screening, nor must the order of the steps be as presented.

The expression plasmids constructed in Example 8 are individually and in combination transformed into BL21 (AI) E. coli cells from a commercial provider, such as Invitrogen (Carlsbad, CA). Transformed cells are grown overnight at 37° C., with shaking, in lysogeny broth (LB) supplemented with 100 μg/mL carbenicillin, to select for cells that contain the CasM expression plasmid.

The following day, cells are diluted 1:100 and then grown at 37° C., with shaking, to an OD600 of 2.0. The cells are then mixed with 4 mL of carbenicillin-containing top Agar (10 g/L tryptone, 5 g/L yeast extract, 10 g/L sodium chloride, 5 g/L agar) and poured onto LB-antibiotic base plates. The top agar also contains 0.2% arabinose to induce expression of the casM, rtcB and CRISPR array coding sequences. 10-fold serial dilutions of MS2 phage (ATCC 15597-B1, Manassas VA) are made in LB and then spotted onto hardened top agar with a multi-channel pipette. Plaque formation is assessed after overnight incubation of the spotted plates at 37° C.

To assess whether the RtcB protein modulates CasM cleavage activity, the relative plaque formation is determined by comparing cells expressing CasM, RtcB, and the CRISPR array targeting MS2 phage; cells expressing CasM and the CRISPR array targeting MS2 phage; and cells expressing CasM and the CRISPR array not targeting MS2 phage; cells expressing CasM and RtcB only.

### Example 10

### Introduction of CasM RNP Complexes into Target Cells

This Example illustrates the design and delivery of CasM and crRNA ribonucleoprotein (RNP) complexes into human cells to enable mRNA cleavage of the human epidermal growth factor receptor (EGFR) gene and subsequent knockdown of EGFR gene expression.

A. Production of CasM Complexes and Transformation into Cells

Mature crRNAs (SEQ ID NOS: 70-165) were designed to target the EGFR locus in the human genome. Each crRNA contained a 5′ 36 nt repeat (SEQ ID NO:51) followed by a 30 nt spacer. crRNAs were designed to target 72 unique sequences complementary to the egfr mRNA within exons 1-3. Sequences were designed such that flanking sequences within 1 bp were not biased by any nucleotide. As negative controls, not predicted to induce cleavage, crRNAs were also designed to target (1) eight genomic sequences upstream of the predicted egfr mRNA; (2) eight sequences complementary to the vegfa mRNA exon 1; and (3) eight sequences identical to the egfr mRNA.

Double-stranded DNA (dsDNA) guide templates containing upstream T7 promoter sequences were created by annealing complementary oligonucleotides (Integrated DNA Technologies, Coralville, IA) at a final concentration of 10 μM in annealing buffer (30 mM HEPES, 300 mM KCL), then incubating at 95° C. for two minutes, and then slowly cooled to approximately 25° C., and incubated for an additional 20 minutes. Following annealing, guides were transcribed with T7 RNA polymerase HiScribe™ T7 High Yield RNA Synthesis Kit™ (New England Biolabs, Ipswich, MA) according to manufacturer's instructions. Next, samples were digested with RNase-free DNase-I (New England Biolabs, Ipswich, MA) according to manufacturer's instructions, then purified using RNAClean XP™ beads (Beckman Coulter, Indianapolis, IN).

For RNAClean XP™ bead purification, 30 μL of sample was combined with 155 μL of 100% isopropanol and 10 μL of 3 M sodium acetate and then mixed thoroughly. Next, 50 μL of RNAClean XP™ beads were incubated on a magnet for three minutes to allow separation of the liquid and beads, and the supernatant was removed. Subsequently, the samples containing crRNA were added to the beads, mixed, incubated at approximately 25° C. for five minutes, then incubated on a magnet for three minutes. Finally, the supernatant was removed, the beads were washed once with 85% ethanol, dried, and then the crRNA was eluted in 20 μL of molecular biology grade water. crRNAs were quantified using ribogreen and then normalized to 1 μg/μL.

To assemble CasM RNPs, 120 pmols of each unique crRNA were added to a well then incubated at 95° C. for two minutes followed by 25° C. for approximately 10 minutes. Next, the denatured crRNA guides were combined with 20 pmol of CasM (SEQ ID NO:39) in RNP assembly buffer (20 mM HEPES; pH 7.4, 10 mM MgCl₂, 150 mM KCl, 5% glycerol) and then incubated at 37° C. for 10 minutes.

B. Transfection of CasM RNP Complexes into Eukaryotic Cells

HeLa cells (ATCC, Manassas, VA) were cultured in suspension in DMEM medium supplemented with 10% FBS and 1× Antibiotic-Antimycotic Solution (Mediatech, Inc., Manassas, VA) at 37° C., 5% CO₂ and 100% humidity. HeLa cells were transfected using the Nucleofector® 96-well Shuttle System (Lonza, Allendale, NJ). Prior to nucleofection, 5 μl of the CasM: crRNA RNPs were assembled in individual wells of a 96-well plate. HeLa cells were transferred to a 50 ml conical centrifuge tube and centrifuged at 200×G for five minutes. The media was aspirated and the cell pellet was washed in calcium and magnesium-free PBS. The cells were centrifuged once more and resuspended in Nucleofector SF™ buffer (Lonza, Allendale, NJ) at a concentration of 5×10⁶ cells/ml. 20 μl of this cell suspension was added to the CasM: crRNA RNPs in the 96 well plate, mixed, and then the entire volume was transferred to a 96-well Nucleocuvette™ Plate. The plate was then loaded into the Nucleofector 96-well Shuttle™ and cells were nucleofected using the 96-CN-114 Nucleofector1. program (Lonza, Allendale, NJ). Immediately following nucleofection, 75 μl of complete DMEM medium was added to each well of the 96-well Nucleocuvette™ Plate. Half of the contents of each well were then transferred to a 96-well tissue culture plate containing 150 μl of complete DMEM medium. This procedure was then repeated in order to plate a duplicate for each well, one which would be used for lysis and genomic DNA analysis, and one for FACS analysis. The cells were cultured at 37° C., 5% CO₂ and 100% humidity for approximately 5 days.

### C. FACS Analysis of CasM Mediated EGFR Knockdown

Fluorescence activated cell sorting (FACS) analysis was performed 5 days after nucleofection of HeLa cells with EGFR-targeting CasM2 RNPs. In brief, 2×10⁵-4×10⁵ cells/well were detached with TrypLE Express (Gibco), stained with 2 μL APC anti-human EGFR (Clone AY13, Sony Biotechnology) in 100 μL total volume and then analyzed using Intellicyte Flow

Cytometer (Intellicyt, Albuquerque, NM). Results from these experiments are shown in Table 16.

TABLE 16
CasM Mediated EGFR knockdown

| | % | | |
|---|---|---|---|
| | EGFR | | |
| | negative | | crRNA |
| Name | cells | transcription | SEQ ID NO. |
| Untransfected reference cell | 2% | n/a | — |
| Intergenic target-1 | 5% | Intergenic | SEQ ID No: 70 |
| Intergenic target-2 | 6% | Intergenic | SEQ ID No: 71 |
| Intergenic target-3 | 9% | Intergenic | SEQ ID No: 72 |
| Intergenic target-4 | 8% | Intergenic | SEQ ID No: 73 |
| Intergenic target-5 | 5% | Intergenic | SEQ ID No: 74 |
| Intergenic target-6 | 6% | Intergenic | SEQ ID No: 75 |
| Intergenic target-7 | 6% | Intergenic | SEQ ID No: 76 |
| Intergenic target-8 | 4% | Intergenic | SEQ ID No: 77 |
| Exon 1 target-1 | 6% | Exon 1/28 | SEQ ID No: 78 |
| Exon 1 target-2 | 5% | Exon 1/28 | SEQ ID No: 79 |
| Exon 1 target-3 | 5% | Exon 1/28 | SEQ ID No: 80 |
| Exon 1 target-4 | 4% | Exon 1/28 | SEQ ID No: 81 |
| Exon 1 target-5 | 6% | Exon 1/28 | SEQ ID No: 82 |
| Exon 1 target-6 | 5% | Exon 1/28 | SEQ ID No: 83 |
| Exon 1 target-7 | 6% | Exon 1/28 | SEQ ID No: 84 |
| Exon 1 target-8 | 6% | Exon 1/28 | SEQ ID No: 85 |
| Exon 1 target-9 | 4% | Exon 1/28 | SEQ ID No: 86 |
| Exon 1 target-10 | 3% | Exon 1/28 | SEQ ID No: 87 |
| Exon 1 target-11 | 3% | Exon 1/28 | SEQ ID No: 88 |
| Exon 1 target-12 | 2% | Exon 1/28 | SEQ ID No: 89 |
| Exon 1 target-13 | 4% | Exon 1/28 | SEQ ID No: 90 |
| Exon 1 target-14 | 4% | Exon 1/28 | SEQ ID No: 91 |
| Exon 1 target-15 | 5% | Exon 1/28 | SEQ ID No: 92 |
| Exon 1 target-16 | 4% | Exon 1/28 | SEQ ID No: 93 |
| Exon 1 target-17 | 6% | Exon 1/28 | SEQ ID No: 94 |
| Exon 1 target-18 | 6% | Exon 1/28 | SEQ ID No: 95 |
| Exon 1 target-19 | 6% | Exon 1/28 | SEQ ID No: 96 |
| Exon 1 target-20 | 5% | Exon 1/28 | SEQ ID No: 97 |
| Exon 1 target-21 | 5% | Exon 1/28 | SEQ ID No: 98 |
| Exon 1 target-22 | 5% | Exon 1/28 | SEQ ID No: 99 |
| Exon 1 target-23 | 6% | Exon 1/28 | SEQ ID No: 100 |
| Exon 1 target-24 | 5% | Exon 1/28 | SEQ ID No: 101 |
| Exon 2 target-1 | 6% | Exon 2/28 | SEQ ID No: 102 |
| Exon 2 target-2 | 7% | Exon 2/28 | SEQ ID No: 103 |
| Exon 2 target-3 | 11% | Exon 2/28 | SEQ ID No: 104 |
| Exon 2 target-4 | 5% | Exon 2/28 | SEQ ID No: 105 |
| Exon 2 target-5 | 6% | Exon 2/28 | SEQ ID No: 106 |
| Exon 2 target-6 | 8% | Exon 2/28 | SEQ ID No: 107 |
| Exon 2 target-7 | 11% | Exon 2/28 | SEQ ID No: 108 |
| Exon 2 target-8 | 10% | Exon 2/28 | SEQ ID No: 109 |
| Exon 2 target-9 | 13% | Exon 2/28 | SEQ ID No: 110 |
| Exon 2 target-10 | 8% | Exon 2/28 | SEQ ID No: 111 |
| Exon 2 target-11 | 10% | Exon 2/28 | SEQ ID No: 112 |
| Exon 2 target-12 | 8% | Exon 2/28 | SEQ ID No: 113 |
| Exon 2 target-13 | 13% | Exon 2/28 | SEQ ID No: 114 |
| Exon 2 target-14 | 16% | Exon 2/28 | SEQ ID No: 115 |
| Exon 2 target-15 | 19% | Exon 2/28 | SEQ ID No: 116 |
| Exon 2 target-16 | 11% | Exon 2/28 | SEQ ID No: 117 |
| Exon 2 target-17 | 10% | Exon 2/28 | SEQ ID No: 118 |
| Exon 2 target-18 | 19% | Exon 2/28 | SEQ ID No: 119 |
| Exon 2 target-19 | 20% | Exon 2/28 | SEQ ID No: 120 |
| Exon 2 target-20 | 25% | Exon 2/28 | SEQ ID No: 121 |
| Exon 2 target-21 | 15% | Exon 2/28 | SEQ ID No: 122 |
| Exon 2 target-22 | 17% | Exon 2/28 | SEQ ID No: 123 |
| Exon 2 target-23 | 14% | Exon 2/28 | SEQ ID No: 124 |
| Exon 2 target-24 | 12% | Exon 2/28 | SEQ ID No: 125 |
| Exon 3 target-1 | 7% | Exon 3/28 | SEQ ID No: 126 |
| Exon 3 target-2 | 7% | Exon 3/28 | SEQ ID No: 127 |
| Exon 3 target-3 | 9% | Exon 3/28 | SEQ ID No: 128 |
| Exon 3 target-4 | 9% | Exon 3/28 | SEQ ID No: 129 |
| Exon 3 target-5 | 8% | Exon 3/28 | SEQ ID No: 130 |
| Exon 3 target-6 | 11% | Exon 3/28 | SEQ ID No: 131 |
| Exon 3 target-7 | 12% | Exon 3/28 | SEQ ID No: 132 |
| Exon 3 target-8 | 12% | Exon 3/28 | SEQ ID No: 133 |
| Exon 3 target-9 | 10% | Exon 3/28 | SEQ ID No: 134 |
| Exon 3 target-10 | 9% | Exon 3/28 | SEQ ID No: 135 |
| Exon 3 target-11 | 11% | Exon 3/28 | SEQ ID No: 136 |
| Exon 3 target-12 | 40% | Exon 3/28 | SEQ ID No: 137 |
| Exon 3 target-13 | 17% | Exon 3/28 | SEQ ID No: 138 |
| Exon 3 target-14 | 15% | Exon 3/28 | SEQ ID No: 139 |
| Exon 3 target-15 | 12% | Exon 3/28 | SEQ ID No: 140 |
| Exon 3 target-16 | 21% | Exon 3/28 | SEQ ID No: 141 |
| Exon 3 target-17 | 48% | Exon 3/28 | SEQ ID No: 142 |
| Exon 3 target-18 | 41% | Exon 3/28 | SEQ ID No: 143 |
| Exon 3 target-19 | 19% | Exon 3/28 | SEQ ID No: 144 |
| Exon 3 target-20 | 9% | Exon 3/28 | SEQ ID No: 145 |
| Exon 3 target-21 | 19% | Exon 3/28 | SEQ ID No: 146 |
| Exon 3 target-22 | 8% | Exon 3/28 | SEQ ID No: 147 |
| Exon 3 target-23 | 8% | Exon 3/28 | SEQ ID No: 148 |
| Exon 3 target-24 | 6% | Exon 3/28 | SEQ ID No: 149 |
| VEGFA target-1 | 6% | Exon 1/8 | SEQ ID No: 150 |
| VEGFA target-2 | 8% | Exon 1/8 | SEQ ID No: 151 |
| VEGFA target-3 | 8% | Exon 1/8 | SEQ ID No: 152 |
| VEGFA target-4 | 7% | Exon 1/8 | SEQ ID No: 153 |
| VEGFA target-5 | 8% | Exon 1/8 | SEQ ID No: 154 |
| VEGFA target-6 | 7% | Exon 1/8 | SEQ ID No: 155 |
| VEGPA target-7 | 6% | Exon 1/8 | SEQ ID No: 156 |
| VEGFA target-8 | 8% | Exon 1/8 | SEQ ID No: 157 |
| Nontargeting target-1 | 5% | Exon 1/28 | SEQ ID No: 158 |
| Nontargeting target-2 | 7% | Exon 1/28 | SEQ ID No: 159 |
| Nontargeting target-3 | 6% | Exon 1/28 | SEQ ID No: 160 |
| Nontargeting target-4 | 6% | Exon 1/28 | SEQ ID No: 161 |
| Nontargeting target-5 | 6% | Exon 1/28 | SEQ ID No: 162 |
| Nontargeting target-6 | 6% | Exon 1/28 | SEQ ID No: 163 |
| Nontargeting target-7 | 7% | Exon 1/28 | SEQ ID No: 164 |
| Nontargeting target-8 | 4% | Exon 1/28 | SEQ ID No: 165 |

The data presented in Table 16 shows that CasM did not produce egfr knockdown when targeted to (1) sequences upstream of the predicted exon 1 start site using SEQ ID NOS: 70-77; (2) an unrelated vascular endothelial growth factor A (vegfa) gene using SEQ ID NOS: 150-157); or (3) the reverse complement of sequences contained in egfr exon 1 mRNA using SEQ ID NOS: 158-165. Conversely, CasM enabled approximately 40% egfr knockdown when targeted to mRNA sequences contained in exon 2 using SEQ ID NOS: 102-125, and exon 3 of egfr using SEQ ID NOS: 126-149.

Although preferred embodiments of the subject methods have been described in some detail, it is understood that obvious variations can be made without departing from the spirit and the scope of the invention as defined by the appended claims.

### BRIEF DESCRIPTION OF THE FIGURES

FIG. 1 depicts a representative CasM operon from Eubacterium siraeum (NCBI Accession No. NZ_DS499551.1).

FIG. 2 shows a structure of a CasM repeat sequence (SEQ ID NO:51).

FIG. 3 shows the results of the in vitro CRISPR array cleavage assay described in the Examples.

FIG. 4 shows a depiction of a synthetic CasM CRISPR array.

FIG. 5 shows a representation of the results of the in vitro CRISPR array cleavage assay results shown in FIG. 3 and described in the Examples.

FIG. 6 shows the results of the CasM ssRNA cleavage assay described in the Examples.

### DETAILED DESCRIPTION OF THE INVENTION

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to a “guide/Cas complex” includes one or more such complexes, reference to “a polynucleotide” includes one or more polynucleotides, etc.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, 2014, Cold Spring Harbor Laboratory Press, ISBN 978-1-936113-81-1; Culture of Animal Cells: A Manual of Basic Technique and Specialized Applications, 6th Edition, R. I. Freshney, 2010, Wiley-Blackwell, ISBN 978-0-470-52812-9; Transgenic Animal Technology, Third Edition: A Laboratory Handbook, 2014, C. A. Pinkert, Elsevier, ISBN 978-0124104907; The Laboratory Mouse, Second Edition, 2012, H. Hedrich, Academic Press, ISBN 978-0123820082; Manipulating the Mouse Embryo: A Laboratory Manual, 2013, R. Behringer, et al., Cold Spring Harbor Laboratory Press, ISBN 978-1936113019; PCR 2: A Practical Approach, 1995, M. J. McPherson, et al., IRL Press, ISBN 978-0199634248; Methods in Molecular Biology (Series), J. M. Walker, ISSN 1064-3745, Humana Press; RNA: A Laboratory Manual, 2010, D. C. Rio, et al., Cold Spring Harbor Laboratory Press, ISBN 978-0879698911; Methods in Enzymology (Series), Academic Press; Molecular Cloning: A Laboratory Manual (Fourth Edition), 2012, M. R. Green, et al., Cold Spring Harbor Laboratory Press, ISBN 978-1605500560; Bioconjugate Techniques, Third Edition, 2013, G. T. Hermanson, Academic Press, ISBN 978-0123822390; Methods in Plant Biochemistry and Molecular Biology, 1997, W. V. Dashek, CRC Press, ISBN 978-0849394805; Plant Cell Culture Protocols (Methods in Molecular Biology), 2012, V. M. Loyola-Vargas, et al., Humana Press, ISBN 978-1617798177; Plant Transformation Technologies, 2011, C. N. Stewart, et al., Wiley-Blackwell, ISBN 978-0813821955; Recombinant Proteins from Plants (Methods in Biotechnology), 2010, C. Cunningham, et al., Humana Press, ISBN 978-1617370212; Plant Genomics: Methods and Protocols (Methods in Molecular Biology), 2009, D. J. Somers, et al., Humana Press, ISBN 978-1588299970; Plant Biotechnology: Methods in Tissue Culture and Gene Transfer, 2008, R. Keshavachandran, et al., Orient Blackswan, ISBN 978-8173716164.

As used herein, “a CasM protein” refers to a CRISPR protein capable of targeting RNA and causing single-strand RNA breaks when guided to a target site by a crRNA, without the necessity of association with a tracrRNA. CasM proteins typically include two or more higher eukaryotic and prokaryotic nucleotide-binding (HEPN) domains found in protein family PF05168 in the C-terminal region of the CasM sequence. CasM proteins show synteny with one or more WYL domain-containing proteins and sometimes with RtcB (RNA 3′-terminal phosphate cyclase, group B) domain-containing proteins. Based on the foregoing characteristics, CasM may be classified as a Class 2 Type VI CRISPR-Cas system because it is a single effector protein containing two HEPN domains used for targeted ssRNA interference. However, CasM has a very low degree of sequence similarity to other Type VI subtypes. Exemplary CasM proteins are shown in SEQ ID NOS: 37-45, and are encoded by polynucleotides shown in SEQ ID NOS: 28-36, respectively. These proteins display approximately 13.59% to 99.82% sequence identity to each other and show less than 8% sequence identity with other known CRISPR-Cas proteins. As used herein, the term “CasM protein” refers to a CasM protein derived from any species, subspecies, or strain of bacteria that encodes the CasM protein, as well as an ortholog of the CasM protein, i.e., a CasM protein from a species other than the species producing the reference CasM protein. For example, CasM orthologs of Eubacterium siraeum CasM, shown in Table 1, display approximately 13.59% to 99.82% sequence identity to each other. Thus, CasM orthologs are identified based on the CasM characteristics detailed herein. Reference to a CasM protein also encompasses a variant of the reference CasM protein, e.g., an active homolog of the reference amino acid sequence. Thus, CasM proteins include, but are not limited to, those proteins depicted in SEQ ID NOS: 37-45, orthologs thereof, or variants thereof. Non-limiting examples of such proteins include CasM proteins from Eubacterium siraeum; Ruminococcus sp., such as from Ruminococcus bicirculans; Ruminococcus flavefaciens, such as, but not limited to, FD-1 and strain XPD3002; Ruminococcus albus such as, but not limited to, strain KH2T6; Ruminococcus sp. isolates, such as but not limited to, isolates 2789STDY5834971, 2789STDY5608892 and 2789STDY5834894.

By “dCasM protein” is meant a deactivated CasM protein lacking activity, such as catalytic and/or binding activity, also termed “dead CasM.” Such molecules lack all or a portion of biological activity, such as nuclease and/or binding activity, and are therefore unable to bind and/or cleave a target nucleic acid of interest, respectively. In some embodiments, these deactivated CasM proteins can be used to regulate genes in a nucleic acid-guided manner. This is accomplished by introducing mutations that inactivate CasM nuclease function and typically involves mutating catalytic residues of the gene encoding CasM. dCasM can be used alone or in fusions to synthetically repress (CRISPR interference or CRISPRi) or activate (CRISPR activation or CRISPRa) gene expression. CRISPRi can work independently of host cellular machineries. In some embodiments a dCasM protein and a customized nucleic acid-targeting nucleic acid, i.e., a cognate nucleic acid guide designed with a complementary region to any gene of interest, are used to direct dCasM to a chosen genomic location. In other embodiments, dCasM can be fused to a transcription factor, such as a repressor, and the fused dCasM-transcription factor can then work in concert with cellular machineries. CRISPRa is carried out by dCasM-transcription factor (activator) fusions.

A “nucleic acid-targeting nucleic acid” (NATNA), as used herein, refers to one or more polynucleotides that guide a protein, such as a CasM protein, to preferentially target a nucleic acid target sequence present in a polynucleotide (relative to a polynucleotide that does not comprise the nucleic acid target sequence). Such NATNAs are also known herein as “cognate nucleic acid guides,” or “cognate guides.” NATNAs can comprise ribonucleotide bases (e.g., RNA), deoxyribonucleotide bases (e.g., DNA), combinations of ribonucleotide bases and deoxyribonucleotide bases (e.g., RNA/DNA), nucleotides, nucleotide analogs, modified nucleotides, and the like, as well as synthetic, naturally occurring, and non-naturally occurring modified backbone residues or linkages. Thus, a NATNA as used herein site-specifically guides a CasM, or a deactivated CasM, to a target nucleic acid. Many such NATNAs are known, such as but not limited to sgRNA (including miniature and truncated single-guide RNAs), crRNA, dual-guide RNA, including but not limited to, crRNA/tracrRNA molecules, as described herein, and the like, the use of which depends on the particular Cas protein. For a non-limiting description of exemplary NATNAs, see, e.g., PCT Publication No. WO 2014/150624 to May et al., published Sep. 29, 2014; PCT Publication No. WO 2015/200555 to May et al., published Mar. 10, 2016; PCT Publication No. WO 2016/201155 to Donohoue et al., published Dec. 15, 2016; PCT Publication No. WO 2017/027423 to Donohoue et al., published Feb. 16, 2017; and PCT Publication No. WO 2016/123230 to May et al., published Aug. 4, 2016.

With reference to a NATNA or a cognate nucleic acid guide, a “spacer,” “spacer sequence,” or “spacer element,” as used herein, refers to the polynucleotide sequence that can specifically hybridize to a target nucleic acid sequence. The spacer element interacts with the target nucleic acid sequence through hydrogen bonding between complementary base pairs (i.e., paired bases). A spacer element binds to a selected nucleic acid target sequence. Accordingly, the spacer element is the nucleic acid target-binding sequence. The spacer element determines the location of a Cas protein's site-specific binding and nucleolytic cleavage. Spacer elements range from approximately 17 to approximately 84 nucleotides in length and have an average length of 36 nucleotides (see, e.g., Marraffini, et al., “CRISPR interference: RNA-directed adaptive immunity in bacteria and archaea,” Nature reviews Genetics (2010) 11:181-190). Variability of the functional length for a spacer element is known in the art (e.g., U.S. Patent Publication 2014/0315985 to May et al., published Oct. 23, 2014). The terms “nucleic acid target binding sequence” and “spacer sequence” are used interchangeably herein.

The term “sgRNA” typically refers to a single-guide RNA (i.e., a single, contiguous polynucleotide sequence) that essentially comprises a crRNA connected at its 3′ end to 5′ end of a tracrRNA through a “loop” sequence (see, e.g., U.S. Published Patent Application No. 2014/0068797 to Doudna et al., published 6 Mar. 2014, incorporated herein by reference in its entirety). sgRNA interacts with a cognate Cas protein essentially as described for tracrRNA/crRNA polynucleotides. Similar to crRNA, sgRNA has a spacer, a region of complementarity to a potential DNA or RNA target sequence, adjacent a second region that forms base-pair hydrogen bonds that form a secondary structure, typically a stem structure. The term includes truncated single-guide RNAs (tru-sgRNAs) of approximately 17-18 nucleotides (nt) (see, e.g., Fu et.al., Nat Biotechnol. (2014) 32:279-284). The term also encompasses functional miniature sgRNAs with expendable features removed, but that retain an essential and conserved module termed the “nexus” located in the portion of sgRNA that corresponds to tracrRNA (not crRNA). See, e.g., U.S. Patent Publication 2014/0315985 to May et al., published Oct. 23, 2014, incorporated herein by reference in its entirety; Briner et al., “Guide RNA Functional Modules Direct Cas9 Activity and Orthogonality,” Molecular Cell (2014) 56:333-339.

As used herein, “dual-guide RNA” refers to a two-component RNA system for a polynucleotide component capable of associating with a cognate Cas protein. A representative CRISPR Class 2 Type II CRISPR-Cas-associated dual-guide RNA includes a Cas-crRNA and Cas-tracrRNA, paired by hydrogen bonds to form secondary structure (see, e.g., U.S. Published Patent Application No. 2014/0068797 to Doudna et al., published 6 Mar. 2014, incorporated herein by reference in its entirety; see also Jinek M., et al., Science 337:816-21 (2012)). A Cas-dual-guide RNA is capable of forming a nucleoprotein complex with a cognate Cas protein, wherein the complex is capable of targeting a nucleic acid target sequence complementary to the spacer sequence.

As used herein, the term “cognate” typically refers to a Cas protein (e.g., CasM protein) and one or more polynucleotides (e.g., a CRISPR-CasM-associated cognate nucleic acid guide) capable of forming a nucleoprotein complex for site-directed binding to a nucleic acid target sequence complementary to the nucleic acid target binding sequence present in one of the one or more polynucleotides.

The terms “wild-type,” “naturally-occurring,” “native,” and “unmodified” are used herein to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in and can be isolated from a source in nature. The wild-type form, appearance, phenotype, or strain serve as the original parent before an intentional modification. Thus, mutant, variant, engineered, recombinant, and modified forms are not wild-type forms.

As used herein, the terms “engineered,” “genetically engineered,” “recombinant,” “modified,” and “non-naturally occurring” are interchangeable and indicate intentional human manipulation.

“Covalent bond,” “covalently attached,” “covalently bound,” “covalently linked,” “covalently connected,” and “molecular bond” are used interchangeably herein, and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to hydrogen bonding, ionic interactions (e.g., Na⁺Cl⁻), van der Waals interactions, and hydrophobic bonds.

As used herein, “hydrogen bonding,” “hydrogen base pairing,” and “hydrogen bonded” are used interchangeably and refer to canonical hydrogen bonding and non-canonical hydrogen bonding including, but not limited to, “Watson-Crick-hydrogen-bonded base pairs” (W—C-hydrogen-bonded base pairs or W—C hydrogen bonding); “Hoogsteen-hydrogen-bonded base pairs” (Hoogsteen hydrogen bonding); and “wobble-hydrogen-bonded base pairs” (wobble hydrogen bonding). W—C hydrogen bonding, including reverse W—C hydrogen bonding, refers to purine-pyrimidine base pairing, that is, adenine: thymine, guanine: cytosine, and uracil: adenine. Hoogsteen hydrogen bonding, including reverse Hoogsteen hydrogen bonding, refers to a variation of base pairing in nucleic acids wherein two nucleobases, one on each strand, are held together by hydrogen bonds in the major groove. This non-W—C hydrogen bonding can allow a third strand to wind around a duplex and form triple-stranded helices. Wobble hydrogen bonding, including reverse wobble hydrogen bonding, refers to a pairing between two nucleotides in RNA molecules that does not follow Watson-Crick base pair rules. There are four major wobble base pairs: guanine: uracil, inosine (hypoxanthine): uracil, inosine-adenine, and inosine-cytosine. Rules for canonical hydrogen bonding and non-canonical hydrogen bonding are known to those of ordinary skill in the art (see, e.g., The RNA World, Third Edition (Cold Spring Harbor Monograph Series), R. F. Gesteland, Cold Spring Harbor Laboratory Press, ISBN 978-0879697396 (2005); The RNA World, Second Edition (Cold Spring Harbor Monograph Series), R. F. Gesteland, et al., Cold Spring Harbor Laboratory Press, ISBN 978-0879695613 (1999); The RNA World (Cold Spring Harbor Monograph Series), R. F. Gesteland, et al., Cold Spring Harbor Laboratory Press, ISBN 978-0879694562 (1993) (see, e.g., Appendix 1: Structures of Base Pairs Involving at Least Two Hydrogen Bonds, I. Tinoco); Principles of Nucleic Acid Structure, W. Saenger, Springer International Publishing AG, ISBN 978-0-387-90761-1 (1988); Principles of Nucleic Acid Structure, First Edition, S. Neidle, Academic Press, ISBN 978-01236950791 (2007)).

“Connect,” “connected,” and “connecting” are used interchangeably herein, and refer to a covalent bond or a non-covalent bond between two macromolecules (e.g., polynucleotides, proteins, and the like). Thus, CasM and a cognate nucleic acid guide are “connected” in a cognate nucleic acid guide/CasM complex.

As used herein, the terms “nucleic acid,” “nucleic acid sequence,” “nucleotide sequence,” “oligonucleotide,” and “polynucleotide” are interchangeable and refer to a polymeric form of nucleotides. The nucleotides may be deoxyribonucleotides (DNA), ribonucleotides (RNA), analogs thereof, or combinations thereof, and may be of any length. Polynucleotides may perform any function and may have any secondary and tertiary structures. The terms encompass known analogs of natural nucleotides and nucleotides that are modified in the base, sugar and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, which are synthetic, naturally occurring, and non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Inc., Woburn, MA) nucleosides, glycol nucleic acid, bridged nucleic acids, and morpholino structures.

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3 to 5 nucleotides at 5′-end or 3′-end sequences of a polynucleotide sequence to inhibit exonuclease degradation. Placement of phosphorothioate bonds throughout an entire oligonucleotide helps reduce degradation by nucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., ucalgary.ca/dnalab/synthesis/-modifications/linkages). A 3′-3′ linkage at a terminus of a polynucleotide stabilizes the polynucleotide to exonuclease degradation by creating an oligonucleotide having two 5′-OH termini but lacking a 3′-OH terminus. Typically, such polynucleotides have phosphoramidite groups on 5′-OH position and a dimethoxytrityl (DMT) protecting group on 3′-OH position. Normally, the DMT protecting group is on 5′-OH and the phosphoramidite is on 3′-OH.

Polynucleotide sequences are displayed herein in the conventional 5′ to 3′ orientation unless otherwise indicated.

As used herein, the term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Sequence-specific binding, as used herein, typically refers to one or more cognate nucleic acid guides (i.e., NATNAs) capable of forming a complex with a protein (e.g., a CasM protein) to cause the protein to bind a nucleic acid sequence (e.g., a RNA or DNA sequence) comprising a nucleic acid target sequence (e.g., a RNA or DNA target sequence) preferentially relative to a second nucleic acid sequence (e.g., a second RNA or DNA sequence) without the nucleic acid target binding sequence (e.g., the RNA or DNA target binding sequence). All components of a binding interaction do not need to be sequence-specific, such as contacts of a protein with phosphate residues in a DNA backbone. Binding interactions can be characterized by a dissociation constant (Kd). “Binding affinity” refers to the strength of the binding interaction. An increased binding affinity is correlated with a lower Kd.

As used herein, a Cas protein (e.g., a CasM protein) is said to “target” a polynucleotide if a cognate nucleic acid/Cas protein nucleoprotein complex associates with, binds and/or cleaves a polynucleotide at the nucleic acid target sequence within the polynucleotide.

As used herein, “single-strand break” (SSB) refers to cleavage of a single strand of RNA or DNA. A “double-strand break” (DSB) refers to both strands of a double-stranded segment of nucleic acid being severed. In some instances, if such a break occurs, one strand can be said to have a “sticky end” wherein nucleotides are exposed and not hydrogen bonded to nucleotides on the other strand. In other instances, a “blunt end” can occur wherein both strands remain fully base paired with each other.

As used herein, the term “recombination” refers to a process of exchange of genetic information between two polynucleotides.

As used herein, “nucleic acid repair,” such as but not limited to DNA repair, encompasses any process whereby cellular machinery repairs damage to a nucleic acid molecule contained in the cell. The damage repaired can include single-strand breaks or double-strand breaks (DSBs). At least three mechanisms exist to repair DSBs: homology-directed repair (HDR), classical non-homologous end joining (c-NHEJ), and microhomology-mediated end joining (MMEJ), all defined below. “Nucleic acid repair” is also used herein to refer to nucleic acid repair resulting from human manipulation, wherein a target locus is modified, e.g., by inserting, deleting, or substituting nucleotides, all of which represent forms of genome editing.

As used herein, the term “homology-directed repair” or “HDR” refers to nucleic acid repair that takes place in cells, for example, during repair of double-strand and single-strand breaks in a nucleic acid molecule, such as DNA. HDR requires nucleotide sequence homology and uses a “donor template” (donor template nucleic acid, such as DNA, polynucleotide donor, or oligonucleotide (used interchangably herein) to repair the sequence where the double-strand break occurred (e.g., DNA target sequence). This results in the transfer of genetic information from, for example, the donor template DNA to the DNA target sequence. HDR may result in alteration of the nucleic acid target sequence (e.g., insertion, deletion, mutation) if the donor template sequence or oligonucleotide sequence differs from the target sequence and part or all of the donor template polynucleotide or oligonucleotide is incorporated into the target sequence. In some embodiments, an entire donor template polynucleotide, a portion of the donor template polynucleotide, or a copy of the donor polynucleotide is copied or integrated at the site of the target sequence.

By “donor polynucleotide” is meant a polynucleotide that can be directed to, and inserted into a target site of interest, such as an integration locus, to modify the target nucleic acid. All or a portion of the donor polynucleotide can be inserted into the target nucleic acid. The donor polynucleotide can be used for repair of the break in the target nucleic acid sequence resulting in the transfer of genetic information (i.e., polynucleotide sequences) from the donor at the site or in close proximity of the break. Accordingly, new genetic information (i.e., polynucleotide sequences) may be inserted or copied at a target site. The donor polynucleotide can be double- or single-stranded RNA, DNA, a vector, plasmid, or the like. Thus, a donor polynucleotide can be an insertion cassette, a recombinase expression vector, and the like. Non-symmetrical polynucleotide donors can also be used that are composed of two oligonucleotides. They are partially complementary, and each can include a flanking region of homology. The donor can be used to insert or replace polynucleotide sequences in a target sequence, for example, to introduce a polynucleotide that encodes a protein or functional RNA (e.g., siRNA), to introduce a protein tag, to modify a regulatory sequence of a gene, or to introduce a regulatory sequence to a gene (e.g. a promoter, an enhancer, an internal ribosome entry sequence, a start codon, a stop codon, a localization signal, or polyadenylation signal), to modify a nucleic acid sequence (e.g., introduce a mutation), and the like.

Targeted nucleic acid modifications using donor polynucleotides for large changes (e.g., more than 100 base pair (bp) insertions or deletions) traditionally use plasmid-based donor templates that contain homology arms flanking the site of alteration. Each arm can vary in length, but is typically longer than about 100 bp, such as 100-1500 bp, e.g., 100 . . . 200 . . . 300 . . . 400 . . . 500 . . . 600 . . . 700 . . . 800 . . . 900 . . . 1000 . . . 1500 bp or any integer between these values. However, these numbers can vary, depending on the size of the donor polynucleotide and the target polynucleotide. This method can be used to generate large modifications, including insertion of reporter genes such as fluorescent proteins or antibiotic resistance markers. For transfection in cells, such as HEK cells, approximately 100-1000 nanograms (ng), e.g., 100 . . . 200 . . . 300 . . . 400 . . . 500 . . . 600 . . . 700 . . . 800 . . . 900 . . . 1000 ng or any integer between these values, of a typical size donor plasmid (e.g., approximately 5 kb) containing a NATNA/Cas vector, can be used for one well in 24-well plate. (See, e.g., Yang et al., “One Step Generation of Mice Carrying Reporter and Conditional Alleles by CRISPR/Cas-Mediated Genome Engineering” Cell (2013) 154:1370-1379).

Single-stranded and partially double-stranded oligonucleotides, such as DNA oligonucleotides, have been used in place of targeting plasmids for short modifications (e.g., less than 50 bp) within a defined locus without cloning. To achieve high HDR efficiencies, single-stranded oligonucleotides containing flanking sequences on each side that are homologous to the target region can be used, and can be oriented in either the sense or antisense direction relative to the target locus. The length of each arm can vary, but the length of at least one arm is typically longer than about 10 bases, such as from 10-150 bases, e.g., . . . 20 . . . 30 . . . 40 . . . 50 . . . 60 . . . 70 . . . 80 . . . 90 . . . 100 . . . 110 . . . 120 . . . 130 . . . 140 . . . 150, or any integer within these ranges. However, these numbers can vary, depending on the size of the donor polynucleotide and the target polynucleotide. In some embodiments, the length of at least one arm is 10 bases or more. In other embodiments, the length of at least one arm is 20 bases or more. In yet other embodiments, the length of at least one arm is 30 bases or more. In some embodiments, the length of at least one arm is less than 100 bases. In further embodiments, the length of at least one arm is greater than 100 bases. In some embodiments, the length of at least one arm is zero bases. For single-stranded oligonucleotide design, typically an oligonucleotide with around 100-150 bp total homology is used. The mutation is introduced in the middle, giving 50-75 bp homology arms for a donor designed to be symmetrical about the target site. In other cases, no homology arms are required, and the donor polynucleotide is inserted using non-homologous repair mechanisms.

A “genomic region” is a segment of a chromosome in the genome of a host cell that is present on either side of the nucleic acid target sequence site or, alternatively, also includes a portion of the nucleic acid target sequence site. The homology arms of the donor polynucleotide have sufficient homology to undergo homologous recombination with the corresponding genomic regions. In some embodiments, the homology arms of the donor polynucleotide share significant sequence homology to the genomic region immediately flanking the nucleic acid target sequence site; it is recognized that the homology arms can be designed to have sufficient homology to genomic regions farther from the nucleic acid target sequence site.

As used herein the terms “classical non-homologous end joining” or “c-NHEJ” refer to the repair of double-strand breaks in DNA by direct ligation of one end of the break to the other end of the break without a requirement for a donor template DNA. NHEJ in the absence of a donor template DNA often results in small insertions or deletions of nucleotides at the site of the double-strand break, also referred to as “indels.” This DNA repair pathway is genetically defined and requires the activity of Ligase IV, DNA-PKcs, Polμ, Pola, and the Ku70/80 heterodimer, among other proteins (see, e.g., Sfeir and Symington, Trends Biochem Sci (2015) 40:701-714).

“Microhomology-mediated end joining (MMEJ),” a form of alternative nonhomologous end-joining (alt-NHEJ), is another pathway for repairing double-strand breaks in DNA. MMEJ is associated with deletions flanking a DSB and involves alignment of microhomologous sequences internal to the broken ends before joining. The proposed mechanism entails 5′-3′ resection of the DNA ends at a DSB, annealing of the microhomologies (1-16 nucleotides of homology), removal of heterologous flaps, gap filling DNA synthesis, and ligation. MMEJ is genetically defined and requires the activity of CtIP, PARP1, Polθ, Lig1 and Lig3, among other proteins (see, e.g., Sfeir and Symington, “Microhomology-Mediated End Joining: A Back-up Survival Mechanism or Dedicated Pathway?” Trends Biochem Sci (2015) 40:701-714).

Alternative mechanisms of nucleic acid insertion that do not require sequence homology between the donor and the target sequence can also be used for nucleic acid insertion. These mechanisms involve various components of the cellular repair machinery and it is to be understood that the scope of the invention is not bound by the use of any particular mechanism for insertion of nucleic acid after target nucleic acid is cut or nicked by a site-specific polynucleotide.

“Gene,” as used herein, refers to a polynucleotide sequence comprising exon(s) and related regulatory sequences. A gene may further comprise intron(s) and/or untranslated region(s) (UTR(s)).

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product(s).” Expression may include splicing the mRNA in a eukaryotic cell, if the polynucleotide is derived from genomic DNA.

As used herein, the term “modulate” refers to a change in the quantity, degree or amount of a function. For example, a cognate nucleic acid guide/CasM protein complex, as disclosed herein, may modulate the activity of a promoter sequence by binding to a nucleic acid target sequence at or near the promoter. Depending on the action occurring after binding, the cognate nucleic acid guide/CasM protein complex can induce, enhance, suppress, or inhibit transcription of a gene operatively linked to the promoter sequence. Thus, “modulation” of gene expression includes both gene activation and gene repression.

Modulation can be assayed by determining any characteristic directly or indirectly affected by the expression of the target gene. Such characteristics include, e.g., changes in RNA or protein levels, protein activity, product levels, expression of the gene, or activity level of reporter genes. Accordingly, the terms “modulating expression,” “inhibiting expression,” and “activating expression” of a gene can refer to the ability of a cognate guide/CasM protein complex to change, activate, or inhibit transcription of a gene.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes. An expression vector typically comprises an expression cassette.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used herein, a “targeting vector” is a recombinant DNA or RNA construct typically comprising tailored DNA or RNA arms, homologous to genomic DNA or RNA derived therefrom, that flank elements of a target gene or nucleic acid target sequence (e.g., a SSB or DSB). A targeting vector comprises a donor polynucleotide. Elements of the target sequence can be modified in a number of ways including deletions and/or insertions. A defective target gene can be replaced by a functional target gene, or in the alternative a functional gene can be knocked out. Optionally, the donor polynucleotide of a targeting vector comprises a selection cassette comprising a selectable marker that is introduced into the target gene. Targeting regions (i.e., nucleic acid target sequences) adjacent or within a target gene or region can be used to affect regulation of gene expression.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like.

Regulatory elements include those that direct constitutive, inducible, and repressible expression of a nucleotide sequence in many types of host cells and those that direct expression of the nucleotide sequence only in certain host cells (e.g., tissue-specific regulatory sequences). In some embodiments, a vector comprises one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart, M., et al., Cell 41:521-530 (1985)), the SV40 promoter, the dihydrofolate reductase promoter, the β-actin promoter, the phosphoglycerol kinase (PGK) promoter, and the EF1α promoter. It will be appreciated by those skilled in the art that the design of an expression vector may depend on such factors as the choice of the host cell to be transformed, the level of expression desired, and the like. A vector can be introduced into host cells to thereby produce transcripts, proteins, or peptides, including fusion proteins or peptides, encoded by nucleic acids as described herein.

As used herein the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Accordingly, some polynucleotide elements may be operably linked but not contiguous.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

As used herein, the term “sequence identity” generally refers to the percent identity of bases or amino acids determined by comparing a first polynucleotide or polypeptide to a second polynucleotide or polypeptide using algorithms having various weighting parameters. Sequence identity between two polypeptides or two polynucleotides can be determined using sequence alignment by various methods and computer programs (e.g., BLAST, CS-BLAST, FASTA, HMMER, L-ALIGN, etc.), available through the worldwide web at sites including GENBANK (ncbi.nlm.nih.gov/genbank/) and EMBL-EBI (ebi.ac.uk.). Sequence identity between two polynucleotides or two polypeptide sequences is generally calculated using the standard default parameters of the various methods or computer programs. Generally, Cas proteins, such as CasM homologs, for use herein will have at least about 75% or more sequence identity to the wild-type or naturally occurring sequence of the Cas protein of interest, such as about 80%, such as about 85%, 90%, 91%, 92%, 93%, 94%, 95%, 96%, 97%, 98%, 99%, or complete identity. CasM orthologs can vary widely from the reference sequence. For example, CasM orthologs shown in Table 1 display approximately 13.59% to 99.82% sequence identity to each other. Thus, CasM orthologs are identified based on the CasM characteristics detailed herein.

As used herein, “hybridization,” “hybridize,” or “hybridizing” is the process of combining two complementary single-stranded DNA or RNA molecules so as to form a single double-stranded molecule (DNA/DNA, DNA/RNA, RNA/RNA) through hydrogen base pairing. Hybridization stringency is typically determined by the hybridization temperature and the salt concentration of the hybridization buffer; e.g., high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tₘ; moderate stringency, approximately 0.16M to approximately 0.33M salt, hybridization temperature 20° C. to 29° C. below Tₘ; and low stringency, approximately 0.33M to approximately 0.82M salt, hybridization temperature 40° C. to 48° C. below Tₘ. Tₘ of duplex nucleic acids is calculated by standard methods well-known in the art (see, e.g., Maniatis, T., et al., Molecular Cloning: A Laboratory Manual, Cold Spring Harbor Laboratory Press: New York (1982); Casey, J., et al., Nucleic Acids Research 4:1539-1552 (1977); Bodkin, D. K., et al., Journal of Virological Methods 10 (1): 45-52 (1985); Wallace, R. B., et al., Nucleic Acids Research 9 (4): 879-894 (1981)). Algorithm prediction tools to estimate Tₘ are also widely available. High stringency conditions for hybridization typically refer to conditions under which a polynucleotide complementary to a target sequence predominantly hybridizes with the target sequence, and substantially does not hybridize to non-target sequences. Typically, hybridization conditions are of moderate stringency, preferably high stringency.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to an amino acid polymer that has been modified through, for example, acetylation, disulfide bond formation, glycosylation, lipidation, phosphorylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology (see, e.g., standard texts set forth above). Further, essentially any polypeptide or polynucleotide can be custom ordered from commercial sources.

The terms “fusion protein” and “chimeric protein,” as used herein, refer to a single protein created by joining two or more proteins, protein domains, or protein fragments that do not naturally occur together in a single protein. For example, a fusion protein can contain a first domain from a CasM protein and a second domain from a different Cas protein. The modification to include such domains in fusion proteins may confer additional activity on the modified site-directed polypeptides. Such activities can include nuclease activity, methyltransferase activity, demethylase activity, DNA or RNA repair activity, DNA or RNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity) that modifies a polypeptide associated with nucleic acid target sequence (e.g., a histone). A fusion protein can also comprise epitope tags (e.g., histidine tags, FLAG® (Sigma Aldrich, St. Louis, MO) tags, Myc tags), reporter protein sequences (e.g., glutathione-S-transferase, beta-galactosidase, luciferase, green fluorescent protein, cyan fluorescent protein, yellow fluorescent protein), and/or nucleic acid binding domains (e.g., a DNA binding domain, an RNA binding domain). A fusion protein can also comprise activator domains (e.g., heat shock transcription factors, NFKB activators) or repressor domains (e.g., a KRAB domain). As described by Lupo, A., et al., Current Genomics 14 (4): 268-278 (2013), the KRAB domain is a potent transcriptional repression module and is located in the amino-terminal sequence of most C2H2 zinc finger proteins (see, e.g., Margolin, J., et al., Proceedings of the National Academy of Sciences of the United States of America 91:4509-4513 (1994); Witzgall, R., et al., Proceedings of the National Academy of Sciences of the United States of America 91:4514-4518 (1994)). The KRAB domain typically binds to co-repressor proteins and/or transcription factors via protein-protein interactions, causing transcriptional repression of genes to which KRAB zinc finger proteins (KRAB-ZFPs) bind (see, e.g., Friedman J. R., et al., Genes & Development 10:2067-2678 (1996)). In some embodiments, linker nucleic acid sequences are used to join the two or more proteins, protein domains, or protein fragments.

A “moiety,” as used herein, refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description. In some embodiments, a 5′ terminus, a 3′ terminus, or a 5′ terminus and a 3′ terminus (e.g., a non-native 5′ terminus and/or a non-native 3′ terminus in a first stem element) can comprise one or more moieties.

As used herein, the term “isolated” can refer to a nucleic acid or polypeptide that, by the hand of a human, exists apart from its native environment and is therefore not a product of nature. Isolated means substantially pure. An isolated nucleic acid or polypeptide can exist in a purified form and/or can exist in a non-native environment such as, for example, in a recombinant cell.

As used herein, a “host cell” generally refers to a biological cell. A cell is the basic structural, functional and/or biological unit of an organism. A cell can originate from any organism having one or more cells. Examples of host cells include, but are not limited to: a prokaryotic cell, eukaryotic cell, a bacterial cell, an archaeal cell, a cell of a single-cell eukaryotic organism, a protozoal cell, a cell from a plant (e.g., cells from plant crops (such as soy, tomatoes, sugar beets, pumpkin, hay, cannabis, tobacco, plantains, yams, sweet potatoes, cassava, potatoes, wheat, sorghum, soybean, rice, corn, maize, oil-producing Brassica (e.g., oil-producing rapeseed and canola), cotton, sugar cane, sunflower, millet, and alfalfa), fruits, vegetables, grains, seeds, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. agardh, and the like), seaweeds (e.g., kelp), a fungal cell (e.g., a yeast cell or a cell from a mushroom), an animal cell, a cell from an invertebrate animal (e.g., fruit fly, cnidarian, echinoderm, nematode, and the like), a cell from a vertebrate animal (e.g., fish, amphibian, reptile, bird, or mammal), a cell from a mammal (e.g., a pig, a cow, a goat, a sheep, a rodent, a rat, a mouse, a non-human primate, a human, and the like). Furthermore, a cell can be a stem cell or a progenitor cell.

As used herein, “stem cell” refers to a cell that has the capacity for self-renewal, i.e., the ability to go through numerous cycles of cell division while maintaining the undifferentiated state. Stem cells can be totipotent, pluripotent, multipotent, oligopotent, or unipotent. Stem cells can be embryonic, fetal, amniotic, adult, or induced pluripotent stem cells.

As used herein, “induced pluripotent stem cells” refers to a type of pluripotent stem cell that is artificially derived from a non-pluripotent cell, typically an adult somatic cell, by inducing expression of specific genes.

“Plant,” as used herein, refers to whole plants, plant organs, plant tissues, germplasm, seeds, plant cells, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to roots, stems, shoots, leaves, pollens, seeds, tumor tissue, and various forms of cells and culture (e.g., single cells, protoplasts, embryos, and callus tissue). The plant tissue may be in plant or in a plant organ, tissue or cell culture. “Plant organ” refers to plant tissue or a group of tissues that constitute a morphologically and functionally distinct part of a plant.

“Subject,” as used herein, refers to any member of the phylum Chordata, including, without limitation, humans and other primates, including non-human primates such as rhesus macaques, chimpanzees and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as male and female. In some embodiments, a host cell is derived from a subject (e.g., stem cells, progenitor cells, or tissue-specific cells). In some embodiments, the subject is a non-human subject.

As used herein, “transgenic organism” refers to an organism whose genome is genetically modified. The term includes the progeny (any generation) of a transgenic organism, provided that the progeny has the genetic modification.

### CRISPR Systems

The CRISPR (Clustered Regularly Interspaced Short Palindromic Repeats) genomic locus is found in the genomes of many prokaryotes (e.g., bacteria and archaea). CRISPR loci provide resistance to foreign invaders (e.g., virus, phage) in prokaryotes. In this way, the CRISPR system functions as a type of immune system to help defend prokaryotes against foreign invaders. There are three main stages in CRISPR-Cas immune systems: (1) acquisition, (2) expression, and (3) interference. Acquisition involves cleaving the genome of invading viruses and plasmids and integrating segments (termed protospacers) of the genomic DNA into the CRISPR locus of the host organism. The segments that are integrated into the host genome are known as spacers, which mediate protection from subsequent attack by the same (or sufficiently related) virus or plasmid. Expression involves transcription of the CRISPR locus and subsequent enzymatic processing to produce short mature CRISPR RNAs, each containing a single spacer sequence. Interference is induced after the CRISPR RNAs associate with Cas proteins to form effector complexes, which are then targeted to complementary protospacers in foreign genetic elements to induce nucleic acid degradation.

Currently, two classes of CRISPR systems have been described, Class 1 and Class 2, based upon the genes encoding the effector module, i.e., the proteins involved in the interference stage. Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III and Type IV systems. Class 2 systems comprise Type II, Type V and Type VI systems.

To date, there are six types (Types I-VI) and 19 subtypes of CRISPR systems categorized within these classes (Makarova et al., Nature Reviews Microbiology (2015) 13:1-15; Shmakov et al., Nature Reviews Microbiology (2017) 15:169-182).

CRISPR loci are currently characterized as including a number of short repeating sequences referred to as “repeats.” Repeats can form hairpin structures and/or repeats can be unstructured single-stranded sequences. The repeats occur in clusters. Repeats frequently diverge between species. Repeats are regularly interspaced with unique intervening sequences, referred to as “spacers,” resulting in a repeat-spacer-repeat locus architecture. Spacers are identical to or are homologous with known foreign invader sequences. In some instances, a spacer-repeat unit encodes a crisprRNA (crRNA). A crRNA refers to the mature form of the spacer-repeat unit. A crRNA contains a spacer sequence that is involved in targeting a target nucleic acid (e.g., possibly as a surveillance mechanism against foreign nucleic acid). Thus, crRNA has a region of complementarity to a potential DNA or RNA target sequence and in some cases, e.g., in currently characterized Type II systems, a second region that forms base-pair hydrogen bonds with a transactivating CRISPR RNA (tracrRNA) to form a secondary structure, typically to form at least a stem structure. In this context, the tracrRNA and a crRNA interact through a number of base-pair hydrogen bonds to form secondary RNA structures. Complex formation between tracrRNA/crRNA and a Cas protein results in conformational change of the Cas protein that facilitates binding to DNA, nuclease activities of the Cas protein, and crRNA-guided site-specific DNA cleavage by the nuclease. For a Cas protein/tracrRNA/crRNA complex to cleave a DNA target sequence, the DNA target sequence is adjacent to a cognate protospacer adjacent motif (PAM).

A CRISPR locus comprises polynucleotide sequences encoding for CRISPR Associated Genes (cas) genes. Cas genes are involved in the biogenesis and/or the interference stages of crRNA function. Cas genes display extreme sequence (e.g., primary sequence) divergence between species and homologs. Some Cas genes comprise homologous secondary and/or tertiary structures. Cas genes are typically named according to the organism from which they are derived. For example, Cas genes in Staphylococcus epidermidis can be referred to as Csm-type, Cas genes in Streptococcus thermophilus can be referred to as Csn-type, and Cas genes in Pyrococcus furiosus can be referred to as Cmr-type.

The integration stage of a CRISPR system refers to the ability of the CRISPR locus to integrate new spacers into the crRNA array upon being infected by a foreign invader. Acquisition of the foreign invader spacers can help confer immunity to subsequent attacks by the same foreign invader. Integration typically occurs at the leader end of the CRISPR locus. Cas proteins are involved in integration of new spacer sequences. Integration proceeds similarly for some types of CRISPR systems (e.g., Types I-III).

Mature crRNAs are processed from a longer polycistronic CRISPR locus transcript (i.e., pre-crRNA array). A pre-crRNA array comprises a plurality of crRNAs. The repeats in the pre-crRNA array are recognized by cas genes. Cas genes bind to the repeats and cleave the repeats. This action can liberate the plurality of crRNAs. crRNAs can be subjected to further events to produce the mature crRNA form such as trimming (e.g., with an exonuclease). A crRNA may comprise all, some, or none of the CRISPR repeat sequence.

Interference refers to the stage in the CRISPR system that is functionally responsible for combating infection by a foreign invader. CRISPR interference follows a similar mechanism to RNA interference (RNAi: e.g., wherein a target RNA is targeted (e.g., hybridized) by a short interfering RNA (siRNA)), which results in target RNA degradation and/or destabilization. Currently characterized CRISPR systems perform interference of a target nucleic acid by coupling crRNAs and Cas genes, thereby forming CRISPR ribonucleoproteins (RNPs). crRNA of the RNP guides the RNP to foreign invader nucleic acid, (e.g., by recognizing the foreign invader nucleic acid through hybridization). Hybridized target foreign invader nucleic acid-crRNA units are subjected to cleavage by Cas proteins. Target nucleic acid interference typically requires a protospacer adjacent motif (PAM) in a target nucleic acid.

By a “CRISPR-Cas system” as used herein, is meant any of the various CRISPR-Cas classes, types, and subtypes. Class 1 systems comprise Type I, Type III, and Type IV systems. Type I systems are currently characterized as having a Cas3 protein that has helicase activity and cleavage activity. Type I systems are further divided into several subtypes that have a defined combination of signature genes and distinct features of operon organization.

To date, it appears that all Type III systems possess a cas10 gene, which encodes a multidomain protein containing a Palm domain (a variant of the RNA recognition motif (RRM)) that is homologous to the core domain of numerous nucleic acid polymerases and cyclases and that is the largest subunit of Type III crRNA-effector complexes. All Type III loci also encode the small subunit protein, one Cas5 protein and typically several Cas7 proteins. Type III is also further divided into several subtypes.

Type IV systems encode a minimal multisubunit crRNA-effector complex comprising a partially degraded large subunit, Csf1, Cas5, Cas7, and in some cases, a putative small subunit. Type IV systems lack cas1 and cas2 genes. Type IV systems do not have subtypes, but there are two distinct variants. One Type IV variant has a DinG family helicase, whereas a second Type IV variant lacks a DinG family helicase, but has a gene encoding a small α-helical protein. An example of an organism with a Type IV system is Acidithiobacillus ferrooxidans.

Class 2 systems comprise Type II, Type V, and Type VI systems. Type II systems include cas1, cas2 and cas9 genes. There are two strands of RNA in Type II systems, a crRNA and a tracrRNA, that hybridizes to a complementary region of pre-crRNA causing maturation of the pre-crRNA to crRNA. The duplex formed by the tracrRNA and crRNA is recognized by, and associates with a multidomain protein, Cas9, encoded by the cas9 gene, which combines the functions of the crRNA-effector complex with target DNA cleavage. Cas9 is directed to a target nucleic acid by a sequence of the crRNA that is complementary to, and hybridizes with, a sequence in the target nucleic acid.

In Type V systems, nucleic acid target sequence binding involves a Cas12a protein and the crRNA, as does the nucleic acid target sequence cleavage. In Type V systems, the RuvC-like nuclease domain of Cas12a protein cleaves both strands of the nucleic acid target sequence in a sequential fashion (Swarts, et al., Mol. Cell (2017) 66:221-233.e4), producing 5′ overhangs, which contrasts with the blunt ends generated by Cas9 protein cleavage.

The Cas12a protein cleavage activity of Type V systems does not require hybridization of crRNA to tracrRNA to form a duplex; rather Type V systems use a single crRNA that has a stem-loop structure forming an internal duplex. Cas12a protein binds the crRNA in a sequence- and structure-specific manner by recognizing the stem loop and sequences adjacent to the stem loop, most notably the nucleotides 5′ of the spacer sequence, which hybridizes to the nucleic acid target sequence. This stem-loop structure is typically in the range of 15 to 19 nucleotides in length. Substitutions that disrupt this stem-loop duplex abolish cleavage activity, whereas other substitutions that do not disrupt the stem-loop duplex do not abolish cleavage activity.

Type VI systems include the Cas13a protein (also known as Class 2 candidate 2 protein, or C2c2) which does not share sequence similarity with other CRISPR effector proteins (see Abudayyeh, et al., Science (2016) 353: aaf5573). Cas13a proteins have two HEPN domains and possess single-stranded RNA cleavage activity. Cas13a proteins are similar to Cas12a proteins in requiring a crRNA for nucleic acid target sequence binding and cleavage, but not requiring tracrRNA. Also, similar to Cas12a protein, the crRNA for Cas13a proteins forms a stable hairpin, or stem-loop structure, that aids in association with the Cas13a protein. Type VI systems have a single polypeptide RNA endonuclease that utilizes a single crRNA to direct RNA cleavage in a target-dependent fashion. Additionally, after hybridizing to the target RNA complementary to the spacer, Cas13a protein becomes a promiscuous RNA endonuclease exhibiting non-specific endonuclease activity toward any single-stranded RNA in a sequence independent manner (see East-Seletsky, et al., Nature (2016) 538:270-273).

As is readily apparent, the discovery and characterization of CRISPR systems is currently evolving.

### Production of CRISPR Components

In all of the embodiments described herein, the various components can be produced by synthesis, or for example, using expression cassettes encoding CasM, a cognate guide, etc. The various components can be provided to a cell or used in vitro. These components can be present on a single cassette or multiple cassettes, in the same or different constructs. Expression cassettes typically comprise regulatory sequences functional in host cells into which they are introduced. Regulatory sequences are involved in one or more of the following: regulation of transcription, post-transcriptional regulation, and regulation of translation. Expression cassettes can be present in expression vectors and introduced into a wide variety of host cells including bacterial cells, yeast cells, plant cells, and mammalian cells.

In one aspect, all or a portion of the various components for use herein are produced in vectors, including expression vectors, comprising polynucleotides encoding therefor. Vectors useful for producing components for use in the present methods include plasmids, viruses (including phage), and integratable nucleic acid fragments (i.e., fragments integratable into the host genome by homologous recombination). A vector replicates and functions independently of the host genome, or may, in some instances, integrate into the genome itself. Suitable replicating vectors will contain a replicon and control sequences derived from species compatible with the intended expression host cell. In some embodiments, polynucleotides encoding one or more of the various components are operably linked to an inducible promoter, a repressible promoter, or a constitutive promoter. Expression vectors can also include polynucleotides encoding protein tags (e.g., poly-His tags, hemagglutinin tags, fluorescent protein tags, bioluminescent tags, nuclear localization tags). The coding sequences for such protein tags can be fused to the coding sequences or can be included in an expression cassette, for example, in a targeting vector.

General methods for construction of expression vectors are known in the art. Expression vectors for most host cells are commercially available. There are several commercial software products designed to facilitate selection of appropriate vectors and construction thereof, such as insect cell vectors for insect cell transformation and gene expression in insect cells, bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation and gene expression and methods to easily enable cloning of such polynucleotides. SnapGene™ (GSL Biotech LLC, Chicago, Ill.; snapgene.com/resources/plasmid_files/your_time_is_valuable/), for example, provides an extensive list of vectors, individual vector sequences, and vector maps, as well as commercial sources for many of the vectors.

Several expression vectors have been designed for expressing guide polynucleotides. See, e.g., Shen et al. Nat. Methods (2014) 11:399-402. Additionally, vectors and expression systems are commercially available, such as from New England Biolabs (Ipswich, MA) and Clontech Laboratories (Mountain View, CA). Vectors can be designed to simultaneously express a target-specific NATNA using a U2 or U6 promoter, a CasM and/or dCasM, and if desired, a marker protein, for monitoring transfection efficiency and/or for further enriching/isolating transfected cells by flow cytometry.

For example, the various components can be incorporated into mammalian vectors for use in mammalian cells. A large number of mammalian vectors suitable for use with the systems of the present invention are commercially available (e.g., from Life Technologies, Grand Island, NY; NeoBiolab, Cambridge, MA; Promega, Madison, WI; DNA2.0, Menlo Park, CA; Addgene, Cambridge, MA).

Vectors derived from mammalian viruses can also be used for expressing the various components of the present methods in mammalian cells. These include vectors derived from viruses such as adenovirus, papovirus, herpesvirus, polyomavirus, cytomegalovirus, lentivirus, retrovirus, vaccinia and Simian Virus 40 (SV40) (see, e.g., Kaufman, R. J., Molec. Biotech. (2000) 16:151-160; Cooray et al., Methods Enzymol. (2012) 507:29-57). Regulatory sequences operably linked to the components can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Commonly used promoters are constitutive mammalian promoters CMV, EF1a, SV40, PGK1 (mouse or human), Ubc, CAG, CaMKIIa, and beta-Act, and others known in the art (Khan, K. H. Advanced Pharmaceutical Bulletin (2013) 3:257-263). Furthermore, mammalian RNA polymerase III promoters, including H1 and U6, can be used.

Numerous mammalian cell lines have been utilized for expression of gene products including HEK 293 (Human embryonic kidney) and CHO (Chinese hamster ovary). These cell lines can be transfected by standard methods (e.g., using calcium phosphate or polyethyleneimine (PEI), or electroporation). Other typical mammalian cell lines include, but are not limited to: HeLa, U2OS, 549, HT1080, CAD, P19, NIH 3T3, L929, N2a, Human embryonic kidney 293 cells, MCF-7, Y79, SO—Rb50, Hep G2, DUKX-X11, J558L, and Baby hamster kidney (BHK) cells.

Vectors can be introduced into and propagated in a prokaryote. Prokaryotic vectors are well known in the art. Typically a prokaryotic vector comprises an origin of replication suitable for the target host cell (e.g., oriC derived from E. coli, pUC derived from pBR322, pSC101 derived from Salmonella), 15A origin (derived from p15A) and bacterial artificial chromosomes). Vectors can include a selectable marker (e.g., genes encoding resistance for ampicillin, chloramphenicol, gentamicin, and kanamycin). Zeocin™ (Life Technologies, Grand Island, NY) can be used as a selection in bacteria, fungi (including yeast), plants and mammalian cell lines. Accordingly, vectors can be designed that carry only one drug resistance gene for Zeocin for selection work in a number of organisms. Useful promoters are known for expression of proteins in prokaryotes, for example, T5, T7, Rhamnose (inducible), Arabinose (inducible), and PhoA (inducible). Furthermore, T7 promoters are widely used in vectors that also encode the T7 RNA polymerase. Prokaryotic vectors can also include ribosome binding sites of varying strength, and secretion signals (e.g., mal, see, tat, ompC, and pelB). In addition, vectors can comprise RNA polymerase promoters for the expression of NATNAs. Prokaryotic RNA polymerase transcription termination sequences are also well known (e.g., transcription termination sequences from Streptococcus pyogenes).

Expression of proteins in prokaryotes is typically carried out in E. coli with vectors containing constitutive or inducible promoters directing the expression of either fusion or non-fusion proteins.

In some embodiments, a vector is a yeast expression vector comprising one or more components of the above-described methods. Examples of vectors for expression in Saccharomyces cerivisae include, but are not limited to, the following: pYepSec1, pMFa, pJRY88, pYES2, and picZ. Methods for gene expression in yeast cells are known in the art (see, e.g., Methods in Enzymology, Volume 194, “Guide to Yeast Genetics and Molecular and Cell Biology, Part A,” (2004) Christine Guthrie and Gerald R. Fink (eds.), Elsevier Academic Press, San Diego, CA). Typically, expression of protein-encoding genes in yeast requires a promoter operably linked to a coding region of interest plus a transcriptional terminator. Various yeast promoters can be used to construct expression cassettes for expression of genes in yeast.

### CasM Proteins

CasM, a new CRISPR-Cas protein, is described herein. CasM displays nucleic acid binding activity and produces breaks, such as singe-strand breaks (SSBs) or DSBs, when brought into proximity with a nucleic acid target sequence, e.g., by association with a cognate nucleic acid guide, such as a cognate crRNA. As shown in the Examples herein, CasM targets RNA and is capable of cleaving ssRNA, such as when delivered to a genomic target when complexed with a crRNA, without the necessity of association with a tracrRNA. CasM proteins typically include two or more higher eukaryotic and prokaryotic nucleotide-binding (HEPN) domains found in protein family PF05168, in the C-terminal region of the CasM sequence. CasM proteins show synteny with one or more WYL domain-containing proteins and sometimes with RtcB (RNA 3′-terminal phosphate cyclase, group B) domain-containing proteins. Based on the foregoing characteristics, CasM may be classified as a Class 2 Type VI CRISPR-Cas system. However, CasM has a very low degree of sequence similarity to other Type VI subtypes.

Exemplary CasM proteins are shown in SEQ ID NOS: 37-45, and are encoded by polynucleotides shown in SEQ ID NOS: 28-36, respectively. These proteins display approximately 13.59% to 99.82% sequence identity to each other and show less than 8% sequence identity with other known CRISPR-Cas proteins. CasM has been found in several species and isolates including, without limitation, Eubacterium siraeum; Ruminococcus sp., such as from Ruminococcus bicirculans; Ruminococcus flavefaciens, such as, but not limited to, FD-1 and strain XPD3002; Ruminococcus albus such as, but not limited to, strain KH2T6; Ruminococcus sp. isolates, such as but not limited to, isolates 2789STDY5834971, 2789STDY5608892 and 2789STDY5834894. However, is it to be understood that the term “CasM” refers to a protein derived from any species, subspecies or strain of bacteria that encodes a CasM protein, as well as orthologs thereof, or variants thereof. Representative CasM proteins include, but are not limited to, those proteins depicted as SEQ ID NOS: 37-45 (see Table 1), orthologs thereof, or variants thereof. CasM proteins are approximately 800 to approximately 1000 amino acids in length.

TABLE 1
Representative CasM Proteins

| Species/Isolate | SEQ ID NO |
|---|---|
| Eubacterium siraeum | SEQ ID NO: 37 |
| Ruminococcus sp., isolate 2789STDY5834971 | SEQ ID NO: 38 |
| Ruminococcus bicirculans | SEQ ID NO: 39 |
| Ruminocccus sp., isolate 2789STDY5608892 | SEQ ID NO: 40 |
| Ruminococcus sp. CAG:57 | SEQ ID NO: 41 |
| Ruminococcus flavefaciens FD-1 | SEQ ID NO: 42 |
| Ruminococcus albus strain KH2T6 | SEQ ID NO: 43 |
| Ruminococcus flavefaciens strain XPD3002 | SEQ ID NO: 44 |
| Ruminococcus sp., isolate 2789STDY5834894 | SEQ ID NO: 45 |

Analysis of these CasM protein sequences indicates the presence of two HEPN domains in the C-terminal region of the sequences. The HEPN domain is often involved in nucleic acid binding and can function as a metal-independent RNase in certain instances.

CasM systems display strong synteny with an open reading frame in WYL domain-(protein family PF13280) containing proteins. The sequences for WYL domains in various species that encode CasM proteins are shown as SEQ ID NOS: 52-59 (see Table 2). WYL domains share similarities with CRISPR-associated Rossman fold (CARF) domains and are thought to bind ligands derived from host-virus conflict and regulate CRISPR-Cas systems. A WYL domain protein (sll7009) has been shown to be a negative regulator of the I-D CRISPR-Cas system in Synechocystis sp. (Hein et al., RNA Biol. (2013) 10:852-864. In some instances, the WYL-containing protein contains at least two WYL domains. These duplications are consistent with the hypothesized multimeric assembly of these ligand-binding domains (Schumacher et al., EMBO J. (2002) 21:1210-1218). The N-termini of these WYL domains contain homology to transcriptional repressor CopG and the ParD anti-toxin domain. For use in eukaryotes, the WYL domain-containing proteins can be modified with a N- or C-terminal nuclear localization signal sequence (NLS). SEQ ID NOS: 61-68 present exemplary WYL domain-containing proteins modified with a seven amino acid C-terminal NLS tag derived from the SV40 Large T-antigen.

TABLE 2
WYL domain sequences in various CasM-containing species

| Species/Isolate | SEQ ID NO |
|---|---|
| Eubacterium siraeum | SEQ ID NO: 52 |
| Ruminococcus sp., isolate 2789STDY5834971 | SEQ ID NO: 53 |
| Ruminococcus bicirculans | SEQ ID NO: 54 |
| Ruminococcus sp., isolate 2789STDY5608892 | SEQ ID NO: 55 |
| Ruminococcus sp. CAG: 57 | SEQ ID NO: 56 |
| Ruminococcus flavefaciens FD-1 | SEQ ID NO: 57 |
| Ruminococcus albus strain KH2T6 | SEQ ID NO: 58 |
| Ruminococcus flavefaciens strain XPD3002 | SEQ ID NO: 59 |

RtcB (RNA 3′-terminal phosphate cyclase, group B) is a protein domain superfamily and a RtcB homolog (SEQ ID NO. 60) proximal to the CasM loci has been identified. It has previously been reported that the CARF domain has sequence similarity with the N-terminal domain of the RtcR protein, which acts as the regulator of the Rtc RNA repair system. The Rtc system is comprised of 3′-terminal phosphate cyclase RtcA and the RNA ligase RtcB. The RtcB domain-containing proteins can be modified with a N- or C-terminal NLS for use in eukaryotes. A RtcB domain with an associated NLS derived from the SV40 Large T-antigen is shown in SEQ ID NO:69.

A modified CasM protein can have a low degree of sequence identity, a moderate degree of sequence identity, or a high degree of sequence identity over its length to a reference CasM protein, depending on the intended function of the CasM in question. By a “high degree of sequence identity” is meant approximately 90% sequence identity to 100% sequence identity, for example, about 90% . . . 95% . . . 98% sequence identity or higher. A “moderate degree of sequence identity” is typically between about 80% sequence identity to about 85% sequence identity, for example, about 80% identity or higher, such as about 85% sequence identity. A “low degree of sequence identity” is typically between about 50% identity and 75% identity, for example, about 50% identity, preferably about 60% identity to about 75% identity.

In some embodiments, the amino acid sequence of the reference CasM protein may be modified by deletion, insertion, or substitution of one or more amino acid residues (either conservative or non-conservative in nature), such that the activity of the CasM protein is either largely retained, enhanced, or reduced. These modifications may be deliberate, as through site-directed mutagenesis, or may be accidental, such as through mutations of hosts that produce the proteins or errors due to PCR amplification.

Conservative substitutions are generally those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, an aspartate with a glutamate, a threonine with a serine, or a similar conservative replacement of an amino acid with a structurally related amino acid, will not have a major effect on the desired biological activity. For example, the CasM protein may include up to about 5-10 conservative or non-conservative amino acid substitutions, or even up to about 15-100 or more, e.g., 50 or more, conservative or non-conservative amino acid substitutions, or any number between 5-100, so long as the desired function of the molecule remains intact.

In other embodiments, it may be desirable to modify one or more catalytic domains in order to render a nuclease-deactivated CasM protein, also termed “catalytically inactive,” “catalytically dead CasM,” “dead CasM,” or “dCasM,” such that the protein either fails to produce nucleic acid breaks, and/or binds a target sequence but does not cleave it. Such molecules lack all or a portion of nuclease activity and are unable to cleave a nucleic acid of interest and can therefore be used to regulate genes in a nucleic acid-guided manner. These dCasM proteins can be used alone or in fusions to synthetically repress (CRISPRi) or activate (CRISPRa) gene expression.

The CasM proteins can either be directly isolated and purified from bacteria, or synthetically or recombinantly produced using polynucleotides encoding the same.

### CasM Polynucleotides

Nucleic acid sequences encoding representative CasM proteins are shown in SEQ ID NOS: 28-36 (see Table 3) and these polynucleotides can be used to produce CasM proteins as described herein.

TABLE 3
Representative CasM DNA Sequences

| Species/Isolate | SEQ ID NO |
|---|---|
| Eubacterium siraeum | SEQ ID NO: 28 |
| Ruminococcus sp., isolate 2789STDY5834971 | SEQ ID NO: 29 |
| Ruminococcus bicirculans | SEQ ID NO: 30 |
| Ruminococcus sp., isolate 2789STDY5608892 | SEQ ID NO: 31 |
| Ruminococcus sp. CAG: 57 | SEQ ID NO: 32 |
| Ruminococcus flavefaciens FD-1 | SEQ ID NO: 33 |
| Ruminococcus albus strain KH2T6 | SEQ ID NO: 34 |
| Ruminococcus flavefaciens strain XPD3002 | SEQ ID NO: 35 |
| Ruminococcus sp., isolate 2789STDY5834894 | SEQ ID NO: 36 |

These polynucleotides can be designed to encode native CasM proteins, such as the proteins depicted in SEQ ID NOS: 37-45 (see Table 1); homologs thereof, including orthologs found in other species; or other variants thereof. Moreover, a casM polynucleotide sequence can be modified to have a low degree of sequence identity, a moderate degree of sequence identity, or a high degree of sequence identity, over its length to a reference casM polynucleotide, depending on the intended function of the encoded CasM in question. By “a high degree of sequence identity” is meant approximately 90% sequence identity to 100% sequence identity, for example, about 90% . . . 95% . . . 98% sequence identity or higher. A “moderate degree of sequence identity” is typically between about 80% sequence identity to about 85% sequence identity, for example, about 80% identity or higher, such as about 85% sequence identity. A “low degree of sequence identity” is typically between about 50% identity and 75% identity, for example, about 50% identity, preferably about 60% identity to about 75% identity.

In some embodiments, the polynucleotide sequences are modified to enhance expression in a selected host cell. Codon usage bias refers to differences in the frequency of occurrence of synonymous codons in coding DNA. For example, for the 20 standard amino acids in the genetic code, there are 64 different codons (61 codons encoding for amino acids, and 3 stop codons). The overabundance in the number of codons allows several amino acids to be encoded by more than one codon. The genetic codes of different organisms are often biased towards the usage of one of the several codons that encode a particular amino acid. Thus, a greater frequency of one codon will be found than expected by chance in particular organisms. Accordingly, in order to enhance expression in a particular host cell, it is often desirable to manipulate polynucleotides to include codons that are biased for expression in the selected host cell. Several software packages are available online for this purpose. For example, a database from Integrated DNA Technologies, Coralville, IA (idtdna.com/CodonOpt), is a tool for producing modified sequences for expression in dozens of organisms. GeneScript, Piscataway, NJ, also provides modification tools through the OptimumGene™ algorithm (genscript.com/codon opt.html?src=google&gclid=CIX3uoqexdICFRSUfgodu3sAIQ). See also, U.S. Pat. No. 8,326,547.

Typically, polynucleotide sequences modified for expression in particular host cells will display from about 50%-99% sequence identity to the native sequences, such as 60%-95%, e.g. 65% . . . 70% . . . 75% . . . 80% . . . 85% . . . 90% . . . 95% or more sequence identity, or any integer between these ranges, to the native sequences.

Using these tools, polynucleotide sequences can be modified for expression in any commonly used host cell, such as but not limited to, bacterial cells and eukaryotic cells, including without limitation, bacterial cells such as E. coli, Lactoccocus lactis, Pseudomonas systems, Streptomyces systems, Bacillus subtilis systems, Brevibacillus systems, coryneform bacteria, and halophilic bacteria; algal cells; yeast and other fungal cells; plant cells; mammalian cells such as human cells; insect cells, and the like.

SEQ ID NOS: 1-9 show representative CasM-encoding polynucleotide sequences modified for expression in E. coli cells (see Table 4). SEQ ID NOS: 10-18 show representative CasM-encoding polynucleotide sequences modified for expression in human cells (see Table 5). SEQ ID NOS: 19-27 show CasM-encoding polynucleotide sequences modified for expression in Zea mays cells (see Table 6).

TABLE 4
Representative casM DNA Sequences
Modified for Expression in E. coli

| Species/Isolate | SEQ ID NO |
|---|---|
| Eubacterium siraeum | SEQ ID NO: 1 |
| Ruminococcus sp., isolate 2789STDY5834971 | SEQ ID NO: 2 |
| Ruminococcus bicirculans | SEQ ID NO: 3 |
| Ruminococcus sp., isolate 2789STDY5608892 | SEQ ID NO: 4 |
| Ruminococcus sp. CAG: 57 | SEQ ID NO: 5 |
| Ruminococcus flavefaciens FD-1 | SEQ ID NO: 6 |
| Ruminococcus albus strain KH2T6 | SEQ ID NO: 7 |
| Ruminococcus flavefaciens strain XPD3002 | SEQ ID NO: 8 |
| Ruminococcus sp., isolate 2789STDY5834894 | SEQ ID NO: 9 |

TABLE 5
Representative casM DNA Sequences
Modified for Expression in Human Cells

| Species/Isolate | SEQ ID NO |
|---|---|
| Eubacterium siraeum | SEQ ID NO: 10 |
| Ruminococcus sp., isolate 2789STDY5834971 | SEQ ID NO: 11 |
| Ruminococcus bicirculans | SEQ ID NO: 12 |
| Ruminococcus sp., isolate 2789STDY5608892 | SEQ ID NO: 13 |
| Ruminococcus sp. CAG: 57 | SEQ ID NO: 14 |
| Ruminococcus flavefaciens FD-1 | SEQ ID NO: 15 |
| Ruminococcus albus strain KH2T6 | SEQ ID NO: 16 |
| Ruminococcus flavefaciens strain XPD3002 | SEQ ID NO: 17 |
| Ruminococcus sp., isolate 2789STDY5834894 | SEQ ID NO: 18 |

TABLE 6
Representative casM DNA Sequences
Modified for Expression in Zea mays

| Species/Isolate | SEQ ID NO |
|---|---|
| Eubacterium siraeum | SEQ ID NO: 19 |
| Ruminococcus sp., isolate 2789STDY5834971 | SEQ ID NO: 20 |
| Ruminococcus bicirculans | SEQ ID NO: 21 |
| Ruminococcus sp., isolate 2789STDY5608892 | SEQ ID NO: 22 |
| Ruminococcus sp. CAG: 57 | SEQ ID NO: 23 |
| Ruminococcus flavefaciens FD-1 | SEQ ID NO: 24 |
| Ruminococcus albus strain KH2T6 | SEQ ID NO: 25 |
| Ruminococcus flavefaciens strain XPD3002 | SEQ ID NO: 26 |
| Ruminococcus sp., isolate 2789STDY5834894 | SEQ ID NO: 27 |

The casM polynucleotides can also be modified to include sequences encoding N- or C-terminal nuclear localization signal sequences (NLS), such as for expression in eukaryotic cells. Such sequences are known, and include, without limitation, an NLS tag derived from the SV40 Large T-antigen. Such as tag is present at the C-terminus of the proteins shown in SEQ ID NOS: 61-69 (i.e., the last seven amino acids in these sequences).

The casM polynucleotides can be used to recombinantly produce CasM proteins using methods well known in the art.

### CasM Complexes

CasM proteins can be complexed to a cognate nucleic acid guide (cognate guide/CasM complex) in order to deliver CasM in proximity with a target nucleic acid sequence. A cognate guide, such as a crRNA, is a polynucleotide that site-specifically guides a CasM nuclease, or a deactivated CasM nuclease, to a target nucleic acid region. The binding specificity is determined jointly by the complementary region on the cognate guide and a short DNA motif (protospacer adjacent motif or PAM) juxtaposed to the complementary region. The spacer present in the guide specifically hybridizes to a target nucleic acid sequence and determines the location of a Cas protein's site-specific binding and nucleolytic cleavage.

Cognate guide/CasM complexes can be produced using methods well known in the art. For example, the guide components of the complexes can be produced in vitro and CasM components can be recombinantly produced and then the guides and CasM proteins can be complexed together using methods known in the art. Additionally, cell lines constitutively expressing CasM proteins can be developed and can be transfected with the guide components, and complexes can be purified from the cells using standard purification techniques, such as but not limited to affinity, ion exchange and size exclusion chromatography. See, e.g., Jinek M., et al., “A programmable dual-RNA-guided DNA endonuclease in adaptive bacterial immunity,” Science (2012) 337:816-821.

Alternatively, the components, i.e., the cognate guides and casM polynucleotides may be provided separately to a cell, e.g., using separate constructs, or together, in a single construct, or in any combination, and complexes can be purified as above.

Methods of designing particular guides, such as for use in the complexes, are known. See, e.g., Briner et al., “Guide RNA Functional Modules Direct Cas9 Activity and Orthogonality,” Molecular Cell (2014) 56:333-339. To do so, the genomic sequence for the gene to be targeted is first identified. The exact region of the selected gene to target will depend on the specific application. For example, in order to activate or repress a target gene using, for example, Cas activators or repressors, cognate guide/CasM complexes can be targeted to the promoter driving expression of the gene of interest. For genetic knockouts, guides are commonly designed to target 5′ constitutively expressed exons which reduces the chances or removal of the targeted region from mRNA due to alternative splicing. Exons near the N-terminus can be targeted because frameshift mutations here will increase the likelihood of the production of a nonfunctional protein product. Alternatively, cognate guides can be designed to target exons that code for known essential protein domains. In this regard, non-frameshift mutations such as insertions or deletions are more likely to alter protein function when they occur in protein domains that are essential for protein function. For gene editing using HDR, the target sequence should be close to the location of the desired edit. In this case, the location where the edit is desired is identified and a target sequence is selected nearby.

The guides can be delivered to a cell. If the cell constitutively expresses a CasM nuclease, the CasM nuclease will then be recruited to the target site to cleave the target nucleic acid. If the cell does not express a CasM nuclease, complexes of cognate guide/CasM can be delivered to the cells to make breaks in the genome, thereby triggering the repair pathways in the cells.

Treated cells are then screened using methods well known in the art, such as using high-throughput screening techniques including, but not limited to, fluorescence-activated cell sorting (FACS)-based screening platforms, microfluidics-based screening platforms, and the like. These techniques are well known in the art. See, e.g., Wojcik et al., Int. J. Molec. Sci. (2015) 16:24918-24945. The cells can then be expanded and re-transfected with additional cognate guide/CasM complexes to introduce further diversity and this process can be repeated iteratively until a population with the desired properties is obtained. Single cell clones are sorted from the population, expanded and sequenced to recover the mutations that resulted in the desired function.

### Applications of CasM

Due to its RNA-targeting abilities, CasM can be used to edit RNA and in some embodiments, to treat diseases caused by toxic RNA or improperly spliced RNA.

In some embodiments cognate guide/CasM complexes, such as, but not limited to crRNA/CasM complexes, are capable of sequence-specific ssRNA activity. Recognition and cleavage of an initial ssRNA complementary to the crRNA target sequence activates the CasM protein to carry out endonuclease activity toward any single-stranded RNA in a sequence-independent manner. The sequence-specific recognition of RNA of the crRNA/CasM complex facilitates the target knockdown of gene transcripts perturbing translation of a specific protein. The non-specific endonuclease activity of an activated crRNA/CasM complex in a cellular environment can result in cell death due to depletion of RNA encoding for essential gene transcripts. Thus, the specific RNA targeting and collateral endonuclease activity of an activated crRNA/CasM complex enables the sequence-specific selection of cells expressing a RNA transcript.

Thus, in further aspects, CasM complexes, such as, but not limited to crRNA/CasM complexes, can be used in methods of screening and killing cells, such as bacterial cells, that have not been modified by a DNA targeting nuclease (i.e., a Type II Cas9 nuclease). This method comprises contacting a NATNA/Cas9 complex to a locus of interest in a population of cells. Contacting the NATNA/Cas9 complex with the locus results in DNA cleavage and subsequent repair of the break by the endogenous cellular repair machine and the introduction of indels at the break site. The targeting of the NATNA/Cas9 complex to a targeted locus that encodes an RNA transcript results in indels in an RNA transcript sequence. This modified RNA transcript sequence is different compared to a transcript from an unmodified cell (a wild-type cell). A crRNA/CasM complex can then be targeted to the unmodified transcript, wherein crRNA/CasM recognition of the unmodified transcript results in activation of the sequence independent, single-stranded RNA targeting activity of the CasM protein and subsequent cell death. Alternatively, this method can be adapted to screen for the incorporation of a donor-polynucleotide into NATNA/Cas9 break site.

In another aspect, CasM complexes can by targeted to a eukaryotic exon coding region to cause exon skipping. This method comprises contacting a crRNA/deactivated CasM complex, such as, but not limited to a crRNA/dCasM complex, with either a donor site (5′ end of an intron), a branch site (proximal to the 3′ end of an intron), or an acceptor site (5′ of an exon) of a pre-mRNA. Contacting the crRNA/dCasM complex to the various regions involved in exon splice events prevents the proper splicing of one of more exons together and causes the target exon to be “skipped”, and thus is not included in the mature mRNA and therefore omitted from the translated polypeptide sequence.

In yet another aspect, CasM complexes are used for the detection of one or more target molecules in vitro. This method comprises contacting a cognate guide/CasM complex, such as a crRNA complex, with a ssRNA target of interest within a pool of nucleic acids. The crRNA/CasM complex can be added to a sample potential containing the ssRNA target of interest, in combination with a quenched fluorescent RNA reporter, for example a RNA hexamer with a 6-Carboxyfluorescein at the 5′ end and a Iowa Black® FQ quencher (Integrated DNA Technologies, Coralville, IA) at 3′. Contacting of the crRNA/CasM complex with the ssRNA target, activates the CasM protein to carry out collateral cleavage of the quenched fluorescent RNA reporter where cleavage of the reporter and resulting in an increase fluorescence that can by read out using a spectrophotometer. The gain in fluorescence is used as a measure of the presents of a ssRNA target of interest.

In another aspect, CasM can be used for the targeted cleavage of an endogenous mRNA transcript while simultaneously delivering an exogenous mRNA transcript in cells. This method comprises contacting a cognate guide/CasM complex, with a disease-associated endogenous mRNA transcript, while simultaneously delivering of a mRNA coding for the non-disease exogenous polypeptide into a cell. Thus, the disease-associated phenotype is repressed while the non-disease phenotype is restored.

The CasM proteins described herein can also be used with associated cognate guides in order to activate or repress a target gene, to knockout a gene, to produce a nonfunctional protein product, or to alter protein function. The present invention includes methods of modulating in vitro or in vivo transcription using the various components and complexes described herein. In one embodiment, a cognate guide/CasM protein complex can repress gene expression by interfering with transcription when the cognate guide directs nucleic acid target binding of the complex to the promoter region of the gene. Use of the complexes to reduce transcription also includes complexes wherein the CasM protein is fused to a known down-regulator of a target gene (e.g., a repressor polypeptide). For example, expression of a gene is under the control of regulatory sequences to which a repressor polypeptide can bind. A cognate guide can direct nucleic acid target-binding of a repressor protein complex to the sequences encoding the regulatory sequences or adjacent the regulatory sequences such that binding of the repressor protein complex brings the repressor protein into operable contact with the regulatory sequences. Similarly, CasM can be fused to an activator polypeptide to activate or increase expression of a gene under the control of regulatory sequences to which an activator polypeptide can bind.

In one embodiment, CasM can be fused with a nuclease, or a mutant or an active portion thereof, as well as a cognate guide, in order to bring the nuclease into proximity with a target nucleic acid sequence, wherein the nuclease can produce a single-strand or double-strand break. In this way, a locus-specific cut in a target nucleic acid can be achieved using a cognate guide in combination with CasM, and the nuclease of interest. For example, it may be desirable to associate CasM with a restriction endonuclease in order to cleave at a particular restriction site in a target nucleic acid sequence. The restriction endonuclease can be selected from any of the various types of restriction endonucleases, such as, but not limited to, type I, II, III or IV. See, e.g., PCT Publication No. WO 2013/098244 to Brouns et al., published 4 Jul. 2013, for methods of producing complexes between a Cas protein and a restriction endonuclease.

Using the methods described herein, any desired nucleic acid sequence, and in particular RNA sequences, for modification can be targeted, including without limitation, protein coding mRNA sequences, in order to reduce or restore the function of the gene product; regions that have a propensity to incorporate nucleotide sequences from a donor template, termed “HDR hotspots” herein; safe harbor regions, i.e., regions where nucleotide sequences can be inserted without disrupting neighboring gene function; non-coding regulatory regions in nucleic acid sequences; and the like.

Protein coding sequences, including RNA such as mRNA, for targeting by the methods described herein include, but are not limited to, mammalian antibodies (ABs) (IgG, IgA, IgM, IgE), antibody fragments such as Fc regions, antibody Fab regions, antibody heavy chains, antibody light chains, antibody CDRs, nanobodies, chimeric antibodies and other IgG domains; T cell receptors (TCR); endonucleases and exonucleases, such as TALENS, CRISPR nucleases such as Cas9, Cas3, Cpf1, ZnFN, meganucleases, nuclease domains such as HNH domain, RuvC domain; recombinases such as Cre, Tre, Brec1, Flp, γ-integrase, IntI4 integrase, XerD recombinase, HP1 integrase; DNA topoisomerases; transposons such as the Tc1/mariner family, Tol2, piggyBac, Sleeping beauty; RAG proteins; retrotransposons such as LTR-retrotransposons and non-LTR retrotransposons (Alu, SINE, LINE); enzymes including but not limited to arginases, glycosydases, proteases, kinases, and glycosylation enzymes such as glycosyltransferase; anticoagulants such as protein C, Protein S and antithrombin; coagulants such as thrombin; nucleases such as DNAses, RNAses, helicases, GTPases; DNA or RNA binding proteins; reporter molecules, such as Green Fluorescent Protein (GFP); cell penetrating peptides and their fusions with cargo proteins; membrane proteins such as GPCRs, pain receptors such as TRP channels and ion channels; cell surface receptors including but not limited to EGFR, FGFR, VEGFR, IGFR and ephrin receptor; cell adhesion molecules like integrins and cadherins; ion channels; rhodopsins; immunoreceptors such as CD28, CD80, PD-1, PD-L1, CTLA-4, CXCR4, CXCR5, B2M, TRACA, TRBC; proteins known to be involved with genetic defects; secreted proteins including but not limited to hormones, cytokines, growth factors; vaccine antigens such as viral proteins from human immunodeficiency virus (HIV), Dengue, cytomegalovirus (CMV), Ebola, Zika and oncolytic viruses; snake toxin proteins and peptides including but not limited to phospholipases and metalloproteases; ribosomal cyclic peptides.

The present invention also encompasses genome engineering methods for preventing or treating diseases, disorders, and conditions using the various methods described herein. In one embodiment, a genome engineering method uses the introduction of nucleic acid sequences into an organism or cells of an organism (e.g., patient) to achieve expression of components of the present invention to provide modification of a target function. For example, cells from an organism may be engineered, ex vivo, by (i) introduction of vectors comprising expression cassettes expressing the various components, (ii) direct introduction of a NATNA and/or donor polynucleotides and CasM proteins, or (iii) introduction of combinations of these components. The engineered cells are provided to an organism (e.g., patient) to be treated.

Examples of genome engineering and techniques for therapy are known in the art (see, e.g., Kay, M. A., Nature Reviews Genetics (2011) 12:316-328; Wang et al., Discov. Med. (2014) 18:67-77; Wang et al., Discov. Med. (2014) 18:151-61; “The Clinibook: Clinical Gene Transfer State of the Art,” Odile Cohen-Haguenauer (Editor), EDP Sciences (Oct. 31, 2012), ISBN-10:2842541715).

In some aspects, components of the present invention are delivered using nanoscale delivery systems, such as nanoparticles. Additionally, liposomes and other particulate delivery systems can be used. For example, vectors including the components of the present methods can be packaged in liposomes prior to delivery to the subject or to cells derived therefrom, such as described in U.S. Pat. Nos. 5,580,859; 5,264,618; 5,703,055. Lipid encapsulation is generally accomplished using liposomes that are able to stably bind or entrap and retain nucleic acid.

The methods described herein can also be used to generate non-human genetically modified organisms, such as mice, plants, and the like.

While preferred embodiments of the present invention have been shown and described herein, it will be obvious to those skilled in the art that such embodiments are provided by way of example only. From the above description and the following Examples, one skilled in the art can ascertain essential characteristics of this invention, and without departing from the spirit and scope thereof, can make changes, substitutions, variations, and modifications of the invention to adapt it to various usages and conditions. Such changes, substitutions, variations, and modifications are also intended to fall within the scope of the present disclosure.

### EXPERIMENTAL

Aspects of the present invention are further illustrated in the following Examples. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric. It should be understood that these Examples, while indicating some embodiments of the invention, are given by way of illustration only.

The following Examples are not intended to limit the scope of what the inventors regard as various aspects of the present invention.

### Example 1

### Discovery of a New CRISPR-Associated (Cas) Protein in Silico

This Example describes the in silico discovery of a new Cas protein, termed “CasM,” from genomic sequencing data. The overall approach used was similar to methods described in Shmakov et al., “Discovery and functional characterization of diverse Class 2 CRISPR-Cas systems” Molecular Cell (2015) 60:385-397. In particular a computational pipeline was used to search sequencing data for CRISPR arrays in whole genomes and metagenic contigs.

Every contig or genome in the data set was inspected to determine if it contained a CRISPR array using Minced (github.com/ctSkennerton/minced) and PILERCR (drive5.com/pilercr/).

Any time a CRISPR array was found in a contig or genome, the surrounding DNA sequence (up to 10 kilobases on either side of the CRISPR array) was further inspected for open reading frames (ORFs) using the tool getorf (emboss.sourceforge.net/apps/cvs/emboss/apps/getorf.html).

The primary amino acid sequence of each predicted ORF was analyzed for potential functional domain annotations using the tool HHPred (homology detection & structure prediction by HMM-HMM comparison; toolkit.tuebingen.mpg.de/hhpred). HHPred allows the user to specify which databases to compare the amino acid sequence against to find similar protein domains. The databases searched included PFAM (which includes a large collection of protein families; http://pfam.xfam.org/), PDB (protein databank; wwpdb.org), CDD (conserved domain database; ncbi.nlm.nih.gov/Structure/cdd/cdd.shtml), and KEGG (Kyoto Encyclopedia of Genes and Genomes; genome.jp/kegg/).

Using these tools, ORFs encoding a new putative CRISPR-associated protein, termed “CasM,” was found in several species in the Clostridia family. The ORFs are proximal to a predicted HTH DNA binding protein with homology to a CRISPR-associated WYL domain. See FIG. 1 for a representative map of a CRISPR operon found in Eubacterium siraeum (Genome Accession No. NZ_DS499551.1, coordinates 211.800-220.497). The various CRISPR locus features are described in Table 7.

TABLE 7
CasM CRISPR locus for Eubacterian siraeum (FIG. 1)

101 corresponds to the CasM open reading frame (ORF) (Protein
accession: WP_005358205.1)
102 corresponds to a CRISPR Array
103 corresponds to an ORF containing a RctB RNA ligase domain
(Protein accession: WP_005358214.1)
104 corresponds to an ORF containing a WYL DNA binding domain
(Protein accession: WP_005358216.1)

The results of HHPred analysis were analyzed to determine if the CasM-encoding ORFs had predicted domains commonly found in CRISPR-associated proteins. No annotations were found for CasM, thus indicating that the protein was novel.

The sequences for the various native CasM proteins are shown in SEQ ID NOS: 37-45 (see Table 1) and the native polynucleotides encoding therefor are shown in SEQ ID NOS: 28-36 (see Table 3). This protein has no significant homology to any known protein families or to any Class 2 Cas effectors.

### Example 2

### Codon Modification of Native casM Sequences

This Example describes the process of codon optimizing CasM coding sequences to improve expression in selected host cells.

Native casM nucleotide sequences were retrieved from the reference genomes or metagenomic contigs of the host microbes as described in Example 1. The amino acid sequences of the coding regions were generated with the ExPASy DNA translation tool (web.expasy.org/translate/). Next, these amino acid sequences were entered into the Integrated DNA Technologies (Coralville, IA) Codon Optimization tool (idtdna.com/CodonOpt). “Amino acid” was chosen for the “Sequence Type” option and “Gene” was chosen for the “Product Type” option. For each native casM sequence, codon modifications were performed to increase expression in E. coli, human, and Zea mays cells.

SEQ ID NOS: 1-9 show the modified sequences for use in E. coli (see Table 4). SEQ ID NOS: 10-19 show the modified sequences for use in human cells (see Table 5). SEQ ID NOS: 20-27 show the modified sequences for use in Z. mays cells (see Table 6). Table 8 shows the percent identity of the modified sequences to the native sequences.

TABLE 8
Percent Sequence Identity to Native casM Sequences

| | modified for | modified for | modified for |
|---|---|---|---|
| casM bacterial strain | E. coli cells | human cells | Z. mays cells |
| Eubacterium siraeum | 75% | 77% | 77% |
| Ruminococcus sp., isolate | 77% | 76% | 76% |
| 2789STDY5834971 | | | |
| Ruminococcus bicirculans | 76% | 76% | 77% |
| Ruminococcus sp., isolate | 76% | 75% | 77% |
| 2789STDY5608892 | | | |
| Ruminococcus sp. CAG: 57 | 76% | 77% | 76% |
| Ruminococcus flavefaciens | 76% | 77% | 76% |
| FD-1 | | | |
| Ruminococcus albus strain | 76% | 77% | 77% |
| KH2T6 | | | |
| Ruminococcus flavefaciens | 76% | 77% | 77% |
| strain XPD3002 | | | |
| Ruminococcus sp., isolate | 75% | 76% | 78% |
| 2789STDY5834894 | | | |

### Example 3

### Production of CasM Expression Plasmids for DNA Interference Assays

This Example describes the production of plasmids that express the CasM protein.

The modified casM nucleotide sequences set forth in Example 2 were synthesized in vitro. The DNA sequences were cloned into an appropriate plasmid for expression in E. coli.

For E. coli expression, the E. coli-modified sequences were cloned into a p14A plasmid backbone using appropriate restriction nucleases. The plasmid backbone contained a T7 promoter upstream of the CasM coding sequence to facilitate transcription in cells.

The p14A plasmid backbone also contained a cloning site enabling the insertion of a minimal CRISPR array. The minimal CRISPR array contained one repeat sequence, followed by one spacer sequence, followed by one repeat sequence. The plasmid backbone also contained a T7 promoter upstream of the CRISPR array site, a kanamycin resistance gene, and a ColE1 origin of replication.

Similar techniques are used for preparing plasmids for expression in human and Zea mays cells. Once the plasmids are produced, they are transfected into the selected cell, e.g., F. coli, human, or plant cells (e.g., Zea mays cells).

### Example 4

### Plasmid Interference Assay

This Example describes the use of CasM in an assay to evaluate its ability to cleave double-stranded DNA in the form of a target plasmid. The overall approach is similar to methods used in Burnstein et al., Nature (2016) 542:237-241.

The CasM expression plasmid in Example 3 is transformed into E. coli cells. The cells are grown in a medium containing kanamycin to select only for cells that contain the CasM expression plasmid.

A target plasmid is constructed that contains the spacer sequence contained in the CRISPR array of the CasM expression plasmid. Adjacent to the spacer sequence is a randomized PAM sequence of 7 nucleotides. Plasmid libraries containing randomized PAM sequences are assembled by annealing a DNA oligonucleotide containing a target with a 7 nt randomized PAM region with a primer and extended with Klenow Fragment (New England Biolabs, Ipswitch, MA). The double-stranded DNA is digested with EcoRI and Ncol and ligated into a pUC19 backbone. The ligated library is transformed into E. coli DH5a and cells are harvested, the plasmids extracted and purified. 200 ng of the pooled library is transformed into electro-competent E. coli harboring a CRISPR locus or a control plasmid with no locus. The transformed cells are plated on selective media containing carbenicillin (100 mg L⁻¹) and chloramphenicol (30 mg L⁻¹) for 30 hours at 25° C. Plasmid DNA is extracted and the PAM sequence is amplified with adapters for Illumina sequencing. The 7 nt PAM region is extracted and PAM frequencies calculated for each 7 nt sequence. PAM sequences depleted above the specified threshold are used to generate a sequence logo with WebLogo (weblogo.berkeley.edu). If depleted PAMs are present, this shows that the nuclease is a double-stranded DNA nuclease.

### Example 5

### Targeted Modification of HEK293 Cells Using CasM

This Example illustrates the use of CasM to modify human embryonic kidney (HEK293) cells at specific target locations.

casM polynucleotides are transfected into HEK293 cells constitutively expressing a CasM-GFP fusion (HEK293-CasM-GFP), using the Nucleofector™ 96-well Shuttle System (Lonza, Allendale, NJ) and the following protocol. The casM polynucleotides are designed to target the FUT8 gene. Equal molar amounts of casM polynucleotide components are prepared in an annealing buffer (1.25 mM HEPES, 0.625 mM MgCl₂, 9.375 mM KCl at pH 7.5), incubated for 2 minutes at 95° C., removed from the thermocycler, allowed to equilibrate to room temperature, and dispensed in a 10 μL final volume in a 96-well plate. Culture medium is aspirated from HEK293-CasM-GFP cells, and the cells are washed once with calcium and magnesium-free PBS and then trypsinized by the addition of TrypLE (Life Technologies, Grand Island, NY) followed by incubation at 37° C. for 3-5 minutes. Trypsinized cells are gently pipetted up and down to form a single cell suspension and added to DMEM complete culture medium composed of DMEM culture medium (Life Technologies, Grand Island, NY) containing 10% FBS (Fisher Scientific, Pittsburgh, PA) and supplemented with penicillin and streptomycin (Life Technologies, Grand Island, NY).

The cells are then pelleted by centrifugation for 3 minutes at 200× g, the culture medium aspirated and cells resuspended in PBS. The cells are counted using the Countess™ II Automated Cell Counter (Life Technologies, Grand Island, NY). 2.2×10⁷ cells are transferred to a 50 ml tube and pelleted. The PBS is aspirated and the cells resuspended in Nucleofector™ SF (Lonza, Allendale, NJ) solution to a density of 1×10⁷ cells/mL. 20 μL of the cell suspension are then added to individual wells containing 10 μL of casM polynucleotide components and the entire volume is transferred to the wells of a 96-well Nucleocuvette™ Plate (Lonza, Allendale, NJ). The plate is loaded onto the Nucleofector™ 96-well Shuttle™ (Lonza, Allendale, NJ) and cells are nucleofected using the 96-CM-130 Nucleofector™ program (Lonza, Allendale, NJ). Post-nucleofection, 70 μL DMEM complete culture medium is added to each well and 50 μL of the cell suspension are transferred to a collagen coated 96-well cell culture plate containing 150 μL pre-warmed DMEM complete culture medium. The plate is then transferred to a tissue culture incubator and maintained at 37° C. in 5% CO₂ for 48 hours.

Genomic DNA (gDNA) is prepped using the QuickExtract DNA extraction solution (Illumina, San Diego, CA) pursuant to the manufacturer instructions. Sequencing amplicons of between 150 bp to 200 bp are designed to span the CasM RNP FUT8 target site. Using previously isolated gDNA, a first PCR is performed using Herculase II Fusion DNA Polymerase™ (Agilent, Santa Clara, CA) with primers comprising an adapter sequences and a sequence specific to the region flanking the FUT8 target site. A second PCR is performed using the amplicons of the first round of PCR as template at 1/20th the volume of the PCR reaction volume. The second PCR uses a second set of primers comprising a sequence complementary to the adapter sequence of the first primer pair, a barcode index sequence unique to each sample, and a flow cell adapter sequence. Amplicons are pooled and analyzed on a 2% TBE gel and bands of expected amplicon sizes are gel purified using the QIAEX II Gel extraction kit™ (Qiagen, Venlo, Luxembourg). The concentrations of purified amplicons are evaluated using the double-stranded DNA BR Assay Kit and Qubit system™ (Life Technologies, South San Francisco, CA) and library quality determined using the Agilent DNA100Chip and Agilent Bioanalyzer 2100 system™ (Agilent, Santa Clara, CA). After validation of library quality, the library is sequenced on a MiSeq Benchtop Sequencer™ (Illumina, San Diego, CA) with the MiSeq Reagent Kit v2T_{M} (300 cycles, Illumina, San Diego, CA) per manufacturer instructions for 151 bp paired end reads.

The identity of products in the sequencing data is analyzed based upon the index barcode sequence adapted onto the amplicon in the second round of PCR. A computational script is used to process the MiSeq data by executing the following tasks:
1. Joining paired end reads with the aid of fastq-join (Aronesty 2011: code.google.com/p/ea-utils);
2. Validating the sequence reads for appropriate primer sequences being present at both 5′ and 3′ ends of the read sequence using fastx_barcode_splitter (hannonlab.cshl.edu/fastx_toolkit/index.html); reads lacking correct primer sequences at both ends are discarded.
3. Comparing Read sequences to expected wild type FUT8 sequence; identical read sequences are classified as having the same indel modification.

Other chromosomal loci within HEK293 cells are similarly modified by selection of an appropriate spacer sequence for the CasM RNP. Selection is specific to a specific gene target and the procedure outlined in this Example is readily modifiable by one of ordinary skill in the art for other gene targets.

This procedure can provide data to verify the CasM RNP and to detect nucleic acid-guided nuclease activity at targeted loci in HEK293 cells.

### Example 6

### CasM CRISPR Array Processing Assay

This Example describes the CRISPR array processing activity of a CasM protein. The following method may be practiced with other CasM protein homologs to characterize their CRISPR array processing capabilities.

### A. Identification of the CRISPR Array Repeat Sequence

The CRISPR array of the CasM protein homolog (SEQ ID NO:39) was analyzed in silico and the repeat sequence identified. The in silico structure of a CRISPR repeat sequence (SEQ ID NO:51) associated with the CasM protein (SEQ ID NO:39) as predicted using an RNA folding algorithm (rna.urmc.rochester.edu/RNA structureWeb/Servers/Predict1.html) is shown in FIG. 2 (SEQ ID NO:51). The various CRISPR repeat sequence structural components represented in FIG. 2 are described in Table 9.

TABLE 9
Numerical Indicators Used to Illustrate CasM CRISPR Repeat Sequence
Structural Components (FIG. 2)

```
470 corresponds to a 5′ repeat handle sequence
471 corresponds to a stem-duplex formed by a first stem duplex stand
hybridized to a second stem duplex strand
472 corresponds to a loop sequences
473 corresponds to a 5′ repeat handle sequence
474 corresponds to the 3′ attachment point of a spacer sequence
475 corresponds to the CRISPR repeat processing positions performed
by CasM upon guide binding
  10 indicates the tenth nucleotide position
  20 indicates the twentieth nucleotide position
  30 indicates the thirtieth nucleotide position
```

The repeat sequence was used to design a CRISPR array by incorporating spacer sequences 5′,3′, or both 5′ and 3′ of the repeat sequence. The sequences were used for synthesis as RNA. The synthetic CasM CRISPR array is represented in FIG. 4 and structural components 470-473 are detailed in Table 9. Additional components 490 and 491 correspond to a first 5′ and a first 3′ spacer sequence, respectively. The CRISPR array components are shown in Table 10.

TABLE 10
CRISPR Array Components

| | CRISPR | | |
|---|---|---|---|
| SEQ ID | Array Con- | | Size |
| NO: | figuration | Sequence | (nt) |
| SEQ ID | spacer- | UGAUACUGCUUUGAUGUCAGCAUUGC | 96 |
| NO: 46 | repeat- | AUAUCUACUAUACUGGUGCGAAUUUG | |
| | spacer | CACUAGUCUAAAAUCUAUAACCAUAA | |
| | | GUUCUUCUGCGUUCAUAU | |
| SEQ ID | spacer- | UGAUACUGCUUUGAUGUCAGCAUUGC | 66 |
| NO: 47 | repeat | AUAUCUACUAUACUGGUGCGAAUUUG | |
| | | CACUAGUCUAAAAU | |
| SEQ ID | repeat- | CUACUAUACUGGUGCGAAUUUGCACU | 66 |
| NO: 48 | spacer | AGUCUAAAAUUGAUACUGCUUUGAUG | |
| | | UCAGCAUUGCAUAU | |

*CRISPR repeat sequence is underlined

SEQ ID NO:46 comprises, in a 5′ to 3′ orientation, CRISPR array structural components 490, 470-473, and 491. SEQ ID NO:47 comprises, in a 5′ to 3′ orientation, CRISPR array structural components 490 and 470-473. SEQ ID NO:48 comprises, in a 5′ to 3′ orientation, CRISPR array structural components 470-473 and 491.

Alternative to synthesis, CRISPR arrays may be made via PCR using 3′ overlapping primers containing DNA sequences corresponding to CRISPR array components and incorporation of a T7 promoter sequence 5′ of the CRISPR arrays, followed by in vitro transcription.

### B. CasM Purification

The CasM protein coding sequence was codon-optimized for expression in E. coli and incorporated into a modified pET plasmid backbone downstream of a maltose binding protein (MBP) using appropriate restriction nucleases. The plasmid backbone contained a T7-Lac promoter upstream of the MBP-CasM coding sequence to facilitate transcription in cells. Additionally, the plasmid backbone contained an kanamycin resistance gene and a ColE1 origin of replication.

The CasM expression plasmid was transformed into Rosetta2 (DE3) cells, and cells were grown in two 1 L shake flasks at 37° C. until cells reached an optical density of 0.6, after which protein expression was induced by addition of 0.5 mM IPTG. Cells were then incubated at 16° C. overnight.

Cells were collected via centrifugation and lysed via sonication. Cell debris was pelleted, and the clarified lysate was purified using a combination of HisTrap column chromatography, followed by cleavage of the MBP tag, and finally cation exchange column chromatography. Final purified protein was quantified using a NanoDrop™ 2000 spectrophotometer (ThermoFisher, Waltham, MA), and stored at −80° C.

### C. In Vitro CRISPR Array Processing

Synthetic CRISPR array reagents were resuspended in water to a final concentration of 250 μM and diluted to a working concentration of 250 nM. CRISPR arrays were incubated at 95° C. for two minutes and cooled by 0.5° C./sec in a thermocycler to a final temperature of 25° C.

CasM was diluted to a final concentration of 500 nM in 1× cleavage buffer (20 mM HEPES, 100 mM KCl, 5 mM MgCl₂, and 5% glycerol at pH 7.4). The reaction was initiated by addition of CasM protein to denatured CRISPR arrays in a final reaction volume of 12 μL, followed by incubation at 37° C. for 15 minutes. The reaction was terminated by heat inactivation at 95° C. for 2 minutes, and 6 μL of the reaction was mixed with 6 μL of 2× RNA loading buffer (New England Biolabs, Ipswich, MA). Low Range ssRNA Ladder™ (New England Biolabs, Ipswich, MA) was diluted 125-fold in water and 7 μL were mixed with 7 μL of 2× RNA Loading Dye™ (New England Biolabs, Ipswich, MA) and incubated at 90° C. for 4 minutes and then incubated on ice for 5 minutes. CRISPR array processing reactions and ssRNA ladder were analyzed on a Mini-PROTEAN 15% TBE-Urea™ (Bio-RAD, Hercules, CA) run at 200 V for 1 hour in 1× TBE running buffer. The gel was stained using 2× SYBR gold™ (MilliporeSigma, St. Louis, MI) for 15 minutes and visualized using a Gel Doc™ EZ system™ (Bio-RAD, Hercules, CA). The results of the CRISPR array processing reactions are shown in FIG. 3 and lane order is presented in Table 11.

TABLE 11
CRISPR Array Cleavage Gel Lane Order

| | CRISPR Array | | |
|---|---|---|---|
| Lane | Configuration | SEQ ID NO: | CasM |
| 1 | Low Range ssRNA Ladder | | |
| 2 | spacer-repeat-spacer | SEQ ID NO: 46 | − |
| 3 | spacer-repeat | SEQ ID NO: 47 | − |
| 4 | repeat-spacer | SEQ ID NO: 48 | − |
| 5 | spacer-repeat-spacer | SEQ ID NO: 46 | + |
| 6 | spacer-repeat | SEQ ID NO: 47 | + |
| 7 | repeat-spacer | SEQ ID NO: 48 | + |

The results of the CRISPR array cleavage assays (FIG. 3) demonstrated that the CasM protein is capable of processing a cognate CRISPR array. The three bands shown in Lane 1 correspond to 150, 80 and 50 nucleotide standards of the Low Range ssRNA Ladder™ (New England Biolabs, Ipswich, MA), respectively). Indicator 480 in FIG. 3 corresponds to a processed CasM crRNA comprising a portion of the CRISPR repeat sequence and a spacer sequence. Indicator 481 corresponds to RNA species cleaved from the 5′ end of the CRISPR array following addition of CasM.

The CasM cleaved nucleotides in 5′ region of the repeat element (FIG. 3, comparing Lane 2 to 5; comparing Lane 3 to Lane 6), and exhibited no cleavage 3′ of the repeat element (FIG. 3, comparing Lane 4 to Lane 7). crRNA proceeded from the CasM CRISPR array and therefore had a 5′ repeat element and a spacer element 3′ of the repeat. In the absence of CasM, no cleavage of the crRNA was observed (FIG. 3, Lanes 2, 3, and 4).

Schematics of the crRNA processing regimes are depicted in FIG. 5. In FIG. 5, panel I corresponds to the reaction in FIG. 3, Lane 5; FIG. 5 panel II corresponds to the reaction in FIG. 3, Lane 6; and FIG. 5 panel III corresponds to the reaction in FIG. 3, Lane 7. The various components represented in FIG. 5 are described in Table 12.

TABLE 12
Numerical Indicators Used to Illustrate the Results of the
in vitro CRISPR Array Cleavage Assay (FIG. 5)

492 corresponds to a spacer-repeat-spacer CRISPR array (SEQ ID.
NO: 46)
493 corresponds to a spacer-repeat CRISPR array (SEQ ID. NO: 47)
494 corresponds to a repeat-spacer CRISPR array (SEQ ID. NO: 48)
495 corresponds to a CasM protein
496 corresponds to a processed crRNA
497 corresponds to a RNA species cleaved from the 5′ end of the
CRISPR array
498 corresponds to a processed CRISPR repeat sequence
499 corresponds to a RNA species cleaved from the 5′ end of the
CRISPR repeat sequence

### Example 7

### CasM ssRNA Cleavage Assay

This Example illustrates the use of a crRNA/CasM protein complex to carry out ssRNA cleavage. The following method may be practiced with other CasM protein and crRNA to cleave ssRNA targets.

A. Generation of ssRNA Target

A ssRNA target was generated via PCR amplification of a 224 nucleotide target sequence from a plasmid. A T7 promoter sequence was incorporated into the S′ end of the reverse PCR primer (SEQ ID NO:50) for transcription. The primers used for ssRNA target DNA template are presented in Table 13.

TABLE 13
ssRNA Target DNA Template Primers

| SEQ ID NO: | Name | Sequence |
|---|---|---|
| SEQ ID NO: 49 | Forward | CGAAATTAATACGACTCACTATA |
| | primer | GGTTTCGATTATGCGGCCGTGT |
| SEQ ID NO: 50 | Reverse | AGGAGATATACCATGGGCAGCA |
| | primer | |

*T7 Promoter sequence underlined.

The primers were present at a concentration of 400 nM each. PCR reactions were performed using Q5 Hot Start High-Fidelity 2× Master Mix™ (New England Biolabs, Ipswich, MA) following the manufacturer's instructions with 10 ng of plasmid template. PCR assembly reactions were carried out using the following thermal cycling conditions: 98° C. for 2 minutes; cycles of 10 seconds at 98° C.; 15 seconds at 60° C.; 30 seconds at 72° C.; and a final extension at 72° C. for 2 minutes. DNA product quality was evaluated after the PCR reaction by agarose gel electrophoresis (1.5%, SYBR® Safe; Life Technologies, Grand Island, NY).

Between 0.1-0.5 μg of the amplified ssRNA target DNA template was used as a template for transcription using T7 High Yield RNA Synthesis Kit™ (New England Biolabs, Ipswich, MA) for approximately 16 hours at 37° C. Transcription reactions were treated with DNase I (New England Biolabs, Ipswich, MA) and purified using GeneJet RNA Cleanup and Concentration Kit™ (Life Technologies, Grand Island, NY). The quality of the transcribed RNA was checked by agarose gel electrophoresis (2%, SYBR® Safe; Life Technologies, Grand Island, NY) and quantified using the Quant-iT™ RNA Assay Kit™ (ThermoFisher, Waltham, MA).

B. Designing CasM crRNA

The 224 nucleotide ssRNA target sequence was probed in silico for a 30 nucleotide target sequence. The target sequence was appended in silico to the 3′ end of the CasM crRNA repeat sequence and the crRNA sequence was provided to a commercial manufacturer for synthesis.

C. ssRNA Cleavage Assay

Synthetic crRNA reagents were resuspended in water to a final concentration of 250 μM and diluted to a suitable working concentration of 250 nM. In vitro transcribed ssRNA target was diluted to 43 ng/μL in water. Both the crRNA and the ssRNA target reagents were separately incubated at 95° C. for two minutes and cooled by 0.5° C./sec in a thermocycler to a final temperature of 25° C. The CasM protein was diluted to various concentrations in water and 1× cleavage buffer. Denatured crRNA was added at various concentrations to the CasM protein and incubated in a thermocycler for 10 minutes at 37° C. The cleavage reactions were initiated by the addition of the ssRNA target to a final concentration of 56.4 nM in a final reaction volume of 12 μL. The concentration of each component in the various reactions is shown in Table 14.

TABLE 14
ssRNA Targeting Reaction Component Concentrations

| | | | | Molar ratio |
|---|---|---|---|---|
| | | nM | nM ssRNA | CasM:crRNA:ssRNA |
| Reaction | nM CasM | crRNA | target | target |
| 1 | 11.3 | 33.8 | 56.4 | 0.2:0.6:1 |
| 2 | 22.5 | 67.6 | 56.4 | 0.4:1.2:1 |
| 3 | 33.8 | 101.5 | 56.4 | 0.6:1.8:1 |
| 4 | 45.1 | 135.3 | 56.4 | 0.8:2.4:1 |
| 5 | 56.4 | 169.1 | 56.4 | 1:3:1 |
| 6 | 112.7 | 338.2 | 56.4 | 2:6:1 |
| 7 | 225.5 | 676.4 | 56.4 | 4:12:1 |
| 8 | 338.2 | 1014.6 | 56.4 | 6:18:1 |
| 9 | 450.9 | 1352.8 | 56.4 | 8:24:1 |
| 10 | 563.7 | 1691.0 | 56.4 | 10:30:1 |
| 11 | 0.0 | 0.0 | 56.4 | 0:3:1 |
| 12 | 56.4 | 0.0 | 56.4 | 1:0:1 |
| 13 | 0.0 | 169.1 | 56.4 | 0:3:1 |
| 14 | 0.0 | 169.1 | 0.0 | 1:3.0 |

Samples were mixed and centrifuged briefly before being incubated for 1 hour at 37° C. Reactions were terminated by incubating the reaction at 95° C. for 2 minutes followed by the addition of 100 U/μL of Proteinase K (New England Biolabs, Ipswich, MA), 4 M urea, 5 μM DTT, 50 μM EDTA and incubation at 37° C. for 15 minutes. 7 μL of each reaction was mixed with 6 μL of 2× RNA Loading Dye (New England Biolabs, Ipswich, MA) and incubated at 90° C. for two minutes. Low Range ssRNA Ladder™ (New England Biolabs, Ipswich, MA) was diluted 125-fold in water and 7 μL were mixed with 7 μL of 2× RNA Loading Dye™ (New England Biolabs, Ipswich, MA) and incubated at 90° C. for 4 minutes and then incubated on ice for 5 minutes. Cleavage reactions and ssRNA ladder were analyzed on a Mini-PROTEAN 15% TBE-Urea™ (BioRAD, Hercules, CA), run at 200 V for 1 hour in 1× TBE running buffer. Gel was stained using 2× SYBR gold™ (Life Technologies, Grand Island, NY) for 15 minutes and visualized with using a Gel Doc EZ system™ (BioRAD, Hercules, CA). The results of the crRNA cleavage assay are shown in FIG. 6 and the components of each lane shown in Table 15. Numerical indicator 500 corresponds to the ssRNA target. Numerical indicator 501 corresponds to a ssRNA target hybridized to the spacer sequences of the CasM crRNA. Numerical indicator 502 corresponds to an unprocessed CasM crRNA. Numerical indicator 503 corresponds to the processed CasM crRNA species.

TABLE 15
ssRNA Cleavage Gel Lane Order

| | Molar ratio |
|---|---|
| | CasM:crRNA:ssRNA |
| Lane | target |
| 1 | 0.2:0.6:1 |
| 2 | 0.4:1.2:1 |
| 3 | 0.6:1.8:1 |
| 4 | 0.8:2.4:1 |
| 5 | 1:3:1 |
| 6 | 2:6:1 |
| 7 | 4:12:1 |
| 8 | 6:18:1 |
| 9 | 8:24:1 |
| 10 | 10:30:1 |
| 11 | ssRNA Ladder |
| 12 | 0:3:1 |
| 13 | 1:0:1 |
| 14 | 0:3:1 |
| 15 | 1:3:0 |

The results of the ssRNA cleavage assay shown in FIG. 6 demonstrated that a CasM: crRNA protein complex was capable of ssRNA target cleavage. The results of this procedure demonstrate that increasing the amount of CasM: crRNA complexes resulted in decreased amounts of ssRNA target (FIG. 6, indicator 500) visualized on the gel (FIG. 6, Lanes 1-10).

### Example 8

### Production of CasM and RtcB Expression Plasmids for MS2 Phage Drop Plaque Assays

This Example describes the production of plasmids for the expression of CasM, RtcB (RNA 3′-terminal phosphate cyclase, group B), and a corresponding CRISPR array in E. coli for use in a MS2 phage drop plaque assay. The following method can be practiced with other CasM, RtcB, and CRISPR array homologs.

The casM and rtcB nucleotide sequence from Eubacterium siraeum (SEQ ID NO: 37 and SEQ ID NO:60, respectively) are selected and codon optimized for expression in E. coli. The E. coli-modified sequences are cloned into a p14A plasmid backbone using appropriate restriction nucleases. The plasmid backbone contains a T7 promoter upstream of each protein coding sequence to facilitate transcription in cells. Two control plasmids, one containing only the casM gene sequence under the control of a T7 promoter and the other plasmid only containing the rtcB gene sequence under the control of a T7 promoter, can also constructed.

A spacer sequence that has homology with the MS2 phage genome is engineered in silico flanked 5′ and 3′ by the Eubacterium siraeum CasM CRISPR repeat sequence. A non-targeting spacer with no homology to the MS2 phage or E. coli genome, is similarly engineered as a control. Both sequences are subcloned into separate plasmids between an upsteam T7 promoter sequence and a downstream transcription terminator sequence.

### Example 9

### MS2 Phage Drop Plaque Assay

This Example describes the use of CasM and RtcB in an assay to evaluate the ability of the RtcB protein to modulate CasM's sequence-specific and collateral nuclease activity in E. coli. The method set forth herein is adapted from Smargon et al., Molec. Cell (2017) 65:618-630. Not all of the following steps are required for screening, nor must the order of the steps be as presented.

The expression plasmids constructed in Example 8 are individually and in combination transformed into BL21 (AI) E. coli cells from a commercial provider, such as Invitrogen (Carlsbad, CA). Transformed cells are grown overnight at 37° C., with shaking, in lysogeny broth (LB) supplemented with 100 μg/mL carbenicillin, to select for cells that contain the CasM expression plasmid.

The following day, cells are diluted 1:100 and then grown at 37° C., with shaking, to an OD600 of 2.0. The cells are then mixed with 4 mL of carbenicillin-containing top Agar (10 g/L tryptone, 5 g/L yeast extract, 10 g/L sodium chloride, 5 g/L agar) and poured onto LB-antibiotic base plates. The top agar also contains 0.2% arabinose to induce expression of the casM, rtcB and CRISPR array coding sequences. 10-fold serial dilutions of MS2 phage (ATCC 15597-B1, Manassas VA) are made in LB and then spotted onto hardened top agar with a multi-channel pipette. Plaque formation is assessed after overnight incubation of the spotted plates at 37° C.

To assess whether the RtcB protein modulates CasM cleavage activity, the relative plaque formation is determined by comparing cells expressing CasM, RtcB, and the CRISPR array targeting MS2 phage; cells expressing CasM and the CRISPR array targeting MS2 phage; and cells expressing CasM and the CRISPR array not targeting MS2 phage; cells expressing CasM and RtcB only.

### Example 10

### Introduction of CasM RNP Complexes into Target Cells

This Example illustrates the design and delivery of CasM and crRNA ribonucleoprotein (RNP) complexes into human cells to enable mRNA cleavage of the human epidermal growth factor receptor (EGFR) gene and subsequent knockdown of EGFR gene expression.

A. Production of CasM Complexes and Transformation into Cells

Mature crRNAs (SEQ ID NOS: 70-165) were designed to target the EGFR locus in the human genome. Each crRNA contained a 5′ 36 nt repeat (SEQ ID NO:51) followed by a 30 nt spacer. crRNAs were designed to target 72 unique sequences complementary to the egfr mRNA within exons 1-3. Sequences were designed such that flanking sequences within 1 bp were not biased by any nucleotide. As negative controls, not predicted to induce cleavage, crRNAs were also designed to target (1) eight genomic sequences upstream of the predicted egfr mRNA; (2) eight sequences complementary to the vegfa mRNA exon 1; and (3) eight sequences identical to the egfr mRNA.

Double-stranded DNA (dsDNA) guide templates containing upstream T7 promoter sequences were created by annealing complementary oligonucleotides (Integrated DNA Technologies, Coralville, IA) at a final concentration of 10 μM in annealing buffer (30 mM HEPES, 300 mM KCL), then incubating at 95° C. for two minutes, and then slowly cooled to approximately 25° C., and incubated for an additional 20 minutes. Following annealing, guides were transcribed with T7 RNA polymerase HiScribe™ T7 High Yield RNA Synthesis Kit™ (New England Biolabs, Ipswich, MA) according to manufacturer's instructions. Next, samples were digested with RNase-free DNase-I (New England Biolabs, Ipswich, MA) according to manufacturer's instructions, then purified using RNAClean XP™ beads (Beckman Coulter, Indianapolis, IN).

For RNAClean XP™ bead purification, 30 μL of sample was combined with 155 μL of 100% isopropanol and 10 μL of 3 M sodium acetate and then mixed thoroughly. Next, 50 μL of RNAClean XP™ beads were incubated on a magnet for three minutes to allow separation of the liquid and beads, and the supernatant was removed. Subsequently, the samples containing crRNA were added to the beads, mixed, incubated at approximately 25° C. for five minutes, then incubated on a magnet for three minutes. Finally, the supernatant was removed, the beads were washed once with 85% ethanol, dried, and then the crRNA was eluted in 20 μL of molecular biology grade water. crRNAs were quantified using ribogreen and then normalized to 1 μg/μL.

To assemble CasM RNPs, 120 pmols of each unique crRNA were added to a well then incubated at 95° C. for two minutes followed by 25° C. for approximately 10 minutes. Next, the denatured crRNA guides were combined with 20 pmol of CasM (SEQ ID NO:39) in RNP assembly buffer (20 mM HEPES; pH 7.4, 10 mM MgCl₂, 150 mM KCl, 5% glycerol) and then incubated at 37° C. for 10 minutes.

B. Transfection of CasM RNP Complexes into Eukaryotic Cells

HeLa cells (ATCC, Manassas, VA) were cultured in suspension in DMEM medium supplemented with 10% FBS and 1× Antibiotic-Antimycotic Solution (Mediatech, Inc., Manassas, VA) at 37° C., 5% CO₂ and 100% humidity. HeLa cells were transfected using the Nucleofector® 96-well Shuttle System (Lonza, Allendale, NJ). Prior to nucleofection, 5 μl of the CasM: crRNA RNPs were assembled in individual wells of a 96-well plate. HeLa cells were transferred to a 50 ml conical centrifuge tube and centrifuged at 200×G for five minutes. The media was aspirated and the cell pellet was washed in calcium and magnesium-free PBS. The cells were centrifuged once more and resuspended in Nucleofector SF™ buffer (Lonza, Allendale, NJ) at a concentration of 5×10⁶ cells/ml. 20 μl of this cell suspension was added to the CasM: crRNA RNPs in the 96 well plate, mixed, and then the entire volume was transferred to a 96-well Nucleocuvette™ Plate. The plate was then loaded into the Nucleofector 96-well Shuttle™ and cells were nucleofected using the 96-CN-114 Nucleofector1. program (Lonza, Allendale, NJ). Immediately following nucleofection, 75 μl of complete DMEM medium was added to each well of the 96-well Nucleocuvette™ Plate. Half of the contents of each well were then transferred to a 96-well tissue culture plate containing 150 μl of complete DMEM medium. This procedure was then repeated in order to plate a duplicate for each well, one which would be used for lysis and genomic DNA analysis, and one for FACS analysis. The cells were cultured at 37° C., 5% CO₂ and 100% humidity for approximately 5 days.

### C. FACS Analysis of CasM Mediated EGFR Knockdown

Fluorescence activated cell sorting (FACS) analysis was performed 5 days after nucleofection of HeLa cells with EGFR-targeting CasM2 RNPs. In brief, 2×10⁵-4×10⁵ cells/well were detached with TrypLE Express (Gibco), stained with 2 μL APC anti-human EGFR (Clone AY13, Sony Biotechnology) in 100 μL total volume and then analyzed using Intellicyte Flow

Cytometer (Intellicyt, Albuquerque, NM). Results from these experiments are shown in Table 16.

TABLE 16
CasM Mediated EGFR knockdown

| | % | | |
|---|---|---|---|
| | EGFR | | |
| | negative | | crRNA |
| Name | cells | transcription | SEQ ID NO. |
| Untransfected reference cell | 2% | n/a | — |
| Intergenic target-1 | 5% | Intergenic | SEQ ID No: 70 |
| Intergenic target-2 | 6% | Intergenic | SEQ ID No: 71 |
| Intergenic target-3 | 9% | Intergenic | SEQ ID No: 72 |
| Intergenic target-4 | 8% | Intergenic | SEQ ID No: 73 |
| Intergenic target-5 | 5% | Intergenic | SEQ ID No: 74 |
| Intergenic target-6 | 6% | Intergenic | SEQ ID No: 75 |
| Intergenic target-7 | 6% | Intergenic | SEQ ID No: 76 |
| Intergenic target-8 | 4% | Intergenic | SEQ ID No: 77 |
| Exon 1 target-1 | 6% | Exon 1/28 | SEQ ID No: 78 |
| Exon 1 target-2 | 5% | Exon 1/28 | SEQ ID No: 79 |
| Exon 1 target-3 | 5% | Exon 1/28 | SEQ ID No: 80 |
| Exon 1 target-4 | 4% | Exon 1/28 | SEQ ID No: 81 |
| Exon 1 target-5 | 6% | Exon 1/28 | SEQ ID No: 82 |
| Exon 1 target-6 | 5% | Exon 1/28 | SEQ ID No: 83 |
| Exon 1 target-7 | 6% | Exon 1/28 | SEQ ID No: 84 |
| Exon 1 target-8 | 6% | Exon 1/28 | SEQ ID No: 85 |
| Exon 1 target-9 | 4% | Exon 1/28 | SEQ ID No: 86 |
| Exon 1 target-10 | 3% | Exon 1/28 | SEQ ID No: 87 |
| Exon 1 target-11 | 3% | Exon 1/28 | SEQ ID No: 88 |
| Exon 1 target-12 | 2% | Exon 1/28 | SEQ ID No: 89 |
| Exon 1 target-13 | 4% | Exon 1/28 | SEQ ID No: 90 |
| Exon 1 target-14 | 4% | Exon 1/28 | SEQ ID No: 91 |
| Exon 1 target-15 | 5% | Exon 1/28 | SEQ ID No: 92 |
| Exon 1 target-16 | 4% | Exon 1/28 | SEQ ID No: 93 |
| Exon 1 target-17 | 6% | Exon 1/28 | SEQ ID No: 94 |
| Exon 1 target-18 | 6% | Exon 1/28 | SEQ ID No: 95 |
| Exon 1 target-19 | 6% | Exon 1/28 | SEQ ID No: 96 |
| Exon 1 target-20 | 5% | Exon 1/28 | SEQ ID No: 97 |
| Exon 1 target-21 | 5% | Exon 1/28 | SEQ ID No: 98 |
| Exon 1 target-22 | 5% | Exon 1/28 | SEQ ID No: 99 |
| Exon 1 target-23 | 6% | Exon 1/28 | SEQ ID No: 100 |
| Exon 1 target-24 | 5% | Exon 1/28 | SEQ ID No: 101 |
| Exon 2 target-1 | 6% | Exon 2/28 | SEQ ID No: 102 |
| Exon 2 target-2 | 7% | Exon 2/28 | SEQ ID No: 103 |
| Exon 2 target-3 | 11% | Exon 2/28 | SEQ ID No: 104 |
| Exon 2 target-4 | 5% | Exon 2/28 | SEQ ID No: 105 |
| Exon 2 target-5 | 6% | Exon 2/28 | SEQ ID No: 106 |
| Exon 2 target-6 | 8% | Exon 2/28 | SEQ ID No: 107 |
| Exon 2 target-7 | 11% | Exon 2/28 | SEQ ID No: 108 |
| Exon 2 target-8 | 10% | Exon 2/28 | SEQ ID No: 109 |
| Exon 2 target-9 | 13% | Exon 2/28 | SEQ ID No: 110 |
| Exon 2 target-10 | 8% | Exon 2/28 | SEQ ID No: 111 |
| Exon 2 target-11 | 10% | Exon 2/28 | SEQ ID No: 112 |
| Exon 2 target-12 | 8% | Exon 2/28 | SEQ ID No: 113 |
| Exon 2 target-13 | 13% | Exon 2/28 | SEQ ID No: 114 |
| Exon 2 target-14 | 16% | Exon 2/28 | SEQ ID No: 115 |
| Exon 2 target-15 | 19% | Exon 2/28 | SEQ ID No: 116 |
| Exon 2 target-16 | 11% | Exon 2/28 | SEQ ID No: 117 |
| Exon 2 target-17 | 10% | Exon 2/28 | SEQ ID No: 118 |
| Exon 2 target-18 | 19% | Exon 2/28 | SEQ ID No: 119 |
| Exon 2 target-19 | 20% | Exon 2/28 | SEQ ID No: 120 |
| Exon 2 target-20 | 25% | Exon 2/28 | SEQ ID No: 121 |
| Exon 2 target-21 | 15% | Exon 2/28 | SEQ ID No: 122 |
| Exon 2 target-22 | 17% | Exon 2/28 | SEQ ID No: 123 |
| Exon 2 target-23 | 14% | Exon 2/28 | SEQ ID No: 124 |
| Exon 2 target-24 | 12% | Exon 2/28 | SEQ ID No: 125 |
| Exon 3 target-1 | 7% | Exon 3/28 | SEQ ID No: 126 |
| Exon 3 target-2 | 7% | Exon 3/28 | SEQ ID No: 127 |
| Exon 3 target-3 | 9% | Exon 3/28 | SEQ ID No: 128 |
| Exon 3 target-4 | 9% | Exon 3/28 | SEQ ID No: 129 |
| Exon 3 target-5 | 8% | Exon 3/28 | SEQ ID No: 130 |
| Exon 3 target-6 | 11% | Exon 3/28 | SEQ ID No: 131 |
| Exon 3 target-7 | 12% | Exon 3/28 | SEQ ID No: 132 |
| Exon 3 target-8 | 12% | Exon 3/28 | SEQ ID No: 133 |
| Exon 3 target-9 | 10% | Exon 3/28 | SEQ ID No: 134 |
| Exon 3 target-10 | 9% | Exon 3/28 | SEQ ID No: 135 |
| Exon 3 target-11 | 11% | Exon 3/28 | SEQ ID No: 136 |
| Exon 3 target-12 | 40% | Exon 3/28 | SEQ ID No: 137 |
| Exon 3 target-13 | 17% | Exon 3/28 | SEQ ID No: 138 |
| Exon 3 target-14 | 15% | Exon 3/28 | SEQ ID No: 139 |
| Exon 3 target-15 | 12% | Exon 3/28 | SEQ ID No: 140 |
| Exon 3 target-16 | 21% | Exon 3/28 | SEQ ID No: 141 |
| Exon 3 target-17 | 48% | Exon 3/28 | SEQ ID No: 142 |
| Exon 3 target-18 | 41% | Exon 3/28 | SEQ ID No: 143 |
| Exon 3 target-19 | 19% | Exon 3/28 | SEQ ID No: 144 |
| Exon 3 target-20 | 9% | Exon 3/28 | SEQ ID No: 145 |
| Exon 3 target-21 | 19% | Exon 3/28 | SEQ ID No: 146 |
| Exon 3 target-22 | 8% | Exon 3/28 | SEQ ID No: 147 |
| Exon 3 target-23 | 8% | Exon 3/28 | SEQ ID No: 148 |
| Exon 3 target-24 | 6% | Exon 3/28 | SEQ ID No: 149 |
| VEGFA target-1 | 6% | Exon 1/8 | SEQ ID No: 150 |
| VEGFA target-2 | 8% | Exon 1/8 | SEQ ID No: 151 |
| VEGFA target-3 | 8% | Exon 1/8 | SEQ ID No: 152 |
| VEGFA target-4 | 7% | Exon 1/8 | SEQ ID No: 153 |
| VEGFA target-5 | 8% | Exon 1/8 | SEQ ID No: 154 |
| VEGFA target-6 | 7% | Exon 1/8 | SEQ ID No: 155 |
| VEGPA target-7 | 6% | Exon 1/8 | SEQ ID No: 156 |
| VEGFA target-8 | 8% | Exon 1/8 | SEQ ID No: 157 |
| Nontargeting target-1 | 5% | Exon 1/28 | SEQ ID No: 158 |
| Nontargeting target-2 | 7% | Exon 1/28 | SEQ ID No: 159 |
| Nontargeting target-3 | 6% | Exon 1/28 | SEQ ID No: 160 |
| Nontargeting target-4 | 6% | Exon 1/28 | SEQ ID No: 161 |
| Nontargeting target-5 | 6% | Exon 1/28 | SEQ ID No: 162 |
| Nontargeting target-6 | 6% | Exon 1/28 | SEQ ID No: 163 |
| Nontargeting target-7 | 7% | Exon 1/28 | SEQ ID No: 164 |
| Nontargeting target-8 | 4% | Exon 1/28 | SEQ ID No: 165 |

The data presented in Table 16 shows that CasM did not produce egfr knockdown when targeted to (1) sequences upstream of the predicted exon 1 start site using SEQ ID NOS: 70-77; (2) an unrelated vascular endothelial growth factor A (vegfa) gene using SEQ ID NOS: 150-157); or (3) the reverse complement of sequences contained in egfr exon 1 mRNA using SEQ ID NOS: 158-165. Conversely, CasM enabled approximately 40% egfr knockdown when targeted to mRNA sequences contained in exon 2 using SEQ ID NOS: 102-125, and exon 3 of egfr using SEQ ID NOS: 126-149.

Although preferred embodiments of the subject methods have been described in some detail, it is understood that obvious variations can be made without departing from the spirit and the scope of the invention as defined by the appended claims.

1. An isolated CasM protein capable of producing a single-strand break at an RNA target site when guided to the RNA target site by a cognate nucleic acid guide.

2. (canceled)

3. The CasM protein of claim 1, wherein the protein comprises an amino acid sequence selected from the group consisting of the amino acid sequence of SEQ ID NOS: 37-44 or 45; an ortholog of the amino acid sequence of SEQ ID NOS: 37-44 and 45; and a variant of the amino acid sequence of SEQ ID NOS: 37-44 or 45.

4. The CasM protein of claim 3, wherein the protein comprises the amino acid sequence of SEQ ID NOS: 37-44 or 45.

5. A complex comprising:
a CasM protein according to claim 1; and
a cognate nucleic acid guide.

6. The complex of claim 5, wherein the cognate nucleic acid guide comprises RNA.

7. (canceled)

8. The complex of claim 5, wherein the cognate nucleic acid guide comprises a repeat sequence and a spacer sequence, wherein the repeat sequence and the spacer sequence do not naturally occur together.

9. (canceled)

10. An isolated polynucleotide encoding the CasM protein of claim 1.

11. The polynucleotide of claim 10, wherein the polynucleotide comprises the sequence of SEQ ID NOS: 28-35 or 36.

12. A modified polynucleotide encoding a CasM protein capable of producing a single strand break at a RNA target site when guided to the RNA target site by a cognate nucleic acid guide, wherein the polynucleotide is modified relative to its native sequence.

13.-15. (canceled)

16. The modified polynucleotide of claim 12, wherein the polynucleotide comprises the sequence of SEQ ID NOS: 1-8 or 9.

17. The modified polynucleotide of claim 12, wherein the polynucleotide is modified for expression in a eukaryotic cell.

18. (canceled)

19. (canceled)

20. The modified polynucleotide of claim 12, wherein the polynucleotide comprises the sequence of SEQ ID NOS: 10-17 or 18.

21. The modified polynucleotide of claim 12, wherein the polynucleotide comprises a sequence encoding a N- or C-terminal nuclear localization signal sequence (NLS).

22. The modified polynucleotide of claim 17, wherein the polynucleotide is modified for expression in a plant cell.

23. The modified polynucleotide of claim 22, wherein the plant cell is a Zea mays cell.

24. The modified polynucleotide of claim 23, wherein the polynucleotide comprises the sequence of SEQ ID NOS: 20-26 or 27.

25. A recombinant vector comprising:
a polynucleotide comprising a coding sequence for a CasM protein according to claim 1; and
at least one control element operably linked to said polynucleotide, whereby the CasM coding sequence in said polynucleotide is capable of being transcribed and translated in a host cell.

26. (canceled)

27. (canceled)

28. A host cell comprising the recombinant vector of claim 25.

29.-32. (canceled)

33. A method of directing a CasM protein to a selected nucleic acid target sequence, the method comprising:
contacting the selected nucleic acid target sequence with a complex according to claim 5, whereby the CasM protein is delivered to the nucleic acid target sequence.

34.-40. (canceled)
